(19) United States
(12) Patent Application Publication
(10) Pub. No.: US 2026/0338652 A1
(43) Pub. Date: Oct. 29, 2026

(54) METHODS FOR ANALYZING MIXED COMPOSITIONS OF PROTEIN AND PROTEIN CONJUGATES

(71) Applicant: KODIAK SCIENCES INC., Palo Alto, CA (US)

(72) Inventors: Wah Yuen To, San Mateo, CA (US); Carrie Jiali Su, San Mateo, CA (US)

(21) Appl. No.: 19/490,298

(22) PCT Filed: Jun. 6, 2024
(86) PCT No.: PCT/US2024/032771

§ 371 Date: Dec. 4, 2025

Related U.S. Application Data
(60) Provisional application No. 63/506,764, filed on Jun. 7, 2023.

(51) Int. Cl. G01N 1/40; B01D 15/12; B01D 15/18; B01D 15/34; B01D 15/36; G01N 30/02; G01N 30/14; G01N 30/46

(52) U.S. Cl. CPC G01N 1/405; B01D 15/125; B01D 15/1871; B01D 15/34; B01D 15/362; G01N 1/4077; G01N 30/14; G01N 30/46; G01N 2001/4088; G01N 2030/027

(57) ABSTRACT

Provided herein are methods and apparatuses for analyzing mixed formulations of proteins and protein conjugates thereof. Some embodiments of the protein can be conjugated to a moiety, such as a HEMA-PC polymer. Some embodiments of the antibody conjugates can retain or enhance antibody activity. The antibody and conjugate thereof can be particularly useful for treating diabetic retinopathy.

### INCORPORATION BY REFERENCE TO RELATED APPLICATIONS

This application claims the benefit of U.S. Provisional Application Ser. No. 63/506,764, filed Jun. 7, 2023, which is hereby incorporated by reference in its entirety.

### SEQUENCE LISTING

The present application is being filed along with a Sequence Listing in electronic format. The Sequence Listing is provided as a file entitled KDIAK211WO_SEQLIST.xml, created Jun. 4, 2024, which is 196,690 bytes in size. The information in the electronic format of the Sequence Listing is incorporated herein by reference in its entirety.

### FIELD

The present disclosure relates to methods and apparatuses for analyzing mixed formulations and compositions comprising a mixture of unconjugated and conjugated proteins (e.g., antibodies and conjugates thereof).

### BACKGROUND

There are a variety of systems available for people to purify proteins in the biopharmaceutical context. Often, the various systems have specific pros and cons.

### SUMMARY

Provided herein is a method of analyzing a mixed formulation sample, the method comprising: i. providing a mixed formulation that comprises: a. a first antibody that is an anti-VEGF-A antibody conjugated to a polymer; and b. a second antibody that is an anti-VEGF-A antibody that is not conjugated to the polymer; ii. a first run, wherein the mixed formulation is run through a prefiltration step to provide a first filtered mixed formulation, wherein the first filtered mixed formulation is run through a cation exchange chromatography (CEX) column to provide a second filtered mixed formulation; iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation; iv. a specific ionic strength of the solvent is used to allow partitioning of the mixed formulation in the CEX column; and wherein the method allows analysis of the mixed formulation based on the differential charge and size variant of the constituents.

Also provided is a method of analyzing a mixed therapeutic composition sample, the method comprising: i. providing a mixed therapeutic composition that comprises: a. a first antibody that is an anti-VEGF-A antibody conjugated to a polymer; and b. a second antibody that is an anti-VEGF-A antibody that is not conjugated to the polymer; ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; iii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition; iv. a specific ionic strength of the solvent is used to allow partitioning of the mixed therapeutic composition in the CEX column; and wherein the method allows analysis of the mixed therapeutic composition based on the differential charge and size variant of the constituents.

Also provided is a method of analyzing a mixed therapeutically effective formulation sample, the method comprising: i. providing a mixed therapeutically effective formulation that comprises: a. a first antibody that is an anti-VEGF-A antibody conjugated to a polymer; and b. a second antibody that is an anti-VEGF-A antibody that is not conjugated to the polymer; ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation; iv. a specific ionic strength of the solvent is used to allow partitioning of the mixed therapeutically effective formulation in the CEX column; and wherein the method allows analysis of the mixed therapeutically effective formulation based on the differential charge and size variant of the constituents. Also provided is an apparatus comprising a tandem series of CEX and SEC HPLC columns for use in analyzing a mixed formulation sample comprising a) a first antibody that is an anti-VEGF-A antibody conjugated to a polymer and b) a second antibody that is an anti-VEGF-A antibody that is not conjugated to the polymer, wherein the purification comprises; i. a first run, wherein the mixed formulation is run through a prefiltration step to provide a first filtered mixed formulation, wherein the first filtered mixed formulation is run through a cation exchange chromatography (CEX) column to provide a second filtered mixed formulation; and ii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation. Further provided herein is an apparatus comprising a tandem series of CEX and SEC HPLC columns for use in analyzing a mixed therapeutic composition sample comprising a) a first antibody that is an anti-VEGF-A antibody conjugated to a polymer and b) a second antibody that is an anti-VEGF-A antibody that is not conjugated to the polymer, wherein the purification comprises; i. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; and ii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition. Further provided herein is an apparatus comprising a tandem series of CEX and SEC HPLC columns for use in analyzing a mixed therapeutically effective formulation sample comprising a) a first antibody that is an anti-VEGF-A antibody conjugated to a polymer and b) a second antibody that is an anti-VEGF-A antibody that is not conjugated to the polymer, wherein the purification comprises; i. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; and ii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation. Provided herein is a method of analyzing a mixed formulation sample, the method comprising: i. providing a mixed formulation that comprises: a. a first antibody that is an anti-VEGF-A antibody conjugated to a polymer; and b. a second antibody that is an anti-VEGF-A antibody that is not conjugated to the polymer; ii. a first run, wherein the mixed formulation is run through a prefiltration step to provide a first filtered mixed formulation, wherein the first filtered mixed formulation is run through a cation exchange chromatography (CEX) column to provide a second filtered mixed formulation; and iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation; and wherein the method reduces the presence of antibody aggregates in the formulation; and wherein the polymer of the first antibody comprises a phosphorylcholine containing polymer, wherein the polymer is covalently bonded to the first antibody at a cysteine outside a variable region of the first antibody, and wherein said cysteine replaces a non-cysteine amino acid that occurs in a same position in a sequence, wherein the first antibody comprises a light chain and heavy chain, said heavy chain comprising an Fc region, wherein the cysteine is in the Fc region of the heavy chain, wherein the sequence of the heavy chain comprises SEQ ID NO: 1 (with or without the C-terminal lysine), and wherein the sequence of the light chain comprises SEQ ID NO: 2; wherein the antibody conjugate has the following structure:

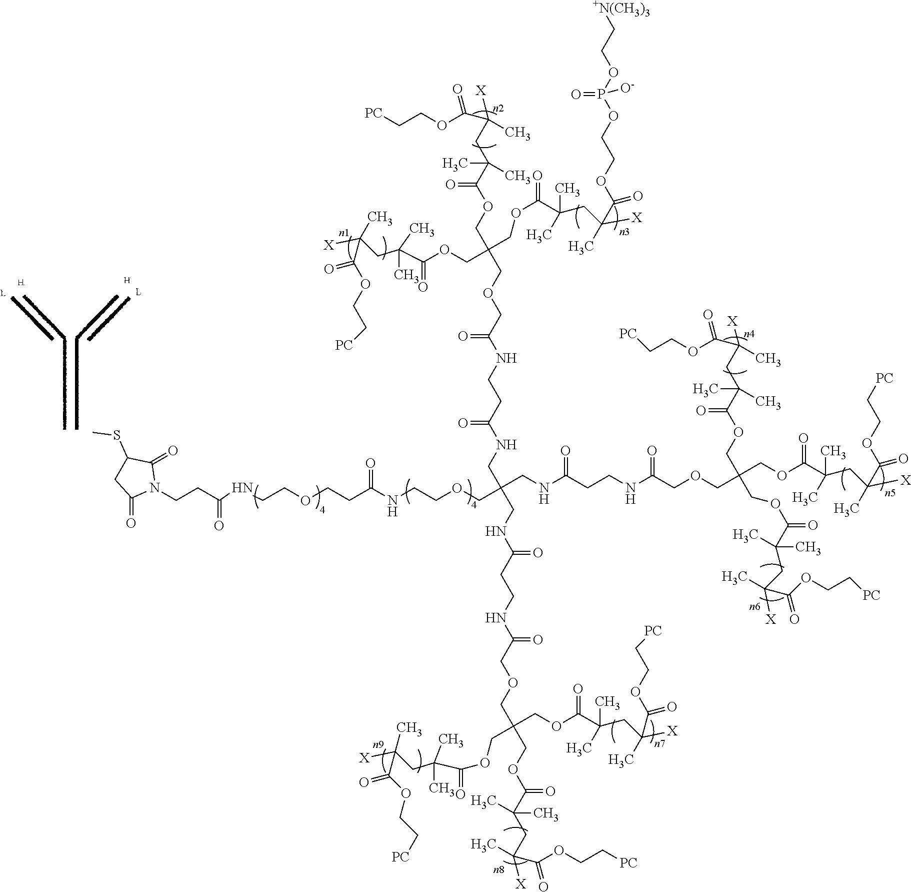

where X is a) —OR where R is —H, Methyl, ethyl, propyl, or isopropyl, b) —H, or c) a halide; (or optionally where X is a) —OR where R is —H, Methyl, ethyl, propyl, or isopropyl, b) —H, c) any halogen, including —Br, —Cl, or —I, d) —SCN, or e) —NCS;) wherein: each heavy chain of the first antibody is denoted by the letter H, and each light chain of the first antibody is denoted by the letter L; the polymer is bonded to the first antibody through the sulfhydryl of a cysteine at position 449, as numbered in SEQ ID NO: 1, which bond is depicted on one of the heavy chains; PC is

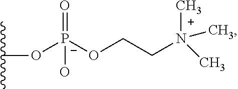

where the curvy line indicates the point of attachment to the rest of the polymer; and n1, n2, n3, n4, n5, n6, n7, n8 and n9 are the same or different such that the sum of n1, n2, n3, n4, n5, n6, n7, n8 and n9 is 2500 plus or minus 15%; and wherein, the second antibody comprises a light chain and heavy chain, said heavy chain comprising an Fc region, wherein a cysteine is in the Fc region of the heavy chain, wherein the sequence of the heavy chain comprises SEQ ID NO: 1, and wherein the sequence of the light chain comprises SEQ ID NO: 2; and wherein the mixed formulation sample travels sequentially through a prefilter, the CEX column, and the SEC HPLC column, wherein the tandem CEX and SEC HPLC columns are a Shodex SP825 column with internal diameter (i.d.)×length dimensions of 9.0×75 mm and a TSKgel G3000SWxl column with i.d.×length dimensions of 7.8×300 mm column arrangement; wherein the isocratic running conditions comprise a flow rate of 0.5 ml/min, and the buffer comprises 20 mM sodium acetate, pH 5 any amount between 50 mM NaCl and 5M NaCl; wherein the percentage of the second antibody is any percentage between 0% and 20%; wherein the mixed formulation sample comprises any amount between 25 ug and 1340 ug of protein; wherein the method further comprises assessment for high molecular weight aggregates within the purified mixed formulation by methods comprising SEC profile analysis and SDS PAGE gel; and wherein the method further comprises assessment of the percentage of the second antibody in the purified mixed formulation by SEC profile analysis; wherein the first run separates the first antibody from the second antibody and antibody aggregates, wherein the step for elution of CEX bound free protein during the second run comprises a pulse of high salt at 1M NaCl, wherein during the second run, the fourth filtered mixed formulation is run through a prefiltration step to provide a fifth filtered mixed formulation, wherein the fifth filtered mixed formulation is run through a CEX column to provide a six filtered mixed formulation, wherein the sixth mixed formulation is run through a SEC HPLC column; and wherein running the filtered mixed sample through a size exclusion exchange SEC HPLC column separates the second antibody from antibody aggregates.

Also provided is a method of analyzing a mixed formulation sample, the method comprising: i. providing a mixed formulation that comprises: a. a fusion protein conjugated to a polymer; and b. a second protein that is not conjugated to the polymer; ii. a first run, wherein the mixed formulation is run through a prefiltration step to provide a first filtered mixed formulation, wherein the first filtered mixed formulation is run through a cation exchange chromatography (CEX) column to provide a second filtered mixed formulation; iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation; a specific ionic strength of the solvent is used to allow partitioning of the mixed formulation in the CEX column; and wherein the method allows analysis of the mixed formulation based on the differential charge and size variant of the constituents. Also provided herein is a method of analyzing a mixed therapeutic composition sample, the method comprising: i. providing a mixed therapeutic composition that comprises: a. a fusion protein conjugated to a polymer; and b. a second protein that is not conjugated to the polymer; ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; iii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition; iv. a specific ionic strength of the solvent is used to allow partitioning of the mixed therapeutic composition in the CEX column; and wherein the method allows analysis of the mixed therapeutic composition based on the differential charge and size variant of the constituents. Further provided herein is a method of analyzing a mixed therapeutically effective formulation sample, the method comprising: i. providing a mixed therapeutically effective formulation that comprises: a. a fusion protein conjugated to a polymer; and b. a second protein that is not conjugated to the polymer; ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation; iv. a specific ionic strength of the solvent is used to allow partitioning of the mixed therapeutically effective formulation in the CEX column; and wherein the method allows analysis of the mixed therapeutically effective formulation based on the differential charge and size variant of the constituents.

Also provided is an apparatus comprising a tandem series of CEX and SEC HPLC columns for use in purifying a mixed formulation sample comprising a) a first fusion protein conjugated to a polymer and b) a second protein that is not conjugated to the polymer, wherein the purification comprises; i. a first run, wherein the mixed formulation is run through a prefiltration step to provide a first filtered mixed formulation, wherein the first filtered mixed formulation is run through a cation exchange chromatography (CEX) column to provide a second filtered mixed formulation; and ii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation. Provided herein is an apparatus comprising a tandem series of CEX and SEC HPLC columns for use in purifying a mixed therapeutic composition sample comprising a) a first fusion protein conjugated to a polymer and b) a second protein that is not conjugated to the polymer, wherein the purification comprises; i. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; and ii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition. Also provided is an apparatus comprising a tandem series of CEX and SEC HPLC columns for use in purifying a mixed therapeutically effective formulation sample comprising a) a first fusion protein conjugated to a polymer and b) a second protein that is not conjugated to the polymer, wherein the purification comprises; i. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; and ii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation.

Provided herein is a method of analyzing a mixed formulation sample, the method comprising: i. providing a mixed formulation that comprises: a. a first antibody that is an anti-VEGF-A antibody conjugated to a polymer; and b. a second antibody that is an anti-VEGF-A antibody that is not conjugated to the polymer; ii. a first run, wherein the mixed formulation is run through a prefiltration step to provide a first filtered mixed formulation, wherein the first filtered mixed formulation is run through a cation exchange chromatography (CEX) column to provide a second filtered mixed formulation; iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation; iv. a specific ionic strength of the solvent is used to allow partitioning of the mixed formulation in the CEX column; and wherein the method allows analysis of the mixed formulation based on the differential charge and size variant of the constituents. Also provided is a method of analyzing a mixed therapeutic composition sample, the method comprising: i. providing a mixed therapeutic composition that comprises: a. a first antibody that is an anti-VEGF-A antibody conjugated to a polymer; and b. a second antibody that is an anti-VEGF-A antibody that is not conjugated to the polymer; ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; iii.a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition; iv. a specific ionic strength of the solvent is used to allow partitioning of the mixed therapeutic composition in the CEX column; and wherein the method allows analysis of the mixed therapeutic composition based on the differential charge and size variant of the constituents. Further provided is a method of analyzing a mixed therapeutically effective formulation sample, the method comprising: i. providing a mixed therapeutically effective formulation that comprises: a. a first antibody that is an anti-VEGF-A antibody conjugated to a polymer; and b. a second antibody that is an anti-VEGF-A antibody that is not conjugated to the polymer; ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation; iv. a specific ionic strength of the solvent is used to allow partitioning of the mixed therapeutically effective formulation in the CEX column; and wherein the method allows analysis of the mixed therapeutically effective formulation based on the differential charge and size variant of the constituents.

Provided herein is a method of analyzing a mixed formulation sample, the method comprising: i. providing a mixed formulation that comprises: a. a first antibody that is an anti-VEGF-A antibody conjugated to a polymer; and b. a second antibody that is an anti-VEGF-A antibody that is not conjugated to the polymer; ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column; iii. a first run, wherein the mixed formulation is injected and run through the system of ii. iv. a second run, wherein a concentrated salt is injected to elute the bound fraction of the CEX column to be separated by the SEC column; wherein, a specific ionic strength of the solvent is used to allow partitioning of the mixed formulation in the CEX column; and wherein the method allows analysis of the mixed formulation based on the differential charge and size variant of the constituents. Also provided is a method of analyzing a mixed therapeutic composition sample, the method comprising: i. providing a mixed therapeutic composition that comprises: a. a first antibody that is an anti-VEGF-A antibody conjugated to a polymer; and b. a second antibody that is an anti-VEGF-A antibody that is not conjugated to the polymer; ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column; iii. a first run, wherein the mixed therapeutic composition is injected and run through the system of ii. iv. a second run, wherein a concentrated salt is injected to elute the bound fraction of the CEX column to be separated by the SEC column; wherein, a specific ionic strength of the solvent is used to allow partitioning of the mixed therapeutic composition in the CEX column; and wherein the method allows analysis of the mixed therapeutic composition based on the differential charge and size variant of the constituents. Further provided is a method of analyzing a mixed therapeutically effective formulation sample, the method comprising: i. providing a mixed therapeutically effective formulation that comprises: a. a first antibody that is an anti-VEGF-A antibody conjugated to a polymer; and b. a second antibody that is an anti-VEGF-A antibody that is not conjugated to the polymer; ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column; iii. a first run, wherein the mixed therapeutically effective formulation is injected and run through the system of ii. iv. a second run, wherein a concentrated salt is injected to elute the bound fraction of the CEX column to be separated by the SEC column; wherein, a specific ionic strength of the solvent is used to allow partitioning of the mixed therapeutically effective formulation in the CEX column; and wherein the method allows analysis of the mixed therapeutically effective formulation based on the differential charge and size variant of the constituents.

Provided herein is a method of analyzing a mixed formulation sample, the method comprising: i. providing a mixed formulation that comprises a combination of two protein moieties, wherein the first protein moiety (A) is conjugated to a polymer, and the second protein (B) moiety is not conjugated to a polymer; ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column; iii. a first run, wherein the mixed formulation is injected and run through the system of ii. iv. a second run, wherein a concentrated salt is injected to elute the bound fraction of the CEX column to be separated by the SEC column; wherein, a specific ionic strength of the solvent is used to allow partitioning of the mixed formulation in the CEX column; and wherein the method allows analysis of the mixed formulation based on the differential charge and size variant of the constituents. Also provided is a method of analyzing a mixed therapeutic composition sample, the method comprising: i. providing a mixed therapeutic composition that comprises a combination of two protein moieties, wherein the first protein moiety (A) is conjugated to a polymer, and the second protein (B) moiety is not conjugated to a polymer; ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column; iii. a first run, wherein the mixed therapeutic composition is injected and run through the system of ii. iv. a second run, wherein a concentrated salt is injected to elute the bound fraction of the CEX column to be separated by the SEC column; wherein, a specific ionic strength of the solvent is used to allow partitioning of the mixed therapeutic composition in the CEX column; and wherein the method allows analysis of the mixed therapeutic composition based on the differential charge and size variant of the constituents. Further provided is a method of analyzing a mixed therapeutically effective formulation sample, the method comprising: i. providing a mixed therapeutically effective formulation that comprises a combination of two protein moieties, wherein the first protein moiety (A) is conjugated to a polymer, and the second protein (B) moiety is not conjugated to a polymer; ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column; iii. a first run, wherein the mixed therapeutically effective formulation is injected and run through the system of ii. iv. a second run, wherein a concentrated salt is injected to elute the bound fraction of the CEX column to be separated by the SEC column; v, wherein, a specific ionic strength of the solvent is used to allow partitioning of the mixed therapeutically effective formulation in the CEX column; and wherein the method allows analysis of the mixed therapeutically effective formulation based on the differential charge and size variant of the constituents.

Also provided is a method of analyzing a mixed formulation sample, the method comprising: i. providing a mixed formulation comprising a combination of two protein moieties, wherein the first protein moiety (A) is conjugated to a polymer, and the second protein moiety (B) is not conjugated to a polymer; ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein the method allows analysis of the mixed formulation based on the differential charge and size variant of the constituents. Provided herein is a method of analyzing a mixed therapeutic composition sample, the method comprising: i. providing a mixed therapeutic composition comprising a combination of two protein moieties, wherein the first protein moiety (A) is conjugated to a polymer, and the second protein moiety (B) is not conjugated to a polymer; ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein the method allows analysis of the mixed therapeutic composition based on the differential charge and size variant of the constituents. Further provided is a method of analyzing a mixed therapeutically effective formulation sample, the method comprising: i. providing a mixed therapeutically effective formulation comprising a combination of two protein moieties, wherein the first protein moiety (A) is conjugated to a polymer, and the second protein moiety (B) is not conjugated to a polymer; ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein the method allows analysis of the mixed therapeutically effective formulation based on the differential charge and size variant of the constituents.

Provided herein is an apparatus comprising a tandem HPLC system for use in analyzing a mixed formulation sample comprising a combination of two protein moieties, wherein the first protein moiety (A) is conjugated to a polymer, and the second protein moiety (B) is not conjugated to a polymer, wherein the apparatus comprises: a) a HPLC pump connected to an auto-injector; b) a prefilter; c) cation-exchanger column (CEX); d) a switch valve enabling the effluent of the CEX to be directed to multiple possible targets, wherein the apparatus allows analysis of the mixed formulation based on the differential charge and size variant of the constituents. Also provided is an apparatus comprising a tandem HPLC system for use in analyzing a mixed therapeutic composition sample comprising a combination of two protein moieties, wherein the first protein moiety (A) is conjugated to a polymer, and the second protein moiety (B) is not conjugated to a polymer, wherein the apparatus comprises: a) a HPLC pump connected to an auto-injector; b) a prefilter; c) cation-exchanger column (CEX); d) a switch valve enabling the effluent of the CEX to be directed to multiple possible targets, wherein the apparatus allows analysis of the mixed therapeutic composition based on the differential charge and size variant of the constituents. Further provided is an apparatus comprising a tandem HPLC system for use in analyzing a mixed therapeutically effective formulation sample comprising a combination of two protein moieties, wherein the first protein moiety (A) is conjugated to a polymer, and the second protein moiety (B) is not conjugated to a polymer, wherein the apparatus comprises: a) a HPLC pump connected to an auto-injector; b) a prefilter; c) cation-exchanger column (CEX); d) a switch valve enabling the effluent of the CEX to be directed to multiple possible targets, wherein the apparatus allows analysis of the mixed therapeutically effective formulation based on the differential charge and size variant of the constituents.

Provided herein is a method of analyzing a mixed formulation sample, the method comprising: i. providing a mixed formulation comprising a combination of two protein moieties, wherein the first protein moiety (A) is conjugated to a polymer, and the second protein moiety (B) is not conjugated to a polymer; and ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein an additional pump is used to pulse elute the CEX-bound fraction upon wherein the method allows analysis of the mixed formulation based on the differential charge and size variant of the constituents; and wherein the sample is analyzed via a single continuous chromatography run. Also provided is a method of analyzing a mixed therapeutic composition sample, the method comprising: i. providing a mixed therapeutic composition comprising a combination of two protein moieties, wherein the first protein moiety (A) is conjugated to a polymer, and the second protein moiety (B) is not conjugated to a polymer; and ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein an additional pump is used to pulse elute the CEX-bound fraction upon wherein the method allows analysis of the mixed therapeutic composition based on the differential charge and size variant of the constituents; and wherein the sample is analyzed via a single continuous chromatography run. Provided herein is a method of analyzing a mixed therapeutically effective formulation sample, the method comprising: i. providing a mixed therapeutically effective formulation comprising a combination of two protein moieties, wherein the first protein moiety (A) is conjugated to a polymer, and the second protein moiety (B) is not conjugated to a polymer; and ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein an additional pump is used to pulse elute the CEX-bound fraction upon wherein the method allows analysis of the mixed therapeutically effective formulation based on the differential charge and size variant of the constituents; and wherein the sample is analyzed via a single continuous chromatography run.

Also provided is an apparatus comprising a tandem HPLC system for use in purifying a mixed formulation sample comprising a combination of two protein moieties, wherein the first protein moiety (A) is conjugated to a polymer, and the second protein moiety (B) is not conjugated to a polymer, wherein the apparatus comprises: a) a HPLC pump connected to an auto-injector; b) a prefilter; c) cation-exchanger column (CEX); d) a switch valve enabling the effluent of the CEX to be directed to multiple possible targets, wherein the apparatus allows analysis of the mixed formulation based on the differential charge and size variant of the constituents; wherein the sample is analyzed via a single continuous chromatography run. Provided herein is an apparatus comprising a tandem HPLC system for use in purifying a mixed therapeutic composition sample comprising a combination of two protein moieties, wherein the first protein moiety (A) is conjugated to a polymer, and the second protein moiety (B) is not conjugated to a polymer, wherein the apparatus comprises: a) a HPLC pump connected to an auto-injector; b) a prefilter; c) cation-exchanger column (CEX); d) a switch valve enabling the effluent of the CEX to be directed to multiple possible targets, wherein the apparatus allows analysis of the mixed therapeutic composition based on the differential charge and size variant of the constituents; wherein the sample is analyzed via a single continuous chromatography run. Also provided is an apparatus comprising a tandem HPLC system for use in purifying a mixed therapeutically effective formulation sample comprising a combination of two protein moieties, wherein the first protein moiety (A) is conjugated to a polymer, and the second protein moiety (B) is not conjugated to a polymer, wherein the apparatus comprises: a) a HPLC pump connected to an auto-injector; b) a prefilter; c) cation-exchanger column (CEX); d) a switch valve enabling the effluent of the CEX to be directed to multiple possible targets, wherein the apparatus allows analysis of the mixed therapeutically effective formulation based on the differential charge and size variant of the constituents; wherein the sample is analyzed via a single continuous chromatography run.

Also provided herein is a method of analyzing a mixed formulation sample, the method comprising: i. providing a mixed formulation comprising a combination of two protein moieties, wherein the first protein moiety (A) is conjugated to a polymer, and the second protein moiety (B) is not conjugated to a polymer; and ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein the switch valve can direct the CEX effluent to: 1) a bypass loop; 2) a first SEC column; or 3) a second SEC column; and wherein the method allows analysis of the mixed formulation based on the differential charge and size variant of the constituents, wherein the sample is analyzed via a single continuous chromatography run, and wherein the conjugated sample is directed to the bypass loop and the unconjugated sample is directed to the first or second SEC column. Also provided is a method of analyzing a mixed therapeutic composition sample, the method comprising: i. providing a mixed therapeutic composition comprising a combination of two protein moieties, wherein the first protein moiety (A) is conjugated to a polymer, and the second protein moiety (B) is not conjugated to a polymer; and ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein the switch valve can direct the CEX effluent to: 1) a bypass loop; 2) a first SEC column; or 3) a second SEC column; and wherein the method allows analysis of the mixed therapeutic composition based on the differential charge and size variant of the constituents, wherein the sample is analyzed via a single continuous chromatography run, and wherein the conjugated sample is directed to the bypass loop and the unconjugated sample is directed to the first or second SEC column. Further provided is a method of analyzing a mixed therapeutically effective formulation sample, the method comprising: i. providing a mixed therapeutically effective formulation comprising a combination of two protein moieties, wherein the first protein moiety (A) is conjugated to a polymer, and the second protein moiety (B) is not conjugated to a polymer; and ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets; wherein the switch valve can direct the CEX effluent to: 1) a bypass loop; 2) a first SEC column; or 3) a second SEC column; and wherein the method allows analysis of the mixed therapeutically effective formulation based on the differential charge and size variant of the constituents, wherein the sample is analyzed via a single continuous chromatography run, and wherein the conjugated sample is directed to the bypass loop and the unconjugated sample is directed to the first or second SEC column.

Provided herein is an apparatus comprising a tandem series of HPLC system for use in analyzing a mixed formulation sample comprising (A) a first protein conjugated to a polymer and (B) a second protein that is not conjugated to the polymer, wherein, the HPLC system comprises: a) a HPLC pump connected to an auto-injector; b) a prefilter; c) cation-exchanger column (CEX); d) a switch valve enabling the effluent of the CEX to be directed to multiple possible targets, wherein the switch valve can direct the CEX effluent to: 1) a bypass loop; 2) a first SEC column; or 3) a second SEC column; and wherein the apparatus allows analysis of the mixed formulation based on the differential charge and size variant of the constituents, wherein the sample is analyzed via a single continuous chromatography run; and wherein the conjugated sample is directed to the bypass loop and the unconjugated sample is directed to the first or second SEC column. Also provided is an apparatus comprising a tandem series of HPLC system for use in analyzing a mixed therapeutic composition sample comprising (A) a first protein conjugated to a polymer and (B) a second protein that is not conjugated to the polymer, wherein, the HPLC system comprises: a) a HPLC pump connected to an auto-injector; b) a prefilter; c) cation-exchanger column (CEX); d) a switch valve enabling the effluent of the CEX to be directed to multiple possible targets, wherein the switch valve can direct the CEX effluent to: 1) a bypass loop; 2) a first SEC column; or 3) a second SEC column; and wherein the apparatus allows analysis of the mixed therapeutic composition based on the differential charge and size variant of the constituents, wherein the sample is analyzed via a single continuous chromatography run; and wherein the conjugated sample is directed to the bypass loop and the unconjugated sample is directed to the first or second SEC column. Further provided is an apparatus comprising a tandem series of HPLC system for use in analyzing a mixed therapeutically effective formulation sample comprising (A) a first protein conjugated to a polymer and (B) a second protein that is not conjugated to the polymer, wherein, the HPLC system comprises: a) a HPLC pump connected to an auto-injector; b) a prefilter; c) cation-exchanger column (CEX); d) a switch valve enabling the effluent of the CEX to be directed to multiple possible targets, wherein the switch valve can direct the CEX effluent to: 1) a bypass loop; 2) a first SEC column; or 3) a second SEC column; and wherein the apparatus allows analysis of the mixed therapeutically effective formulation based on the differential charge and size variant of the constituents, wherein the sample is analyzed via a single continuous chromatography run; and wherein the conjugated sample is directed to the bypass loop and the unconjugated sample is directed to the first or second SEC column.

### BRIEF DESCRIPTION OF THE DRAWINGS

FIG. 1 depicts a schematic of mixed sample preparation and run 1 and run 2 of the tandem HPLC system.

FIGS. 2A-2B depict schematics of options for the HPLC and column systems. FIG. 2A depicts an arrangement including an IEX column and a single SEC column, whereas FIG. 2B depicts a schematic including an IEX with a switch valve downstream that allows for selection of one of two different SEC columns with 1 SEC column for run 1 and 1 SEC column for run 2.

FIG. 3 depicts the operation of the tandem HPLC column system in run 1 and run 2.

FIGS. 4A-4C depict SEC profiles of the CEX-SEC tandem series analysis for loading known amounts of purified OG1953 (conjugated) and OG1950 (unconjugated) separately, where OG1953 and OG1950 were not combined in a preformulated mixture. FIG. 4A depicts SEC profiles in stack plots for samples 1-6. FIG. 4B depicts SEC profile data organized into overlay plots, and FIG. 4C depicts a summary of the sample information.

FIGS. 5A-5C depicts SEC profiles of the CEX-SEC tandem series analysis of OG1953+7.5% OG1950 (OG1953_R50037) where OG1953 and OG1950 were combined in a preformulated mixture. FIG. 5A depicts SEC profiles in stack plots for samples 1-6. FIG. 5B depicts SEC profile data organized into overlay plots, and FIG. 5C depicts a summary of the sample information.

FIGS. 6A-6C depict SEC profiles showing identification of OG1950 peak and quantitative recovery of OG1950 from R50037 (7.5%). FIG. 6A depicts SEC profiles in stack plots for samples 1-5. FIG. 6B depicts SEC profile data organized into overlay plots, and FIG. 6C depicts a summary of the sample information.

FIGS. 7A-7B depict evaluation of the SEC calibration standard using 2D method (QC9661, QC9729B, & QC9742). FIG. 7A depicts SEC profiles in stack plots for samples 1-5. FIG. 7B depicts SEC profile data organized into overlay plots.

FIG. 8A-8C depicts SEC profiles of an analysis of the impact of running buffer with different NaCl concentration on OG1953 and OG1950 partitioning (R50037 or 7.5% OG1950) in the Tandem series of CEX and SEC column.

FIGS. 9A-9C depict SEC profiles showing identification of a potential histidine formulation peak in R50037 sample using the 2D method with 214 nm detection. FIG. 9A depicts SEC profiles in stack plots for samples 1-5. FIG. 9B depicts SEC profile data organized into overlay plots, and FIG. 9C depicts a summary of the sample information.

FIGS. 10A-10B depict SEC profiles showing carry over evaluation after the system was stripped with various buffers and the residual bound material was assessed.

FIGS. 11A-11E depict a summary of the sensitivity limit of detection (LOD), linearity and recovery data of from run 1 and run 2 of sets of OG1950 and R50037 samples with varying protein amounts loaded. FIG. 11A-B depict SEC profiles for OG1950PUR samples where 25-250 ug of protein were injected. FIG. 11C depicts SEC profiles for R50035 (7.5%) samples where 100-1340 ug of protein was injected. FIG. 11D depicts details for the samples in FIGS. 11A-B and graphs plotting OG1950 monomer or aggregate peak against load. FIG. 11E depicts details for the samples in FIG. 11C and additional SEC profiles for the samples in run 2.

FIG. 12A (Top left) #1 mixture was first cloudy but becomes cleared upon addition of 20 μl buffer. (Top right) Mild precipitation was observed upon microfuge centrifugation at 5K rpm for 5 min. (Bottom) All other formulation setups (#2-#5) show no cloudiness at all. Buffer B143-1 is 50 mM Sodium Acetate pH 5, 0.025% PS20.

FIG. 12B depicts 2D method analysis showing elution peak(s) of free protein at the expected retention time of 21.8 min, whereas the conjugate is eluted as a broad peak at ~15.6 min. This was confirmed by (a) SEC calibration standard; (b) 15% mix formulation standard (R50031); and (c) injection OG1950 free protein standard injection. The percentage on the right of each chromatogram represents the partition of CEX-unbound (conjugate) to CEX-bound fraction (free IgG), they are in agreement with the expected percent free protein as shown in FIG. 12D.

FIG. 12C (Top) depicts overlay of all curves from FIG. 12B to show overlap of eluted IgG peaks which were aligned with both the standard IgG and IgG standard from the SEC calibration standard. (Bottom left) Offset overlay of various injections for clear illustration of successful separation between unbound CEX conjugate fraction and the CEX bound IgG fraction. (Bottom right) is a close-up view to show the aggregated form of IgG that is clearly detected as shown in the arrows.

FIG. 12D depicts a peak area comparison of CEX bound (free protein) to unbound fraction (conjugate) showing agreement for the detected values in (X) as compared to the expected values in (Y). X shows the detected values from the Tandem method peak analysis, where the values can be calculated either using the detected peak area or the peak area can be converted to protein mass with Beer's law. Converting the detected peak area into protein mass allows for calculating the injection recovery, which can be compared to the Y values. The Y values are the input based on protein mass during the formulation preparation. Comparison of column X and Y determines recovery.

FIG. 12E depicts method analysis showing elution peak(s) of free protein at the expected retention time of about 25.3 min whereas the conjugate eluted at 15-15.2 min as a broad peak. This was confirmed by (a) OG468 Fab standard injection; and (b) SEC calibration standard.

FIG. 12F (Top) depicts overlay of all curves from FIG. 12E showing overlap of eluted OG468 Fab peaks which are aligned with the standard OG468 standard that was eluted later than the IgG standard though elutes later than the 44 kDa standard of the SEC calibration standard. (Bottom) Offset overlay of various injections for clear illustration of separation between unbound CEX conjugate fraction and the CEX bound Fab fraction.

FIG. 12G depicts peak area comparison of CEX bound (free protein) to unbound fraction (conjugate) showing agreement to the expected ratios, see column (X) as compared to column (Y). X and Y can be calculated and compared as described in the description of FIG. 12D above. For sample #5, there was only 6M GuHCl elution as OG468 Fab was found to bind tightly to CEX column and unable to be eluted with 1M NaCl and only recovered by 6M GuHCl elution; which was later repeated for #3&4 with 2M NaCl as elution buffer but was only about 60% and 75% eluted for samples #3 and #4, respectively. However, the accumulated total eluted CEX bound fraction was in agreement with the expected number as shown in column (Y).

FIG. 13A depicts an HPLC system configuration with a multiple column combination comprising a 3-way valve, where an additional optional switch valve may be present before or after the IEX column.

FIG. 13B depicts additional details regarding the configuration of the 3-way valve where the effluent of the IEX column can be directed to 1) a bypass loop; 2) a Shodex-806M SEC column; or 3) a TSK G3000 SEC column.

FIG. 13C depicts additional detail regarding the flow path options for each of 1-3 of FIG. 13B.

FIG. 14A depicts a summary of the sample information for the samples used to test HPLC system configuration with a multiple column combination described in FIG. 14. Samples include SEC calibration standards, OG1950 antibody, and two representative OG1953 drug product formulation prototypes, one with 20% OG1950+80% OG1953, and the other with 15% OG1950+75% OG1953.

FIG. 14B depicts the column flow path matrix for samples described in FIG. 14A, where each unique column combination is designated with a different SOP391 version (v1-v6).

FIGS. 15A-15C depict analysis of the R50032 formulation (20% OG1950+80% OG1953) via the SOP391_v1 featuring the tandem CEX+SEC HPLC method without the 3-way switch valve. FIG. 15A depicts stack plots at 280 nm for the blank, OG1950 standard, and CEX bound and unbound fractions. FIG. 15B depicts an overlay plot of the peaks shown in FIG. 15A. FIG. 15C depicts mass recovery from the CEX-unbound fraction and mass recovery from the individual peaks of the CEX-bound fraction, as well as total recovery.

FIGS. 16A-16F depict analysis of the R50032 and R50037 (7.5% OG1950+92.5% OG1953) formulations using the SOP391_v2 operation mode using the 3-way switch valve. FIG. 16A depicts the flow path through the 3-way valve in SOP391_v2 where the CEX-bound (E) and CEX-unbound (L) fractions pass through the TSK G3000 SEC column. FIG. 16B depicts the individual peaks observed in the E and L fractions. FIGS. 16C and 16D depict overlay graphs of the individual peaks shown in FIG. 16B. The arrows in FIG. 16C-16D indicate a peak at 16.966 minutes which accounts for 5.2% of the total CEX-bound fraction. FIG. 16E depicts a summary of the injection queue including run time (minutes), injection volume (μl), and acquisition method for the E and L fractions of the R50032 and R50037 formulations. FIG. 16F depicts a summary of the peak data including retention time (minutes) area, % area, height for the E and L fractions of the R50032 and R50037 formulations.

FIGS. 17A-17D depict analysis of the R50032 formulations using the SOP391_v3 operation mode using the 3-way switch valve, where the CEX-unbound fraction (L) was directed via bypass instead of flow through SEC column. FIG. 17A depicts the flow path through the 3-way valve in SOP391_v3 where the CEX-unbound (L) fraction is directed through the bypass and the CEX-bound (E) fraction is analyzed using the TSK G3000 SEC column. FIG. 17B depicts the individual peaks observed in the E and L fractions and compares the peaks of the E and L fractions between the SOP391_v2 and SOP391_v3 operation modes. FIG. 17C depicts overlay graphs of the individual peaks shown in FIG. 17B. FIG. 17D depicts a summary of the injection queue including run time (minutes), injection volume (μl), and acquisition method for the E and L fractions of the R50032 formulations.

FIGS. 18A-18D depict analysis of the R50032 formulations using the SOP391_v4 operation mode using the 3-way switch valve. FIG. 18A depicts the flow path through the 3-way valve in SOP391_v4 where the CEX-unbound fraction (L) passes through a Shodex 806M_HQ column and the CEX-bound (E) fraction is analyzed using a TSK G3000 column. FIG. 18B depicts the individual peaks observed in the E and L fractions. FIG. 18C depicts overlay graphs of the individual peaks shown in FIG. 18B. FIG. 18D depicts a summary of the injection queue including run time (minutes), injection volume (μl), and acquisition method for the E and L fractions of the R0032 formulations.

FIGS. 19A-19B depict evaluation of the overloading issue seen in R50032 using the SOP391_v4 operation mode. FIG. 19A depicts individual peaks seen in X: the tandem HPLC method using a CEX column and Shodex 806M_HQ SEC column where the SEC column was overloaded; and Y: where the R50032 formulation was analyzed on the Shodex 806M_HQ SEC column without CEX column operation. FIG. 19B depict an overlay graph of the individual peaks shown in FIG. 19A.

FIGS. 20A-20C depict analysis of the R50032 formulations using the SOP391_v5 and SOP391_v6 operation modes using the 3-way switch valve. FIG. 20A depicts the flow path through the 3-way valve in SOP391_v5 where the CEX-unbound fraction (L) passes through the bypass loop and the CEX-bound (E) fraction is analyzed by a Shodex 806M_HQ column. FIG. 20A also depicts the flow path through the 3-way valve in SOP391_v6 where both the CEX-unbound fraction (L) and the CEX-bound (E) fraction are analyzed using a Shodex 806M_HQ column. FIG. 20B depicts the individual peaks observed in the E and L fractions and compares the peaks of the E and L fractions between the SOP391_v5 and SOP391_v6 operation modes. FIG. 20C depicts overlay graphs of the individual peaks shown in FIG. 20B. The red arrows seen in FIG. 20B-20C indicate an equivalent to the fraction of SOP391_v1 seen at the 16 minute peak here eluted at approximately 20 minutes.

FIG. 21A depicts a comparison of the individual peak and total recovery data between the SOP391_v1-v6 operational modes for the R50032 formulation.

FIG. 21B depicts a comparison between the 16 minute peak fraction observed in SOP391_v1 compared to; X: SOP391_v2-v4 where the 16 minute peak was further separated into two populations by the TSK G3000 SEC column; and Y: SOP391_v5-v6 where the 16 min peak coeluted with the bulk of OG1953 conjugate.

FIGS. 22A-22B depict a study using the tandem HPLC method of SOP391_v3 evaluating stability of OG1950PUR and R50032 samples. FIG. 22A depicts the individual peaks observed in the CEX-unbound (L) and CEX-bound (E) fractions of an OG1950PUR sample stored at 37° C. for 7 months, an R50032 formulation stored at 5° C. for 3 months, and an R50032 formulation stored at 25° C. for 3 months. FIG. 22B depicts overlay graphs of the individual peaks of FIG. 22A, where X indicates the peaks for the R50032 formulations stored at 5° C. and 5° C. for 3 months, and Y indicates the OG1950PUR sample stored at 37° C. for 7 months.

FIG. 23 depicts an overview of the principle and operation of the tandem CEX-SEC method where the KSI-301DP formulation can comprise OG1953, OG1953′, OG1950, aggregated OG1950, and insoluble components within a buffer. The prefilter step removes the insoluble components. The OG1950 and aggregates bind the CEX column and the OG1953 is not bound to the column and can be analyzed by SEC. A pulse of 1M NaCl can elute the OG1950 and aggregates from the CEX column and the OG1950, aggregated OG1950, and any OG1953 can be distinguished by SEC.

FIG. 24 depicts a plan for a program to evaluate the stability of the OG1953 bioconjugate containing 7.5, 10, 15, and 20% free OG1950 protein at different temperatures over time. The study evaluates the tandem analytical HPLC method performance in terms of recovery, robustness, specificity, reproducibility, and accuracy.

FIG. 25 depicts a visual assessment of the samples at 1, 2, 3, and 6 months at 5, 25, and 37° C. Arrows indicate samples with cloudy appearance.

FIG. 26 depicts an assessment of protein concentration of samples stored at 5, 25, and 37° C. for 1, 2, and 6 months with an analysis of changes in protein concentration over time.

FIGS. 27A-27C depict an overview of all sample recovery and elution profiles. FIG. 27A depicts total mass recovery over time and temperature (top and bottom panel) for CEX-bound and CEX-unbound samples where close to 100% mass recovery was achieved for all samples. FIG. 27B depicts chromatograms for the CEX-unbound showing OG1953 conjugate peak (LP1) with instability population designated LP2. FIG. 27C depicts chromatograms for the CEX-bound fractions showing a 16 minute peak that splits into A and B and a P1, P1, and M which were OG1950 free protein peaks.

FIGS. 28A-28D depict a comparison of LP2, 16 minute peak and monomeric free protein for sample #4 and a representative clinical formulation. FIG. 28A depicts a summary of the results obtained for sample #4, OG1953 KSI-301DP 103A, OG1953 KSI-301DS 101 samples stored under different storage conditions including mass recovery and percent 280 nm signal for the individual peaks identified, where X highlights the 16 minute A and B peaks. FIG. 28B depicts a chromatogram of the CEX-unbound fraction comparing the LP1 and LP2 peaks of the OG1953 KSI-301DP 103A frozen and 6M 25° C. samples. FIG. 28C-28D depict chromatograms of the CEX-bound fraction indicating the 16 minute peak and Z describing monomer form protein (M) peak.

FIGS. 29A-29D depict kinetic analysis of various peak fractions of sample #4 at different times and temperatures. FIG. 29A depicts a summary of the analysis of the all the peaks detected in the CEX-bound and CEX-unbound fractions of sample #4. FIG. 29B depicts a summary of the degradation and/or aggregation rates for all peaks at 25° C. FIG. 29C depicts the kinetic curve for all peaks at each sampling including CEX-unbound run peaks (LP1, and LP2) and CEX-bound run peaks (16 minute peak, P2 (dimer), P1, M (monomer). FIG. 29D depicts the accompanying chromatogram for the CEX-bound run peaks.

FIGS. 30A-30D depict kinetic analysis of various peal fractions of sample #3 (15%) and #9 (20% iodoacetamide (IAM)) at different time and temperatures. FIG. 30A depicts a summary of the peak analysis of all the peaks detected in the CEX-bound and CEX-unbound fractions of sample #3, and a summary of the degradation and/or aggregation rates for all peaks at 25° C. FIG. 30B depicts the kinetic curve for all peaks at each sampling of the #3 sample including CEX-unbound run peaks (LP1, and LP2) and CEX-bound run peaks (16 minute peak, P2 (dimer), P1, M (monomer). FIG. 30C depicts a summary of the peak analysis of all the peaks detected in the CEX-bound and CEX-unbound fractions of sample #9, and a summary of the degradation and/or aggregation rates for all peaks at 25° C. FIG. 30D depicts the kinetic curve for all peaks at each sampling of sample #9 including CEX-unbound run peaks (LP1, and LP2) and CEX-bound run peaks (16 minute peak, P2 (dimer), P1, M (monomer).

FIGS. 31A-31B depict a projection of two year stability of samples #3, 4, and 9 at different temperatures. FIG. 31A depicts a summary of the peak percent of all detected peaks in the CEX-inbound and CEX-bound fractions for samples #3, 4, and 9. FIG. 31B depicts projections for the percent of total impurities (e.g., aggregates and/or degradation products) over a two year period for samples #3, 4, and 9.

FIG. 32 depicts a summary of the mass recovery data for all injections of samples #3, 4, and 9 at different times and temperatures.

FIGS. 33A-33B depict graphs for 100% B buffer pulse elution profiles of the 20% antibody with 80% bioconjugate using pulse elution profiles #3-7. FIG. 33A depicts individual peaks and FIG. 33B depicts overlay plots.

FIGS. 34A-34B depict data analysis summary (FIG. 34A) and chromatogram comparisons of various pulse elution profiles (FIG. 34B).

FIG. 35 depicts OG1786.

FIG. 36 depicts OG1802.

FIGS. 37A-37C depict updates to the projections of FIG. 37A-37C, where additional samplings were performed at 12 and 15 months for 5° C. and 25° C., respectively.

FIGS. 38A-38D depict some embodiments of continuous 80 min tandem separation method with PhotoDiol Array (PDA) detection set at 200-350 nm. FIG. 38A depicts the 2D contour view of elution time versus wavelength; FIG. 38B depicts the extracted wavelength profile at 280 nm and the peak identification of the various eluted fractions collected for further characterization using SDS-PAGE analysis followed by Silver Staining, and results were shown in FIG. 38C as a non-reducing gel and FIG. 38D as a reducing gel.

FIGS. 39A-39C depict KSI-301 stability data up to 6 months under different temperature conditions; −20±5° C. (FIG. 39A), 5±3° C. (FIG. 39B), and 25±2° C./60±5% RH (relative humidity) (FIG. 39C).

FIG. 40 depicts KSI-501DS Batches 1-3 Lot Release Data.

### DETAILED DESCRIPTION

Some embodiments provided herein are methods and apparatuses for tandem HPLC. In some embodiments, the methods and apparatuses for tandem HPLC allow for one to analyze a mixed protein formulation.

Some embodiments provided herein are methods and apparatuses for tandem HPLC combining ion exchange chromatography (IEX), such as cation exchange chromatography (CEX) and size exclusion chromatography (SEC). The methods and apparatuses for tandem HPLC allow for one to analyze a formulation comprising a protein conjugated to a polymer, and a protein not conjugated to a polymer.

Provided herein are methods and apparatuses for analyzing a mixed formulation comprising a fast-acting component comprising an unconjugated protein and a longer-term acting component comprising protein conjugated to a polymer. In some embodiments, the methods and apparatuses provided herein are part of any suitable process for producing and purifying a mixed composition of some conjugated and unconjugated protein.

Several surprises have been discovered when mixing the ABC™ conjugate with free protein formulation in general. Firstly, there was the presence of unpaired engineered cysteine residues present in the protein moiety that posts potential disulfide shuffling mediated protein aggregation, especially between the free and unconjugated protein fraction or with the bioconjugate at high protein concentration; secondly, there was a surprise finding that such mixture resulted in white turbid solution under certain condition such as (1) the solution pH was at or close to the isoelectric point (pI) of the free unconjugated protein, (2) concentration of the conjugate and/or free protein was too high, or (3) presence of certain excipients such as histidine. Turbid solution is not desirable for injectable pharmaceutical in general, especially for intravitreal injectable medicines. In some embodiments, the methods and apparatuses described herein contribute to the development of a stable and clear solution with up to 6 months stability and projected to have at least 12 months long term stability at 2-8° C.

OG1953 bioconjugate can be polydisperse due to the covalently conjugated polymer moiety, it can contain small population of molecules with molecular weights that overlap with the free unconjugated protein fraction, especially the aggregates of the free protein. Furthermore, the protein moiety can also possess unique charge variation. Both the size and charge variation pose complex analytical challenges for the existing analytical methods such as CEX-HPLC and SEC-HPLC method for monitoring the molecules based on charge and size, respectively. The situation can be further exacerbated over time and stress temperature as the impurities such as degradants or aggregates of the conjugate and protein overlap with each other rendering it technically impossible to use either method alone with good resolution for such analysis.

In some embodiments, the methods and apparatuses described herein can provide one or more of the following advantages: (1) Leverage the existing CEX-HPLC and SEC-HPLC analytical methods for either the conjugate or free unconjugated protein; (2) it can be used for both batch release and stability indicating; (3) it can be performed under non-destructive and native buffer condition to prevent in-process modification and artifact; (4) other than simple sample dilution, there should be no further sample manipulation nor purification prior the sample analysis (5) the method can meet the requirements for GMP validation such as accuracy, robustness, reproducibility, and specificity; (6) the method can be implemented and set up in an analytical laboratory equipped with common instrumentation and trained technical experts in the art; (7) the method can provide good resolution and quantitative information for various degradation products and/or aggregates (e.g., impurities), (8) the method can provide kinetic recovery (100% recovery) for the injected sample, which also means the method should have minimal carry-over issue; (9) the method can also have good dynamic range of operation such as tolerance to running buffer pH, salt concentration, sample loading, flow rate, operation temperature, etc.; (10) the method can be further integrated seamlessly with other analytical instrumentation and methods, for example the MALS detector, icIEF, Mass spectrometry, fluorescent detector, etc.; (11) buffer running condition that minimizes the disulfide shuffling, therefore the running buffer can be at the acidic range such as pH 4-6.5.

In some embodiments, the methods and apparatuses described herein can provide one or more of the following advantages: (1) isocratic chromatographic running condition to take advantage of the existing gradient elution characteristic of CEX-HPLC method, which the bulk of OG1953 conjugate is eluted with minimal to no premature leaching of bound OG1950 free protein and impurities such as degradants, aggregates, or fragments. Without being limited by theory, OG1953 conjugate binds weaker to the CEX column through the protein moiety due to the charge shielding effect of the large polymer, whereas the free unconjugated protein and its protein aggregates elutes later, it should also noted that smaller polymers that exert less shielding effect on conjugated protein moiety could binds tighter to the CEX column and delays elution as compared to the bulk of the conjugate, this phenomena can be seen as the prolonged trailing of CEX or SEC elution after the bulk conjugate elution peak; (2) rapid synchronous and mass transfer of the CEX-bound fraction for SEC separation based on size, the general principal of SEC chromatography is based on the synchronous sample application with a sample volume no more than 5% of total SEC column volume, hence a sufficiently high salt elution pulse that elutes all CEX bound fraction at smallest possible volume can be used for efficient SEC separation. In the event of asynchronous elution such as insufficient salt concentration or too shallow elution speed, it could compromise the SEC resolution and separation efficiency.

In some embodiments, an advantageous feature of the current tandem chromatography method is the instant finger-printing ability. In some embodiments, the first dimension separation is a group specific chromatography such as ion exchanger chromatography (IEX) for charge specific separation (e.g. CEX or AEX), affinity chromatography based on specific group (e.g. protein A, protein G, protein L, M2 resin for flag-tag proteins, metal chelating resins such as Nickle-NTA for His-tag protein, dye-affinity resin, lectin-affinity resin for specific carbohydrate separation, hydrophobic interaction resin, ligand specific antibody-affinity capturing resin, etc.). In some embodiments, the second dimensional separation can be by size with SEC, which the SEC provides instant identity finger print profile for both the unbound and thereafter bound fraction of the group specific column in a single operation. In some embodiments, this is conceptually different from the conventional concept of two dimensional chromatography which aim to simply separate a complex mixture of analytes for a unique finger-print. In some embodiments, the current tandem HPLC method aims to provide convenient, simple and yet efficient instant finger printing of the first dimensional separation, when combining the effluent of the SEC with other detection instrumentations and techniques, it can be widely used in other areas of application.

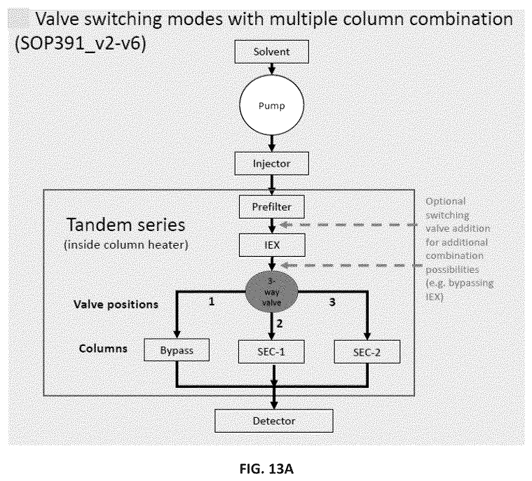

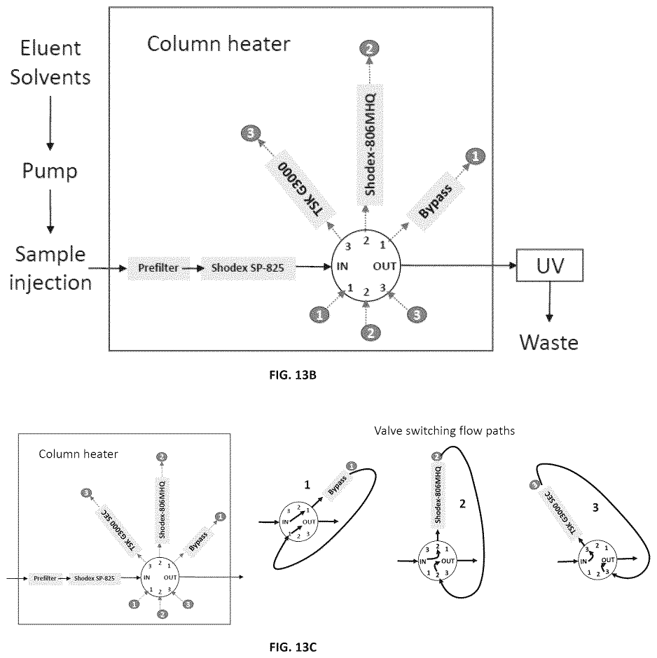

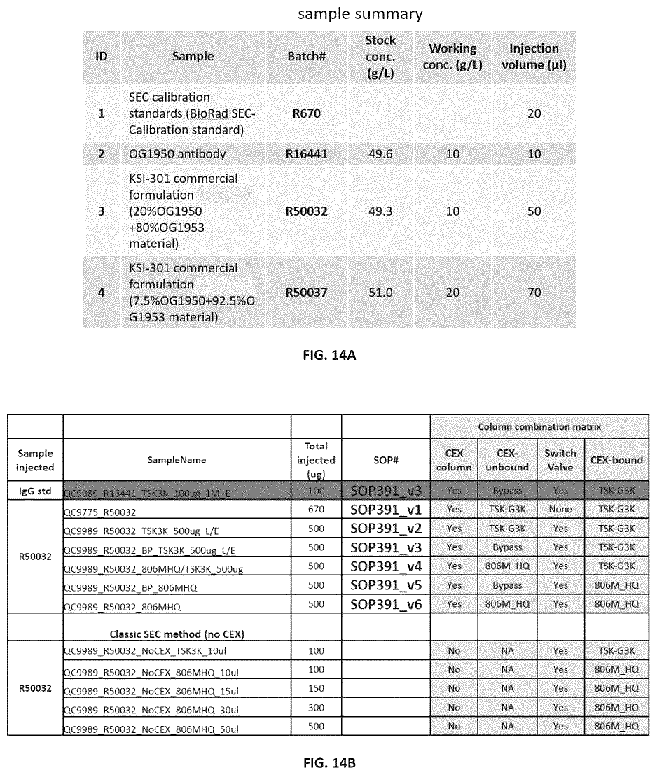

In some embodiments, the method can also be operated under interruptive and uninterrupted (continuous) mode. In some embodiments, interruptive mode can have separate chromatographic runs for the sample injection run and pulse elution of the first dimensional bound fraction, therefore it produces two separate chromatograms for a single sample analysis. In some embodiments, the major disadvantage of such operation can include one or more of: (1) undesirable baseline ramping signal in the beginning of each run, (2) unnecessary regulatory justification for integrating peaks from completely different chromatographic runs, (3) potential chromatographic signal discrepancy alignment issue. In some embodiments, there is also advantage when only the first dimensional column bound fraction is needed, one can design a significantly shortened method with the aid of a multi-column valve with a bypass loop as shown in FIG. 13A, with this operation mode, the unbound fraction can be discarded using the bypass loop and not pass through the SEC column. In some embodiments, for uninterrupted mode, a more sophisticated instrument can require to equip with two pumps and able to operate with small residual volume to deliver a salt pulse for synchronous elution of first dimensional bound fraction, failure to meet this requirement can result in undesirable baseline shift. In some embodiments, the advantage of operating in uninterrupted mode is that all elution peak of unbound and bound fractions of the first dimensional column can be displayed in a single chromatogram.

In some embodiments, understanding of the molecules in the mix formulation is useful for the success of using the current tandem HPLC method. In some embodiments, the first dimensional column allows the bulk of interference or matrix effect to be removed. In some embodiments, the unbound fraction contains undesirable contaminants such as sample formulation buffer and free unconjugated polymer (OG1802). In some embodiments, selection of the salt concentration and pH of the mobile phase confers specificity of separation. In some embodiments, the salt concentration and pH of the mobile phase can be manipulated to control how thorough the interference is to be removed. In some embodiments, too aggressive salt and pH condition can result in premature elution of the bound fraction and can result in compromised method performance.

Some embodiments provided herein can include one or more of the following: (1) Simple, effective and seamless integration of the existing chromatography methods that resulted in high resolution separation of various peaks in a complex mixture formulation that is previously impossible with individual methods alone; (2) Real time online identity and purity method that allows instant identification of the various fractions prior and post addition to the combined formulation; (3) Its sensitivity, robustness, accuracy and reproducibility can be adapted for GMP validation of pharmaceutical manufacturing; (4) It provides a single integrated method that can allow defining, detection and quantification of various degradation products and/or aggregates under native running condition similar to both the large scale purification process and the final drug substance and drug product formulation buffer; (5) It can be used for both lot release and stability indicating; (6) it is versatile and is able to integrate seamlessly with other analytical techniques that complement and expand the current power of resolution to gain even more insight into the property of the mixed population in the pharmaceutical formulation.

In some embodiments, when used in the context of a pharmaceutical composition (e.g. OG1953 drug substance or drug product), the following advantages can be achieved: (1) under optimized chromatography condition, it can allow kinetic recovery of all injected samples with high resolution for all major products peaks including OG1953 conjugate (LP1) and monomeric IgG of OG1950 (M), and their degradation products and/or aggregates (e.g., impurities), which can include OG1953 related degradation products such as LP2 and ‘16 min peak’, OG1950 related aggregates such as dimer (P2) and an alternative aggregated form of P1; (2) it is broadly applicable to a panel of different formulation with 1%, 7.5%, 10%, 15%, 20%, 49% and 89% free antibody mixed with various percent of OG1953 conjugate; furthermore, it can also be applicable to other molecules such as Fab with similar performance; (3) it allows defining the degradation products and/or aggregates, and formation kinetic over time and temperature in a long term stability study, which allows setting specification and prediction model to project the long term shelf-life.

Provided herein are methods and apparatuses mixed formulations of antibodies and conjugates thereof and other proteins and protein conjugates by tandem HPLC. In some embodiments, the conjugate can be used for the treatment of certain conditions, such as diabetic retinopathy and/or age-related macular degeneration.

In some embodiments, a method of analyzing a mixed formulation sample is provided. In some embodiments, the method comprises providing a mixed formulation that comprises a combination of two protein moieties. In some embodiments, the first protein moiety (A) is conjugated to a polymer, and the second protein (B) moiety is not conjugated to a polymer. In some embodiments, the method comprises loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column. In some embodiments, the method comprises a first run. In some embodiments, during the first run, the mixed formulation is injected and run through the tandem CEX-SEC system. In some embodiments, the method comprises a second run. In some embodiments, during the second run, a concentrated salt is injected to elute the bound fraction of the CEX column to be separated by the SEC column. In some embodiments, a specific ionic strength of the solvent is used to allow partitioning of the mixed formulation in the CEX column. In some embodiments, the method allows analysis of the mixed formulation based on the differential charge and size variant of the constituents.

These and additional embodiments are provided below, following the definition section.

### Definitions

All terms can have their customary and ordinary meaning to one of ordinary skill int the art, in view of the present disclosure.

The term “percent composition” refers to the percent amount (in mass or concentration units) of a component present in a composition. Percent composition is calculated by determining the amount of a component in mass units (e.g., μg) or in concentration units (e.g., mg/mL), dividing that amount by the total amount of all components in the composition in the corresponding unit, and multiplying by 100. For compositions and formulations of a conjugate and an unconjugated protein described herein, the amount of the unconjugated protein can be divided by the total amount of the protein mass in the solution (excluding the contribution from the polymer component of the conjugate to the mass of the conjugate) to obtain a percent composition.

As used herein, “% total molar amount” denotes the proportion (in percent) of the amount (in moles or a molar concentration) of one component of a composition relative to the amount(s) (in moles or a molar concentration) of one or more other component of the composition, that together make up the whole (100%). It is understood that percent composition and % total molar amount is effectively interchangeable where the molecular weight of all of the relevant components are known.

“Molar ratio” refers to the ratio of the amount (in moles) of free protein (e.g. OG1950) to conjugated protein (e.g. OG1953) in the formulation (based on the protein portion of the conjugate).

As used herein “unconjugated” and “free” with reference to a protein, protein moiety, or antibody are used interchangeably to denote the protein or antibody that is not conjugated to a polymer (e.g., not conjugated to a phosphorylcholine-containing polymer).

A “neovascular disorder” is a disorder or disease state characterized by altered, dysregulated or unregulated angiogenesis. Examples of neovascular disorders include neoplastic transformation (e.g. cancer) and ocular neovascular disorders including diabetic retinopathy and age-related macular degeneration.

In some embodiments, “impurities” refer to product related impurities such as degraded, aggregated, unconjugated (e.g., where the product of interest is conjugated), or modified proteins. In some embodiments, product unrelated impurities such as host cell protein, endotoxin, host cell DNA, are not considered “impurities” as defined herein.

An “ocular neovascular” disorder is a disorder characterized by altered, dysregulated or unregulated angiogenesis in the eye of a patient. Such disorders include optic disc neovascularization, iris neovascularization, retinal neovascularization, choroidal neovascularization, corneal neovascularization, vitreal neovascularization, glaucoma, pannus, pterygium, macular edema, diabetic retinopathy, diabetic macular edema, vascular retinopathy, retinal degeneration, uveitis, inflammatory diseases of the retina, and proliferative vitreoretinopathy.

The term antibody includes intact antibodies and binding fragments thereof. A binding fragment refers to a molecule other than an intact antibody that comprises a portion of an intact antibody that binds the antigen to which the intact antibody binds. Examples of binding fragments include Fv, Fab′, Fab′-SH, F(ab′) 2; diabodies; linear antibodies; single-chain antibody molecules (e.g. scFv); and multispecific antibodies formed from antibody fragments. scFv antibodies are described in Houston J S. 1991. Methods in Enzymol. 203:46-96. In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

Specific binding of an antibody to its target antigen(s) means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody or fusion protein binds one and only one target.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the antibodies or fusion proteins have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcR binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, bispecific antibodies can be made in which the two binding sites are different (see, e.g., Songsivilai S, Lachmann P C. 1990. Bispecific antibody: a tool for diagnosis and treatment of disease. Clin Exp Immunol. 79:315-321; Kostelny S A, Cole M S, Tso J Y. 1992. Formation of bispecific antibody by the use of leucine zippers. J Immunol. 148:1547-1553). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FRI, CDR1, FR2, CDR2, FR3, CDR3 and FR4. For convenience, the variable heavy CDRs can be referred to as CDRH1, CDRH2 and CDRH3; the variable light chain CDRs can be referred to as CDRL1, CDRL2 and CDRL3. The assignment of amino acids to each domain is in accordance with the definitions of Kabat E A, et al. 1987 and 1991. Sequences of Proteins of Immunological Interest (National Institutes of Health, Bethesda, MD) or Chothia C, Lesk A M. 1987. Canonical Structures for the Hypervariable Regions of Immunoglobulins. J Mol Biol 196:901-917; Chothia C, et al. 1989. Conformations of Immunoglobulin Hypervariable Regions. Nature 342:877-883. Kabat also provides a widely used numbering convention (Kabat numbering) in which corresponding residues between different heavy chain variable regions or between different light chain variable regions are assigned the same number. Although Kabat numbering can be used for antibody constant regions, EU numbering is more commonly used, as is the case in this application. Although specific sequences are provided for exemplary antibodies disclosed herein, it will be appreciated that after expression of protein chains one to several amino acids at the amino or carboxy terminus of the light and/or heavy chain, particularly a heavy chain C-terminal lysine residue, can be missing or derivatized in a proportion or all of the molecules.

The term “epitope” refers to a site on an antigen to which an antibody or extracellular trap segment binds. An epitope on a protein can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody (or Fab fragment) bound to its antigen to identify contact residues.

Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50%. In some embodiments the test antibody inhibits binding of the reference antibody by 75%, 90%, or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

As used herein, “VEGF Trap” or similar term denotes the VEGF binding domains (e.g., VEGFR1 domain 2, VEGFR2 domain 3). This fragment allows for the protein to work as a VEGF trap, preventing VEGF from binding to cellularly expressed VEGF receptors. An example of this sequence can be found in Table 3. In some embodiments, the VEGF Trap only includes VEGFR1 domain 2, VEGFR2 domain 3. Various embodiments of Trap proteins are known in the art and can be found, for example in U.S. Pub. No. 20150376271, the entirety of which, with respect to various VEGF Trap embodiments (which are VEGFR proteins or fragments thereof) and fusions thereof, is incorporated herein by reference. In some embodiments, the term “VEGF Trap” or similar term refers to a full length extracellular region or any portion thereof, or combination of portions from different VEGF receptors that can antagonize signaling between at least one VEGF and VEGFR.

In some embodiments, Size exclusion chromatography (SEC) can also be called gel permeation chromatography (GPC). In some embodiments, affinity chromatography column can also be called group specific affinity column.

The term “patient” includes human and other mammalian subjects that receive either prophylactic or therapeutic treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Sequence identities of other sequences can be determined by aligning sequences using algorithms, such as BESTFIT, FASTA, and TFASTA in the Wisconsin Genetics Software Package Release 7.0, Genetics Computer Group, 575 Science Dr., Madison, WI, using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

Compositions or methods “comprising” one or more recited elements can include other elements not specifically recited. For example, a composition that comprises antibody can contain the antibody alone or in combination with other ingredients.

The term “antibody-dependent cellular cytotoxicity”, or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcy receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term opsonization also known as “antibody-dependent cellular phagocytosis”, or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement can also result in deposition of complement components on the target cell surface that facilitate ADCC by binding complement receptors (e.g., CR3) on leukocytes.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. No. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly, a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (which can be as defined by Kabat) from a mouse antibody, they can also be made with less than all CDRs (e.g., at least 3, 4, or 5 CDRs from a mouse antibody) (e.g., De Pascalis R, Iwahashi M, Tamura M, et al. 2002. Grafting “Abbreviated” Complementary-Determining Regions Containing Specificity-Determining Residues Essential for Ligand Contact to Engineer a Less Immunogenic Humanized Monoclonal Antibody. J Immunol. 169:3076-3084; Vajdos F F, Adams C W, Breece T N, Presta L G, de Vos A M, Sidhu, S S. 2002. Comprehensive functional maps of the antigen-binding site of an anti-ErbB2 antibody obtained with shotgun scanning mutagenesis. J Mol Biol. 320:415-428; Iwahashi M, Milenic D E, Padlan E A, et al. 1999. CDR substitutions of a humanized monoclonal antibody (CC49): Contributions of individual CDRs to antigen binding and immunogenicity. Mol Immunol. 36:1079-1091; Tamura M, Milenic D E, Iwahashi M, et al. 2000. Structural correlates of an anticarcinoma antibody: Identification of specificity-determining regions (SDRs) and development of a minimally immunogenic antibody variant by retention of SDRs only. J Immunol. 164:1432-1441).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that can contribute to B- or T-cell epitopes, for example exposed residues (Padlan E A. 1991. A possible procedure for reducing the immunogenicity of antibody variable domains while preserving their ligand-binding properties. Mol Immunol. 28:489-98) with residues from the corresponding positions of a human antibody sequence. The result is an antibody in which the CDRs are entirely or substantially from a non-human antibody and the variable region frameworks of the non-human antibody are made more human-like by the substitutions. A human antibody can be isolated from a human, or otherwise result from expression of human immunoglobulin genes (e.g., in a transgenic mouse, in vitro or by phage display). Methods for producing human antibodies include the trioma method of Östberg L, Pursch E. 1983. Human×(mouse×human) hybridomas stably producing human antibodies. Hybridoma 2:361-367; Östberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. Nos. 5,877,218, 5,871,907, 5,858,657, 5,837,242, 5,733,743 and 5,565,332.

“Polymer” refers to a series of monomer groups linked together. A polymer is composed of multiple units of a single monomer (a homopolymer) or different monomers (a heteropolymer). High MW polymers are prepared from monomers that include, but are not limited to, acrylates, methacrylates, acrylamides, methacrylamides, styrenes, vinyl-pyridine, vinyl-pyrrolidone and vinyl esters such as vinyl acetate. Additional monomers are useful in high MW polymers. When two different monomers are used, the two monomers are called “comonomers,” meaning that the different monomers are copolymerized to form a single polymer. The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group.

“Initiator” refers to a compound capable of initiating a polymerization using monomers or comonomers. The polymerization can be a conventional free radical polymerization or a controlled/“living” radical polymerization, such as Atom Transfer Radical Polymerization (ATRP), Reversible Addition-Fragmentation-Termination (RAFT) polymerization or nitroxide mediated polymerization (NMP). The polymerization can be a “pseudo” controlled polymerization, such as degenerative transfer. When the initiator is suitable for ATRP, it contains a labile bond which can be homolytically cleaved to form an initiator fragment, I, being a radical capable of initiating a radical polymerization, and a radical scavenger, I′, which reacts with the radical of the growing polymer chain to reversibly terminate the polymerization. The radical scavenger I′ is typically a halogen, but can also be an organic moiety, such as a nitrile. In some embodiments, the initiator contains one of more 2-bromoisobutyrate groups as sites for polymerization via ATRP.

A “chemical linker” refers to a chemical moiety that links two groups together, such as a half-life extending moiety and a protein. The linker can be cleavable or non-cleavable. Cleavable linkers can be hydrolyzable, enzymatically cleavable, pH sensitive, photolabile, or disulfide linkers, among others. Other linkers include homobifunctional and heterobifunctional linkers. A “linking group” is a functional group capable of forming a covalent linkage comprising one or more bonds to a bioactive agent. Non-limiting examples include those illustrated in Table 1 of WO2013059137 (incorporated by reference).

The term “reactive group” refers to a group that is capable of reacting with another chemical group to form a covalent bond, i.e. is covalently reactive under suitable reaction conditions, and generally represents a point of attachment for another substance. The reactive group is a moiety, such as maleimide or succinimidyl ester, is capable of chemically reacting with a functional group on a different moiety to form a covalent linkage. Reactive groups generally include nucleophiles, electrophiles and photoactivatable groups.

“Phosphorylcholine,” also denoted as “PC,” refers to the following:

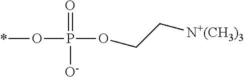

where * denotes the point of attachment. The phosphorylcholine is a zwitterionic group and includes salts (such as inner salts), and protonated and deprotonated forms thereof.

“Phosphorylcholine containing polymer” is a polymer that contains phosphorylcholine. “Zwitterion containing polymer” refers to a polymer that contains a zwitterion.

Poly(acryloyloxyethyl phosphorylcholine) containing polymer refers to a polymer containing 2-(acryloyloxy)ethyl-2-(trimethylammonium)ethyl phosphate (HEA-PC shown below in Example 6) as monomer.

Poly(methacryloyloxyethyl phosphorylcholine) containing polymer refers to a polymer containing 2-(methacryloyloxy)ethyl-2-(trimethylammonium)ethyl phosphate (HEMA-PC or MPC) as monomer (see below):

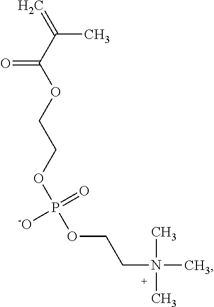

As used herein, “MPC” and “HEMA-PC” are interchangeable.

“Molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight, or the use of light scattering techniques, ultracentrifugation or viscometry to determine weight average molecular weight. In some embodiments, the molecular weight is measured by SEC-MALS (size exclusion chromatography-multi angle light scattering). In some embodiments, the multi-angle light scattering includes 18-angle MALS. In some embodiments, the multi-angle light scattering includes 3-angle and 18-angle MALS. In some embodiments, the polymeric reagents are typically polydisperse (i.e., number average molecular weight and weight average molecular weight of the polymers are not equal), and can possess low polydispersity values of, for example, less than about 1.5, as judged, for example, by the PDI value derived from the SEC-MALS measurement. In some embodiments, the polydispersities (PDI) are in the range of about 1.4 to about 1.2. In some embodiments the PDI is less than about 1.15, 1.10, 1.05, or 1.03.

The phrase “a” or “an” entity refers to one or more of that entity; for example, a compound refers to one or more compounds or at least one compound. As such, the terms “a” (or “an”), “one or more”, and “at least one” can be used interchangeably herein.

“About” means variation one might see in measurements taken among different instruments, samples, and sample preparations.

“Protected,” “protected form,” “protecting group” and “protective group” refer to the presence of a group (i.e., the protecting group) that prevents or blocks reaction of a particular chemically reactive functional group in a molecule under certain reaction conditions. Protecting groups vary depending upon the type of chemically reactive group being protected as well as the reaction conditions to be employed and the presence of additional reactive or protecting groups in the molecule, if any. Suitable protecting groups include those such as found in the treatise by Greene et al., “Protective Groups In Organic Synthesis,” 3rd Edition, John Wiley and Sons, Inc., New York, 1999.

“Alkyl” refers to a straight or branched, saturated, aliphatic radical having the number of carbon atoms indicated. For example, C1-C6 alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together.

The term “lower” referred to above and hereinafter in connection with organic radicals or compounds respectively defines a compound or radical which can be branched or unbranched with up to and including 7 or up to and including 4 and (as unbranched) one or two carbon atoms.

“Alkylene” refers to an alkyl group, as defined above, linking at least two other groups, i.e., a divalent hydrocarbon radical. The two moieties linked to the alkylene can be linked to the same atom or different atoms of the alkylene. For instance, a straight chain alkylene can be the bivalent radical of —(CH2) n, where n is 1, 2, 3, 4, 5 or 6. Alkylene groups include, but are not limited to, methylene, ethylene, propylene, isopropylene, butylene, isobutylene, sec-butylene, pentylene and hexylene.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O) R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. The term “alkyl” includes groups such as haloalkyl (e.g., —CF₃ and —CH₂CF₃) and acyl (e.g., —C(O)CH₃, —C(O)CF₃, —C(O)CH₂OCH₃, and the like). In some embodiments, the substituted alkyl and heteroalkyl groups have from 1 to 4 substituents. In some embodiments, the substituted alkyl and heteroalkyl groups have 1, 2 or 3 substituents. Exceptions are those perhalo alkyl groups (e.g., pentafluoroethyl and the like).

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, not but limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O) CH₃, —C(O) CF₃, —C(O) CH₂OCH₃, and the like).

“Alkoxy” refers to alkyl group having an oxygen atom that either connects the alkoxy group to the point of attachment or is linked to two carbons of the alkoxy group. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

“Carboxyalkyl” means an alkyl group (as defined herein) substituted with a carboxy group. The term “carboxycycloalkyl” means a cycloalkyl group (as defined herein) substituted with a carboxy group. The term alkoxyalkyl means an alkyl group (as defined herein) substituted with an alkoxy group. The term “carboxy” employed herein refers to carboxylic acids and their esters.

“Haloalkyl” refers to alkyl as defined above where some or all of the hydrogen atoms are substituted with halogen atoms. Halogen (halo) represents chloro or fluoro, but can also be bromo or iodo. For example, haloalkyl includes trifluoromethyl, fluoromethyl, 1,2,3,4,5-pentafluoro-phenyl, etc. The term “perfluoro” defines a compound or radical which has all available hydrogens that are replaced with fluorine. For example, perfluorophenyl 1,2,3,4,5-pentafluorophenyl, perfluoromethyl refers to 1,1,1-trifluoromethyl, and perfluoromethoxy refers to 1,1,1-trifluoromethoxy.

“Fluoro-substituted alkyl” refers to an alkyl group where one, some, or all hydrogen atoms have been replaced by fluorine.

“Cytokine” is a member of a group of protein signaling molecules that can participate in cell-cell communication in immune and inflammatory responses. Cytokines are typically small, water-soluble glycoproteins that have a mass of about 8-35 kDa.

“Cycloalkyl” refers to a cyclic hydrocarbon group that contains from about 3 to 12, from 3 to 10, or from 3 to 7 endocyclic carbon atoms. Cycloalkyl groups include fused, bridged and spiro ring structures.

“Endocyclic” refers to an atom or group of atoms which comprise part of a cyclic ring structure.

“Exocyclic” refers to an atom or group of atoms which are attached but do not define the cyclic ring structure.

“Cyclic alkyl ether” refers to a 4 or 5 member cyclic alkyl group having 3 or 4 endocyclic carbon atoms and 1 endocyclic oxygen or sulfur atom (e.g., oxetane, thietane, tetrahydrofuran, tetrahydrothiophene); or a 6 to 7 member cyclic alkyl group having 1 or 2 endocyclic oxygen or sulfur atoms (e.g., tetrahydropyran, 1,3-dioxane, 1,4-dioxane, tetrahydrothiopyran, 1,3-dithiane, 1,4-dithiane, 1,4-oxathiane).

“Alkenyl” refers to either a straight chain or branched hydrocarbon of 2 to 6 carbon atoms, having at least one double bond. Examples of alkenyl groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can also have from 2 to 3, 2 to 4, 2 to 5, 3 to 4, 3 to 5, 3 to 6, 4 to 5, 4 to 6 and 5 to 6 carbons. The alkenyl group is typically monovalent, but can be divalent, such as when the alkenyl group links two moieties together.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene.

“Alkynyl” refers to either a straight chain or branched hydrocarbon of 2 to 6 carbon atoms, having at least one triple bond. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can also have from 2 to 3, 2 to 4, 2 to 5, 3 to 4, 3 to 5, 3 to 6, 4 to 5, 4 to 6 and 5 to 6 carbons. The alkynyl group is typically monovalent, but can be divalent, such as when the alkynyl group links two moieties together.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, butynylene, sec-butynylene, pentynylene and hexynylene.

“Cycloalkyl” refers to a saturated or partially unsaturated, monocyclic, fused bicyclic or bridged polycyclic ring assembly containing from 3 to 12 ring atoms, or the number of atoms indicated. Monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C3-8cycloalkyl includes cyclopropyl, cyclobutyl, cyclopentyl, cyclohexyl, cyclooctyl, and norbornane.

“Cycloalkylene” refers to a cycloalkyl group, as defined above, linking at least two other groups, i.e., a divalent hydrocarbon radical. The two moieties linked to the cycloalkylene can be linked to the same atom or different atoms of the cycloalkylene. Cycloalkylene groups include, but are not limited to, cyclopropylene, cyclobutylene, cyclopentylene, cyclohexylene, and cyclooctylene.

“Heterocycloalkyl” refers to a ring system having from 3 ring members to about 20 ring members and from 1 to about 5 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heterocycle includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

“Heterocycloalkylene” refers to a heterocyclalkyl group, as defined above, linking at least two other groups. The two moieties linked to the heterocycloalkylene can be linked to the same atom or different atoms of the heterocycloalkylene.

“Aryl” refers to a monocyclic or fused bicyclic, tricyclic or greater, aromatic ring assembly containing 6 to 16 ring carbon atoms. For example, aryl can be phenyl, benzyl or naphthyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C2-C3-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g. methylenedioxy or ethylenedioxy. Oxy-C2-C3-alkylene is also a divalent substituent attached to two adjacent carbon atoms of phenyl, e.g. oxyethylene or oxypropylene. An example for oxy-C2-C3-alkylene-phenyl is 2,3-dihydrobenzofuran-5-yl.

In some embodiments the aryl is naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

Examples of substituted phenyl groups as R are, e.g. 4-chlorophen-1-yl, 3,4-dichlorophen-1-yl, 4-methoxyphen-1-yl, 4-methylphen-1-yl, 4-aminomethylphen-1-yl, 4-methoxyethylaminomethylphen-1-yl, 4-hydroxyethylaminomethylphen-1-yl, 4-hydroxyethyl-(methyl)-aminomethylphen-1-yl, 3-aminomethylphen-1-yl, 4-N-acetylaminomethylphen-1-yl, 4-aminophen-1-yl, 3-aminophen-1-yl, 2-aminophen-1-yl, 4-phenyl-phen-1-yl, 4-(imidazol-1-yl)-phenyl, 4-(imidazol-1-ylmethyl)-phen-1-yl, 4-(morpholin-1-yl)-phen-1-yl, 4-(morpholin-1-ylmethyl)-phen-1-yl, 4-(2-methoxyethylaminomethyl)-phen-1-yl and 4-(pyrrolidin-1-ylmethyl)-phen-1-yl, 4-(thiophenyl)-phen-1-yl, 4-(3-thiophenyl)-phen-1-yl, 4-(4-methylpiperazin-1-yl)-phen-1-yl, and 4-(piperidinyl)-phenyl and 4-(pyridinyl)-phenyl optionally substituted in the heterocyclic ring.

“Arylene” refers to an aryl group, as defined above, linking at least two other groups. The two moieties linked to the arylene are linked to different atoms of the arylene. Arylene groups include, but are not limited to, phenylene.

“Arylene-oxy” refers to an arylene group, as defined above, where one of the moieties linked to the arylene is linked through an oxygen atom. Arylene-oxy groups include, but are not limited to, phenylene-oxy.

Similarly, substituents for the aryl and heteroaryl groups are varied and are selected from: -halogen, —OR′, —OC(O) R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH2)=NH, —NR′C(NH2)=NH, —NH—C(NH2)=NR′, —S(O)R′, —S(O)2R′, —S(O)2NR′R″, —N3, —CH(Ph)2, perfluoro (C1-C4)alkoxy, and perfluoro (C1-C4)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl.

Two of the substituents on adjacent atoms of the aryl or heteroaryl ring can optionally be replaced with a substituent of the formula -T-C(O)—(CH₂)q-U-, wherein T and U are independently —NH—, —O—, —CH₂— or a single bond, and q is an integer of from 0 to 2. Alternatively, two of the substituents on adjacent atoms of the aryl or heteroaryl ring can optionally be replaced with a substituent of the formula -A-(CH₂)r-B-, wherein A and B are independently —CH₂—, —O—, —NH—, —S—, —S(O)—, —S(O) 2-, —S(O) 2NR′— or a single bond, and r is an integer of from 1 to 3. One of the single bonds of the new ring so formed can optionally be replaced with a double bond. Alternatively, two of the substituents on adjacent atoms of the aryl or heteroaryl ring can optionally be replaced with a substituent of the formula —(CH₂)s-X—(CH₂)t-, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C1-C6)alkyl.

“Heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. In some embodiments, quinolinyl represents 2-, 3- or 4-quinolinyl. In some embodiments, isoquinolinyl represents 1-, 3- or 4-isoquinolinyl. In some embodiments, benzopyranyl, benzothiopyranyl can represent 3-benzopyranyl or 3-benzothiopyranyl, respectively. In some embodiments, thiazolyl can represent 2- or 4-thiazolyl. In some embodiments, triazolyl can be 1-, 2- or 5-(1,2,4-triazolyl). In some embodiments, tetrazolyl can be 5-tetrazolyl.

In some embodiments, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

The term “heteroalkyl” refers to an alkyl group having from 1 to 3 heteroatoms such as N, O and S. Additional heteroatoms can also be useful, including, but not limited to, B, Al, Si and P. The heteroatoms can also be oxidized, such as, but not limited to, —S(O)-and-S(O) 2-. For example, heteroalkyl can include ethers, thioethers, alkyl-amines and alkyl-thiols.

The term “heteroalkylene” refers to a heteroalkyl group, as defined above, linking at least two other groups. The two moieties linked to the heteroalkylene can be linked to the same atom or different atoms of the heteroalkylene.

“Electrophile” refers to an ion or atom or collection of atoms, which can be ionic, having an electrophilic center, i.e., a center that is electron seeking, capable of reacting with a nucleophile. An electrophile (or electrophilic reagent) is a reagent that forms a bond to its reaction partner (the nucleophile) by accepting both bonding electrons from that reaction partner.

“Nucleophile” refers to an ion or atom or collection of atoms, which can be ionic, having a nucleophilic center, i.e., a center that is seeking an electrophilic center or capable of reacting with an electrophile. A nucleophile (or nucleophilic reagent) is a reagent that forms a bond to its reaction partner (the electrophile) by donating both bonding electrons. A “nucleophilic group” refers to a nucleophile after it has reacted with a reactive group. Non limiting examples include amino, hydroxyl, alkoxy, haloalkoxy and the like.

“Maleimido” refers to a pyrrole-2,5-dione-1-yl group having the structure:

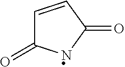

which upon reaction with a sulfhydryl (e.g., a thio alkyl) forms an —S-maleimido group having the structure

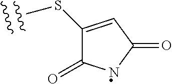

where “•” indicates the point of attachment for the maleimido group and “ [symbol] ” indicates the point of attachment of the sulfur atom the thiol to the remainder of the original sulfhydryl bearing group.

For the purpose of this disclosure, “naturally occurring amino acids” found in proteins and polypeptides are L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine, and or L-valine. “Non-naturally occurring amino acids” found in proteins are any amino acid other than those recited as naturally occurring amino acids. Non-naturally occurring amino acids include, without limitation, the D isomers of the naturally occurring amino acids, and mixtures of D and L isomers of the naturally occurring amino acids. Other amino acids, such as N-alpha-methyl amino acids (e.g. sarcosine), 4-hydroxyproline, desmosine, isodesmosine, 5-hydroxylysine, epsilon-N-methyllysine, 3-methylhistidine, although found in naturally occurring proteins, are considered to be non-naturally occurring amino acids found in proteins for the purpose of this disclosure as they are generally introduced by means other than ribosomal translation of mRNA.

“Linear” in reference to the geometry, architecture or overall structure of a polymer, refers to polymer having a single polymer arm.

“Branched,” in reference to the geometry, architecture or overall structure of a polymer, refers to a polymer having 2 or more polymer “arms” extending from a core structure contained within an initiator. The initiator can be employed in an atom transfer radical polymerization (ATRP) reaction. A branched polymer can possess 2 polymer chains (arms), 3 polymer arms, 4 polymer arms, 5 polymer arms, 6 polymer arms, 7 polymer arms, 8 polymer arms, 9 polymer arms or more. Each polymer arm extends from a polymer initiation site. Each polymer initiation site is capable of being a site for the growth of a polymer chain by the addition of monomers. For example and not by way of limitation, using ATRP, the site of polymer initiation on an initiator is typically an organic halide undergoing a reversible redox process catalyzed by a transition metal compound such as cuprous halide. In some embodiments, the halide is a bromine.

“Pharmaceutically acceptable excipient” refers to an excipient that can be included in compositions and that causes no significant adverse toxicological effect on the patient and is approved or approvable by the FDA for therapeutic use, particularly in humans. Non-limiting examples of pharmaceutically acceptable excipients include water, NaCl, normal saline solutions, lactated Ringer's, normal sucrose, normal glucose and the like. In any embodiment, a pharmaceutically acceptable carrier can be acceptable for administering directly into the eye of a patient (e.g., acceptable for intravitreal administration).

Therapeutic proteins are administered in an effective regime meaning a dosage, route of administration and frequency of administration that delays the onset, reduces the severity, inhibits further deterioration, and/or ameliorates at least one sign or symptom of a disorder. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

The “biological half-life” of a substance is a pharmacokinetic parameter which specifies the time required for one half of the substance to be removed from a tissue or an organism following introduction of the substance.

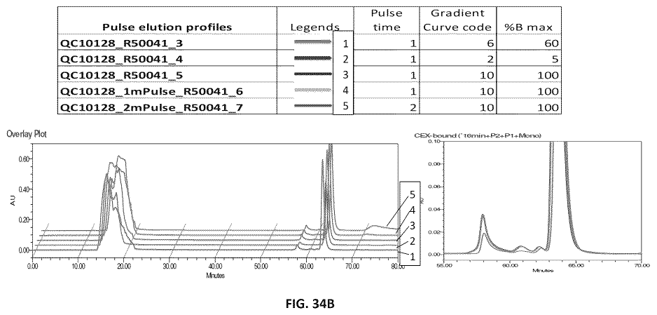

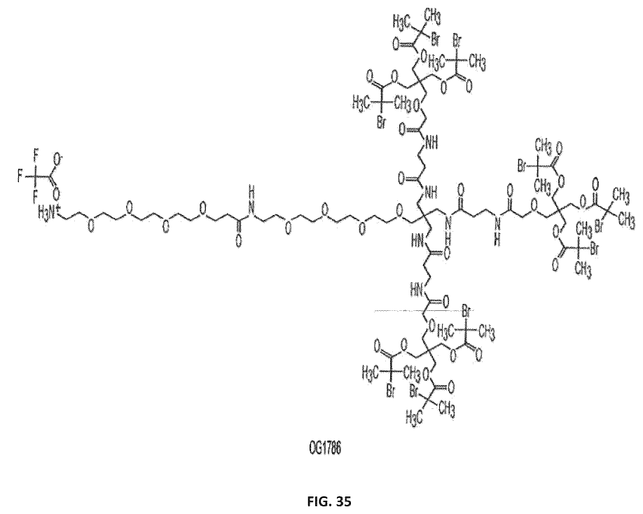

“OG1786” is a 9-arm initiator used for polymer synthesis with the structure shown in FIG. 35, which depicts that salt form of OG1786 with trifluororacetic acid. OG1786 can be used as other salts are used or as the free base.

“OG1801” is an approximately (+/−15%) 750 kDa polymer (either by Mn or Mp) made using OG1786 as an intiator for ATRP synthesis using the monomer HEMA-PC.

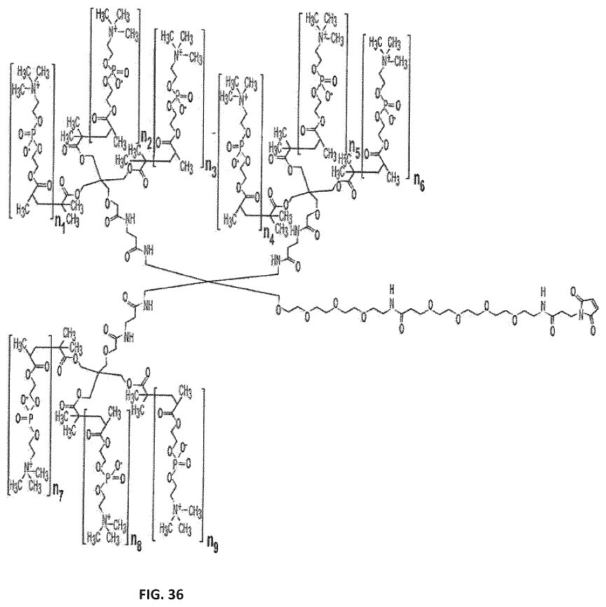

“OG1802” is OG1801 with a maleimide functionality added and is shown in FIG. 36, wherein each of n1, n2, n3, n4, n5, n6, n7, n8 and n9 is an integer (positive) (from 0 up to about 3000) such that the total molecular weight of the polymer is (Mw) 750,000±15% daltons.

Multi-angle light scattering (MALS) is a technique of analyzing macromolecules where the laser light impinges on the molecule, the oscillating electric field of the light induces an oscillating dipole within it. This oscillating dipole will re-radiate light and can be measured using a MALS detector such as Wyatt miniDawn TREOS. The intensity of the radiated light depends on the magnitude of the dipole induced in the macromolecule which in turn is proportional to the polarizability of the macromolecule, the larger the induced dipole, and hence, the greater the intensity of the scattered light. Therefore, in order to analyze the scattering from a solution of such macromolecules, one should know their polarizability relative to the surrounding medium (e.g., the solvent). This can be determined from a measurement of the change, Δn, of the solution's refractive index n with the molecular concentration change, Δc, by measuring the dn/dc (=Δn/Δc) value using a Wyatt Optilab T-rEX differential refractometer. Two molar weight parameters that MALS determination employ are number average molecular weight (Mn) and weight average molecular weight (Mw) where the polydispersity index (PDI) equals Mw divided by Mn. SEC also allows another average molecular weight determination of the peak molecular weight Mp which is defined as the molecular weight of the highest peak at the SEC.

The PDI is used as a measure of the broadness of a molecular weight distribution of a polymer and bioconjugate which is derived from conjugation of a discrete protein (e.g. OG1950) to a polydisperse biopolymer (e.g., OG1802). For a protein sample, its polydispersity is close to 1.0 due to the fact that it is a product of translation where every protein molecule in a solution is expected to have almost the same length and molar mass. In contrast, due to the polydisperse nature of the biopolymer where the various length of polymer chains are synthesized during the polymerization process, it is very important to determine the PDI of the sample as one of its quality attribute for narrow distribution of molecular weight.

Size exclusion chromatography (SEC) is a chromatography technique in which molecules in solution are separated by their size. Typically, an aqueous solution is applied to transport the sample through the column which is packed with resins of various pore sizes. The resin is expected to be inert to the analyte when passing through the column and the analytes separate from each other based on their unique size and the pore size characteristics of the selected column.

Coupling the SEC with MALS or SEC/MALS provides accurate distribution of molar mass and size (root mean square radius) as opposed to relying on a set of SEC calibration standards. This type of arrangement has many advantages over traditional column calibration methods. Since the light scattering and concentration are measured for each eluting fraction, the molar mass and size can be determined independently of the elution position. This is particularly relevant for species with non-globular shaped macromolecules such as the biopolymers (OG1802) or bioconjugates (OG1953); such species typically do not elute in a manner that might be described by a set of column calibration standards.

In some embodiments, a SEC/MALS analysis includes a Waters HPLC system with Alliance 2695 solvent delivery module and Waters 2996 Photodiole Array Detector equipped with a Shodex SEC-HPLC column (7.8×300 mm). This is connected online with a Wyatt miniDawn TREOS and Wyatt Optilab T-rEX differential refractometer. The Empower software from Waters can be used to control the Waters HPLC system and the ASTRA V 6.1.7.16 software from Wyatt can be used to acquire the MALS data from the Wyatt miniDawn TREOS, dn/dc data from the T-rEX detector and the mass recovery data using the A280 absorbance signal from the Waters 2996 Photodiole Array detector. SEC can be carried out at 1 ml/min in 1×PBS pH 7.4, upon sample injection, the MALS and RI signals can be analyzed by the ASTRA software for determination of absolute molar mass (Mp, Mw, Mn) and polydisperse index (PDI). In addition, the calculation also involves the input dn/dc values for polymer and protein as 0.142 and 0.183, respectively. For OG1953 bioconjugates dn/dc value, the dn/dc is calculated based on the weighted MW of the polymer and the protein to be about 0.148 using the formula below:

Conjugate dn/dc=0.142×[MWpolymer/(MWpolymer+MWprotein)]+0.183×[MWprotein/(MWpolymer+MWprotein)]
where MWpolymer for OG1802 is 800 kDa and the MWprotein for OG1950 is 146 kDa.

“Isocratic” refers to a mobile phase kept constant and uniform during a chromatography run.

### Antibody Compositions and Methods

In some embodiments, a formulation (or therapeutically acceptable composition) that is a first protein that includes a first protein or protein moiety that is conjugated to a polymer (e.g., a phosphorylcholine-containing polymer) and a second protein or protein moiety that is unconjugated is provided. In some embodiments, the first protein is an antibody and the second protein is an antibody. Both antibodies can be therapeutic antibodies. In some embodiments, the formulation or composition is for treating an eye disorder in a subject. In some embodiments, a formulation or composition sample includes at least a polymer (e.g., a phosphorylcholine-containing polymer) and an unconjugated protein (e.g., an unconjugated antibody). As used herein, formulation (e.g., mixed formulation) and composition (e.g., mixed therapeutically acceptable composition) can be used interchangeably. In some embodiments, a formulation or therapeutically acceptable composition is safe for human use (e.g., administering to a human). In some embodiments, a formulation or therapeutically acceptable composition is not an intermediate product generated during manufacture of a final product (e.g., that may be suitable for use in a human). In some embodiments, the formulation is a mixed formulation sample. In some embodiments the terms formulation and mixed formulation sample are used interchangeably. As used herein, “protein” and “protein moiety” can be used interchangeably.

In some embodiments, provided herein is a formulation (or therapeutically acceptable composition) comprising any two proteins that can be the same or different in function, wherein one is conjugated to a polymer and the other is not conjugated to the polymer (or conjugated to any polymer or conjugated to any effective amount of a polymer). The formulation (or therapeutically acceptable composition) can be for the treatment of an eye disorder. In some embodiments, both of the proteins are therapeutic proteins for the treatment of an eye disorder. In some embodiments, one or both of the proteins are therapeutics, antibodies and/or therapeutic antibodies. In some embodiments, one antibody is conjugated to a polymer and the other antibody is not conjugated to a polymer. In some embodiments, the antibody may be synthesized. In some embodiments, the antibody may be a native sequence antibody. In some embodiments, the antibody may be a Fab fragment. In some embodiments, the antibody may be a Trap fragment. In some embodiments, the antibody may be a fusion protein such as a Trap-antibody fusion protein. In some embodiments, the antibody may be a peptide fragment. In some embodiments, a non-antibody scaffold protein can be used instead of an antibody.

Provided herein is a formulation (or therapeutically acceptable composition) comprising: a first molar amount of a conjugate comprising a first protein or protein moiety conjugated to a phosphorylcholine-containing polymer (the “conjugated protein”); a second molar amount of a second protein or protein moiety that is not conjugated to a phosphorylcholine-containing polymer (the “unconjugated protein”); and a pharmaceutically acceptable carrier, wherein the formulation, or composition, comprises the second protein at about 1% or more of a total molar amount of the conjugate and the second protein, wherein the total molar amount comprises a sum of the first molar amount and the second molar amount, wherein the formulation, or composition, has a pH that is about 0.5 pH units away (e.g., above or below) or more from the isoelectric point (pI) of the second protein. As used herein, “molar amount” denotes a measure of the molar quantity of a molecule. In some embodiments, molar amount is a molar concentration (e.g., M, mM, μM, nM, etc.). In some embodiments, molar amount is expressed in units of moles (e.g., moles, millimoles, micromoles, etc.). As used herein, the isoelectric point (pI) of a protein has its customary and ordinary meaning to one of ordinary skill in the art, in view of the present disclosure. The pI denotes the pH at which the protein carries no net charge. The pI can be a previously known value for the same or similar protein, or be determined based on a model, or empirically. In some embodiments, the pI is a theoretically determined pI. In some embodiments, the pI is an empirically determined pI.

Low-viscosity formulation (or therapeutically acceptable composition) of a protein conjugate (e.g., a protein conjugated to a phosphorylcholine-containing polymer) are also provided. A high concentration of the protein conjugate in the formulation, or composition, can raise the viscosity of the formulation, or composition. In some embodiments, lowering the viscosity of the formulation or composition (while maintaining the total amount of active protein) improves one or more of manufacturability, handling, and injectability, for example when delivering the formulation or composition with a syringe to a site of treatment (e.g., intraocular administration).

In any of the methods and apparatuses of the present disclosure, mixed formulations (or therapeutically acceptable compositions) (e.g., having a polymer or polymer-conjugated protein and unconjugated protein) can include the unconjugated protein or protein moiety (e.g., protein that is not conjugated to the phosphorylcholine-containing polymer) at any suitable % molar amount of the total molar amount of the polymer/polymer conjugate and the unconjugated protein or protein moiety. In some embodiments, the formulations (or therapeutically acceptable compositions) comprise the second protein or protein moiety (the unconjugated protein) at about 1% or more of a total molar amount of the conjugate and the second protein, wherein the total molar amount comprises a sum of the first molar amount of the conjugate and the second molar amount of the second protein. For example, where the combined concentration of the conjugate and the unconjugated protein is 100 μM, the unconjugated protein at 1% of the total molar amount is at 1 μM, and the conjugate is at 99 μM. In some embodiments, the formulations (or therapeutically acceptable composition) comprise the second protein (the unconjugated protein) at, or at about 1% or more, about 2% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, or about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, or a percentage in a range defined by any two of the preceding values (e.g., about 1-95%, 1-90%, 1-80%, 1-95%, 1-50%, 5-50%, 10-40%, 15-35%, 15-25%, 25-35%, 25-40%, 40-95%, 50-80%, etc.) of a total molar amount of the conjugate and the second protein, wherein the total molar amount comprises a sum of the first molar amount of the conjugate and the second molar amount of the second protein. In some embodiments, the formulations (or therapeutically acceptable composition) comprise the second protein (the unconjugated protein) at between about 5% and about 50%, or between about 15% and about 30% of a total molar amount of the conjugate and the second protein, wherein the total molar amount comprises a sum of the first molar amount of the conjugate and the second molar amount of the second protein. In some embodiments, the formulation (or therapeutically acceptable composition) comprises the second protein (the unconjugated protein) at about 20% of a total molar amount of the conjugate and the second protein, wherein the total molar amount comprises a sum of the first molar amount of the conjugate and the second molar amount of the second protein. In some embodiments, the formulation (or therapeutically acceptable composition) comprises the second protein (the unconjugated protein) at about 30% of a total molar amount of the conjugate and the second protein, wherein the total molar amount comprises a sum of the first molar amount of the conjugate and the second molar amount of the second protein. In some embodiments, any formulation or composition provided herein comprises the second protein (the unconjugated protein) at more than 5% of a total molar amount of the conjugate and the second protein, wherein the total molar amount comprises a sum of the first molar amount of the conjugate and the second molar amount of the second protein. In some embodiments, any formulation or composition provided herein comprises the second protein (the unconjugated protein) at more than 10% of a total molar amount of the conjugate and the second protein, wherein the total molar amount comprises a sum of the first molar amount of the conjugate and the second molar amount of the second protein. In some embodiments, any composition or formulation herein includes two or more (e.g., 2, 3, 4, 5 or more) different second proteins (or unconjugated proteins), where the second molar amount is the sum of the molar amounts of the two or more different second proteins.

In some embodiments a formulation (or therapeutically acceptable composition) includes: a conjugate comprising a first protein or protein moiety conjugated to a phosphorylcholine-containing polymer, wherein the polymer has 9 arms and a molecular weight of between 600,000 and 1,000,000 Da, wherein the polymer is present in the formulation at about 100 mg/mL or more; and a second protein or protein moiety that is not conjugated to a polymer, wherein the second protein or protein moiety is present in the formulation at 5-15 mg/mL. In some embodiments, the first and second proteins are a therapeutic protein. In some embodiments, the first protein and second protein are the same (e.g., are at least, or at least about 85%, 90%, 95%, 97%, 99%, or are about 100% identical in amino acid sequence). In some embodiments, the first protein and second protein are different proteins.

In some embodiments, a mixed formulation (or therapeutically acceptable composition) includes: a first molar amount of a conjugate comprising a first protein or protein moiety conjugated to a polymer; and a second molar amount of a second protein or protein moiety that is not conjugated to a polymer, wherein the formulation comprises the second protein at about 1% or more of a total molar amount of the first protein and the second protein, wherein the total molar amount comprises a sum of the first molar amount and the second molar amount. In some embodiments, the formulation comprises the second protein at about 1-90%, about 5-90%, about 5-80%, about 10-95%, about 15-30%, about 5-50%, or about 10-40%, of the total molar amount of the conjugate and the second protein. In some embodiments, the polymer is a phosphorylcholine-containing polymer. In some embodiments, the formulation comprises the second protein at about 5-50%, or about 15-30% of the total molar amount of the conjugate and the second protein. In some embodiments, the polymer is a phosphorylcholine-containing polymer.

In some embodiments, a mixed formulation (or therapeutically acceptable composition) includes: a conjugate comprising a first protein or protein moiety conjugated to a polymer; and a second protein that is not conjugated to a polymer, wherein a first molar amount of the conjugate and a second molar amount of the second protein or protein moiety has been combined in the formulation such that the second molar amount is about 1% or more (e.g., about 5-90%, 15-25%, 25-35%, 25-40%, etc.) of a total molar amount of the conjugate and the second protein or protein moiety, wherein the total molar amount comprises a sum of the first molar amount and the second molar amount. In some embodiments, the mixed formulation or composition is prepared by combining the first molar amount of the conjugate with the second molar amount of the second protein that is not conjugated to a polymer, such that the second molar amount is at the specified percentage of the sum of the first molar amount and the second molar amount (e.g., specified percentage of the total molar amount). In some embodiments, the second molar amount is about 1-90%, about 5-90%, about 5-80%, about 10-95%, about 15-30%, about 5-50%, or about 10-40%, of the total molar amount of the conjugate and the second protein. In some embodiments, the second molar amount is about 5-50% of the total molar amount of the conjugate and the second protein. In some embodiments, the second molar amount is about 15-30% of the total molar amount of the conjugate and the second protein. In some embodiments, the polymer is a phosphorylcholine-containing polymer.

In some embodiments, the mixed formulation or composition has been prepared by combining the conjugate at a percent composition of about 1% or more (e.g., about 5-93%, 15-25%, 25-35%, 25-40%, etc.) of the second protein relative to the total protein mass weight concentration of the first protein and the second protein, where the remainder of the total protein mass weight concentration includes the first protein. For example, for a total mass weight concentration of 50 mg/mL, the mixed therapeutically acceptable composition can be prepared by combining an amount of the second protein that corresponds to 10 mg/mL in the final composition (at percent composition of 20%) with an amount of the conjugate that corresponds to 40 mg/mL of the first protein (as the conjugate, excluding any contribution of the polymer to the mass weight concentration calculation) in the final composition.

In some embodiments, the conjugate includes a first protein or protein moiety conjugated to a polymer, wherein the polymer includes one or more of: polyethylene glycol (PEG), branched PEG, PolyPEG® (Warwick Effect Polymers; Coventry, UK), polysialic acid (PSA), starch, hydroxylethyl starch (HES), hydroxyalkyl starch (HAS), carbohydrate, polysaccharides, pullulane, chitosan, hyaluronic acid, chondroitin sulfate, dermatan sulfate, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyoxazoline, polyethylene-co-maleic acid anyhydride, polystyrene-co-maleic acid anhydride, poly(l-hydroxymethyethylene hydroxymethylformal) (PHF), a zwitterionic polymer, a phosphorylcholine containing polymer and a polymer comprising MPC, Poly (Glyx-Sery), Hyaluronic acid (HA), Heparosan polymers (HEP), Fleximers, Dextran, and Poly-sialic acids (PSA).

### General

Provided herein are methods and apparatus for analyzing mixed formulations (or therapeutically acceptable compositions) of protein and protein conjugated to polymer by tandem HPLC. In some embodiments, the proteins are antibodies. In some embodiments, the antibodies are anti-VEGF antibodies and conjugates thereof. In some embodiments, the antibodies themselves are different from other anti-VEGF agents and provide superior results over other anti-VEGF agents. In some embodiments, the anti-VEGF antibody conjugate displays a surprising superiority over other antibodies and/or the expectation of the activity other antibody conjugates.

Historically, conjugating a molecule to a protein often resulted in a decrease in the protein's binding interaction to its intended target. In some embodiments of the present disclosure, when conjugating to a location that is outside of the active site, the same level of decrease as might have been expected is not necessarily observed. The evidence provided herein shows the opposite effect as to what may have been expected. In some embodiments, and without intending to be limited by theory, the conjugate can be superior to the antibody alone. For example, the interaction of a ligand and its specific receptor is often driven through the stereospecific interaction of the ligand and the receptor, as directed by the interactions of the hydrophilic amino acids on the ligand with the hydrophilic amino acids on the receptor, and water molecules are front and center in those interactions. At the same time, this hydrophilic stereospecificity is further enhanced by de-emphasizing and/or suppressing non-specific hydrophobic interactions that might generally be mediated/created by hydrophobic-to-hydrophobic amino acids.

In some embodiments, an anti-VEGF antibody conjugate is provided that is capable of blocking at least 90% of an interaction between a VEGF ligand (“VEGFL”) and a VEGF-receptor (“VEGFR”). For example, it can block at least 90, 91, 92, 93, 94, 95, 96, 97, 98, 99, or effectively all of the interaction between VEGFR and VEGFL. In some embodiments, the noted blocking occurs at saturating concentrations. In some embodiments, an anti-VEGF antibody conjugate is provided that blocks at least 95% of an interaction between a VEGF ligand and a VEGF-receptor. Indeed, this result was unexpected in that while the addition of a polymer to an antibody (to form an antibody conjugate), could be expected to have some or no detrimental impact on binding/activity of the antibody, it was unexpected that it would actually improve the blocking ability of the antibody in this manner.

In some embodiments, the antibodies or conjugates thereof inhibit at least 70, 80, 85, 86, 87, 88, 89, 90, 91, 92, 93, 94, 95, 96, 97, 98, 99, or 100% of the activity and/or interaction between VEGFR and VEGFL. In some embodiments, the IC50 value can be 0.1, 1, 2, 3, 4, 5, 6, 7, 8, 9, 10, 20, 30, 40, 50, 100 nM or less than any one or more of the preceding values. In some embodiments, the KD can be 2*10\hat{ }−13, 1*10\hat{ }−13, 1*10\hat{ }−12, 1*10\hat{ }−11, 1*10-\hat{ }10M or less than any one of the preceding values. In some embodiments, the IC50 value can be 1, 5, 10, 20, 30, 40, 50, 60, 70 80, 90, 100, 200, 300, 400, 500, 600, 700, 800, 900, 1,000, 1,100, 1,200, 1,300, or less than any one of the preceding values.

In some embodiments, an anti-VEGF antibody is provided that blocks at least 90% of an interaction between a VEGF ligand and a VEGF-receptor. For example, it can block at least 91, 92, 93, 94, 95, 96, 97, 98, 99, or effectively all of the interaction between VEGFR and VEGFL.

In some embodiments, other antibodies, such as Lucentis® (ranibizumab) or Avastin® (bevacizumab) can be conjugated to one or more of the polymers as described herein, by one or more of the processes described herein. In some embodiments, any antibody, or fragment thereof, can be conjugated to one or more of the polymers as described herein, by one or more of the processes described herein.

In some embodiments the antibody comprises a heavy chain amino acid variable region that comprises SEQ ID NO: 1 (with or without the C-terminal lysine) and a light chain amino acid variable region that comprises SEQ ID NO: 2. In some embodiments, the antibody is conjugated to one or more of the polymers provided herein. In some embodiments, the conjugated antibody is at least 90% identical to SEQ ID NO: 1 and/or 2. In some embodiments, the antibody contains the 6 CDRs within SEQ ID NO:1 and SEQ ID NO: 2, as well as a point mutation of L443C (EU numbering, or 449C in SEQ ID NO: 1). In some embodiments, the conjugated antibody is at least 90% identical to SEQ ID NO: 1 and/or 2 and includes the following mutations: L234A, L235A, and G237A (EU numbering), and at least one of the following mutations: Q347C (EU numbering) or L443C (EU numbering).

In some embodiments an antibody that binds to VEGF-A is provided. The antibody comprises: a CDRH1 that is the CDRH1 in SEQ ID NO: 1, a CDRH2 that is the CDRH2 in SEQ ID NO: 1, a CDRH3 that is the CDRH3 in SEQ ID NO: 1, a CDRL1 that is the CDRL1 in SEQ ID NO: 2, a CDRL2 that is the CDRL2 in SEQ ID NO: 2, a CDRL3 that is the CDRL3 in SEQ ID NO: 2, at least one of the following mutations: L234A, L235A, and G237A (EU numbering), and at least one of the following mutations: Q347C (EU numbering) or L443C (EU numbering).

As will be appreciated by one of skill in the art, in light of the present specification, any of the antibodies provided herein can be conjugated to any of the polymers provided herein and/or any antibody provided herein can have a cysteine added such that it allows for site specific conjugation to a polymer.

“VEGF” or “vascular endothelial growth factor” is a human vascular endothelial growth factor that affects angiogenesis or an angiogenic process. In particular, the term VEGF means any member of the class of growth factors that (i) bind to a VEGF receptor such as VEGFR-1 (Flt-1), VEGFR-2 (KDR/Flk-1), or VEGFR-3 (FLT-4); (ii) activates a tyrosine kinase activity associated with the VEGF receptor; and (iii) thereby affects angiogenesis or an angiogenic process.

The VEGF family of factors is made up of five related glycoproteins: VEGF-A (also known as VPE), -B, -C, -D and PGF (placental growth factor). Of these, VEGF-A is the most well studied and is the target of anti-angiogenic therapy. Ferrara et al, (2003) Nat. Med. 9:669-676. VEGF-A exists as a number of different isotypes which are generated both by alternative splicing and proteolysis: VEGF-A206, VEGF-A189, VEGF-A165, and VEGF-A121. The isoforms differ in their ability to bind heparin and non-signaling binding proteins called neuropilins. The isoforms are all biologically active as dimers.

The various effects of VEGF are mediated by the binding of a VEGF, e.g., VEGF-A (P15692), -B (P49766), -C(P49767) and -D (Q43915), to receptor tyrosine kinases (RTKs). The VEGF family receptors belong to class V RTKs and each carry seven Ig-like domains in the extracellular domain (ECD). In humans, VEGF binds to three types of RTKs: VEGFR-1 (Flt-1) (P17948), VEGFR-2 (KDR, Flk-1) (P935968) and VEGFR-3 (Flt-4) (P35916). Unless otherwise apparent from the context reference to a VEGF means any of VEGF-A, -B, -C, -D, and PGF, in any of the natural isoforms or natural variants or induced variants having at least 90, 95, 98 or 99% or 100% sequence identity to a natural form. In some embodiments, such VEGFs are human VEGFs. Likewise reference to a VEGFR means any of VEGR-1, R-2 or R-3, including any natural isoform or natural variant, or an induced variant having at least 90, 95, 98 or 99% or 100% sequence identity to a natural sequences.

VEGF antagonist therapies have been approved for the treatment of certain cancers and wet AMD. Bevacizumab (AVASTIN, Genentech/Roche) is a humanized mouse monoclonal antibody that binds to and neutralizes human VEGF, in particular to all isoforms of VEGF-A and to bioactive proteolytic fragments of VEGF-A. See, e.g., Ferrara N, Hillan K J, Gerber H P, Novotny W. 2004. Discovery and development of bevacizumab, an anti-VEGF antibody for treating cancer. Nat Rev Drug Discov. 3 (5): 391-400. Bevacizumab has been approved for the treatment of certain cancers. The protein sequence of the heavy and light chains of bevacizumab (DrugBank DB00112) are set forth in SEQ ID NO: 3 (heavy) and SEQ ID NO: 4 (light).

Bevacizumab variable light chain CDRs are CDRL1: SASQDISNYLN (SEQ ID NO: 12), CDRL2: FTSSLHS (SEQ ID NO: 13) and CDRL3: QQYSTVPWT (SEQ ID NO: 14). Bevacizumab variable heavy chain CDRs are CDRH1: GYTFTNYGMN, CDRH2: WINTYTGEPTYAADFKR (SEQ ID NO: 10), and CDRH3: YPHYYGSSHWYFDV. CDRs are defined by Kabat except CDRH1 uses the composite Kabat/Chothia definition. In some embodiments, a cysteine can be added to the Bevacizumab sequence and the antibody (and/or a variant that includes the 6 CDRs of Bevacizumab) can be conjugated to any one or more of the polymers provided herein.

Another anti-VEGF molecule, derived from the same mouse monoclonal antibody as bevacizumab has been approved as a treatment for wet AMD: ranibizumab (LUCENTIS® (ranibizumab), Genentech/Roche). Ranibizumab is an antibody fragment or Fab. Ranibizumab was produced by affinity maturation of the variable heavy and light chains of bevacizumab. The sequence of the heavy and light chains of ranibizumab (as published by Novartis) is set forth in SEQ ID NO: 5 and 6 respectively. In some embodiments, a cysteine can be added to the ranibizumab sequence and the antibody (and/or a variant that includes the 6 CDRs of ranibizumab) can be conjugated to any one or more of the polymers provided herein.

The Ranibizumab CDRS are the same as Bevacizumab except where an improvement was added after affinity maturation: Ranibizumab variable light chain CDRs are CDRL1: SASQDISNYLN (SEQ ID NO: 12), CDRL2: FTSSLHS (SEQ ID NO: 13) and CDRL3: QQYSTVPWT (SEQ ID NO: 14). Ranibizumab variable heavy chain CDRs are CDRH1: GYDFTHYGMN (SEQ ID NO: 9), CDRH2: WINTYTGEPTYAADFKR (SEQ ID NO: 10), and CDRH3: YPYYYGTSHWYFDV (SEQ ID NO: 11).

In some embodiments, an antibody conjugate is presented having an anti-VEGF-A antibody bonded at a cysteine outside a variable region of the antibody to a phosphorylcholine containing polymer, wherein the cysteine has been added via recombinant DNA technology. In some embodiments, the polymer is bonded to a single cysteine. In some embodiments, “added by recombinant DNA technology” means that the cysteine residue replaces a non-cysteine amino acid that occurs in the same position in a known or existing antibody or in a consensus antibody sequence. Thus, for example where the antibody is an IgG1 and the heavy chain possess a leucine at EU position 443, the leucine is replaced via recombinant DNA technology with a cysteine (L443C, EU numbering, or 449C in SEQ ID NO: 1). Correspondingly, the native IgG1 sequence at EU position 347 is Q (glutamine) and the Q is replaced with cysteine via recombinant DNA technology to yield Q347C.

In some embodiments, the anti-VEGF-A antibody comprises a light chain and a heavy chain where the heavy chain has an Fc region. In some embodiments, the cysteine is in the Fc region and the anti-VEGF-A antibody is an immunoglobulin G (IgG). In some embodiments, the anti-VEGF-A heavy chain has CDRH1: GYDFTHYGMN (SEQ ID NO: 9), CDRH2: WINTYTGEPTYAADFKR (SEQ ID NO: 10), and CDRH3: YPYYYGTSHWYFDV (SEQ ID NO: 11), and position 221 (via sequential counting as in SEQ ID NO: 3) is T, and the anti-VEGF-A light chain has CDRL1: SASQDISNYLN (SEQ ID NO: 12), CDRL2: FTSSLHS (SEQ ID NO: 13), and CDRL3: QQYSTVPWT (SEQ ID NO: 14), and Kabat position 4 is L.

In some embodiments, the anti-VEGF-A heavy chain isotype is IgG1. In some embodiments, the IgG1 constant domain has one or more mutations relative to an IgG1 constant domain (e.g. constant region of SEQ ID NO: 3) to modulate effector function. In some embodiments, the effector function mutations are one or more of the following: (EU numbering) E233X, L234X, L235X, G236X, G237X, A327X, A330X, and P331X wherein X is any natural or unnatural amino acid. In some embodiments, the mutations are selected from the group comprising (EU numbering): E233P, L234V, L234A, L235A, G237A, A327G, A330S, and P331S. In some embodiments, antibody conjugate has the following mutations (EU numbering): L234A, L235A, and G237A.

In some embodiments, the cysteine residue is in the anti-VEGF-A heavy chain and is Q347C (EU numbering) or L443C (EU numbering). In some embodiments, the cysteine residue is L443C (EU numbering, or 449C in SEQ ID NO: 1). In some embodiments, the sequence of the anti-VEGF-A heavy chain is SEQ ID NO: 1 (with or without the C-terminal lysine) and the sequence of the anti-VEGF-A light chain is SEQ ID NO: 2.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an anti-HTRA1 antibody. In some embodiments, the anti-HTRA1 antibody includes a heavy chain comprising a complementarity determining region 1 CDRH1: FYHVH (SEQ ID NO: SEQ ID NO:140), CDRH2: SIYTSGYTEYASALES (SEQ ID NO:141), and CDRH3: EGLQRVGVLDA (SEQ ID NO:142) or EGLQRVGVFDA (SEQ ID NO:143) or EGLQRVGVMDA (SEQ ID NO:144), and a light chain comprising a CDRL1: RSSQSLLDEAGETYLA (SEQ ID NO:145), CDRL2: EVSLLES (SEQ ID NO:146), and CDRL3: QQATYFPYT (SEQ ID NO:147). In some embodiments, the anti-HTRA1 antibody includes a heavy chain comprising a complementarity determining region 1 CDRH1: GFSLTFYH (SEQ ID NO: SEQ ID NO:148), CDRH2: IYTSGYT (SEQ ID NO:149), and CDRH3: AREGLQRVGVFDA (SEQ ID NO:150) or AREGLQRVGVMDA (SEQ ID NO: 151) or AREGLQRVGVLDA (SEQ ID NO:152), and a light chain comprising a CDRL1: QSLLDEAGETY (SEQ ID NO:153), CDRL2: EV, and CDRL3: QQATYFPYT (SEQ ID NO: 147). In some embodiments, the anti-HTRA1 antibody includes a heavy chain comprising a complementarity determining region 1 CDRH1: GFSLTFY (SEQ ID NO: SEQ ID NO:154), CDRH2: YTSGY (SEQ ID NO:155), and CDRH3: EGLQRVGVLDA (SEQ ID NO:142) or EGLQRVGVFDA (SEQ ID NO:143) or EGLQRVGVMDA (SEQ ID NO:144), and a light chain comprising a CDRL1: RSSQSLLDEAGETYLA (SEQ ID NO:145), CDRL2: EVSLLES (SEQ ID NO:146), and CDRL3: QQATYFPYT (SEQ ID NO:147). In some embodiments, the heavy chain comprises an amino acid sequence at least 80%, at least 85%, at least 90%, at least 95%, at least 97% or about 100%, or a percentage in a range defined by any two of the preceding values (e.g., 80-100%, 85-95%, 90-97%, etc.) identical to any one of the VH sequences set forth in TABLE 1, and the light chain comprises an amino acid sequence at least 80%, at least 85%, at least 90%, at least 95%, at least 97% or about 100%, or a percentage in a range defined by any two of the preceding values (e.g., 80-100%, 85-95%, 90-97%, etc.) identical to the VL sequence set forth in TABLE 2. In some embodiments, the heavy chain comprises any one of the VH sequences set forth in TABLE 1, and the light chain comprises the VL sequence set forth in TABLE 2.

TABLE 1

| ID | Sequence |
|---|---|
| HC17 | QVQLVESGGGVVQPGGSLRLSCAASGFSLTFYHVHWVRQAPGKGLEWVASIYTSGYTEYASA |
| | LESRFTISRDTSKSTLYLQMNSLRAEDTAVYYCAREGLQRVGVFDAWGQGTLVTVSS |
| | (SEQ ID NO: 156) |
| HC37 | QVQLVESGGGVVQPGGSLRLSCAASGFSLTFYHVHWVRQAPGKGLEWVASIYTSGYTEYASA |
| | LESRFTISRDTSKSTLYLQMNSLRAEDTAVYYCAREGLQRVGVMDAWGQGTLVTVSS |
| | (SEQ ID NO: 157) |
| HC39 | QVQLVESGGGVVQPGGSLRLSCAASGFSLTFYHVHWVRQAPGKGLEWVASIYTSGYTEYASA |
| | LESRFTISRDTSKSTLYLQMNSLRAEDTAVYYCAREGLQRVGVLDAWGQGTLVTVSS |
| | (SEQ ID NO: 158) |
| HC40 | EVQL VESGGGVVQPGGSLRLSCAASGFSLTFYHVHWVRQAPGKGLEWVASIYTSGYTEYASAL |
| | ESRFTISRDTSKSTLYLQMNSLRAEDTAVYYCAREGLQRVGVMDAWGQGTLVTVSS |
| | (SEQ ID NO: 159) |
| HC41 | EVQLVESGGGVVQPGGSLRLSCAASGFSLTFYHVHWVRQAPGKGLEWVASIYTSGYTEYASAL |
| | ESRFTISRDTSKSTLYLQMNSLRAEDTAVYYCAREGLQRVGVLDAWGQGTLVTVSS |
| | (SEQ ID NO: 160) |

TABLE 2

| ID | Sequence |
|---|---|
| LC | DIQLTQSPSSLSASVGDRVTITCRSSQSLLDEAGETYLAWFQQ |
| 21 | KPGKAPKLLIYEVSLLESGVPSRFSGSGSGTDFTLTISSLQPE |
| | DFATYYCQQATYFPYTFGQGTKVEIK |
| | (SEQ ID NO: 161) |

In some embodiments, the first or second antibody of the mixed formulation (or therapeutically acceptable composition) comprises a Complement Factor D (CFD) antibody. In some embodiments, the CFD antibody includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 129 (or a heavy chain variable region thereof), and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 130 (or a light chain variable region thereof). In some embodiments, the CFD antibody heavy chain can comprise the sequence of SEQ ID NO: 129 (with or without the C-terminal lysine). In some embodiments, the CFD antibody light chain can comprise the sequence of SEQ ID NO: 130.

In some embodiments, the first or second antibody of the mixed formulation (or therapeutically acceptable composition) comprises a fusion protein that is a VEGF-A trap fused to an IL-6 antibody. In some embodiments, the VEGF-A trap fused to an IL-6 antibody can comprise a heavy chain comprising SEQ ID NO: 131 (with or without the C-terminal lysine). In some embodiments, the VEGF-A trap fused to an IL-6 antibody can comprise a light chain comprising SEQ ID NO: 132.

In some embodiments, the VEGF Trap comprises human VEGFR1 domain 2 and human VEGFR2 domain 3. In some embodiments, the VEGF Trap includes the amino acid sequence of SEQ ID NO:133. In some embodiments, the VEGF Trap comprises an amino acid sequence at least 80%, at least 85%, at least 90%, at least 95%, at least 97%, at least 99%, or about 100%, or a percentage in a range defined by any two of the preceding values (e.g., 80-100%, 85-95%, 90-97%, etc.) identical to SEQ ID NO:133 form in TABLE 3.

TABLE 3

| | SEQ ID |
|---|---|
| Sequence | NO: |
| SDTGRPFVEMYSEIPEIIHMTEGRELVIPCRVTSPNITVTL | 133 |
| KKFPLDTLIPDGKRIIWDSRKGFIISNATYKEIGLLTCEAT | |
| VNGHLYKTNYLTHRQTNTIIDVVLSPSHGIELSVGEKLVLN | |
| CTARTELNVGIDENWEYPSSKHQHKKLVNRDLKTQSGSEMK | |
| KFLSTLTIDGVTRSDQGLYTCAASSGLMTKKNSTFVRVHEK | |

In some embodiments a fusion construct includes a heavy chain that includes a CDRH1 that is the CDRH1 in SEQ ID NO: 105; a CDRH2 that is the CDRH2 in SEQ ID NO: 105; a CDRH3 that is the CDRH3 in SEQ ID NO: 105; a CDRL1 that is the CDRL1 in SEQ ID NO: 106; a CDRL2 that is the CDRL2 in SEQ ID NO: 106; and a CDRL3 that is the CDRL3 in SEQ ID NO: 106. In some embodiments, the fusion construct includes a heavy chain comprising a complementarity determining region 1 (CDRH1): PFAMH (SEQ ID NO: 134), CDRH2: KISPGGSWTYYSDTVTD (SEQ ID NO: 135), and CDRH3: QAWGYYALDI (SEQ ID NO: 136); and a light chain comprising CDRL1: SASISVSYLY (SEQ ID NO: 137), CDRL2: DDSSLAS (SEQ ID NO: 138), and CDRL3: QQWSGYPYT (SEQ ID NO: 139). In some embodiments, the heavy chain comprises an amino acid sequence at least 80%, at least 85%, at least 90%, at least 95%, at least 97% at least 99%, or about 100%, or a percentage in a range defined by any two of the preceding values (e.g., 80-100%, 85-95%, 90-97%, etc.) identical to SEQ ID NO:105, and the light chain comprises an amino acid sequence at least 80%, at least 85%, at least 90%, at least 95%, at least 97% at least 99%, or about 100%, or a percentage in a range defined by any two of the preceding values (e.g., 80-100%, 85-95%, 90-97%, etc.) identical to SEQ ID NO:106. In some embodiments, the heavy chain comprises the amino acid sequence of SEQ ID NO: 105 (with or without the C-terminal lysine), and the light chain comprises the amino acid sequence of SEQ ID NO:106.

In some embodiments, the phosphorylcholine containing polymer comprises 2-(methacryloyloxyethyl)-2′-(trimethylammonium)ethyl phosphate (MPC) monomers as set forth below:

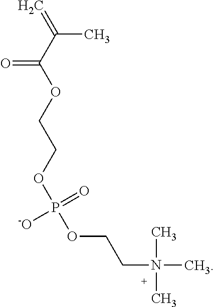

Such that the polymer comprises the following repeating units:

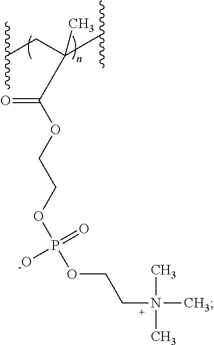

where n is an integer from 1 to 3000 and the wavy lines indicate the points of attachment between monomer units in the polymer.

In some embodiments, the polymer has three or more arms, or is synthesized with an initiator comprising 3 or more polymer initiation sites. In some embodiments, the polymer has 2, 3, 4, 5, 6, 7, 8, 9, 10, 11 or 12 arms, or is synthesized with an initiator comprising 2, 3, 4, 5, 6, 7, 8, 9, 10, 11, or 12 polymer initiation sites. More preferably, the polymer has 3, 6, or 9 arms, or is synthesized with an initiator comprising 3, 6, or 9 polymer initiation sites. In some embodiments, the polymer has 9 arms, or is synthesized with an initiator comprising 9 polymer initiation sites.

In some embodiments, the polymer that is added has a molecular weight between about 300,000 and about 1,750,000 Da (SEC-MALs). In some embodiments, the polymer has a molecular weight between about 500,000 and about 1,000,000 Da. In some embodiments, the polymer has a molecular weight of between about 600,000 to about 1,000,000 Da. In some embodiments, the polymer has a molecular weight of between about 750,000 to about 850,000 Da. In some embodiments, the polymer has a molecular weight of between about 800,000 to about 850,000 Da. In some embodiments, the polymer has a molecular weight of between about 750,000 to about 800,000 Da. In some embodiments, the polymer has a molecular weight in the range of about 700,000 to about 800,000 Da.

In some embodiments, any of the antibodies described herein can be further conjugated to a polymer to form a bioconjugate. The molecular weight of the bioconjugate (in total, SEC-MALs) can be between about 350,000 and 2,000,000 Daltons, for example, between about 450,000 and 1,900,000 Daltons, between about 550,000 and 1,800,000 Daltons, between about 650,000 and 1,700,000 Daltons, between about 750,000 and 1,600,000 Daltons, between about 850,000 and 1,500,000 Daltons, between about 900,000 and 1,400,000 Daltons, between about 950,000 and 1,300,000 Daltons, between about 900,000 and 1,000,000 Daltons, between about 1,000,000 and 1,300,000 Daltons, between about 850,000 and 1,300,000 Daltons, between about 850,000 and 1,000,000 Daltons, and between about 1,000,000 and 1,200,000 Daltons.

In some embodiments, the antibody conjugate is purified. In some embodiments, the polymer is aspect of the antibody conjugate is polydisperse, i.e. the polymer PDI is not 1.0. In some embodiments, the PDI is less than 1.5. In some embodiments, the PDI is less than 1.4. In some embodiments, the PDI is less than 1.3. In some embodiments the PDI is less than 1.2. In some embodiments the PDI is less than 1.1.

In some embodiments, the antibody conjugate has an anti-VEGF-A immunoglobulin G (IgG) bonded to a polymer, which polymer comprises MPC monomers, wherein the sequence of the anti-VEGF-A heavy chain is SEQ ID NO: 1 (with or without the C-terminal lysine), and the sequence of the anti-VEGF-A light chain is SEQ ID NO: 2, and wherein the antibody is bonded only at C449 in SEQ ID NO: 1 to the polymer. In some embodiments, the polymer has 9 arms and has a molecular weight of between about 600,000 to about 1,000,000 Da.

In some embodiments, the antibody conjugate has an anti-VEGF-A immunoglobulin G (IgG) bonded to a polymer, which polymer comprises MPC monomers, wherein the sequence of the anti-VEGF-A heavy chain is SEQ ID NO: 1 (with or without the C-terminal lysine), and the sequence of the anti-VEGF-A light chain is SEQ ID NO: 2, and wherein the antibody is bonded only at C443 (EU numbering, or 449C in SEQ ID NO: 1) to the polymer. In some embodiments, the polymer has 9 arms and has a molecular weight of between about 600,000 to about 1,000,000 Da.

In some embodiments, the antibody conjugate has the following structure:

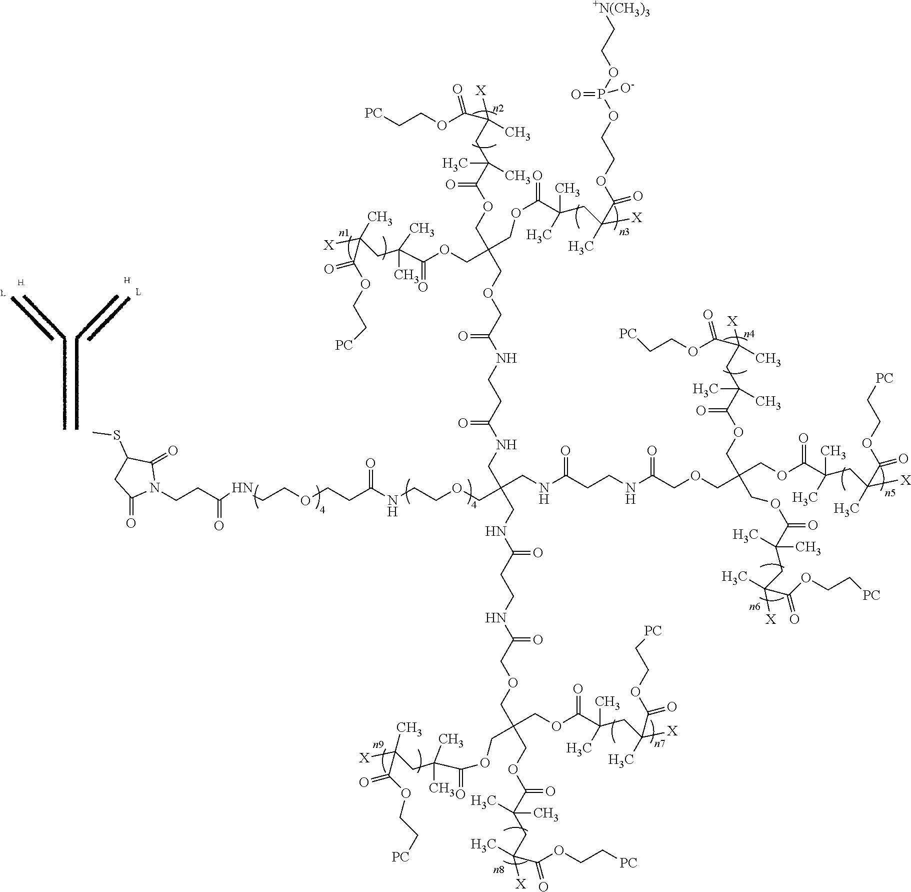

where X is a) —OR where R is H, methyl, ethyl, propyl, or isopropyl, b) —H, c) any halogen, including —Br, —Cl, or —I, d) —SCN, or e) —NCS; wherein: each heavy chain of the first antibody is denoted by the letter H, and each light chain of the first antibody is denoted by the letter L; the polymer is bonded to the first antibody through the sulfhydryl of a cysteine at position 449, as numbered in SEQ ID NO: 1, which bond is depicted on one of the heavy chains; PC is

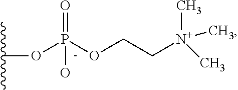 [SMILES: *OP(=O)(O)OCC[N+](C)(C)C]

where the curvy line indicates the point of attachment to the rest of the polymer; and n1, n2, n3, n4, n5, n6, n7, n8 and n9 are the same or different such that the sum of n1, n2, n3, n4, n5, n6, n7, n8 and n9 is 2500 plus or minus 15%. In some embodiments, X is a) —OR where R is —H, Methyl, ethyl, propyl, or isopropyl, b) —H, or c) a halide. In some embodiments, the sum of n1, n2, n3, n4, n5, n6, n7, n8 and n9 is about 1500 to about 3500 plus or minus about 10% to about 20%.

In some embodiments, the antibody conjugate is present in a liquid formulation. In some embodiments, the antibody conjugate is combined with a pharmaceutically acceptable carrier.

In some embodiments, an anti-VEGF-A antibody is presented. The anti-VEGF-A antibody heavy chain has at least the following CDR sequences: CDRH1: GYDFTHYGMN (SEQ ID NO: 9), CDRH2: WINTYTGEPTYAADFKR (SEQ ID NO: 10), and CDRH3: YPYYYGTSHWYFDV (SEQ ID NO: 11). In some embodiments, the anti-VEGF-A heavy chain has those CDRs and in addition has threonine (T) at position 221 (via sequential counting as in SEQ ID NO: 3). In some embodiments, the anti-VEGF-A light chain has at least the following CDRs: CDRL1: SASQDISNYLN (SEQ ID NO: 12), CDRL2: FTSSLHS (SEQ ID NO: 13) and CDRL3: QQYSTVPWT (SEQ ID NO: 14). In some embodiments, the anti-VEGF-A antibody has those CDRs and in addition has leucine (L) at Kabat position 4. In some embodiments, the isotype of the anti-VEGF-A antibody heavy chain, is IgG1 and has a CH1, hinge, CH2 and CH3 domains. In some embodiments the light chain isotype is kappa.

In some embodiments, the IgG1 domain of the anti-VEGF-A antibody has one or more mutations to modulate effector function, such as ADCC, ADCP, and CDC. In some embodiments, the IgG1 mutations reduce effector function. In some embodiments the amino acids to use for effector function mutations include (EU numbering) E233X, L234X, L235X, G236X, G237X, G236X, D270X, K322X, A327X, P329X, A330X, A330X, P331X, and P331X, in which X is any natural or non-natural amino acid. In some embodiments, the mutations include one or more of the following: E233P, L234V, L234A, L235A, G237A, A327G, A330S and P331S (EU numbering). In some embodiments, the anti-VEGF-A heavy chain has the following mutations (EU numbering): L234A, L235A and G237A. In some embodiments, the number of effector function mutations relative to a natural human IgG1 sequence is no more than 10. In some embodiments the number of effector function mutations relative to a natural human IgG1 sequence is no more than 5, 4, 3, 2 or 1. In some embodiments, the antibody has decreased Fc gamma binding and/or complement C1q binding, such that the antibody's ability to result in an effector function is decreased. This can be especially advantageous for ophthalmic indications/disorders.

In some embodiments, the anti-VEGF-A antibody comprises one or more of the following amino acid mutations: L234A, L235A, G237A (EU numbering), and L443C (EU numbering, or 449C in SEQ ID NO: 1).

In some embodiments, the anti-VEGF-A antibody is or is part of a human immunoglobulin G (IgG1).

In some embodiments, the VEGF-A antibody comprises a heavy chain constant domain that comprises one or more mutations that reduce an immune-mediated effector function.

In some embodiments an anti-VEGF-A antibody is provided. The anti-VEGF-antibody comprises a heavy chain that comprises a CDRH1 comprising the sequence GYDFTHYGMN (SEQ NO: 9), ID a CDRH2 comprising the sequence WINTYTGEPTYAADFKR (SEQ ID NO: 10), a CDRH3 comprising the sequence YPYYYGTSHWYFDV (SEQ ID NO: 11), a CDRL1 comprising the sequence SASQDISNYLN (SEQ ID NO: 12), a CDRL2 comprising the sequence FTSSLHS (SEQ ID NO: 13), and a CDRL3 comprising the sequence QQYSTVPWT (SEQ ID NO: 14).

Alternatively, the IgG domain can be IgG2, IgG3 or IgG4 or a composite in which a constant regions is formed from more than one of these isotypes (e.g., CHI region from IgG2 or IgG4, hinge, CH2 and CH3 regions from IgG1). Such domains can contain mutations to reduce and/or modulate effector function at one or more of the EU position mentioned for IgG1. Human IgG2 and IgG4 have reduced effector functions relative to human IgG1 and IgG3.

The anti-VEGF-A heavy chain has a cysteine residue added as a mutation by recombinant DNA technology which can be used to conjugate a half-life extending moiety. In some embodiments, the mutation is (EU numbering) Q347C (EU numbering) and/or L443C (EU numbering, or 449C in SEQ ID NO: 1). In some embodiments, the mutation is L443C (EU numbering, or 449C in SEQ ID NO: 1). In some embodiments, the stoichiometry of antibody to polymer is 1:1; in other words, a conjugate has one molecule of antibody conjugated to one molecule of polymer.

The half-life of the anti-VEGF-A antibodies can be extended by attachment of a “half-life (“half life”) extending moieties” or “half-life (“half life”) extending groups”. Half-life extending moieties include peptides and proteins which can be expressed in frame with the biological drug of issue (or conjugated chemically depending on the situation) and various polymers which can be attached or conjugated to one or more amino acid side chain or end functionalities such as—SH, —OH, —COOH, —CONH2, —NH2, or one or more N- and/or O-glycan structures. Half-life extending moieties generally act to increase the in vivo circulatory half-life of biologic drugs.

Examples of peptide/protein half-life extending moieties include Fc fusion (Capon D J, Chamow S M, Mordenti J, et al. Designing CD4 immunoadhesions for AIDS therapy. Nature. 1989. 337:525-31), human serum albumin (HAS) fusion (Yeh P, Landais D, Lemaitre M, et al. Design of yeast-secreted albumin derivatives for human therapy: biological and antiviral properties of a serum albumin-CD4 genetic conjugate. Proc Natl Acad Sci USA. 1992. 89:1904-08), carboxy terminal peptide (CTP) fusion (Fares F A, Suganuma N. Nishimori K, et al. Design of a long-acting follitropin agonist by fusing the C-terminal sequence of the chorionic gonadotropin beta subunit to the follitropin beta subunit. Proc Natl Acad Sci USA. 1992. 89:4304-08), genetic fusion of non-exact repeat peptide sequence (XTEN) fusion (Schellenberger V, Wang C W, Geething N C, et al. A recombinant polypeptide extends the in vivo half-life of peptides and proteins in a tunable manner. Nat Biotechnol. 2009. 27:1186-90), elastin like peptide (ELPylation) (MCpherson D T, Morrow C, Minehan D S, et al. Production and purification of a recombinant elastomeric polypeptide, G (VPGVG19-VPGV, from Escherichia coli. Biotechnol Prog. 1992. 8:347-52), human transferrin fusion (Prior C P, Lai C-H, Sadehghi H et al. Modified transferrin fusion proteins. Patent WO2004/020405. 2004), proline-alanine-serine (PASylation) (Skerra A, Theobald I, Schlapsky M. Biological active proteins having increased in vivo and/or vitro stability. Patent WO2008/155134 A1. 2008), homo-amino acid polymer (HAPylation) (Schlapschy M, Theobald I, Mack H, et al. Fusion of a recombinant antibody fragment with a homo-amino acid polymer: effects on biophysical properties and prolonged plasma half-life. Protein Eng Des Sel. 2007. 20:273-84) and gelatin like protein (GLK) fusion (Huang Y-S, Wen X-F, Zaro J L, et al. Engineering a pharmacologically superior form of granulocyte-colony-stimulating-factor by fusion with gelatin-like protein polymer. Eur J. Pharm Biopharm. 2010. 72:435-41).

Examples of polymer half-life extending moieties include polyethylene glycol (PEG), branched PEG, PolyPEG® (Warwick Effect Polymers; Coventry, UK), polysialic acid (PSA), starch, hydroxylethyl starch (HES), hydroxyalkyl starch (HAS), carbohydrate, polysaccharides, pullulane, chitosan, hyaluronic acid, chondroitin sulfate, dermatan sulfate, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyoxazoline, polyethylene-co-maleic acid anyhydride, polystyrene-co-maleic acid anhydride, poly(l-hydroxymethyethylene hydroxymethylformal) (PHF), a zwitterionic polymer, a phosphorylcholine containing polymer and a polymer comprising MPC, Poly (Glyx-Sery), Hyaluronic acid (HA), Heparosan polymers (HEP), Fleximers, Dextran, and Poly-sialic acids (PSA).

In one embodiment a half-life extending moiety can be conjugated to an antibody via free amino groups of the protein using N-hydroxysuccinimide (NHS) esters. Reagents targeting conjugation to amine groups can randomly react to ϵ-amine group of lysines, α-amine group of N-terminal amino acids, and δ-amine group of histidines. In some embodiments, the conjugate includes a polymer conjugated to a cysteine, or free amino groups of the protein, e.g., using N-hydroxysuccinimide (NHS) esters. In some embodiments, the conjugate includes a polymer conjugated to ϵ-amine group of lysines, α-amine group of N-terminal amino acids, and/or δ-amine group of histidines.

However, the anti-VEGF-A antibodies disclosed herein have many amine groups available for polymer conjugation. Conjugation of polymers to free amino groups, thus, might negatively impact the ability of the antibody proteins to bind to VEGF.

In some embodiments, a half-life extending moiety is coupled to one or more free SH groups using any appropriate thiol-reactive chemistry including, without limitation, maleimide chemistry, or the coupling of polymer hydrazides or polymer amines to carbohydrate moieties of the antibody after prior oxidation. In some embodiments maleimide coupling is used In some embodiments, coupling occurs at cysteines naturally present or introduced via genetic engineering.

In some embodiments, polymers are covalently attached to cysteine residues introduced into anti-VEGF-A antibodies by site directed mutagenesis. In some embodiments, the cysteine residues are employed in the Fc portion of the antibody. In some embodiments, the sites to introduce cysteine residues into an Fc region are provided in WO 2013/093809, U.S. Pat. No. 7,521,541, WO 2008/020827, U.S. Pat. Nos. 8,008,453, 8,455,622 and US2012/0213705, incorporated herein by reference for all purposes. In some embodiments, the cysteine mutations are Q347C (EU numbering) and L443C referring to the human IgG heavy chain by EU numbering.

In some embodiments, conjugates of antibody and high MW polymers serving as half-life extenders are provided. In some embodiments, a conjugate comprises an antibody that is coupled to a zwitterionic polymer wherein the polymer is formed from one or more monomer units and wherein at least one monomer unit has a zwitterionic group is provided. In some embodiments, the zwitterionic group is phosphorylcholine.

In some embodiments, one of the monomer units is HEMA-PC. In some embodiments, a polymer is synthesized from a single monomer which is HEMA-PC.

In some embodiments, some antibody conjugates have 2, 3, or more polymer arms wherein the monomer is HEMA-PC. In some embodiments, the conjugates have 2, 3, 4, 5, 6, 7, 8, 9, 10, 11, or 12 polymer arms wherein the monomer is HEMA-PC. In some embodiments, the conjugates have 3, 6 or 9 arms. In some embodiments, the conjugate has 9 arms.

In some embodiments, polymer-antibody conjugates have a polymer portion with a molecular weight of between 100,000 and 1,500,000 Da. In some embodiments, the conjugate has a polymer portion with a molecular weight between 500,000 and 1,000,000 Da. In some embodiments, the conjugate has a polymer portion with a molecular weight between 600,000 to 800,000 Da. In some embodiments, the conjugate has a polymer portion with a molecular weight between 600,000 and 850,000 Da and has 9 arms. When a molecular weight is given for an antibody conjugated to a polymer, the molecular weight will be the addition of the molecular weight of the protein, including any carbohydrate moieties associated therewith, and the molecular weight of the polymer.

In some embodiments, the polymer component of the conjugated protein has a molecular weight of about 100,000 Da or more, e.g., about 150,000 Da or more, about 200,000 Da or more, about 350,000 Da or more, about 400,000 Da or more, about 450,000 Da or more, about 500,000 Da or more, about 550,000 Da or more, about 600,000 Da or more, about 650,000 Da or more, about 700,000 or more, about 750,000 Da or more, about 800,000 Da or more, about 850,000 Da or more, about 900,000 Da or more, about 950,000 Da or more, about 1,000,000 Da or more, or a molecular weight in a range defined by any two of the preceding values (e.g., 100,000-1,000,000 Da, 300,000-950,000 Da, 400,000-800,000 Da, 500,000-750.000 Da, 600,000-700,000 Da, etc.). In some embodiments, the polymer component of the conjugated protein is any of the polymers disclosed herein. In some embodiments, the polymer component of the conjugated protein is OG1801 or OG1802.

In some embodiments, an anti-VEGF-A antibody has a HEMA-PC polymer which has a molecular weight measured by Mw of between about 100 kDa and 1650 kDa is provided. In some embodiments, the molecular weight of the polymer as measured by Mw is between about 500 kDa and 1000 kDa. In some embodiments, the molecular weight of the polymer as measured by Mw is between about 600 kDa to about 900 kDa. In some embodiments, the polymer molecular weight as measured by Mw is 750 kDa plus or minus 15%.

In some embodiments, the polymer is made from an initiator suitable for ATRP having one or more polymer initiation sites. In some embodiments, the polymer initiation site has a 2-bromoisobutyrate site. In some embodiments, the initiator has 3 or more polymer initiation sites. In some embodiments, the initiator has 3, 4, 5, 6, 7, 8, 9, 10, 11 or 12 polymer initiation sites. In some embodiments, the initiator has 3, 6 or 9 polymer initiation sites. In some embodiments, the initiator has 9 polymer initiation sites. In some embodiments, the initiator is OG1786.

The anti-VEGF-A antibodies can be produced by recombinant expression including (i) the production of recombinant DNA by genetic engineering, (ii) introducing recombinant DNA into prokaryotic or eukaryotic cells by, for example and without limitation, transfection, electroporation or microinjection, (iii) cultivating the transformed cells, (iv) expressing antibody, e.g. constitutively or on induction, and (v) isolating the antibody, e.g. from the culture medium or by harvesting the transformed cells, in order to (vi) obtain purified antibody.

The anti-VEGF-A antibodies can be produced by expression in a suitable prokaryotic or eukaryotic host system characterized by producing a pharmacologically acceptable antibody molecule. Examples of eukaryotic cells are mammalian cells, such as CHO, COS, HEK 293, BHK, SK-Hip, and HepG2. Other suitable expression systems are prokaryotic (e.g., E. coli with pET/BL21 expression system), yeast (Saccharomyces cerevisiae and/or Pichia pastoris systems), and insect cells.

A wide variety of vectors can be used for the preparation of the antibodies disclosed herein and are selected from eukaryotic and prokaryotic expression vectors. Examples of vectors for prokaryotic expression include plasmids such as, and without limitation, preset, pet, and pad, wherein the promoters used in prokaryotic expression vectors include one or more of, and without limitation, lac, trc, trp, recA, or araBAD. Examples of vectors for eukaryotic expression include: (i) for expression in yeast, vectors such as, and without limitation, pAO, pPIC, pYES, or pMET, using promoters such as, and without limitation, AOX1, GAP, GAL1, or AUG1; (ii) for expression in insect cells, vectors such as and without limitation, pMT, pAc5, pIB, pMIB, or pBAC, using promoters such as and without limitation PH, p10, MT, Ac5, OpIE2, gp64, or polh, and (iii) for expression in mammalian cells, vectors such as, and without limitation, pSVL, pCMV, pRc/RSV, pcDNA3, or pBPV, and vectors derived from, in one aspect, viral systems such as and without limitation vaccinia virus, adeno-associated viruses, herpes viruses, or retroviruses, using promoters such as and without limitation CMV, SV40, EF-1, UbC, RSV, ADV, BPV, and beta-actin.

### Analysis of Mixed Formulation and Aggregates

In some embodiments, a method is presented for analyzing a mixed formulation sample (or therapeutically acceptable composition). In some embodiments, the method can include providing a mixed formulation sample (or therapeutically acceptable composition sample). In some embodiments, the mixed formulation sample (or therapeutically acceptable composition sample) includes a first antibody that is an anti-VEGF-A antibody conjugated to a polymer and a second antibody that is an anti-VEGF-A antibody that is not conjugated to the polymer. In some embodiments, the method further comprises a first run. In some embodiments, during the first run, the mixed formulation (or therapeutically acceptable composition) is run through a prefiltration step to provide a first filtered mixed formulation. In some embodiments, the first filtered mixed formulation is run through a cation exchange chromatography (CEX) column to provide a second filtered mixed formulation. In some embodiments, the method further comprises a second run. In some embodiments, during the second run, the protein bound to the CEX column is eluted to provide a third filtered mixed formulation. In some embodiments, the third filtered mixed formulation is run through a size exclusion exchange (SEC) high performance liquid chromatography (HPLC) column to provide a fourth filtered mixed formulation. In some embodiments, a specific ionic strength of the solvent is used to allow partitioning of the mixed formulation (or therapeutically acceptable composition) in the CEX column. In some embodiments, the method allows analysis of the mixed formulation (or therapeutically acceptable composition) based on the differential charge and size variant of the constituents.

In some embodiments the mixed formulation (or therapeutically acceptable composition) comprises a first antibody conjugated to a polymer and a second antibody that is not conjugated to a polymer, where one or both antibodies are not anti-VEGF-A antibodies. In some embodiments, the first and second antibody of the mixed formulation (or therapeutically acceptable composition) are the same antibody. In some embodiments, the first and second antibody of the mixed formulation are different antibodies. In some embodiments, the mixed formulation (or therapeutically acceptable composition) includes a first protein moiety conjugated to a polymer and a second protein moiety not conjugated to a polymer. In some embodiments the protein moieties are not antibodies. In some embodiments, the mixed formulation (or therapeutically acceptable composition) includes a mix of antibody and other protein moieties. In some embodiments, the first and second protein or antibody moieties are both conjugated to a polymer. In some embodiments, the first and second protein or antibody moieties are conjugated to the same polymer. In some embodiments, the first and second protein or antibody moieties are conjugated to different polymers. In some embodiments, the first and second antibody or protein moieties are not conjugated to a polymer. In some embodiments, the mixed formulation (or therapeutically acceptable composition) comprises more than two antibodies or protein moieties.

In some embodiments, the proteins or antibodies of the mixed formulation (or therapeutically acceptable composition) are present in a desired percent composition. In some embodiments, the percent composition is calculated by determining the amount of the one component of the mixed formulation (or therapeutically acceptable composition) in mass units (e.g., ug) and dividing that amount by the total amount of all components in the composition in mass units, and multiplying by 100.

In some embodiments, the proteins or antibodies of the mixed formulation (or therapeutically acceptable composition) are present in a desired molar ratio of unconjugated to conjugated protein or antibody.

In some embodiments, the percent of the conjugated to unconjugated protein (e.g., % total molar amount) is calculated by (1) measuring the conjugated protein moiety and unconjugated protein moiety in mg/mL; (2) converting the mg/mL values of the conjugated protein moiety and unconjugated protein moiety into a molecular weight measured in kDa; and (3) dividing the molecular weight of each of the conjugated protein moiety and unconjugated protein moiety by the total molecular weight of the conjugated protein moiety and unconjugated protein moiety in the composition, and multiplied by 100 to achieve a percent of the total molar amount for each.

Any of the formulations and compositions provided herein, in some embodiments, can be defined as a percent composition (e.g., in mass weight concentration) of one component relative to the total mass weight concentration of the proteins (e.g., excluding any contribution of a polymer that may be conjugated thereto) in the composition. In some embodiments, a formulation or composition defined in % total molar amount of the second protein (e.g., the unconjugated protein) can be defined in percent composition (e.g., in mass weight concentration) of the second protein relative to the total mass weight concentration of the first and second proteins, given the relevant molecular weight of each protein. In some embodiments, percent composition is measured in mass weight concentration (in other words, gram per liter or milligram per milliliter) of the free protein relative to the total mass weight concentration of the proteins (e.g., excluding any contribution of a polymer that may be conjugated thereto) in the mixture.

In some embodiments, a mixed therapeutically acceptable composition includes: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein relative to the total protein mass weight concentration of the first protein and the second protein in the composition is about 1% or more (e.g., about 5-93%, 15-25%, 25-35%, 25-40%, etc.).

In some embodiments, a mixed therapeutically acceptable composition includes: a conjugate comprising a first protein conjugated to a polymer; and a second protein that is not conjugated to a polymer, wherein the second protein at a percent composition relative to the total protein mass weight concentration of the first protein and the second protein in the composition of about 1% or more (e.g., about 5-93%, 15-25%, 25-35%, 25-40%, etc.) has been combined with the conjugate, wherein the remainder of the total protein mass weight concentration comprises the first protein. In some embodiments, the mixed composition has been prepared by combining the second protein at a percent composition of about 1% or more (e.g., about 5-93%, 15-25%, 25-35%, 25-40%, etc.) relative to the total protein mass weight concentration of the first protein and the second protein, with the conjugate such that the first protein at a percent composition of at a remainder of the total protein mass weight concentration. For example, for a total mass weight concentration of 50 mg/mL, the mixed therapeutically acceptable composition can be prepared by combining an amount of the second protein that corresponds to 10 mg/mL in the final composition (percent composition of 20%) with an amount of the conjugate that corresponds to 40 mg/mL of the first protein (as the conjugate, excluding any contribution of the polymer to the mass weight concentration calculation) in the final composition.

In any embodiment herein, the second protein that is not conjugated to a polymer (e.g., a phosphorylcholine-containing polymer) can be referred to as the unconjugated protein or protein moiety, and the first protein that is conjugated to a polymer (e.g., a phosphorylcholine-containing polymer) can be referred to as the conjugated protein or protein moiety.

In some embodiments, the percent composition of the unconjugated protein or protein moiety (e.g., unconjugated antibody) is between 5% and 6%, with the remainder comprising the conjugated protein moiety or protein moiety. As used herein, “the remainder” denotes the portion of the total protein mass weight concentration of the composition (excluding any contribution of a polymer conjugated to the first protein to the mass weight concentration) that is not the unconjugated protein (e.g., the second protein), where the percent composition of the unconjugated protein (e.g., the second protein) and the remainder adds up to 100% of the total protein mass weight concentration. For example, between 5% and 6% of the total mass weight concentration of the total protein concentration in the mixture is the unconjugated protein, and between 94% and 95% of the total mass weight concentration of the proteins in the mixture is the conjugated protein, where the percentages add up to 100%. In some embodiments, the percent composition of the unconjugated protein (e.g., unconjugated antibody) is between 6% and 7%, with the remainder comprising the conjugated protein. In some embodiments, the percent composition of the unconjugated protein (e.g., unconjugated antibody) is between 7% and 8%, with the remainder comprising the conjugated protein. In some embodiments, the percent composition of the unconjugated protein (e.g., unconjugated antibody) is between 8% and 9%, with the remainder comprising the conjugated protein. In some embodiments, the percent composition of the unconjugated protein (e.g., unconjugated antibody) is between 9% and 10%, with the remainder comprising the conjugated protein. In some embodiments, the percent composition of the unconjugated protein (e.g., unconjugated antibody) is between 10% and 11%, with the remainder comprising the conjugated protein. In some embodiments, the percent composition of the unconjugated protein (e.g., unconjugated antibody) is between 11% and 12%, with the remainder comprising the conjugated protein. In some embodiments, the percent composition of the unconjugated protein (e.g., unconjugated antibody) is between 12% and 13%, with the remainder comprising the conjugated protein. In some embodiments, the percent composition of the unconjugated protein (e.g., unconjugated antibody) is between 13% and 14%, with the remainder comprising the conjugated protein. In some embodiments, the percent composition of the unconjugated protein (e.g., unconjugated antibody) is between 14% and 15%, with the remainder comprising the conjugated protein. In some embodiments, the percent composition of the unconjugated protein (e.g., unconjugated antibody) is between 15% and 16%, with the remainder comprising the conjugated protein. In some embodiments, the percent composition of the unconjugated protein (e.g., unconjugated antibody) is between 16% and 17%, with the remainder comprising the conjugated protein. In some embodiments, the percent composition of the unconjugated protein (e.g., unconjugated antibody) is between 17% and 18%, with the remainder comprising the conjugated protein. In some embodiments, the percent composition of the unconjugated protein (e.g., unconjugated antibody) is between 18% and 19%, with the remainder comprising the conjugated protein. In some embodiments, the percent composition of the unconjugated protein (e.g., unconjugated antibody) is between 19% and 20%, with the remainder comprising the conjugated protein. In some embodiments, the percent composition of the unconjugated protein (e.g., unconjugated antibody) is between 20% and 21%, with the remainder comprising the conjugated protein. In some embodiments, the percent composition of the unconjugated protein (e.g., unconjugated antibody) is between 21% and 22%, with the remainder comprising the conjugated protein. In some embodiments, the percent composition of the unconjugated protein (e.g., unconjugated antibody) is between 22% and 23%, with the remainder comprising the conjugated protein. In some embodiments, the percent composition of the unconjugated protein (e.g., unconjugated antibody) is between 23% and 24%, with the remainder comprising the conjugated protein. In some embodiments, the percent composition of the unconjugated protein (e.g., unconjugated antibody) is between 24% and 25%.

In some embodiments, the percent composition of the unconjugated protein or protein moiety (e.g., unconjugated antibody or unconjugated fusion construct) is between 25% and 26%, with the remainder comprising the conjugated protein or protein moiety (e.g., conjugated antibody or conjugated fusion construct). In some embodiments, the percent composition of the unconjugated protein (e.g., unconjugated antibody) is between 26% and 27%, with the remainder comprising the conjugated protein. In some embodiments, the percent composition of the unconjugated protein (e.g., unconjugated antibody) is between 27% and 28%, with the remainder comprising the conjugated protein. In some embodiments, the percent composition of the unconjugated protein (e.g., unconjugated antibody) is between 28% and 29%, with the remainder comprising the conjugated protein. In some embodiments, the percent composition of the unconjugated protein (e.g., unconjugated antibody) is between 29% and 30%, with the remainder comprising the conjugated protein. In some embodiments, the percent composition of the unconjugated protein (e.g., unconjugated antibody) is between 30% and 31%, with the remainder comprising the conjugated protein. In some embodiments, the percent composition of the unconjugated protein (e.g., unconjugated antibody) is between 31% and 32%, with the remainder comprising the conjugated protein. In some embodiments, the percent composition of the unconjugated protein (e.g., unconjugated antibody) is between 32% and 33%, with the remainder comprising the conjugated protein. In some embodiments, the percent composition of the unconjugated protein (e.g., unconjugated antibody) is between 33% and 34%, with the remainder comprising the conjugated protein. In some embodiments, the percent composition of the unconjugated protein (e.g., unconjugated antibody) is between 34% and 35%, with the remainder comprising the conjugated protein. In some embodiments, the percent composition of the unconjugated protein (e.g., unconjugated antibody) is between 35% and 36%, with the remainder comprising the conjugated protein. In some embodiments, the percent composition of the unconjugated protein (e.g., unconjugated antibody) is between 36% and 37%, with the remainder comprising the conjugated protein. In some embodiments, the percent composition of the unconjugated protein (e.g., unconjugated antibody) is between 37% and 38%, with the remainder comprising the conjugated protein. In some embodiments, the percent composition of the unconjugated protein (e.g., unconjugated antibody) is between 38% and 39%, with the remainder comprising the conjugated protein. In some embodiments, the percent composition of the unconjugated protein (e.g., unconjugated antibody) is between 39% and 40%, with the remainder comprising the conjugated protein. In some embodiments, the percent composition of the unconjugated protein (e.g., unconjugated antibody) is between 40% and 41%, with the remainder comprising the conjugated protein. In some embodiments, the percent composition of the unconjugated protein (e.g., unconjugated antibody) is between 41% and 42%, with the remainder comprising the conjugated protein. In some embodiments, the percent composition of the unconjugated protein (e.g., unconjugated antibody) is between 42% and 43%, with the remainder comprising the conjugated protein. In some embodiments, the percent composition of the unconjugated protein (e.g., unconjugated antibody) is between 43% and 44%, with the remainder comprising the conjugated protein. In some embodiments, the percent composition of the unconjugated protein (e.g., unconjugated antibody) is between 44% and 45%.

In some embodiments, the percent composition of the unconjugated protein or protein moiety (e.g., unconjugated antibody or fusion construct) is between 45% and 46%, with the remainder comprising the conjugated protein or protein moiety (e.g., conjugated antibody or unconjugated fusion construct). In some embodiments, the percent composition of the unconjugated protein (e.g., unconjugated antibody) is between 46% and 47%, with the remainder comprising the conjugated protein. In some embodiments, the percent composition of the unconjugated protein (e.g., unconjugated antibody) is between 47% and 48%, with the remainder comprising the conjugated protein. In some embodiments, the percent composition of the unconjugated protein (e.g., unconjugated antibody) is between 48% and 49%, with the remainder comprising the conjugated protein. In some embodiments, the percent composition of the unconjugated protein (e.g., unconjugated antibody) is between 49% and 50%, with the remainder comprising the conjugated protein. In some embodiments, the percent composition of the unconjugated protein (e.g., unconjugated antibody) is between 50% and 51%, with the remainder comprising the conjugated protein. In some embodiments, the percent composition of the unconjugated protein (e.g., unconjugated antibody) is between 51% and 52%, with the remainder comprising the conjugated protein. In some embodiments, the percent composition of the unconjugated protein (e.g., unconjugated antibody) is between 52% and 53%, with the remainder comprising the conjugated protein. In some embodiments, the percent composition of the unconjugated protein (e.g., unconjugated antibody) is between 53% and 54%, with the remainder comprising the conjugated protein. In some embodiments, the percent composition of the unconjugated protein (e.g., unconjugated antibody) is between 54% and 55%, with the remainder comprising the conjugated protein. In some embodiments, the percent composition of the unconjugated protein (e.g., unconjugated antibody) is between 55% and 56%, with the remainder comprising the conjugated protein. In some embodiments, the percent composition of the unconjugated protein (e.g., unconjugated antibody) is between 56% and 57%, with the remainder comprising the conjugated protein. In some embodiments, the percent composition of the unconjugated protein (e.g., unconjugated antibody) is between 57% and 58%, with the remainder comprising the conjugated protein. In some embodiments, the percent composition of the unconjugated protein (e.g., unconjugated antibody) is between 58% and 59%, with the remainder comprising the conjugated protein. In some embodiments, the percent composition of the unconjugated protein (e.g., unconjugated antibody) is between 59% and 60%, with the remainder comprising the conjugated protein. In some embodiments, the percent composition of the unconjugated protein (e.g., unconjugated antibody) is between 60% and 61%, with the remainder comprising the conjugated protein. In some embodiments, the percent composition of the unconjugated protein (e.g., unconjugated antibody) is between 61% and 62%, with the remainder comprising the conjugated protein. In some embodiments, the percent composition of the unconjugated protein (e.g., unconjugated antibody) is between 62% and 63%, with the remainder comprising the conjugated protein. In some embodiments, the percent composition of the unconjugated protein (e.g., unconjugated antibody) is between 63% and 64%, with the remainder comprising the conjugated protein. In some embodiments, the percent composition of the unconjugated protein (e.g., unconjugated antibody) is between 64% and 65%.

In some embodiments, the percent composition of the unconjugated protein or protein moiety (e.g., unconjugated antibody or unconjugated fusion construct) is between 65% and 66%, with the remainder comprising the conjugated protein or protein moiety (e.g., conjugated antibody or conjugate fusion construct). In some embodiments, the percent composition of the unconjugated protein (e.g., unconjugated antibody) is between 66% and 67%, with the remainder comprising the conjugated protein. In some embodiments, the percent composition of the unconjugated protein (e.g., unconjugated antibody) is between 67% and 68%, with the remainder comprising the conjugated protein. In some embodiments, the percent composition of the unconjugated protein (e.g., unconjugated antibody) is between 68% and 69%, with the remainder comprising the conjugated protein. In some embodiments, the percent composition of the unconjugated protein (e.g., unconjugated antibody) is between 69% and 70%, with the remainder comprising the conjugated protein. In some embodiments, the percent composition of the unconjugated protein (e.g., unconjugated antibody) is between 70% and 71%, with the remainder comprising the conjugated protein. In some embodiments, the percent composition of the unconjugated protein (e.g., unconjugated antibody) is between 71% and 72%, with the remainder comprising the conjugated protein. In some embodiments, the percent composition of the unconjugated protein (e.g., unconjugated antibody) is between 72% and 73%, with the remainder comprising the conjugated protein. In some embodiments, the percent composition of the unconjugated protein (e.g., unconjugated antibody) is between 73% and 74%, with the remainder comprising the conjugated protein. In some embodiments, the percent composition of the unconjugated protein (e.g., unconjugated antibody) is between 74% and 75%, with the remainder comprising the conjugated protein. In some embodiments, the percent composition of the unconjugated protein (e.g., unconjugated antibody) is between 75% and 76%, with the remainder comprising the conjugated protein. In some embodiments, the percent composition of the unconjugated protein (e.g., unconjugated antibody) is between 76% and 77%, with the remainder comprising the conjugated protein. In some embodiments, the percent composition of the unconjugated protein (e.g., unconjugated antibody) is between 77% and 78%, with the remainder comprising the conjugated protein. In some embodiments, the percent composition of the unconjugated protein (e.g., unconjugated antibody) is between 78% and 79%, with the remainder comprising the conjugated protein. In some embodiments, the percent composition of the unconjugated protein (e.g., unconjugated antibody) is between 79% and 80%, with the remainder comprising the conjugated protein. In some embodiments, the percent composition of the unconjugated protein (e.g., unconjugated antibody) is between 80% and 81%, with the remainder comprising the conjugated protein. In some embodiments, the percent composition of the unconjugated protein (e.g., unconjugated antibody) is between 81% and 82%, with the remainder comprising the conjugated protein. In some embodiments, the percent composition of the unconjugated protein (e.g., unconjugated antibody) is between 82% and 83%, with the remainder comprising the conjugated protein. In some embodiments, the percent composition of the unconjugated protein (e.g., unconjugated antibody) is between 83% and 84%, with the remainder comprising the conjugated protein. In some embodiments, the percent composition of the unconjugated protein (e.g., unconjugated antibody) is between 84% and 85%.

In some embodiments, the percent composition of the unconjugated protein or protein moiety (e.g., unconjugated antibody or unconjugated fusion construct) is between 85% and 86%, with the remainder comprising the conjugated protein or protein moiety (e.g., conjugated antibody or conjugated fusion construct). In some embodiments, the percent composition of the unconjugated protein (e.g., unconjugated antibody) is between 86% and 87%, with the remainder comprising the conjugated protein. In some embodiments, the percent composition of the unconjugated protein (e.g., unconjugated antibody) is between 87% and 88%, with the remainder comprising the conjugated protein. In some embodiments, the percent composition of the unconjugated protein (e.g., unconjugated antibody) is between 88% and 89%, with the remainder comprising the conjugated protein. In some embodiments, the percent composition of the unconjugated protein (e.g., unconjugated antibody) is between 89% and 90%, with the remainder comprising the conjugated protein.

In some embodiments, the percent composition of the unconjugated protein or protein moiety (e.g., unconjugated antibody or unconjugated fusion construct) is between 5% and 25%, with the remainder comprising the conjugated protein or protein moiety (e.g., conjugated antibody or conjugated fusion construct). In some embodiments, the percent composition of the unconjugated protein (e.g., unconjugated antibody) is between 5% and 10%, with the remainder comprising the conjugated protein. In some embodiments, the percent composition of the unconjugated protein (e.g., unconjugated antibody) is between 5% and 12.5%, with the remainder comprising the conjugated protein. In some embodiments, the percent composition of the unconjugated protein (e.g., unconjugated antibody) is between 5% and 15%, with the remainder comprising the conjugated protein. In some embodiments, the percent composition of the unconjugated protein (e.g., unconjugated antibody) is between 5% and 17.5%. In some embodiments, the percent composition of the unconjugated protein (e.g., unconjugated antibody) is between 5% and 20%, with the remainder comprising the conjugated protein. In some embodiments, the percent composition of the unconjugated protein (e.g., unconjugated antibody) is between 5% and 25%, with the remainder comprising the conjugated protein. In some embodiments, the percent composition of the unconjugated protein (e.g., unconjugated antibody) is between 5% and 30%, with the remainder comprising the conjugated protein. In some embodiments, the percent composition of the unconjugated protein (e.g., unconjugated antibody) is between 5% and 35%, with the remainder comprising the conjugated protein. In some embodiments, the percent composition of the unconjugated protein (e.g., unconjugated antibody) is between 5% and 45%, with the remainder comprising the conjugated protein. In some embodiments, the percent composition of the unconjugated protein (e.g., unconjugated antibody) is between 5% and 50%, with the remainder comprising the conjugated protein. In some embodiments, the percent composition of the unconjugated protein (e.g., unconjugated antibody) is between 5% and 55%, with the remainder comprising the conjugated protein. In some embodiments, the percent composition of the unconjugated protein (e.g., unconjugated antibody) is between 5% and 60%, with the remainder comprising the conjugated protein. In some embodiments, the percent composition of the unconjugated protein (e.g., unconjugated antibody) is between 5% and 65%, with the remainder comprising the conjugated protein. In some embodiments, the percent composition of the unconjugated protein (e.g., unconjugated antibody) is between 5% and 70%, with the remainder comprising the conjugated protein. In some embodiments, the percent composition of the unconjugated protein (e.g., unconjugated antibody) is between 5% and 75%, with the remainder comprising the conjugated protein. In some embodiments, the percent composition of the unconjugated protein (e.g., unconjugated antibody) is between 5% and 80%, with the remainder comprising the conjugated protein. In some embodiments, the percent composition of the unconjugated protein (e.g., unconjugated antibody) is between 5% and 85%, with the remainder comprising the conjugated protein. In some embodiments, the percent composition of the unconjugated protein (e.g., unconjugated antibody) is between 5% and 90%.

In some embodiments, the percent composition of the unconjugated protein or protein moiety (e.g., unconjugated antibody or unconjugated fusion construct) is between 10% and 12.5%, with the remainder comprising the conjugated protein or protein moiety (e.g., conjugated antibody or conjugated fusion construct). In some embodiments, the percent composition of the unconjugated protein (e.g., unconjugated antibody) is between 10% and 15%, with the remainder comprising the conjugated protein. In some embodiments, the percent composition of the unconjugated protein (e.g., unconjugated antibody) is between 10% and 17.5%, with the remainder comprising the conjugated protein. In some embodiments, the percent composition of the unconjugated protein (e.g., unconjugated antibody) is between 10% and 20%, with the remainder comprising the conjugated protein. In some embodiments, the percent composition of the unconjugated protein (e.g., unconjugated antibody) is between 15% and 25%, with the remainder comprising the conjugated protein. In some embodiments, the percent composition of the unconjugated protein (e.g., unconjugated antibody) is between 10% and 25%, with the remainder comprising the conjugated protein. In some embodiments, the percent composition of the unconjugated protein (e.g., unconjugated antibody) is between 10% and 30%, with the remainder comprising the conjugated protein. In some embodiments, the percent composition of the unconjugated protein (e.g., unconjugated antibody) is between 10% and 35%, with the remainder comprising the conjugated protein. In some embodiments, the percent composition of the unconjugated protein (e.g., unconjugated antibody) is between 10% and 45%, with the remainder comprising the conjugated protein. In some embodiments, the percent composition of the unconjugated protein (e.g., unconjugated antibody) is between 10% and 50%, with the remainder comprising the conjugated protein. In some embodiments, the percent composition of the unconjugated protein (e.g., unconjugated antibody) is between 10% and 55%, with the remainder comprising the conjugated protein. In some embodiments, the percent composition of the unconjugated protein (e.g., unconjugated antibody) is between 10% and 60%, with the remainder comprising the conjugated protein. In some embodiments, the percent composition of the unconjugated protein (e.g., unconjugated antibody) is between 10% and 65%, with the remainder comprising the conjugated protein. In some embodiments, the percent composition of the unconjugated protein (e.g., unconjugated antibody) is between 10% and 70%, with the remainder comprising the conjugated protein. In some embodiments, the percent composition of the unconjugated protein (e.g., unconjugated antibody) is between 10% and 75%, with the remainder comprising the conjugated protein. In some embodiments, the percent composition of the unconjugated protein (e.g., unconjugated antibody) is between 10% and 80%, with the remainder comprising the conjugated protein. In some embodiments, the percent composition of the unconjugated protein (e.g., unconjugated antibody) is between 10% and 85%, with the remainder comprising the conjugated protein. In some embodiments, the percent composition of the unconjugated protein (e.g., unconjugated antibody) is between 10% and 90%.

In some embodiments, the percent composition of the unconjugated protein or protein moiety (e.g., unconjugated antibody or unconjugated fusion construct) is between 12.5% and 15%, with the remainder comprising the conjugated protein or protein moiety (e.g., the conjugated antibody or conjugated fusion construct). In some embodiments, the percent composition of the unconjugated protein (e.g., unconjugated antibody) is between 12.5% and 17.5%. In some embodiments, the percent composition of the unconjugated protein (e.g., unconjugated antibody) is between 17.5% and 22.5%. In some embodiments, the percent composition of the unconjugated protein (e.g., unconjugated antibody) is between 12.5% and 20%, with the remainder comprising the conjugated protein. In some embodiments, the percent composition of the unconjugated protein (e.g., unconjugated antibody) is between 12.5% and 25%, with the remainder comprising the conjugated protein. In some embodiments, the percent composition of the unconjugated protein (e.g., unconjugated antibody) is between 12.5% and 30%, with the remainder comprising the conjugated protein. In some embodiments, the percent composition of the unconjugated protein (e.g., unconjugated antibody) is between 12.5% and 35%, with the remainder comprising the conjugated protein. In some embodiments, the percent composition of the unconjugated protein (e.g., unconjugated antibody) is between 12.5% and 45%, with the remainder comprising the conjugated protein. In some embodiments, the percent composition of the unconjugated protein (e.g., unconjugated antibody) is between 12.5% and 50%, with the remainder comprising the conjugated protein. In some embodiments, the percent composition of the unconjugated protein (e.g., unconjugated antibody) is between 12.5% and 55%, with the remainder comprising the conjugated protein. In some embodiments, the percent composition of the unconjugated protein (e.g., unconjugated antibody) is between 12.5% and 60%, with the remainder comprising the conjugated protein. In some embodiments, the percent composition of the unconjugated protein (e.g., unconjugated antibody) is between 12.5% and 65%, with the remainder comprising the conjugated protein. In some embodiments, the percent composition of the unconjugated protein (e.g., unconjugated antibody) is between 12.5% and 70%, with the remainder comprising the conjugated protein. In some embodiments, the percent composition of the unconjugated protein (e.g., unconjugated antibody) is between 12.5% and 75%, with the remainder comprising the conjugated protein. In some embodiments, the percent composition of the unconjugated protein (e.g., unconjugated antibody) is between 12.5% and 80%, with the remainder comprising the conjugated protein. In some embodiments, the percent composition of the unconjugated protein (e.g., unconjugated antibody) is between 12.5% and 85%, with the remainder comprising the conjugated protein. In some embodiments, the percent composition of the unconjugated protein (e.g., unconjugated antibody) is between 12.5% and 90%.

In some embodiments, the percent composition of the unconjugated protein or protein moiety (e.g., unconjugated antibody or unconjugated fusion construct) is between 15% and 17.5%, with the remainder comprising the conjugated protein or protein moiety (e.g., conjugated antibody or fusion construct). In some embodiments, the percent composition of the unconjugated protein (e.g., unconjugated antibody) is between 15% and 20%, with the remainder comprising the conjugated protein. In some embodiments, the percent composition of the unconjugated protein (e.g., unconjugated antibody) is between 15% and 25%, with the remainder comprising the conjugated protein. In some embodiments, the percent composition of the unconjugated protein (e.g., unconjugated antibody) is between 15% and 30%, with the remainder comprising the conjugated protein. In some embodiments, the percent composition of the unconjugated protein (e.g., unconjugated antibody) is between 15% and 35%, with the remainder comprising the conjugated protein. In some embodiments, the percent composition of the unconjugated protein (e.g., unconjugated antibody) is between 15% and 45%, with the remainder comprising the conjugated protein. In some embodiments, the percent composition of the unconjugated protein (e.g., unconjugated antibody) is between 15% and 50%, with the remainder comprising the conjugated protein. In some embodiments, the percent composition of the unconjugated protein (e.g., unconjugated antibody) is between 15% and 55%, with the remainder comprising the conjugated protein. In some embodiments, the percent composition of the unconjugated protein (e.g., unconjugated antibody) is between 15% and 60%, with the remainder comprising the conjugated protein. In some embodiments, the percent composition of the unconjugated protein (e.g., unconjugated antibody) is between 15% and 65%, with the remainder comprising the conjugated protein. In some embodiments, the percent composition of the unconjugated protein (e.g., unconjugated antibody) is between 15% and 70%, with the remainder comprising the conjugated protein. In some embodiments, the percent composition of the unconjugated protein (e.g., unconjugated antibody) is between 15% and 75%, with the remainder comprising the conjugated protein. In some embodiments, the percent composition of the unconjugated protein (e.g., unconjugated antibody) is between 15% and 80%, with the remainder comprising the conjugated protein. In some embodiments, the percent composition of the unconjugated protein (e.g., unconjugated antibody) is between 15% and 85%, with the remainder comprising the conjugated protein. In some embodiments, the percent composition of the unconjugated protein (e.g., unconjugated antibody) is between 15% and 90%.

In some embodiments, the percent composition of the unconjugated protein or protein moiety (e.g., unconjugated antibody or unconjugated fusion construct) is between 20% and 25%, with the remainder comprising the conjugated protein or protein moiety (e.g., conjugated antibody or fusion construct). In some embodiments, the percent composition of the unconjugated protein (e.g., unconjugated antibody) is between 25% and 30%, with the remainder comprising the conjugated protein. In some embodiments, the percent composition of the unconjugated protein (e.g., unconjugated antibody) is between 25% and 35%, with the remainder comprising the conjugated protein. In some embodiments, the percent composition of the unconjugated protein (e.g., unconjugated antibody) is between 25% and 45%, with the remainder comprising the conjugated protein. In some embodiments, the percent composition of the unconjugated protein (e.g., unconjugated antibody) is between 25% and 50%, with the remainder comprising the conjugated protein. In some embodiments, the percent composition of the unconjugated protein (e.g., unconjugated antibody) is between 25% and 55%, with the remainder comprising the conjugated protein. In some embodiments, the percent composition of the unconjugated protein (e.g., unconjugated antibody) is between 25% and 60%, with the remainder comprising the conjugated protein. In some embodiments, the percent composition of the unconjugated protein (e.g., unconjugated antibody) is between 25% and 65%, with the remainder comprising the conjugated protein. In some embodiments, the percent composition of the unconjugated protein (e.g., unconjugated antibody) is between 25% and 70%, with the remainder comprising the conjugated protein. In some embodiments, the percent composition of the unconjugated protein (e.g., unconjugated antibody) is between 25% and 75%, with the remainder comprising the conjugated protein. In some embodiments, the percent composition of the unconjugated protein (e.g., unconjugated antibody) is between 25% and 80%, with the remainder comprising the conjugated protein. In some embodiments, the percent composition of the unconjugated protein (e.g., unconjugated antibody) is between 25% and 85%, with the remainder comprising the conjugated protein. In some embodiments, the percent composition of the unconjugated protein (e.g., unconjugated antibody) is between 25% and 90%, with the remainder comprising the conjugated protein.

In some embodiments, the percent composition of the unconjugated protein or protein moiety (e.g., unconjugated antibody or unconjugated fusion construct) is 5%, with the remainder comprising the conjugated protein or protein moiety (e.g., conjugated antibody or conjugated fusion construct). In some embodiments, the percent composition of the unconjugated protein (e.g., unconjugated antibody) is 6%, with the remainder comprising the conjugated protein. In some embodiments, the percent composition of the unconjugated protein (e.g., unconjugated antibody) is 7%, with the remainder comprising the conjugated protein. In some embodiments, the percent composition of the unconjugated protein (e.g., unconjugated antibody) is 8%, with the remainder comprising the conjugated protein. In some embodiments, the percent composition of the unconjugated protein (e.g., unconjugated antibody) is 9%, with the remainder comprising the conjugated protein. In some embodiments, the percent composition of the unconjugated protein (e.g., unconjugated antibody) is 10%, with the remainder comprising the conjugated protein. In some embodiments, the percent composition of the unconjugated protein (e.g., unconjugated antibody) is 11%, with the remainder comprising the conjugated protein. In some embodiments, the percent composition of the unconjugated protein (e.g., unconjugated antibody) is 12%, with the remainder comprising the conjugated protein. In some embodiments, the percent composition of the unconjugated protein (e.g., unconjugated antibody) is 13%, with the remainder comprising the conjugated protein. In some embodiments, the percent composition of the unconjugated protein (e.g., unconjugated antibody) is 14%, with the remainder comprising the conjugated protein. In some embodiments, the percent composition of the unconjugated protein (e.g., unconjugated antibody) is 15%, with the remainder comprising the conjugated protein. In some embodiments, the percent composition of the unconjugated protein (e.g., unconjugated antibody) is 16%, with the remainder comprising the conjugated protein. In some embodiments, the percent composition of the unconjugated protein (e.g., unconjugated antibody) is 17%, with the remainder comprising the conjugated protein. In some embodiments, the percent composition of the unconjugated protein (e.g., unconjugated antibody) is 18%, with the remainder comprising the conjugated protein. In some embodiments, the percent composition of the unconjugated protein (e.g., unconjugated antibody) is 19%, with the remainder comprising the conjugated protein. In some embodiments, the percent composition of the unconjugated protein (e.g., unconjugated antibody) is 20%, with the remainder comprising the conjugated protein. In some embodiments, the percent composition of the unconjugated protein (e.g., unconjugated antibody) is 21%, with the remainder comprising the conjugated protein. In some embodiments, the percent composition of the unconjugated protein (e.g., unconjugated antibody) is 22%, with the remainder comprising the conjugated protein. In some embodiments, the percent composition of the unconjugated protein (e.g., unconjugated antibody) is 23%, with the remainder comprising the conjugated protein. In some embodiments, the percent composition of the unconjugated protein (e.g., unconjugated antibody) is 24%, with the remainder comprising the conjugated protein. In some embodiments, the percent composition of the unconjugated protein (e.g., unconjugated antibody) is 25%.

In some embodiments, the percent composition of the unconjugated protein or protein moiety (e.g., unconjugated antibody or unconjugated fusion construct) is 26%, with the remainder comprising the conjugated protein or protein moiety (e.g., conjugated antibody or conjugated fusion construct). In some embodiments, the percent composition of the unconjugated protein (e.g., unconjugated antibody) is 27%, with the remainder comprising the conjugated protein. In some embodiments, the percent composition of the unconjugated protein (e.g., unconjugated antibody) is 28%, with the remainder comprising the conjugated protein. In some embodiments, the percent composition of the unconjugated protein (e.g., unconjugated antibody) is 29%, with the remainder comprising the conjugated protein. In some embodiments, the percent composition of the unconjugated protein (e.g., unconjugated antibody) is 30%, with the remainder comprising the conjugated protein. In some embodiments, the percent composition of the unconjugated protein (e.g., unconjugated antibody) is 31%, with the remainder comprising the conjugated protein. In some embodiments, the percent composition of the unconjugated protein (e.g., unconjugated antibody) is 32%, with the remainder comprising the conjugated protein. In some embodiments, the percent composition of the unconjugated protein (e.g., unconjugated antibody) is 33%, with the remainder comprising the conjugated protein. In some embodiments, the percent composition of the unconjugated protein (e.g., unconjugated antibody) is 34%, with the remainder comprising the conjugated protein. In some embodiments, the percent composition of the unconjugated protein (e.g., unconjugated antibody) is 35%.

In some embodiments, the percent composition of the unconjugated protein or protein moiety (e.g., unconjugated antibody or unconjugated fusion construct) is 36%, with the remainder comprising the conjugated protein or protein moiety (e.g., conjugated antibody or fusion construct). In some embodiments, the percent composition of the unconjugated protein (e.g., unconjugated antibody) is 37%, with the remainder comprising the conjugated protein. In some embodiments, the percent composition of the unconjugated protein (e.g., unconjugated antibody) is 38%, with the remainder comprising the conjugated protein. In some embodiments, the percent composition of the unconjugated protein (e.g., unconjugated antibody) is 39%, with the remainder comprising the conjugated protein. In some embodiments, the percent composition of the unconjugated protein (e.g., unconjugated antibody) is 40%, with the remainder comprising the conjugated protein. In some embodiments, the percent composition of the unconjugated protein (e.g., unconjugated antibody) is 41%, with the remainder comprising the conjugated protein. In some embodiments, the percent composition of the unconjugated protein (e.g., unconjugated antibody) is 42%, with the remainder comprising the conjugated protein. In some embodiments, the percent composition of the unconjugated protein (e.g., unconjugated antibody) is 43%, with the remainder comprising the conjugated protein. In some embodiments, the percent composition of the unconjugated protein (e.g., unconjugated antibody) is 44%, with the remainder comprising the conjugated protein. In some embodiments, the percent composition of the unconjugated protein (e.g., unconjugated antibody) is 45%.

In some embodiments, the percent composition of the unconjugated protein or protein moiety (e.g., unconjugated antibody or unconjugated fusion construct) is 46%, with the remainder comprising the conjugated protein or protein moiety (e.g., conjugated antibody or conjugated fusion construct). In some embodiments, the percent composition of the unconjugated protein (e.g., unconjugated antibody) is 47%, with the remainder comprising the conjugated protein. In some embodiments, the percent composition of the unconjugated protein (e.g., unconjugated antibody) is 48%, with the remainder comprising the conjugated protein. In some embodiments, the percent composition of the unconjugated protein (e.g., unconjugated antibody) is 49%, with the remainder comprising the conjugated protein. In some embodiments, the percent composition of the unconjugated protein (e.g., unconjugated antibody) is 50%, with the remainder comprising the conjugated protein. In some embodiments, the percent composition of the unconjugated protein (e.g., unconjugated antibody) is 51%, with the remainder comprising the conjugated protein. In some embodiments, the percent composition of the unconjugated protein (e.g., unconjugated antibody) is 52%, with the remainder comprising the conjugated protein. In some embodiments, the percent composition of the unconjugated protein (e.g., unconjugated antibody) is 53%, with the remainder comprising the conjugated protein. In some embodiments, the percent composition of the unconjugated protein (e.g., unconjugated antibody) is 54%, with the remainder comprising the conjugated protein. In some embodiments, the percent composition of the unconjugated protein (e.g., unconjugated antibody) is 55%.

In some embodiments, the percent composition of the unconjugated protein or protein moiety (e.g., unconjugated antibody or unconjugated fusion construct) is 56%, with the remainder comprising the conjugated protein or protein moiety (e.g., conjugated antibody or conjugated fusion construct). In some embodiments, the percent composition of the unconjugated protein (e.g., unconjugated antibody) is 57%, with the remainder comprising the conjugated protein. In some embodiments, the percent composition of the unconjugated protein (e.g., unconjugated antibody) is 58%, with the remainder comprising the conjugated protein. In some embodiments, the percent composition of the unconjugated protein (e.g., unconjugated antibody) is 59%, with the remainder comprising the conjugated protein. In some embodiments, the percent composition of the unconjugated protein (e.g., unconjugated antibody) is 60%, with the remainder comprising the conjugated protein. In some embodiments, the percent composition of the unconjugated protein (e.g., unconjugated antibody) is 51%, with the remainder comprising the conjugated protein. In some embodiments, the percent composition of the unconjugated protein (e.g., unconjugated antibody) is 62%, with the remainder comprising the conjugated protein. In some embodiments, the percent composition of the unconjugated protein (e.g., unconjugated antibody) is 63%, with the remainder comprising the conjugated protein. In some embodiments, the percent composition of the unconjugated protein (e.g., unconjugated antibody) is 64%, with the remainder comprising the conjugated protein. In some embodiments, the percent composition of the unconjugated protein (e.g., unconjugated antibody) is 65%.

In some embodiments, the percent composition of the unconjugated protein or protein moiety (e.g., unconjugated antibody or unconjugated fusion construct) is 66%, with the remainder comprising the conjugated protein or protein moiety (e.g., conjugated antibody or conjugated fusion construct). In some embodiments, the percent composition of the unconjugated protein (e.g., unconjugated antibody) is 67%, with the remainder comprising the conjugated protein. In some embodiments, the percent composition of the unconjugated protein (e.g., unconjugated antibody) is 68%, with the remainder comprising the conjugated protein. In some embodiments, the percent composition of the unconjugated protein (e.g., unconjugated antibody) is 69%, with the remainder comprising the conjugated protein. In some embodiments, the percent composition of the unconjugated protein (e.g., unconjugated antibody) is 70%, with the remainder comprising the conjugated protein. In some embodiments, the percent composition of the unconjugated protein (e.g., unconjugated antibody) is 11%, with the remainder comprising the conjugated protein. In some embodiments, the percent composition of the unconjugated protein (e.g., unconjugated antibody) is 72%, with the remainder comprising the conjugated protein. In some embodiments, the percent composition of the unconjugated protein (e.g., unconjugated antibody) is 73%, with the remainder comprising the conjugated protein. In some embodiments, the percent composition of the unconjugated protein (e.g., unconjugated antibody) is 74%, with the remainder comprising the conjugated protein. In some embodiments, the percent composition of the unconjugated protein (e.g., unconjugated antibody) is 75%.

In some embodiments, the percent composition of the unconjugated protein or protein moiety (e.g., unconjugated antibody or unconjugated) is 76%, with the remainder comprising the conjugated protein or protein moiety (e.g., conjugated antibody or conjugated fusion construct). In some embodiments, the percent composition of the unconjugated protein (e.g., unconjugated antibody) is 77%, with the remainder comprising the conjugated protein. In some embodiments, the percent composition of the unconjugated protein (e.g., unconjugated antibody) is 78%, with the remainder comprising the conjugated protein. In some embodiments, the percent composition of the unconjugated protein (e.g., unconjugated antibody) is 79%, with the remainder comprising the conjugated protein. In some embodiments, the percent composition of the unconjugated protein (e.g., unconjugated antibody) is 80%, with the remainder comprising the conjugated protein. In some embodiments, the percent composition of the unconjugated protein (e.g., unconjugated antibody) is 81%, with the remainder comprising the conjugated protein. In some embodiments, the percent composition of the unconjugated protein (e.g., unconjugated antibody) is 82%, with the remainder comprising the conjugated protein. In some embodiments, the percent composition of the unconjugated protein (e.g., unconjugated antibody) is 83%, with the remainder comprising the conjugated protein. In some embodiments, the percent composition of the unconjugated protein (e.g., unconjugated antibody) is 84%, with the remainder comprising the conjugated protein. In some embodiments, the percent composition of the unconjugated protein (e.g., unconjugated antibody) is 85%.

In some embodiments, the percent composition of the unconjugated protein or protein moiety (e.g., unconjugated antibody or unconjugated fusion construct) is 86%, with the remainder comprising the conjugated protein or protein moiety. In some embodiments, the percent composition of the unconjugated protein (e.g., unconjugated antibody) is 87%, with the remainder comprising the conjugated protein. In some embodiments, the percent composition of the unconjugated protein (e.g., unconjugated antibody) is 88%, with the remainder comprising the conjugated protein. In some embodiments, the percent composition of the unconjugated protein (e.g., unconjugated antibody) is 89%, with the remainder comprising the conjugated protein. In some embodiments, the percent composition of the unconjugated protein (e.g., unconjugated antibody) is 90%.

In some embodiments, the ratio of the molecular weight of the unconjugated protein or protein moiety (e.g., second protein or protein moiety that is not conjugated to a phosphorylcholine-containing polymer) to the polymer in the formulation (or therapeutically acceptable composition) can be any suitable ratio. In some embodiments, the ratio is at most about 1:2, e.g., at most about 1:3, at most about 1:4, at most about 1:5, at most about 1:6, at most about 1:7, at most about 1:8, at most about 1:9 or at most about 1:10, or a ratio in a range defined by any two of the preceding values (e.g., 1:2-1:10, 1:3-1:8, 1:4-1:6). In some embodiments, the ratio is between about 1:4 and 1:6. In some embodiments, the ratio is about 1:5.33.

The protein conjugated to the phosphorylcholine-containing polymer and the unconjugated protein or protein moiety can each be any suitable protein. In some embodiments, the first protein (e.g., the protein conjugated to the phosphorylcholine-containing polymer) and the second protein (e.g., the protein that is not conjugated to a phosphorylcholine-containing polymer) have the same activity or function (e.g., bind the same epitope, inhibit the same target, catalyze the same reaction, etc.). In some embodiments, the first protein (e.g., the protein conjugated to the phosphorylcholine-containing polymer) and the second protein (e.g., the protein that is not conjugated to a phosphorylcholine-containing polymer) are the same protein. In some embodiments, the first protein (e.g., the protein conjugated to the phosphorylcholine-containing polymer) and the second protein (e.g., the protein that is not conjugated to a phosphorylcholine-containing polymer) are at least about 85%, at least about 90%, at least about 95%, at least about 97%, at least about 99%, or about 100%, or by a percentage in a range defined by any two of the preceding values (e.g., 85-100%, 90-99%, 90-95%, etc.) identical to each other in amino acid sequence. In some embodiments, where the first and second proteins each include two or more polypeptide chains, each of the corresponding polypeptide chains can independently have any of the noted sequence identity.

In some embodiments, the first protein (e.g., the protein conjugated to the phosphorylcholine-containing polymer) has a molecular weight (based on the protein portion) of about 50 kDa or more, e.g., about 75 kDa or more, about 100 kDa or more, about 125 kDa or more, about 150 kDa or more, about 175 kDa or more, about 200 kDa or more, about 250 kDa or more, about 300 kDa or more, or a molecular weight in a range defined by any two of the preceding values (e.g., 50-300 kDa, 100-300 kDa, 100-200 kDa, 150-250 kDa, etc.). In some embodiments, the first protein (e.g., the protein conjugated to the phosphorylcholine-containing polymer) has a molecular weight (based on the protein portion) of about 150 kDa. In some embodiments, first protein (e.g., the protein conjugated to the phosphorylcholine-containing polymer) has a molecular weight of about 200 kDa. In some embodiments, the second protein (e.g., the unconjugated protein or protein that is not conjugated to a phosphorylcholine-containing polymer) has a molecular weight of about 50 kDa or more, e.g., about 75 kDa or more, about 100 kDa or more, about 125 kDa or more, about 150 kDa or more, about 175 kDa or more, about 200 kDa or more, about 250 kDa or more, about 300 kDa or more, or a molecular weight in a range defined by any two of the preceding values (e.g., 50-300 kDa, 100-300 kDa, 100-200 kDa, 150-250 kDa, etc.). In some embodiments, the second protein (e.g., the unconjugated protein or protein that is not conjugated to a phosphorylcholine-containing polymer) has a molecular weight of about 150 kDa. In some embodiments, the second protein (e.g., the unconjugated protein or protein that is not conjugated to a phosphorylcholine-containing polymer) has a molecular weight of about 200 kDa.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is a therapeutic protein. In some embodiments, both the first protein (e.g., the protein conjugated to the phosphorylcholine-containing polymer) and the second protein (e.g., the unconjugated protein or protein that is not conjugated to a phosphorylcholine-containing polymer) are therapeutic proteins that are FDA approved as of May 2023. In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an antibody (e.g., therapeutic antibody). Any suitable antibody can be used in the formulations (or therapeutically acceptable compositions). In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is a fusion construct. Any suitable fusion construct can be used in the formulations (or therapeutically acceptable compositions). The antibody or fusion construct of any of the formulation or composition herein may or may not include a C-terminal lysine.

In some embodiments, sample analysis comprises evaluating samples with methods including but not limited to: SEC, SDS PAGE gel, multi angle light scattering (MALS), and imaged capillary isoelectric focusing (icIEF).

In some embodiments, prefiltration involves removal and discarding of insoluble particles such as dust, fiber and insoluble aggregates from the sample. In some embodiments, aggregates comprise insoluble clumps of antibody that have precipitated out of solution.

In some embodiments, a specific ionic strength of the solvent is used to allow partitioning of the mixed formulation (or therapeutically acceptable composition) in the CEX column. In some embodiments, the method allows analysis of the mixed formulation (or therapeutically acceptable composition) based on the differential charge and size variant of the constituents.

In some embodiments, the second unconjugated antibody or protein moiety remains bound to the CEX column after run 1. In some embodiments, the first conjugated antibody or protein moiety flows through the CEX column. In some embodiments, the first conjugated antibody or protein moiety is separated by the SEC column. In some embodiments, the second unconjugated antibody or protein moiety is eluted from the CEX column with a NaCl solution. In some embodiments, 100 uL of 1M NaCl is used to elute the second antibody or protein from the CEX column. In some embodiments, following elution, the second antibody is analyzed by a SEC column. In some embodiments, differences in charge and size between the first and second antibody or protein moiety permit differential partitioning in the CEX and SEC columns. In some embodiments, the differential partitioning of the first and second antibody or protein moiety in the CEX and SEC columns permits separation of the first and second antibody from the mixed formulation (or therapeutically acceptable composition).

In some embodiments, the polymer of the first antibody comprises a phosphorylcholine containing polymer. In some embodiments, the polymer is covalently bonded to the first antibody at a cysteine outside a variable region of the first antibody. In some embodiments, said cysteine replaces a non-cysteine amino acid that occurs in a same position in a sequence. In some embodiments, the first antibody comprises a light chain and heavy chain, said heavy chain comprising an Fc region. In some embodiments, the cysteine is in the Fc region of the heavy chain. In some embodiments, the sequence of the heavy chain comprises SEQ ID NO: 1 (with or without the C-terminal lysine). In some embodiment the sequence of the light chain comprises SEQ ID NO: 2;

In some embodiments, the first antibody conjugated to a phosphorylcholine containing polymer has the following structure:

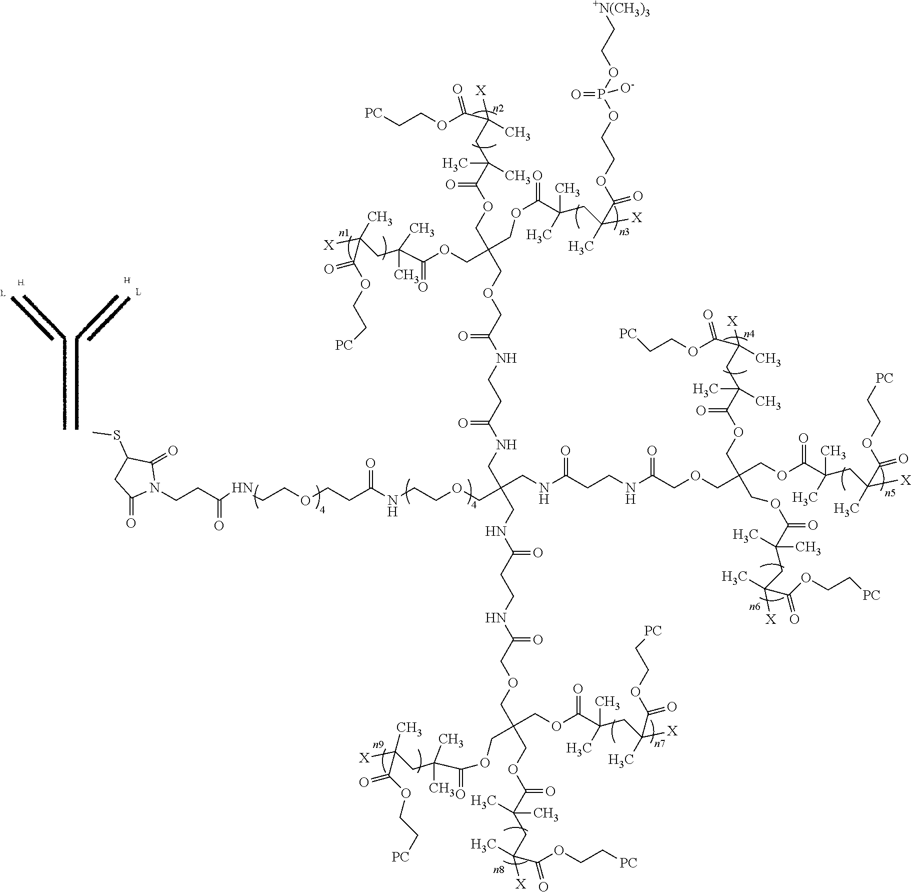

where X is a) —OR where R is —H, Methyl, ethyl, propyl, or isopropyl, b) —H, or c) a halide. In some embodiments, X is a) —OR where R is —H, Methyl, ethyl, propyl, or isopropyl, b) —H, c) any halogen, including —Br, —Cl, or —I, d) —SCN, or e) —NCS.

In some embodiments, the heavy chain of the first antibody is denoted by the letter H, and each light chain of the first antibody is denoted by the letter L. In some embodiments, the polymer is bonded to the first antibody through the sulfhydryl of a cysteine at position 449, as numbered in SEQ ID NO: 1, which bond is depicted on one of the heavy chains;

In some embodiments, PC is

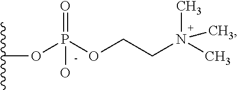

where the curvy line indicates the point of attachment to the rest of the polymer. In some embodiments, n1, n2, n3, n4, n5, n6, n7, n8 and n9 are the same or different such that the sum of n1, n2, n3, n4, n5, n6, n7, n8 and n9 is 2500 plus or minus 15%. In some embodiments, the sum of n1, n2, n3, n4, n5, n6, n7, n8 and n9 is about 1500 to about 3500 plus or minus about 10% to about 20%.

In some embodiments, the second antibody comprises a light chain and heavy chain, said heavy chain comprising an Fc region. In some embodiments, a cysteine is in the Fc region of the heavy chain. In some embodiments, the sequence of the heavy chain comprises SEQ ID NO: 1 (with or without the C-terminal lysine), and wherein the sequence of the light chain comprises SEQ ID NO: 2. In some embodiments, the heavy chain of the first and second antibodies comprises CDRH1: GYDFTHYGMN, CDRH2: WINTYTGEPTYAADFKR, and CDRH3: YPYYYGTSHWYFDV, and position 231 is T (via sequential counting as in SEQ ID NO: 1), and the light chain of the first and second antibodies comprises CDRL1: SASQDISNYLN (SEQ ID NO: 12), CDRL2: FTSSLHS (SEQ ID NO: 13), and CDRL3: QQYSTVPWT (SEQ ID NO: 14), and Kabat position 4 is L.

In some embodiments, the mixed formulation (or therapeutically acceptable composition) sample is loaded into a tandem CEX and SEC HPLC column arrangement. In some embodiments, the formulation (or therapeutically acceptable composition) sample travels sequentially through a prefilter, a CEX column, and a SEC HPLC column. In some embodiments, the CEX column comprises a Shodex SP825 column with internal diameter (i.d.)×length dimensions of 9.0×75 mm. In some embodiments, the SEC HPLC is a TSKgel G3000SWxl column with i.d.×length dimensions of 7.8×300 mm.

In some embodiments, the mixed formulation (or therapeutically acceptable composition) sample is loaded into a tandem CEX and SEC HPLC column arrangement. In some embodiments, the formulation (or therapeutically acceptable composition) sample travels sequentially through a prefilter, a CEX column, and a SEC HPLC column. In some embodiments, the CE column comprises an IEC ProPac WCX-10 column with internal diameter (i.d.)×length dimensions of 4.0 mm (about 0.16 in)×250 mm (about 9.84 in). In some embodiments, the SEC HPLC comprises a TSKgel G4000SWXL column with i.d.×length dimensions of 7.8 mm (about 0.31 in) ID×30 cm (about 11.81 in), 8 μm.

In some embodiments, the column arrangement comprises an ion exchange column (IEX), hydrophobic interaction (HIC), or affinity column and a SEC column. In some embodiments, the IEX column can be a CEX or anion exchange (AEX) column.

In some embodiments, the isocratic running conditions comprise a flow rate of 0.1-5 ml/min. In some embodiments, the isocratic running conditions comprise a flow rate of at 0.5-1 ml/min. In some embodiments, the flow rate can be any number between 0.1-5 ml/min. In some embodiments, the flowrate can be any number between 0.5-1 ml/min. In some embodiments, the buffer comprises 20 mM sodium acetate, pH 3-6. In some embodiments, the isocratic conditions are kept constant and uniform during a chromatography run.

In some embodiments, the buffer comprises 20 mM sodium acetate, pH 5. In some embodiments, the buffer comprises any amount between 50-500 mM NaCl. In some embodiments, the buffer comprises any amount between 90-120 mM NaCl. In some embodiments, the buffer comprises a conductivity of, or of about 2-32 mS/cm. In some embodiments, the buffer comprises a conductivity of, of about, or of at least 2, 3, 4, 5, 6, 7, 8, 9, 10, 12, 15, or 20 mS/cm, or of, of about, or of at most 32, 30, 28, 26, 24, 22, 20, 18, 16, 14, 12, 10, or 8 mS/cm, or a conductivity in range defined by any two of the preceding values (e.g., 2-32 mS/cm, 4-30 mS/cm, 5-15 mS/cm, 7-10 mS/cm, 8-10 mS/cm, etc.). In some embodiments, the buffer comprises a conductivity of, or of about 8.9 mS/cm.

In some embodiments, the method further comprises assessment for high molecular weight aggregates within the purified mixed formulation (or therapeutically acceptable composition) by methods comprising SEC profile analysis and SDS PAGE gel.

In some embodiments, the percent composition of the second antibody relative to the total protein mass weight concentration of the first antibody and the second antibody in the composition is any percentage between 0-93%, preferably between 0% and 50%, or between 5% and 93%. In some embodiments, the percent composition of the second antibody relative to the total protein mass weight concentration of the first antibody and the second antibody in the composition is any percentage between 0% and 50%, or between 5% and 50%. In some embodiments, the percent composition of the second antibody relative to the total protein mass weight concentration of the first antibody and the second antibody in the composition is any percentage between 0% and 25%, or between 5-25%.

In some embodiments, the mixed formulation (or therapeutically acceptable composition) sample comprises any amount between 50 μg and 1340 μg of protein.

In some embodiments, the method further comprises assessment of the percentage of the second antibody in the purified formulation (or therapeutically acceptable composition) by SEC profile analysis.

In some embodiments, the first run separates the first antibody from the second antibody and antibody aggregates.

In some embodiments, the tandem HPLC system further comprises SEC analysis on the second filtered mixed formulation (or therapeutically acceptable composition) of the first run performed by a second SEC HPLC column down-stream of the CEX column, wherein the second SEC HPLC column runs in parallel to the first SEC HPLC column. In some embodiments, the second HPLC column can be a Shodex SB-806 HQ or Shodex 806M-HQ column. In some embodiments, the second SEC HPLC column runs subsequent to the first SEC HPLC column instead of in parallel. In some embodiments, the first SEC HPLC column runs subsequent to the second SEC HPLC column instead of in parallel. In some embodiments, a switch valve allows for selection of the first or second SEC HPLC column.

In some embodiments, the step for elution of CEX bound free protein during the second run comprises a pulse of high salt at 0.5-5M NaCl. In some embodiments, the step for elution of CEX bound free protein during the second run comprises a pulse of high salt at 1M NaCl.

In some embodiments, during the second run, the fourth filtered mixed formulation (or therapeutically acceptable composition) is run through a prefiltration step to provide a fifth filtered mixed formulation (or therapeutically acceptable composition), wherein the fifth filtered mixed formulation (or therapeutically acceptable composition) is run through a CEX column to provide a six filtered mixed formulation (or therapeutically acceptable composition), wherein the sixth mixed formulation (or therapeutically acceptable composition) is run through a SEC HPLC column. In some embodiments, running the filtered mixed sample through a size exclusion exchange SEC HPLC column separates the second antibody from antibody aggregates.

In some embodiments, the effluent of the first and/or second SEC HPLC columns is connected to a to a multi angle light scattering (MALS) detector.

In some embodiments, additional valve is installed downstream of the first and/or second SEC column and the eluted free protein peaks are collected using a fraction collector for icIEF analysis. In some embodiments, the eluted free protein peaks are analyzed using any suitable analytical techniques, including without limitation, icIEF, mass spectrometry, fluorescence detection, photodiode array (PDA) detector.

In some embodiments, an apparatus is provided comprising a tandem series of CEX and SEC HPLC columns for use in analyzing a mixed formulation (or therapeutically acceptable composition) sample. In some embodiments, the mixed formulation (or therapeutically acceptable composition) sample comprises a first antibody that is an anti-VEGF-A antibody conjugated to a polymer and a second antibody that is an anti-VEGF-A antibody that is not conjugated to the polymer. In some embodiments, the analysis comprises a first run. In some embodiments, the mixed formulation (or therapeutically acceptable composition) is run through a prefiltration step to provide a first filtered mixed formulation (or therapeutically acceptable composition). In some embodiments, the first filtered mixed formulation (or therapeutically acceptable composition) is run through a cation exchange chromatography (CEX) column to provide a second filtered mixed formulation (or therapeutically acceptable composition). In some embodiments, the analysis comprises a second run. In some embodiments, during the second run, the protein bound to the CEX column is eluted to provide a third filtered mixed formulation. (or therapeutically acceptable composition) In some embodiments, the third filtered mixed formulation (or therapeutically acceptable composition) is run through a size exclusion exchange (SEC) high performance liquid chromatography (HPLC) column to provide a fourth filtered mixed formulation (or therapeutically acceptable composition).

In some embodiments, the first, second, or first and second antibody comprises a light chain and a heavy chain, said heavy chain comprising an Fc region. In some embodiments, a cysteine of the first, second, or first and second antibody is in the Fc region of the heavy chain.

In some embodiments, the first, second, or first and second antibody is an immunoglobulin G (IgG).

In some embodiments, the heavy chain of the first, second, or first and second antibody comprises: CDRH1: GYDFTHYGMN (SEQ ID NO: 9), CDRH2: WINTYTGEPTYAADFKR (SEQ ID NO: 10), and CDRH3: YPYYYGTSHWYFDV (SEQ ID NO: 11), and the light chain of the first, second, or first and second antibody comprises CDRL1: SASQDISNYLN (SEQ ID NO: 12), CDRL2: FTSSLHS (SEQ ID NO: 13), and CDRL3: QQYSTVPWT (SEQ ID NO: 14).

In some embodiments, the heavy chain isotype of the first, second, or first and second antibody is human IgG1. In some embodiments, the heavy chain constant domain of the first, second, or first and second antibody has one or more mutations relative to the constant domain of human IgG1 to modulate effector function.

In some embodiments, the mutations of the first, second, or first and second antibody are to one or more of the following amino acid positions (EU numbering): E233X, L234X, L235X, G236X, G237X, A327X, A330X, and P331X wherein X is any natural or unnatural amino acid. In some embodiments, the mutations of the first, second, or first and second antibody are selected from the group comprising (EU numbering): E233P, L234V, L234A, L235A, G237A, A327G, A330S, and P331S. In some embodiments, the mutations of the first, second, or first and second antibody comprise the following mutations: L234A, L235A, and G237A (EU numbering).

In some embodiments, the cysteine of the first, second, or first and second antibody is in the antibody heavy chain and is Q347C (EU numbering) or L443C (EU numbering).

In some embodiments, the sequence of the antibody heavy chain of the first, second, or first and second antibody is SEQ ID NO: 1 (with or without the C-terminal lysine) and the sequence of the light chain of the first, second, or first and second antibody is SEQ ID NO: 2.

In some embodiments, the cysteine of the first, second, or first and second antibody is L443C (EU numbering).

In some embodiments, the polymer of the antibody conjugate has three or more arms or is synthesized with an initiator comprising 3 or more polymer initiation sites.

In some embodiments, the polymer of the antibody conjugate has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites.

In some embodiments, the polymer of the antibody conjugate has a polydispersity value (PDI) of less than about 1.2.

In some embodiments, the percent composition of the second antibody relative to the total protein mass weight concentration of the first protein and the second protein in the composition is any of the following: about 7% second antibody, about 10% second antibody; about 12.5% second antibody; about 15% second antibody; about 17.5% second antibody; about 20% second antibody; and about 25% (B).

In some embodiments, provided herein is a composition comprising any two proteins that can be the same or different in function, wherein one is conjugated to a polymer and the other is not conjugated to the polymer (or conjugated to any polymer or conjugated to any effective amount of a polymer). The composition can be for the treatment of an eye disorder. In some embodiments, both of the proteins are therapeutics proteins for the treatment of an eye disorder. In some embodiments, one or both of the proteins are therapeutics, antibodies and/or therapeutic antibodies. In some embodiments, one antibody is conjugated to a polymer and the other antibody is not conjugated to a polymer. In some embodiments, the antibody can be synthesized. In some embodiments, the antibody can be a native sequence antibody. In some embodiments, the antibody can be a Fab fragment. In some embodiments, the antibody can be a Trap fragment. In some embodiments, the antibody can be a fusion protein such as a Trap-antibody fusion protein. In some embodiments, the antibody can be a peptide fragment. In some embodiments, a non-antibody scaffold protein can be used instead of an antibody.

In some embodiments, the composition comprises a mixture of two antibodies by mass weight concentration of 0.1% to 1% unconjugated antibody with the remainder comprising the conjugated antibody. In some embodiments, the composition comprises a mixture of two antibodies by mass weight concentration of 1% to 5% unconjugated antibody with the remainder comprising the conjugated antibody. In some embodiments, the composition comprises a mixture of two antibodies by mass weight concentration of 5% to 10% unconjugated antibody with the remainder comprising the conjugated antibody. In some embodiments, the composition comprises a mixture of the two antibodies by mass weight concentration of 5% to 15% unconjugated antibody with the remainder comprising the conjugated antibody. In some embodiments, the composition comprises a mixture of the two antibodies of the same mass weight concentration, of which 15% to 25% unconjugated antibody with the remainder comprising the conjugated antibody represents 15-25% of unconjugated antibody concentration in mass weight concentration (e.g. gram/liter) of the total protein concentration in the mixture. In some embodiments, the composition comprises a mixture of the two antibodies by mass weight concentration of 25% to 35% unconjugated antibody with the remainder comprising the conjugated antibody. In some embodiments, the composition comprises a mixture of the two antibodies by mass weight concentration of 35% to 45% unconjugated antibody with the remainder comprising the conjugated antibody. In some embodiments, the composition comprises a mixture of the two antibodies by mass weight concentration (mg/mL) of 45% to 50%, 50% to 55%, 55% to 60%, 60% to 65%, 65% to 70%, 70% to 75%, 75% to 80%, 80% to 85%, 85% to 90%, and 90% to 95%.

In some embodiments, a method of analyzing a mixed formulation (or therapeutically acceptable composition) sample is provided. In some embodiments, the method comprises providing a mixed formulation (or therapeutically acceptable composition) that comprises a fusion protein conjugated to a polymer a second protein that is not conjugated to the polymer. In some embodiments, the method further comprises a first run. In some embodiments, during the first run the mixed formulation (or therapeutically acceptable composition) is run through a prefiltration step to provide a first filtered mixed formulation (or therapeutically acceptable composition). In some embodiments, the first filtered mixed formulation (or therapeutically acceptable composition) is run through a cation exchange chromatography (CEX) column to provide a second filtered mixed formulation (or therapeutically acceptable composition). In some embodiments, the method further comprises a second run. In some embodiments, during the second run, the protein bound to the CEX column is eluted to provide a third filtered mixed formulation (or therapeutically acceptable composition). In some embodiments, the third filtered mixed formulation (or therapeutically acceptable composition) is run through a size exclusion exchange (SEC) high performance liquid chromatography (HPLC) column to provide a fourth filtered mixed formulation (or therapeutically acceptable composition). In some embodiments, a specific ionic strength of the solvent is used to allow partitioning of the mixed formulation (or therapeutically acceptable composition) in the CEX column. In some embodiments, the method allows analysis of the mixed formulation (or therapeutically acceptable composition) based on the differential charge and size variant of the constituents.

In some embodiments, an apparatus comprising a tandem series of CEX and SEC HPLC columns for use in purifying a mixed formulation (or therapeutically acceptable composition) sample is provided. In some embodiments, the sample comprises a first fusion protein conjugated to a polymer and a second protein that is not conjugated to the polymer. In some embodiments, the method further comprises a first run. In some embodiments, during the first run the mixed formulation (or therapeutically acceptable composition) is run through a prefiltration step to provide a first filtered mixed formulation (or therapeutically acceptable composition). In some embodiments, the first filtered mixed formulation (or therapeutically acceptable composition) is run through a cation exchange chromatography (CEX) column to provide a second filtered mixed formulation (or therapeutically acceptable composition). In some embodiments, the method further comprises a second run. In some embodiments, during the second run, the protein bound to the CEX column is eluted to provide a third filtered mixed formulation (or therapeutically acceptable composition). In some embodiments, the third filtered mixed formulation (or therapeutically acceptable composition) is run through a size exclusion exchange (SEC) high performance liquid chromatography (HPLC) column to provide a fourth filtered mixed formulation (or therapeutically acceptable composition).

In some embodiments, a method of analyzing a mixed formulation (or therapeutically acceptable composition) sample is provided. In some embodiments, the method comprises providing a mixed formulation (or therapeutically acceptable composition) that comprises a first antibody that is an anti-VEGF-A antibody conjugated to a polymer; and a second antibody that is an anti-VEGF-A antibody that is not conjugated to the polymer. In some embodiments, the method further comprises a first run. In some embodiments, during the first run the mixed formulation (or therapeutically acceptable composition) is run through a prefiltration step to provide a first filtered mixed formulation (or therapeutically acceptable composition). In some embodiments, the first filtered mixed formulation (or therapeutically acceptable composition) is run through a cation exchange chromatography (CEX) column to provide a second filtered mixed formulation (or therapeutically acceptable composition). In some embodiments, the method further comprises a second run. In some embodiments, during the second run, the protein bound to the CEX column is eluted to provide a third filtered mixed formulation (or therapeutically acceptable composition). In some embodiments, the third filtered mixed formulation (or therapeutically acceptable composition) is run through a size exclusion exchange (SEC) high performance liquid chromatography (HPLC) column to provide a fourth filtered mixed formulation (or therapeutically acceptable composition). In some embodiments, a specific ionic strength of the solvent is used to allow partitioning of the mixed formulation (or therapeutically acceptable composition) in the CEX column. In some embodiments, the method allows analysis of the mixed formulation (or therapeutically acceptable composition) based on the differential charge and size variant of the constituents.

In some embodiments, a method of analyzing a mixed formulation sample is provided. In some embodiments, the method comprises providing a mixed formulation that comprises a first antibody that is an anti-VEGF-A antibody conjugated to a polymer and a second antibody that is an anti-VEGF-A antibody that is not conjugated to the polymer. In some embodiments, the method further comprises loading the sample into an HPLC system where the HPLC pump is first connected to an auto-injector followed by a prefilter, which is then followed down-stream of two tandem serially connected columns. In some embodiments, the first column is a cation-exchanger column (CEX) and the second column down-stream of the CEX column is a size exclusion chromatography (SEC) column. In some embodiments, the method comprises a first run. In some embodiments, during the first run, the mixed formulation (or therapeutically acceptable composition) is injected and run through the tandem CEX-SEC system. In some embodiments, during the second run, a concentrated salt is injected to elute the bound fraction of the CEX column to be separated by the SEC column. In some embodiments, a specific ionic strength of the solvent is used to allow partitioning of the mixed formulation (or therapeutically acceptable composition) in the CEX column. In some embodiments, the method allows analysis of the mixed formulation (or therapeutically acceptable composition) based on the differential charge and size variant of the constituents.

In some embodiments, a method of analyzing a mixed formulation (or therapeutically acceptable composition) sample is provided. In some embodiments, the method comprises providing a mixed formulation (or therapeutically acceptable composition) that comprises a combination of two protein moieties. In some embodiments, the first protein moiety (A) is conjugated to a polymer, and the second protein (B) moiety is not conjugated to a polymer. In some embodiments, the method comprises loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column. In some embodiments, the method comprises a first run. In some embodiments, during the first run, the mixed formulation (or therapeutically acceptable composition) is injected and run through the tandem CEX-SEC system. In some embodiments, the method comprises a second run. In some embodiments, during the second run, a concentrated salt is injected to elute the bound fraction of the CEX column to be separated by the SEC column. In some embodiments, a specific ionic strength of the solvent is used to allow partitioning of the mixed formulation (or therapeutically acceptable composition) in the CEX column. In some embodiments, the method allows analysis of the mixed formulation (or therapeutically acceptable composition) based on the differential charge and size variant of the constituents.

In some embodiments, the mixed formulation (or therapeutically acceptable composition) sample comprises protein moiety A and protein moiety B.

In some embodiments, A and B can be the same protein moiety.

In some embodiments, A and B are different protein moieties.

In some embodiments, A and B are antibodies.

In some embodiments, A and/or B can be non-antibody proteins. In some embodiments, non-antibody proteins can include, but are not limited to cytokines, interferons, enzymes, regulatory proteins, growth factors, scaffolds, bone morphogenic proteins, blood factors, anticoagulants, hormones, cell signaling proteins, cell adhesion proteins, cell cycle proteins. In some embodiments, the non-antibody proteins are recombinantly produced.

In some embodiment the MW of protein A is greater or equal to the MW of protein B.

In some embodiments, the MW of protein B is greater or equal to the MW of protein A.

In some embodiments, the molar ratio range of the second protein or antibody not conjugated to a polymer to the first protein or antibody conjugated to a polymer is any number between 0.01 and 55. In some embodiments, the molar ratio range of the second protein or antibody not conjugated to a polymer to the first protein or antibody conjugated to a polymer is any number between 0.01 and 0.05 In some embodiments, the molar ratio range of the second protein or antibody not conjugated to a polymer to the first protein or antibody conjugated to a polymer is any number between 0.01 and 1 In some embodiments, the molar ratio range of the second protein or antibody not conjugated to a polymer to the first protein or antibody conjugated to a polymer is any number between 0.01 and 2 In some embodiments, the molar ratio range of the second protein or antibody not conjugated to a polymer to the first protein or antibody conjugated to a polymer is any number between 0.01 and 3 In some embodiments, the molar ratio range of the second protein or antibody not conjugated to a polymer to the first protein or antibody conjugated to a polymer is any number between 0.01 and 5 In some embodiments, the molar ratio range of the second protein or antibody not conjugated to a polymer to the first protein or antibody conjugated to a polymer is any number between 0.01 and 10 In some embodiments, the molar ratio range of the second protein or antibody not conjugated to a polymer to the first protein or antibody conjugated to a polymer is any number between 0.01 and 15. In some embodiments, the molar ratio range of the second protein or antibody not conjugated to a polymer to the first protein or antibody conjugated to a polymer is any number between 0.01 and 20 In some embodiments, the molar ratio range of the second protein or antibody not conjugated to a polymer to the first protein or antibody conjugated to a polymer is any number between 0.01 and 25 In some embodiments, the molar ratio range of the second protein or antibody not conjugated to a polymer to the first protein or antibody conjugated to a polymer is any number between 0.01 and 30 In some embodiments, the molar ratio range of the second protein or antibody not conjugated to a polymer to the first protein or antibody conjugated to a polymer is any number between 0.01 and 35 In some embodiments, the molar ratio range of the second protein or antibody not conjugated to a polymer to the first protein or antibody conjugated to a polymer is any number between 0.01 and 40. In some embodiments, the molar ratio range of the second protein or antibody not conjugated to a polymer to the first protein or antibody conjugated to a polymer is any number between 0.01 and 45 In some embodiments, the molar ratio range of the second protein or antibody not conjugated to a polymer to the first protein or antibody conjugated to a polymer is any number between 0.01 and 50.

In some embodiments, the molar ratio range of the second protein or antibody not conjugated to a polymer to the first protein or antibody conjugated to a polymer is any number between 0.01 and 16.

In some embodiments, the second protein or antibody is added directly to the drug product of the first antibody or formulation to generate the mixed formulation (or therapeutically acceptable composition).

In some embodiment, the second protein or antibody is added to the CEX pool with the first protein or antibody and undergoes ultrafiltration and diafiltration to generate the mixed formulation (or therapeutically acceptable composition).

In some embodiments, the second protein or antibody undergoes decapping and refolding prior to being added to the CEX pool.

In some embodiments, the decapped and refolded second protein or antibody undergoes further treatment with alkylation agents such as iodoacetamide (IAM) or N-ethylmaleimide (NEM) before being added to the CEX pool.

In some embodiments, the decapping process includes reducing one or more cysteines in a protein to form a decapped protein in a solution. In some embodiments, after reducing the one or more cysteines the decapped protein is reoxidized to restore at least one disulfide linkage in the reduced protein while ensuring that an engineered cysteine residue in the protein remains in a free thiol form to form a reoxidized decapped protein in the solution. In some embodiments, at least one excipient is then added to the solution. In some embodiments, the excipient reduces a polymer induced protein precipitation. In some embodiments, after the excipient is added, a polymer is added to the solution, which is conjugated to the reoxidized decapped protein at the engineered cysteine residue to form a conjugated protein.

In some embodiments, the mixed formulation (or therapeutically acceptable composition) further comprises an additional protein or antibody conjugated to a polymer.

In some embodiments, the polymer has 9 arms. In some embodiments, the polymer has a molecular weight of between about 600,000 to about 1,000,000 Da.

In some embodiments, the antibody is a complement factor D antibody. In some embodiments, the complement factor D antibody comprises a heavy chain and a light chain. In some embodiments, the heavy chain variable region (VH) comprises the amino acid sequence of any one of SEQ ID NOs: 15-47; and the light chain variable region (VL) comprises the amino acid sequence of any one of SEQ ID NOs: 48-80.

In some embodiments, the antibody comprises an antagonist IL-6 antibody fused to a VEGF trap. In some embodiments, the antibody comprises a heavy chain and a light chain. In some embodiments, the heavy chain variable region (VH) comprises the amino acid sequence of any one of SEQ ID NOs: 81-89; and the light chain variable region (VL) comprises the amino acid sequence of any one of SEQ ID NOs: 90-92.

In some embodiments, the heavy and light chains are selected from the group comprising:
(A) the heavy chain of SEQ ID NO:93 (with or without the C-terminal lysine), and the light chain of SEQ ID NO:94;
(B) the heavy chain of SEQ ID NO:95 (with or without the C-terminal lysine), and the light chain of SEQ ID NO:96;
(C) the heavy chain of SEQ ID NO:97 (with or without the C-terminal lysine), and the light chain of SEQ ID NO:98;
(D) the heavy chain of SEQ ID NO:99 (with or without the C-terminal lysine), and the light chain of SEQ ID NO:100;
(E) the heavy chain of SEQ ID NO:101 (with or without the C-terminal lysine), and the light chain of SEQ ID NO:102;
(F) the heavy chain of SEQ ID NO:103 (with or without the C-terminal lysine), and the light chain of SEQ ID NO:104;
(G) the heavy chain of SEQ ID NO: 105 (with or without the C-terminal lysine), and the light chain of SEQ ID NO: 106;
(H) the heavy chain of SEQ ID NO: 107 (with or without the C-terminal lysine), and the light chain of SEQ ID NO: 108;
(I) the heavy chain of SEQ ID NO:109 (with or without the C-terminal lysine), and the light chain of SEQ ID NO:110;
(J) the heavy chain of SEQ ID NO:111 (with or without the C-terminal lysine), and the light chain of SEQ ID NO:112;
(K) the heavy chain of SEQ ID NO:113 (with or without the C-terminal lysine), and the light chain of SEQ ID NO:114;
(L) the heavy chain of SEQ ID NO:115 (with or without the C-terminal lysine), and the light chain of SEQ ID NO:116;
(M) the heavy chain of SEQ ID NO:117 (with or without the C-terminal lysine), and the light chain of SEQ ID NO:118;
(N) the heavy chain of SEQ ID NO:119 (with or without the C-terminal lysine), and the light chain of SEQ ID NO:120;
(O) the heavy chain of SEQ ID NO: 121 (with or without the C-terminal lysine), and the light chain of SEQ ID NO:122;
(P) the heavy chain of SEQ ID NO: 123 (with or without the C-terminal lysine), and the light chain of SEQ ID NO:124;
(Q) the heavy chain of SEQ ID NO:125 (with or without the C-terminal lysine), and the light chain of SEQ ID NO: 126; and
(R) the heavy chain of SEQ ID NO:127 (with or without the C-terminal lysine), and the light chain of SEQ ID NO:128.

In some embodiments, the composition comprises OG1953 with variable amounts of unconjugated mAb and conjugated mAb. In some embodiments, one or more of the proteins of the composition is KSI-501, an antibody conjugate in which the protein is the trap antibody fusion of anti-VEGF and anti-IL-6 unconjugated protein (OG2072) and the conjugated protein (OG2074). In some embodiments, all the monomer pendant groups of the biopolymer of the composition are phosphorylcholine. In some embodiments, a quantity of desired small molecules is conjugated into the biopolymer of the composition. In some embodiments, the biopolymer of the compositions is conjugated and comprises copolymer characteristics that are distinct from the HEMA-PC monomer, in a desired ratio of unconjugated mAb to conjugated mAb.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an anti-VEGF-A antibody, a fusion construct comprising a VEGF Trap fused to the heavy chain of an anti-IL-6 antibody, an anti-IL-6 antibody, a fusion construct comprising a PDGFR extracellular trap fused to the heavy chain of an anti-VEGF-A antibody, a VEGF trap-Fc fusion protein, an anti-HTRA1 antibody, or an anti-complement factor D (CFD) antibody. In some embodiments, both of the first protein (e.g., the protein conjugated to the phosphorylcholine-containing polymer) and the second protein (e.g., the unconjugated protein or protein that is not conjugated to a phosphorylcholine-containing polymer) are an anti-VEGF-A antibody. In some embodiments, both of the first protein (e.g., the protein conjugated to the phosphorylcholine-containing polymer) and the second protein (e.g., the unconjugated protein or protein that is not conjugated to a phosphorylcholine-containing polymer) are a fusion construct comprising a VEGF Trap fused to the heavy chain of an anti-IL-6 antibody. In some embodiments, the anti-VEGF-A antibody is a full-length antibody. In some embodiments, the anti-VEGF-A antibody is or includes an anti-VEGF-A Fab fragment. In some embodiments, the anti-VEGF-A antibody is OG1950, e.g., as described herein and in US patent publication no. 2017/0190766, the entirety of which is incorporated herein by reference.

In some embodiments, the anti-VEGF-A antibody is selected from bevacizumab, ranibizumab, brolucizumab, or faricimab. In some embodiments, the VEGF trap-Fc fusion protein is aflibercept. In some embodiments, the fusion construct is OG2072, e.g., as described herein and in US patent publication no. 2019/0270806, the entirety of which is incorporated herein by reference.

In some embodiments, the mixed formulation comprises KSI-301. In some embodiments, KSI-301 comprises fusion protein OG1950 and its conjugated form with OG1802, OG1953.

In some embodiments, the KSI-301 Drug Product (DP) comprises 50 mM sodium acetate, 0.025% polysorbate 20, pH 4.5-5.6), 50 mg/mL (total protein concentration) containing 15.0-25.0% OG1950, 75.0-85.0% OG1953.

In some embodiments, the mixed formulation comprises KSI-501. In some embodiments, KSI-501 comprises fusion protein OG2072 and its conjugated form with OG1802, OG2074.

In some embodiments, the KSI-501 Drug Substance (DS) comprises 50-60 mM sodium acetate, polysorbate 20, sucrose, pH 4.5-5.6, 50 mg/mL (total protein concentration) containing 20-40% OG2072, 60-80% OG2074.

In some embodiments, first protein (e.g., the protein conjugated to the phosphorylcholine-containing polymer) can be any of the antibodies or fusion proteins described herein. In some embodiments, the second protein (e.g., the unconjugated protein or protein that is not conjugated to a phosphorylcholine-containing polymer) can be any of the antibodies or fusion proteins described herein.

In some embodiments, any of the methods or apparatuses described herein can be used to analyze, purify, and/or characterize a mixed formulation sample comprising (A) a first protein conjugated to a polymer, wherein the first protein can be any of the antibodies or fusion proteins described herein, and (B) a second protein that is not conjugated to the polymer, wherein, the second protein can be any of the antibodies or fusion proteins described herein.

In some embodiments, provided herein is a composition comprising any two proteins that can be the same or different in function, wherein one is conjugated to a polymer and the other is not conjugated to the polymer (or conjugated to any polymer or conjugated to any effective amount of a polymer). The composition can be for the treatment of an eye disorder. In some embodiments, both of the proteins are therapeutics proteins for the treatment of an eye disorder. In some embodiments, one or both of the proteins are therapeutics, antibodies and/or therapeutic antibodies. In some embodiments, one antibody is conjugated to a polymer and the other antibody is not conjugated to a polymer. In some embodiments, the antibody can be synthesized. In some embodiments, the antibody can be a native sequence antibody. In some embodiments, the antibody can be a Fab fragment. In some embodiments, the antibody can be a Trap fragment. In some embodiments, the antibody can be a fusion protein such as a Trap-antibody fusion protein. In some embodiments, the antibody can be a peptide fragment. In some embodiments, a non-antibody scaffold protein can be used instead of an antibody.

### Method of Treatment

In some embodiments, a method is presented for the treatment or prophylaxis of an ocular disease having the step of administering a therapeutic protein selected from the group comprising a protein, an antibody, or an anti-VEGF-A antibody (and conjugates thereof). In some embodiments, any one or more of the antibodies or antibody conjugates provided herein can be used as treatment and/or prophylaxis for an ocular disease. The method includes administering to the subject any one or more of the antibodies or antibody conjugates provided herein.

In some embodiments a method for treatment or prophylaxis of an ocular disease is provided. The method comprises administering an effective dose of any of the antibody and/or antibody conjugates described herein to a subject in need thereof. In some embodiments, the disease can be age-related macular degeneration (AMD) or diabetic macular edema (DME). In some embodiments, the disease can be wet AMD.

In some embodiments, the ocular disease is selected from one or more of the group comprising diabetic retinopathy, choroidal neovascularization (CNV), age-related macular degeneration (AMD), diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy. In some embodiments, the ocular disease is diabetic retinopathy.

In some embodiments, the antibody or antibody conjugate is administered no more frequently than once a month. In some embodiments, the antibody or conjugate thereof is administered two times per month or weekly. In some embodiments, the antibody or conjugate thereof is administered once every two months, once every three months, once every four months, once every five months, once every six months, once every seven months, once every eight months, once every nine months, once every ten months, once every eleven months, or once every twelve months.

In some embodiments, one or more of the compositions provided herein can allow for a reduction in the consequences of high treatment burdens from the use of intravitreal injection of anti-VEGF agents for the treatment of the wet (proliferative) form of age related macular degeneration (AMD). Real world outcomes for patients with wet AMD lag behind the clinical outcomes demonstrated in the phase 3 clinical studies such as the MARINA and ANCHOR studies with Lucentis® (ranibizumab) and the VIEW 1 and VIEW 2 studies with Eylea® (aflibercept). An anti-VEGF therapeutic with a longer ocular residence time such that it can be administered less frequently and therefore with a more patient-tolerable profile can bring real world outcomes closer to phase 3 clinical outcomes for more patients.

In some embodiments, compounds, including antibody conjugates and anti-VEGF-A antibodies described herein are used to treat patients who have background or nonproliferative diabetic retinopathy but have little or no vision impairment. In some embodiments, such patients are dosed less than once a month via intravitreal injection. In some embodiments, such patients are dosed six times a year. In some embodiments, such pateints are dosed no more than four times a year. In some embodiments, the patients are dose no more than three times a year. In some embodiments, the patients are dosed no more than twice a year. In some embodiments, the patients are dosed no more than once a year. In some embodiments, the subject receives the antibody or antibody conjugate via intravitreal injection.

The therapeutic proteins (e.g., proteins, protein conjugates, antibodies and antibody conjugates) described herein can be employed by expression of such polypeptides in vivo in a patient, i.e., gene therapy.

There are two major approaches to getting the nucleic acid (optionally contained in a vector) into the patient's cells: in vivo and ex vivo. For in vivo delivery the nucleic acid is injected directly into the patient, usually at the sites where the therapeutic protein is required, i.e., where biological activity of the therapeutic protein is needed. For ex vivo treatment, the patient's cells are removed, the nucleic acid is introduced into these isolated cells, and the modified cells are administered to the patient either directly or, for example, encapsulated within porous membranes that are implanted into the patient (see, e.g, U.S. Pat. Nos. 4,892,538 and 5,283,187). There are a variety of techniques available for introducing nucleic acids into viable cells. The techniques vary depending upon whether the nucleic acid is transferred into cultured cells in vitro, or transferred in vivo in the cells of the intended host. Techniques suitable for the transfer of nucleic acid into mammalian cells in vitro include the use of liposomes, electroporation, microinjection, transduction, cell fusion, DEAE-dextran, the calcium phosphate precipitation method, etc. Transduction involves the association of a replication-defective, recombinant viral (including retroviral) particle with a cellular receptor, followed by introduction of the nucleic acids contained by the particle into the cell. A commonly used vector for ex vivo delivery of the gene is a retrovirus.

In some embodiments, the in vivo nucleic acid transfer techniques include transfection with viral or non-viral vectors (such as adenovirus, lentivirus, Herpes simplex I virus, or adeno-associated virus (AAV)) and lipid-based systems (useful lipids for lipid-mediated transfer of the gene are, for example, DOTMA, DOPE, and DC-Chol; see, e.g., Tonkinison et al., Cancer Investigation, 14 (1): 54-65 (1996)). In some embodiments the vectors for use in gene therapy are viruses, which include adenoviruses, AAV, lentiviruses, or retroviruses. A viral vector such as a retroviral vector includes at least one transcriptional promoter/enhancer or locus-defining element(s), or other elements that control gene expression by other means such as alternate splicing, nuclear RNA export, or post-translational modification of messenger. In addition, a viral vector such as a retroviral vector includes a nucleic acid molecule that, when transcribed in the presence of a gene encoding the therapeutic protein, is operably linked thereto and acts as a translation initiation sequence. Such vector constructs also include a packaging signal, long terminal repeats (LTRs) or portions thereof, and positive and negative strand primer binding sites appropriate to the virus used (if these are not already present in the viral vector). In addition, such vector typically includes a signal sequence for secretion of the PRO polypeptide from a host cell in which it is placed. In some embodiments, the signal sequence for this purpose is a mammalian signal sequence. In some embodiments, the signal is the native signal sequence for the therapeutic protein. Optionally, the vector construct can also include a signal that directs polyadenylation, as well as one or more restriction sites and a translation termination sequence. By way of example, such vectors will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

In some situations, it is desirable to provide the nucleic acid source with an agent that targets the target cells, such as an antibody specific for a cell-surface membrane protein or the target cell, a ligand for a receptor on the target cell, etc. Where liposomes are employed, proteins that bind to a cell-surface membrane protein associated with endocytosis can be used for targeting and/or to facilitate uptake, e.g., capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262:4429-4432 (1987); and Wagner et al., Proc. Natl. Acad. Sci. USA, 87:3410-3414 (1990). For a review of the currently known gene marking and gene therapy protocols, see, Anderson et al., Science, 256:808-813 (1992). See also WO 93/25673 and the references cited therein.

Suitable gene therapy and methods for making retroviral particles and structural proteins can be found in, e.g., U.S. Pat. No. 5,681,746.

In some embodiments, a method for treatment or prophylaxis of an ocular disease in a mammal is presented in which a nucleic acid molecule that encodes a therapeutic protein selected from the group comprising an anti-VEGF-A antibody is administered.

In some embodiments, the heavy chain is that set forth in SEQ ID NO: 1 (with or without the C-terminal lysine) and the light chain is that set forth in SEQ ID NO: 2. In some embodiments, the nucleic acid molecule is administered via ex vivo gene therapy.

Therapeutic proteins can be incorporated into a pharmaceutical composition with a pharmaceutically acceptable excipient. Pharmaceutical compositions adapted for oral administration can be presented as discrete units such as capsules, as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which can be used include for example water, polyols and sugars. For the preparation of suspensions oils (e.g. vegetable oils) can be used to provide oil-in-water or water in oil suspensions.

Pharmaceutical compositions can be adapted for nasal administration wherein the excipient is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the excipient is a liquid, for administration as a nasal spray or as nasal drops, include aqueous or oil solutions of the active ingredient. Pharmaceutical compositions adapted for administration by inhalation include fine particle dusts or mists which can be generated by means of various types of metered dose pressurized aerosols, nebulizers or insufflators.

Pharmaceutical compositions adapted for parenteral administration include aqueous and non-aqueous sterile injection solution which can contain anti-oxidants, buffers, bacteriostats and solutes which render the formulation (or therapeutically acceptable composition) substantially isotonic with the blood of the intended recipient; and aqueous and non-aqueous sterile suspensions which can include suspending agents and thickening agents. Excipients which can be used for injectable solutions include water, alcohols, polyols, glycerine and vegetable oils, for example. The compositions can be presented in unit-dose or multi-dose containers, for example sealed ampoules and vials, and can be stored in a freeze-dried (lyophilized) condition requiring only the addition of the sterile liquid carried, for example water for injections, immediately prior to use. Extemporaneous injection solutions and suspensions can be prepared from sterile powders, granules and tablets. Pharmaceutical compositions can be substantially isotonic, implying an osmolality of about 250-400 mOsm/kg water.

The pharmaceutical compositions can contain preserving agents, solubilizing agents, stabilizing agents, wetting agents, emulsifiers, sweeteners, colorants, odorants, salts (substances can themselves be provided in the form of a pharmaceutically acceptable salt), buffers, coating agents or antioxidants. They can also contain therapeutically active agents in addition to the substance. The pharmaceutical compositions can be employed in combination with one or more pharmaceutically acceptable excipients. Such excipients can include, but are not limited to, saline, buffered saline (such as phosphate buffered saline), dextrose, liposomes, water, glycerol, ethanol and combinations thereof.

The antibodies and pharmaceutical compositions containing them can be administered in an effective regime for treating or prophylaxis of a patient's disease including, for instance, administration by oral, intravitreal, intravenous, subcutaneous, intramuscular, intraosseous, intranasal, topical, intraperitoneal, and intralesional administration. Parenteral infusions include intramuscular, intravenous, intraarterial, intraperitoneal, or subcutaneous administration or routes among others. In therapy or as a prophylactic, the active agent can be administered to an individual as an injectable composition, for example as a sterile aqueous dispersion. In some embodiments the agent is isotonic or substantially isotonic.

For administration to mammals, and particularly humans, it is expected that the dosage of the active agent is from 0.01 mg/kg body weight, typically around 1 mg/kg. The physician can determine the actual dosage most suitable for an individual which depends on factors including the age, weight, sex and response of the individual, the disease or disorder being treated and the age and condition of the individual being treated. The above dosages are exemplary of the average case. There can, of course, be instances where higher or lower dosages are merited. In some embodiments, the dosage can be 0.5 to 20 mg/eye, e.g., 1, 2, 3, 4, 5, 6, 7, 8, 9, 10, 11, 12, 13, 14, 15, 16, 17, 18, or 19 mg.

This dosage can be repeated as often as appropriate (e.g., weekly, fortnightly, monthly, once every two months, quarterly, twice a year, yearly). If side effects develop the amount and/or frequency of the dosage can be reduced, in accordance with normal clinical practice. In one embodiment, the pharmaceutical composition can be administered once every one to thirty days. In one embodiment, the pharmaceutical composition can be administered twice every thirty days. In one embodiment, the pharmaceutical composition can be administered once a week.

The antibodies and pharmaceutical compositions can be employed alone or in conjunction with other compounds, such as therapeutic compounds or molecules, e.g. anti-inflammatory drugs, analgesics or antibiotics. Such administration with other compounds can be simultaneous, separate or sequential. The components can be prepared in the form of a kit which can comprise instructions as appropriate.

The antibodies and pharmaceutical compositions disclosed herein can be used for treatment or prophylaxis of disease, particularly the ocular diseases or conditions described herein.

So used, the conjugates are typically formulated for and administered by ocular, intraocular, and/or intravitreal injection, and/or juxtascleral injection, and/or subretinal injection and/or subtenon injection, and/or superchoroidal injection and/or subconjunctival and/or topical administration in the form of eye drops and/or ointment. Such antibodies and compositions can be delivered by a variety of methods, e.g. intravitreally as a device and/or a depot that allows for slow release of the compound into the vitreous, including those described in references such as Intraocular Drug Delivery, Jaffe, Ashton, and Pearson, editors, Taylor & Francis (March 2006). In one example, a device can be in the form of a minipump and/or a matrix and/or a passive diffusion system and/or encapsulated cells that release the compound for a prolonged period of time (Intraocular Drug Delivery, Jaffe, Ashton, and Pearson, editors, Taylor & Francis (March 2006).

Formulations (or therapeutically acceptable composition) for ocular, intraocular or intravitreal administration can be prepared by methods and using ingredients known in the art. A main requirement for efficient treatment is proper penetration through the eye. Unlike diseases of the front of the eye, where drugs can be delivered topically, retinal diseases require a more site-specific approach. Eye drops and ointments rarely penetrate the back of the eye, and the blood-ocular barrier hinders penetration of systemically administered drugs into ocular tissue. Accordingly, usually the method of choice for drug delivery to treat retinal disease, such as AMD and CNV, is direct intravitreal injection. Intravitrial injections are usually repeated at intervals which depend on the patient's condition, and the properties and half-life of the drug delivered.

Therapeutic antibodies and related conjugates generally are placed into a container having a sterile access port, for example, an intravenous solution bag or vial having a stopper pierceable by a hypodermic injection needle. Such compositions can also be supplied in the form of pre-filled syringes.

A “stable” formulation is one in which the protein or protein conjugated to a polymer of other half-life extending moiety therein essentially retains its physical stability and/or chemical stability and/or biological activity upon storage. By “stable” is also meant a formulation which exhibits little or no signs of instability, including aggregation and/or deamidation. For example, the formulations provided can remain stable for at least two years, when stored as indicated at a temperature of 5-8° C.

Various analytical techniques for measuring protein stability are available in the art and are reviewed in Peptide and Protein Drug Delivery, 247-301 (Vincent Lee ed., New York, N.Y., 1991) and Jones, 1993 Adv. Drug Delivery Rev. 10:29-90, for examples. Stability can be measured at a selected temperature for a selected time period. In some embodiments the storage of the formulations is stable for at least 6 months, 12 months, 12-18 months, or for 2 or more years.

A protein, such as an antibody or fragment thereof, “retains its physical stability” in a pharmaceutical formulation if it shows no signs of aggregation, precipitation, deamidation and/or denaturation upon visual examination of color and/or clarity, or as measured by UV light scattering or by size exclusion chromatography.

A protein “retains its chemical stability” in a pharmaceutical formulation, if the chemical stability at a given time is such that the protein is considered to still retain its biological activity. Chemical stability can be assessed by detecting and quantifying chemically altered forms of the protein. Chemical alteration can involve size modification (e.g., clipping), which can be evaluated using size exclusion chromatography, SDS-PAGE and/or matrix-assisted laser desorption ionization/time-of-flight mass spectrometry (MALDI/TOF MS), for examples. Other types of chemical alteration include charge alteration (e.g., occurring as a result of deamidation), which can be evaluated by ion-exchange chromatography, for example. An antibody “retains its biological activity” in a pharmaceutical formulation, if the biological activity of the antibody at a given time is within about 10% (within the errors of the assay) of the biological activity exhibited at the time the pharmaceutical formulation was prepared as determined in an antigen binding assay, for example.

A protein-polymer conjugate “retains its chemical stability” the chemical bond between the protein and the polymer is maintained intact, e.g., it is not hydrolyzed or otherwise disrupted. The protein part of the conjugate retains its chemical stability as described above.

By “isotonic” is meant that the formulation of interest has essentially the same osmotic pressure as human blood or the vitreous for intravitreal injections. Isotonic formulations will generally have an osmotic pressure from about 250 to 400 mOsm. Isotonicity can be measured using a vapor pressure or ice-freezing type osmometer, for example.

As used herein, “buffer” refers to a buffered solution that resists changes in pH by the action of its acid-base conjugate components. In some embodiments, the buffer has a pH from about 3.0 to about 8.0; for example, from about 4.5 to 8; or about pH 6 to about 7.5; or about 6.0 to about 7.0, or about 6.5-7.0, or about pH 7.0 to about 7.5; or about 7.1 to about 7.4. A pH of any point in between the above ranges is also contemplated.

In some embodiments, “PBS” phosphate buffered saline, Tris based buffers and histidine based buffers are used. In some embodiments, an acetate buffer (e.g., sodium acetate buffer at pH 4.0-5.0, e.g., at about pH 4.5, or about 5.0) is used. In some embodiments, a composition of formulation of the present disclosure includes a surfactant. In some embodiments, a suitable surfactant includes, without limitation, a polysorbate. In some embodiments, the polysorbate is polysorbate 20, polysorbate 40, or polysorbate 80. In some embodiments, the polysorbate is polysorbate 20. In some embodiments, the polysorbate is present in the composition or formulation at a concentration of about 0.001%-0.1% (w/w), or about 0.01%-0.05%, or about 0.02%-0.03%. In some embodiments, the polysorbate is present in the composition or formulation at a concentration of, of about, or of at least 0.001, 0.002, 0.005, 0.01, 0.02, or 0.025% (w/w) or at, about, or of at most 0.1, 0.09, 0.08, 0.07, 0.06, 0.05, 0.04, 0.03, or 0.025% (w/w), or a percentage (w/w) in a range defined by any two of the preceding values.

In some embodiments, the PBS buffer is made up of at least Na₂HPO₄, KH₂PO₄ and NaCl adjusted so as to provide the appropriate pH. In some embodiments, the buffer can contain other pharmaceutical excipients such as KCl and other salts, detergents and/or preservatives so as to provide a stable storage solution.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

In some embodiments, formulations, to be safe for human use or for animal testing, should have sufficiently low levels of endotoxin. “Endotoxin” is lipopolysaccharide (LPS) derived from the cell membrane of Gram-negative bacteria. Endotoxin is composed of a hydrophilic polysaccharide moiety covalently linked to a hydrophobic lipid moiety (lipid A). Raetz C R, Ulevitch R J, Wright S D, Sibley C H, Ding A, Nathan C F. 1991. Gram-negative endotoxin: an extraordinary lipid with profound effects on eukaryotic signal transduction. FASEB J. 5 (12): 2652-2660. Lipid A is responsible for most of the biological activities of endotoxin, i.e., its toxicity. Endotoxins are shed in large amount upon bacterial cell death as well as during growth and division. They are highly heat-stable and are not destroyed under regular sterilizing conditions. Extreme treatments with heat or pH, e.g., 180-250° C. and over 0.1 M of acid or base must be used (Petsch D, Anspach F. 2000. Endotoxin removal from protein solutions. J Biotechnol. 76:97-119). Such conditions of course would be highly detrimental to biological drugs.

In the biotech and pharmaceutical industries, it is possible to find endotoxin during both production processes and in final products. As bacteria can grow in nutrient poor media, including water, saline and buffers, endotoxins are prevalent unless precautions are taken. Endotoxin injection into an animal or human causes a wide variety of pathophysiological effects, including endotoxin shock, tissue injury and even death. Ogikubo Y, Ogikubo Y, Norimatsu M, Noda K, Takahashi J, Inotsume M, Tsuchiya M, Tamura Y. 2004. Evaluation of the bacterial endotoxin test for quantifications of endotoxin contamination of porcine vaccines. Biologics 32:88-93.

Pyrogenic reactions and shock are induced in mammals upon intravenous injection of endotoxin at low concentrations (1 ng/ml) (Fiske J M, Ross A, VanDerMeid R K, McMichael J C, Arumugham. 2001. Method for reducing endotoxin in Moraxella catarrhalis UspA2 protein preparations. J Chrom B. 753:269-278). The maximum level of endotoxin for intravenous applications of pharmaceutical and biologic product is set to 5 endotoxin units (EU) per kg of body weight per hour by all pharmacopoeias (Daneshiam M, Guenther A, Wendel A, Hartung T, Von Aulock S. 2006. In vitro pyrogen test for toxic or immunomodulatory drugs. J Immunol Method 313:169-175). EU is a measurement of the biological activity of an endotoxin. For example, 100 pg of the standard endotoxin EC-5 and 120 μg of endotoxin from Escherichia coli O111:B4 have activity of 1 EU (Hirayama C, Sakata M. 2002. Chromatographic removal of endotoxin from protein solutions by polymer particles. J Chrom B 781:419-432). Meeting this threshold level has always been a challenge in biological research and pharmaceutical industry (Berthold W, Walter J. 1994. Protein Purification: Aspects of Processes for Pharmaceutical Products. Biologicals 22:135-150; Petsch D, Anspach F B. 2000. Endotoxin removal from protein solutions. J Biotech 76:97-119).

The presence of endotoxin in drugs to be delivered via intravitreal injection is of particular concern. Intravitreal injection of drug (penicillin) was first performed in 1945 by Rycroft. Rycroft B W. 1945. Penicillin and the control of deep intra-ocular infection. British J Ophthalmol 29 (2): 57-87. The vitreous is a chamber where high level of drug can be introduced and maintained for relatively long periods of time. The concentration of drug that can be achieved via intravitreal injection far exceeds what can be generated by topical administration or by systemic administration (e.g. intravenous).

One of the most dangerous complications potentially arising from intravitreal injections is endophthalmitis. Endophthalmitis falls into two classes: infectious and sterile. Infectious endophthalmitis is generally cause by bacteria, fungi or parasites. The symptoms of infectious endophthalmitis include severe pain, loss of vision, and redness of the conjunctiva and the underlying episclera. Infectious endophthalmitis requires urgent diagnosis and treatment. Possible treatments include intravitreal injection of antibiotics and pars plana vitrectomy in some cases. Enucleation can be called for to remove a blind and painful eye. See, e.g., Christy N E, Sommer A. 1979. Antibiotic prophylaxis of postoperative endophthalmitis. Ann Ophthalmol 11 (8): 1261-1265.

Sterile endophthalmitis in contrast does not involve an infectious agent and can be defined as the acute intraocular inflammation of the vitreous cavity that resolves without the need of intravitreal antibiotics and/or vitreoretinal surgery. If a vitreous microbiological study has been done, it needs to be negative culture proven to sustain a diagnosis of sterile endophthalmitis. Marticorena J, Romano V, Gomez-Ulla F. 2012 “Sterile Endophthalmitis after Intravitreal Injections” Med Inflam. 928123.

It has been observed that intravitreal injection of biological drugs contaminated with endotoxin can result in sterile endophthalmitis. Marticorena, et al. Bevacizumab (Avastin) is approved by the Food and Drug Administration for the treatment of glioblastoma and of metastatic colorectal cancer, advanced nonsquamous non-small-cell lung cancer and metastatic kidney cancer. Bevacizumab is also widely used off label as a treatment for wet AMD. Bevacizumab comes from the manufacturer as a 100 mg/4 ml. This solution cannot be directly used for intravitreal injection and should be compounded by a pharmacist. Clusters of sterile endophthalmitis have been observed and are theorized to be cause by inadvertent contamination of bevacizumab by endotoxin by the compounding pharmacist.

Given the dire clinical results of intravitreal injection of endotoxin, the total amount of endotoxin that can be given to a patient via intravitreal dosing is highly limited. In some embodiments, a solution having an antibody or antibody-conjugate is provided having an endotoxin level that does not exceed 5.0 EU/ml. In some embodiments, the endotoxin level does not exceed 1.0 EU/ml. In some embodiments, the endotoxin level does not exceed 0.5 EU/ml. In some embodiments, the endotoxin level does not exceed 0.2 EU/ml. In some embodiments, the endotoxin level does not exceed 2, 1, 0.5, 0.2, 0.1, 0.09, 0.08, 0.07, 0.06, 0.05, 0.04, 0.03, 0.02 or 0.01 EU/ml.

Two commonly used FDA-approved tests for the presence of endotoxin are the rabbit pyrogen test and Limulus Amoebodyte Lysate (LAL) assay (Hoffman S, et al. 2005. International validation of novel pyrogen tests based on human monocytoid cells J. Immunol. Methods 298:161-173; Ding J L, Ho B A. 2001. New era in pyrogen testing. Biotech. 19:277-281). The rabbit pyrogen test was developed in the 1920s and involves monitoring the temperature rise in a rabbit injected with a test solution. However, use of the rabbit pyrogen test has greatly diminished over the years due to expense and long turnaround time. Much more common is the LAL test. LAL is derived from the blood of a horseshoe crab and clots upon exposure to endotoxin.

One of the simplest LAL assays is the LAL gel-clot assay. Essentially, the LAL clotting assay is combined with a serial dilution of the sample in question. Formation of the gel is proportional to the amount of endotoxin in the sample. Serial dilutions are prepared from the sample and each dilution assayed for its ability to form LAL gel. At some point a negative reaction is contained. The amount of endotoxin in the original sample can be estimated from the dilution assay.

Other LAL tests have also been developed, including the turbidimetric LAL assay (Ong K G, Lelan J M, Zeng K F, Barrett G, Aourob M, Grimes C A. 2006. A rapid highly-sensitive endotoxin detection system. Biosensors and Bioelectronics 21:2270-2274) and the chromogenic LAL assay (Haishima Y, Hasegawa C, Yagami T, Tsuchiya T, Matsuda R, Hayashi Y. 2003. Estimation of uncertainty in kinetic-colorimetric assay of bacterial endotoxins. J Pharm Biomed Analysis. 32:495-503). The turbidimetric and chromogenic assays are much more sensitive and quantitative than the simple gel-clot dilution assay.

In some embodiments a method of reducing the amount of endotoxin in a composition having an antibody disclosed herein is provided. The method having the steps of contacting the composition with an affinity chromatography resin that binds to the antibody; eluting the antibody from the affinity chromatography resin to form an affinity chromatography eluent having the antagonist; contacting the affinity chromatography eluent with an ion-exchange resin that binds the antibody; and eluting the antibody from the ion-exchange resin, wherein the antibody eluted from the ion-exchange resin is substantially free from endotoxin.

The above method for reducing the amount of endotoxin, or other method or process recited herein, can be performed in the order described in the steps above or it can optionally be performed by varying the order of the steps or even repeating one or more of the steps. In one embodiment, the method of reducing the amount of endotoxin in a composition is performed in the order of the described steps. In some embodiments, the affinity chromatography resin contacting, washing and eluting steps are repeated in the same order more than one time before contacting the affinity chromatography eluent with the ion exchange resin. The method can also include a filtering step using, for example, a 0.1 micron, 0.22 micron, or 0.44 micron filter, that can be performed on either one or more of the eluents removed after each resin binding step.

In certain instances, the steps of contacting the composition with affinity chromatography resin, washing and eluting the antibody from the affinity chromatography resin can be repeated more than one time before contacting the first eluent with an ion-exchange resin. In one embodiment, the affinity chromatography resin comprises a recombinant Protein A (“rProteinA”) resin. One example of a suitable recombinant Protein A resin is rProteinA Sepharose FF® resin (Amersham, Piscataway, N.J.). In another embodiment, a suitable affinity chromatography resin would comprise a protein G chromatography resin. In other embodiments, a suitable affinity chromatography resin comprises a mixed Protein A/Protein G resin. In other embodiments, a suitable affinity chromatography resin comprises a hydrophobic charge induction resin that comprises a 4-mercaptoethylpyridine ligand such as a MEP HyperCel® resin (BioSepra, Cergy, Saint Christophe, France).

In some embodiments, the ion exchange resin comprises an anion-exchange resin. As will be known by the person skilled in the art, ion exchangers can be based on various materials with respect to the matrix as well as to the attached charged groups. For example, the following matrices can be used, in which the materials mentioned can be more or less cross-linked: POROS XS (Thermo Fisher Scientific, Waltham, MA), agarose based (such as SP35 Praesto Jetted SP35 IEX (Purolite, King of Prussia, PA, SmartSep (YMC, Shimogyo, Japan), cellulose based (such as DEAE Sephacel®), dextran based (such as Sephadex®), silica based and synthetic polymer based. For the anion exchange resin, the charged groups, which are covalently attached to the matrix, may, for example, be diethylaminoethyl, quaternary aminoethyl, and/or quaternary ammonium. In some embodiments the anion-exchange resin comprises a quaternary amine group. An exemplary anion-exchange resin that has a quaternary amine group is a POROS XQ resin (Thermo Fisher Scientific, Waltham, MA).

In other aspects, if the endotoxin levels are higher than desired after subjecting the composition to the aforementioned anion-exchange chromatography step, the composition can in the alternative be subjected to a cation exchange resin. In some embodiments, any endotoxin in the composition should have a differential binding to the ion-exchange resin than the protein in question to allow purification of the protein from the endotoxin. In this regard, endotoxin is negatively charged and will generally bind to an anion exchange resin. If both the protein and the endotoxin bind to the anion exchange resin, purification of one from the other can be effectuated by using a salt gradient to elute the two into different fractions. The relative binding of the protein to a particular resin can also be effected by changing the pH of the buffer relative to the pI of the protein. In some embodiments, cation-exchange chromatography is the sole ion-exchange chromatography employed.

In some embodiments, if the endotoxin levels are too high after the anion exchange resin, the composition can be further subjected to a second ion-exchange step, for example, by contacting the compositions with a cation exchange resin and followed by a wash step, then elution from the ion-exchange resin. In some embodiments, the cation exchange resin comprises a sulfonic group for binding. Exemplary cation exchange resins are SP Sepharose® resin FF (Amersham, Piscataway, N.J.) Poros XS (CEX) (Life Technology, Grand Island, New York).

In some embodiments, after the solution of antibody protein is produced having the specified level of endotoxin, there are a number of steps prior to final formulation of the protein. In some embodiments, a half-life extending moiety is conjugated to the protein. The conjugate is then formulated into a final drug formulation (or therapeutically acceptable composition) which is injected into the patients. In some embodiments, the conjugate is again purified on an ion-exchange resin which can be a cation-exchange resin. In other embodiments, the protein is formulated. In all cases, normal laboratory procedures should be employed to prevent the introduction of endotoxin contaminants into the protein sample or into the protein-polymer conjugate.

In some embodiments, a method or apparatus for analysis of a mixed formulation (or therapeutically acceptable composition) is provided comprising a tandem HPLC system. In some embodiments, the tandem HPLC system can comprise orthogonally combining cation exchange column (CEX) and size exclusion column (SEC) columns. In some embodiments, the tandem HPLC system can comprise a switch valve. In some embodiments, the switch valve can be downstream of the CEX column. In some embodiments, the switch valve is a multi-switch valve. In some embodiments, the switch valve is a 2-way switch valve. In some embodiments, the switch valve is a 3-way switch valve. In some embodiments, the switch valve can direct the effluent of the CEX column to multiple possible targets. In some embodiments the targets can be 1) a bypass loop, 2) a first SEC column, and 3) a second SEC column. In some embodiments, the first SEC column can be a Shodex_806M HQ column. In some embodiments, the second SEC column can be a TSK G3000 column. In some embodiments, separate acquisition methods can be performed for each of 1-3).

In some embodiments, the tandem HPLC system comprises multi-valve multi-SEC column operation. In some embodiments, the multi-valve multi-SEC column operation can be a parallel run. In some embodiments, the parallel run can comprise at least two synchronized HPLC systems or modern high end HPLC system with splitter functions. In some embodiments, tandem HPLC system can be operated in sequential mode with the first SEC run followed by second SEC run in either separate or continuous mode.

In some embodiments, the IEX can be a CEX. In some embodiments, the CEX can be a Shodex SP-825 column.

In some embodiments, the CEX can be a IEC ProPac WCX-10, 4.0 mm ID×250 mm L. In some embodiments, the SEC is a TSKgel G4000SW_{XL} column with ID×length dimensions of 7.8 mm ID×30 cm, 8 μm.

In some embodiments, the tandem HPLC system can be used to define the composition characteristics of a mixed formulation (or therapeutically acceptable composition). In some embodiments, the tandem HPLC system can be used to measure the stability of the components of a mixed formulation (or therapeutically acceptable composition) over time. In some embodiments, the tandem HPLC system can be used to observe and define degradation products and/or aggregates in a mixed formulation (or therapeutically acceptable composition).

In some embodiments, the tandem HPLC system can be further linked to a Multi-angle light scattering detector (MALS). In some embodiments, the tandem HPLC system can be further linked to a UV detector flow cell. In some embodiments, the tandem HPLC system further can comprise a column heater. In some embodiments, an additional valve can be installed downstream of the first and/or second SEC column and the eluted free protein peaks can be collected using a fraction collector for icIEF analysis.

In some embodiments, the mixed formulation (or therapeutically acceptable composition) can comprise conjugated protein, unconjugated protein, aggregated unconjugated protein, insoluble components, and a buffer. In some embodiments, the conjugated protein can be OG1953 fusion protein conjugate. In some embodiments the mixed formulation (or therapeutically acceptable composition) can be KSI-301DP. In some embodiments, the unconjugated protein can be OG1950 fusion protein. In some embodiments, insoluble components can be removed via the prefiltration step of the tandem HPLC method. In some embodiments, the unconjugated protein can bind to the CEX column. In some embodiments, the conjugated protein flows through the CEX column. In some embodiments, the components of the unbound fraction can be determined by SEC analysis. In some embodiments, the components of the unbound fraction can comprise conjugated protein and some unconjugated protein. In some embodiments, the unconjugated protein bound to the CEX column can be eluted by a pulse elution. In some embodiments, the pulse elution can be performed with NaCl. In some embodiments, the pulse elution can be performed with any concentration between 0.5-5M NaCl. In some embodiments, the pulse elution can be performed with 1M NaCl. In some embodiments, the components of the eluted fraction can be determined by SEC analysis. In some embodiments, the components of the eluted fraction can comprise unconjugated protein, aggregated unconjugated protein, and some conjugated protein.

In some embodiments, the mixed formulation (or therapeutically acceptable composition) can comprise a conjugated protein, unconjugated protein, aggregated unconjugated protein, insoluble components, and a buffer. In some embodiments, the conjugated protein can be OG2074 fusion protein conjugate. In some embodiments the mixed formulation (or therapeutically acceptable composition) can be KSI-501DS. In some embodiments, the unconjugated protein can be OG2072 fusion protein. In some embodiments, insoluble components can be removed via the prefiltration step of the tandem HPLC method. In some embodiments, the unconjugated protein can bind to the CEX column. In some embodiments, the conjugated protein flows through the CEX column. In some embodiments, the components of the unbound fraction can be determined by SEC analysis. In some embodiments, the components of the unbound fraction can comprise conjugated protein and some unconjugated protein. In some embodiments, the unconjugated protein bound to the CEX column can be eluted by a pulse elution. In some embodiments, the pulse elution can be performed with NaCl. In some embodiments, the pulse elution can be performed with any concentration between 0.5-5M NaCl. In some embodiments, the pulse elution can be performed with 1M NaCl. In some embodiments, the components of the eluted fraction can be determined by SEC analysis. In some embodiments, the components of the eluted fraction can comprise unconjugated protein, aggregated unconjugated protein, and some conjugated protein.

In some embodiments, a method of analyzing a mixed formulation (or therapeutically acceptable composition) sample is provided, the method comprising: providing a mixed formulation (or therapeutically acceptable composition) that comprises: a first protein conjugated to a polymer; and a second protein that is not conjugated to the polymer. In some embodiments, a first run, wherein the mixed formulation (or therapeutically acceptable composition) is run through a prefiltration step to provide a first filtered mixed formulation (or therapeutically acceptable composition), wherein the first filtered mixed formulation (or therapeutically acceptable composition) is run through a cation exchange chromatography (CEX) column to provide a second filtered mixed formulation (or therapeutically acceptable composition); and a second run, wherein the protein bound to the CEX column is eluted to provide a third filtered mixed formulation (or therapeutically acceptable composition), wherein the third filtered mixed formulation (or therapeutically acceptable composition) is run through a size exclusion exchange (SEC) high performance liquid chromatography (HPLC) column to provide a fourth filtered mixed formulation (or therapeutically acceptable composition); can be combined into a single continuous chromatography run. In some embodiments, the method allows analysis of the mixed formulation (or therapeutically acceptable composition) based on the differential charge and size variant of the constituents.

In some embodiments, a method or apparatus for tandem HPLC comprising CEX-SEC is provided. In some embodiments, the tandem method of HPLC comprising CEX-SEC can be performed as a single continuous chromatography run. In some embodiments, the single continuous chromatography run does not lose resolution of detection of peaks seen when tandem CEX-SEC analysis is performed as two separate chromatography runs.

In some embodiments, the tandem HPLC CEX-SEC method or apparatus can be focused only on analysis of the unconjugated protein its instability impurities such as aggregates or degradation products. In some embodiments, the unconjugated protein can be OG1950 free protein. In some embodiments, the tandem HPLC CEX-SEC method or apparatus can ignore all the analysis of the OG1953 conjugate, CEX-unbound run and/or the 16 min peak in the CEX-bound elution run.

In some embodiments, the unconjugated protein in a mixed formulation (or therapeutically acceptable composition) sample can be analyzed by injecting a mixed formulation (or therapeutically acceptable composition) sample into a tandem HPLC apparatus, running the mixed formulation (or therapeutically acceptable composition) sample through a first a prefilter, and second a CEX column. In some embodiments, the CEX-unbound fraction can be directed through a bypass loop via a switch valve. In some embodiments, the CEX-bound fraction can be eluted and subjected to further analysis. In some embodiments, the analysis of the unconjugated protein in the mixed formulation (or therapeutically acceptable composition) sample can be performed as a single continuous chromatography run.

In some embodiments, a mixed formulation is provided. In some embodiments a mixed composition is provided. In some embodiments, the mixed formulation is a mixed therapeutically effective formulation. In some embodiments, the composition is a mixed therapeutic or mixed therapeutically acceptable composition. In some embodiments, the mixed therapeutic or therapeutically acceptable composition is acceptable for administering directly into the eye of a patient (e.g., acceptable for intravitreal administration).

In some embodiments, the tandem HPLC CEX-SEC method or apparatus can analyze the OG1953 conjugate. In some embodiments, the tandem HPLC CEX-SEC method or apparatus can analyze the OG1950 free protein. In some embodiments, the tandem HPLC CEX-SEC method or apparatus can analyze the OG1950 and/or OG1953 related impurity products. In some embodiments, the tandem HPLC CEX-SEC method or apparatus can define the purity and stability for the clinical formulation.

In some embodiments, the tandem HPLC CEX-SEC method or apparatus can analyze the OG2074 conjugate. In some embodiments, the tandem HPLC CEX-SEC method or apparatus can analyze the OG2072 free protein. In some embodiments, the tandem HPLC CEX-SEC method or apparatus can analyze the OG2072 and/or OG2074 related impurity products. In some embodiments, the tandem HPLC CEX-SEC method or apparatus can define the purity and stability for the clinical formulation.

In some embodiments, the tandem HPLC CEX-SEC method or apparatus performed as a single continuous chromatography run requires only a single chromatography run. In some embodiments, the single chromatography run can comprise an 80 minute run. In some embodiments, the single chromatography run can comprise a run of at least 80 minutes. In some embodiments, the single chromatography run can comprise a run of no more than 80 minutes. In some embodiments, the single chromatography run can comprise a run of about 80 minutes. In some embodiments, the single chromatography run can comprise a single sample injection event. In some embodiments, the tandem HPLC CEX-SEC method or apparatus performed as a single continuous chromatography run can possess regulatory justification that all peaks detected should be included in the 280 nm analysis.

In some embodiments, the tandem HPLC CEX-SEC method or apparatus comprises a single sample injection event. In some embodiments, the tandem HPLC CEX-SEC method or apparatus comprises multiple sample injection events.

In some embodiments, the tandem HPLC CEX-SEC method or apparatus performed as a single continuous chromatography run can be integrated with an HPLC system equipped with the Multi-angle light scattering (MALS) analysis. In some embodiments, the MALs analysis is tandem MALS. In some embodiments, the tandem HPLC CEX-SEC method or apparatus performed as a single continuous chromatography run can comprise TANDEM MALS instead of SEC-MALS analysis. In some embodiments, tandem-MALS can represent the third dimensional analysis beyond charge and size of the existing tandem (2D) method. In some embodiments, the tandem HPLC CEX-SEC method or apparatus performed as a single continuous chromatography run can also be implemented seamlessly with the previous example of multi-column selection valve configuration. In some embodiments, some instrumental modification can be necessary for flow/pressure regulation to minimize baseline signal drift in the single continuous chromatography run.

In some embodiments, a flow splitter can be installed before the SEC column. In some embodiments, a flow splitter is installed before the first or second SEC column. In some embodiments, the flow splitter can allow appropriate amount of loaded mixed formulation (or therapeutically acceptable composition) sample to enter the SEC column for optimal resolution. In some embodiments, a flow splitter is used to adjust the injected amount appropriately for the conjugate analysis. In some embodiments, the flow splitter provides single sample injection into the SEC column.

In some embodiments, any of the apparatus options can be provided as a system claim.

In some embodiments, any one of the above steps can have further steps added between them. In some embodiments, any one or more of the above steps can be repeated. In some embodiments, any one or more of the above steps can be performed concurrently or out of the order provided herein. In some embodiments, in some embodiments, the IEX or CEX column following the prefilter can be bypassed by an optional switch valve. In some embodiments, analysis of the IEX or CEX-bound fraction can be bypassed by the switch valve. In some embodiments, analysis of the IEX or CEX-unbound fraction can be bypassed by the switch valve. In some embodiments, the IEX or CEX-bound fraction can be directed to an SEC column and the IEX or CEX-unbound fraction can be bypassed by the switch valve. In some embodiments, the IEX or CEX-unbound fraction can be directed to an SEC column and the IEX or CEX-bound fraction can be bypassed by the switch valve.

Additional embodiments of the present disclosure are provided in the following numbered arrangements.
1. A method of analyzing a mixed formulation sample, the method comprising:
   i. providing a mixed formulation that comprises:
      a. a first antibody that is an anti-VEGF-A antibody conjugated to a polymer; and
      b. a second antibody that is an anti-VEGF-A antibody that is not conjugated to the polymer;
   ii. a first run, wherein the mixed formulation is run through a prefiltration step to provide a first filtered mixed formulation, wherein the first filtered mixed formulation is run through a cation exchange chromatography (CEX) column to provide a second filtered mixed formulation;
   iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation;
   iv. a specific ionic strength of the solvent is used to allow partitioning of the mixed formulation in the CEX column; and
      wherein the method allows analysis of the mixed formulation based on the differential charge and size variant of the constituents.
2. A method of analyzing a mixed therapeutic composition sample, the method comprising:
   i. providing a mixed therapeutic composition that comprises:
      a. a first antibody that is an anti-VEGF-A antibody conjugated to a polymer; and
      b. a second antibody that is an anti-VEGF-A antibody that is not conjugated to the polymer;
   ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition;
   iii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition;
   iv. a specific ionic strength of the solvent is used to allow partitioning of the mixed therapeutic composition in the CEX column; and
      wherein the method allows analysis of the mixed therapeutic composition based on the differential charge and size variant of the constituents.
3. A method of analyzing a mixed therapeutically effective formulation sample, the method comprising:
   i. providing a mixed therapeutically effective formulation that comprises:
      a. a first antibody that is an anti-VEGF-A antibody conjugated to a polymer; and
      b. a second antibody that is an anti-VEGF-A antibody that is not conjugated to the polymer;
   ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation;
   iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation;
   iv. a specific ionic strength of the solvent is used to allow partitioning of the mixed therapeutically effective formulation in the CEX column; and
      wherein the method allows analysis of the mixed therapeutically effective formulation based on the differential charge and size variant of the constituents.
4. The method any one of the previous arrangements, wherein the polymer of the first antibody comprises a phosphorylcholine containing polymer, wherein the polymer is covalently bonded to the first antibody at a cysteine outside a variable region of the first antibody, and wherein said cysteine replaces a non-cysteine amino acid that occurs in a same position in a sequence, wherein the first antibody comprises a light chain and heavy chain, said heavy chain comprising an Fc region, wherein the cysteine is in the Fc region of the heavy chain, wherein the sequence of the heavy chain comprises SEQ ID NO: 1 (with or without the C-terminal lysine), and wherein the sequence of the light chain comprises SEQ ID NO: 2;
   wherein the first antibody has the following structure:

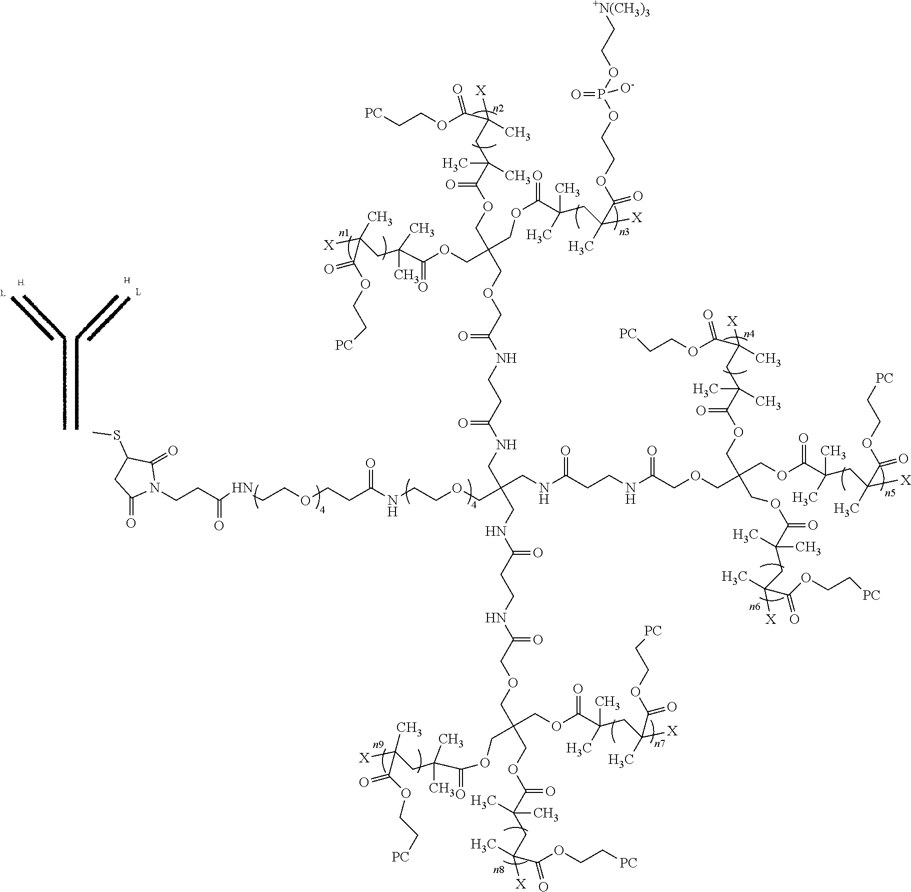

where X is a) —OR where R is —H, Methyl, ethyl, propyl, isopropyl, b) —H, or c) any halogen, including —Br, —Cl, or —I, d) —SCN, or e) —NCS; (or optionally where X is a) —OR where R is —H, Methyl, ethyl, propyl, or isopropyl, b) —H, or c) a halide;)
wherein:
each heavy chain of the first antibody is denoted by the letter H, and each light chain of the first antibody is denoted by the letter L;
the polymer is bonded to the first antibody through the sulfhydryl of a cysteine at position 449, as numbered in SEQ ID NO: 1, which bond is depicted on one of the heavy chains;
PC is

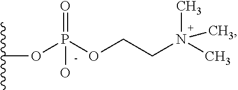

where the curvy line indicates the point of attachment to the rest of the polymer; and
n1, n2, n3, n4, n5, n6, n7, n8 and n9 are the same or different such that the sum of n1, n2, n3, n4, n5, n6, n7, n8 and n9 is 2500 plus or minus 15%.
5. The method of any one of the previous arrangements, wherein the second antibody comprises a light chain and heavy chain, said heavy chain comprising an Fc region, wherein a cysteine is in the Fc region of the heavy chain, wherein the sequence of the heavy chain comprises SEQ ID NO: 1 (with or without the C-terminal lysine), and wherein the sequence of the light chain comprises SEQ ID NO: 2.
6. The method of any one of the previous arrangements, wherein the heavy chain of the first and second antibodies comprises CDRH1: GYDFTHYGMN, CDRH2: WINTYTGEPTYAADFKR, and CDRH3: YPYYYGTSHWYFDV, and position 231 is T (via sequential counting as in SEQ ID NO: 1), and the light chain of the first and second antibodies comprises CDRL1: SASQDISNYLN (SEQ ID NO: 12), CDRL2: FTSSLHS (SEQ ID NO: 13), and CDRL3: QQYSTVPWT (SEQ ID NO: 14), and Kabat position 4 is L.
7. The method of any one of the previous arrangements, wherein the mixed formulation or composition sample is loaded into a tandem CEX and SEC HPLC column arrangement, wherein the formulation or composition sample travels sequentially through a prefilter, a CEX column, and a SEC HPLC column.
8. The method of arrangement 7, wherein the CEX column comprises a Shodex SP825 column with internal diameter (i.d.)×length dimensions of 9.0×75 mm.
9. The method of arrangement 7, wherein the SEC HPLC is a TSKgel G3000SWx1 column with i.d.×length dimensions of 7.8×300 mm.
10. The method of arrangement any one of the previous arrangements, wherein the isocratic running conditions comprise a flow rate of 0.1-5 ml/min.
11. The method of arrangement 10, wherein the isocratic running conditions comprise a flow rate of at 0.5-1 ml/min.
12. The method of arrangement any one of the previous arrangements, wherein the buffer comprises 20 mM sodium acetate, pH 3-6.
13. The method of arrangement 12, wherein the buffer comprises 20 mM sodium acetate, pH 5.
14. The method of arrangement any one of the previous arrangements, wherein the buffer comprises any amount between 50-500 mM NaCl.
15. The method of arrangement 14, wherein the buffer comprises any amount between 90-120 mM NaCl.
16. The method of arrangement any one of the previous arrangements, wherein the buffer comprises a conductivity of about 2-32 mS/cm.
17. The method of arrangement 16, wherein the buffer comprises a conductivity of about 8.9 mS/cm.
18. The method of any one of the previous arrangements, further comprising assessment for high molecular weight aggregates within the purified mixed formulation or composition by methods comprising SEC profile analysis and SDS PAGE gel.
19. The method of any one of the previous arrangements, wherein the percent composition of the second antibody relative to the total protein mass weight concentration of the first antibody and the second antibody in the composition is any percentage between 0-93%.
20. The method of arrangement 19, wherein the percent composition of the second antibody relative to the total protein mass weight concentration of the first antibody and the second antibody in the composition is any percentage between 0% and 50%.
21. The method of arrangement 20, wherein the percent composition of the second antibody relative to the total protein mass weight concentration of the first antibody and the second antibody in the composition is any percentage between 0% and 25%.
22. The method of any one of the previous arrangements, wherein the mixed formulation or composition comprises between 50 μg and 1,340 μg of protein.
23. The method of any one of the previous arrangements, further comprising assessment of the percentage of the second antibody in the purified formulation or composition by SEC profile analysis.
24. The method of any one of the previous arrangements, wherein the first run separates the first conjugated antibody, from the second antibody and antibody aggregates.
25. The method of arrangement 24, further comprising SEC analysis on the second filtered mixed formulation or composition of the first run performed by a second SEC HPLC column down-stream of the CEX column, wherein the second SEC HPLC column runs in parallel or subsequent to the first SEC HPLC column.
26. The method of arrangement 25, wherein the second HPLC column is a Shodex SB-806 HQ or Shodex 806M-HQ column.
27. The method of arrangement 25, wherein a switch valve allows for selection of the first or second SEC HPLC column.
28. The method of any one of the previous arrangements, wherein the step for elution of CEX bound free protein during the second run comprises a pulse of high salt at 0.5-5M NaCl in water or in a buffer at pH 5.
29. The method of arrangement 28, wherein the step for elution of CEX bound free protein during the second run comprises a pulse of high salt at 1M NaCl.
30. The method of arrangement 28, wherein during the second run, the fourth filtered mixed formulation or composition is run through a prefiltration step to provide a fifth filtered mixed formulation or composition, wherein the fifth filtered mixed formulation or composition is run through a CEX column to provide a six filtered mixed formulation or composition, wherein the sixth mixed formulation or composition is run through a SEC HPLC column.
31. The method of arrangement 30, wherein running the filtered mixed sample through a size exclusion exchange SEC HPLC column separates the second antibody from antibody aggregates.
32. The method of arrangement 25, wherein the effluent of the first and/or second SEC HPLC columns is connected to a to a multi angle light scattering (MALS) detector.
33. The method of arrangement 25, wherein an additional valve is installed downstream of the first and/or second SEC column and the eluted free protein peaks are collected using a fraction collector for icIEF analysis.
34. An apparatus comprising a tandem series of CEX and SEC HPLC columns for use in analyzing a mixed formulation sample comprising a) a first antibody that is an anti-VEGF-A antibody conjugated to a polymer and b) a second antibody that is an anti-VEGF-A antibody that is not conjugated to the polymer, wherein the purification comprises;
   i. a first run, wherein the mixed formulation is run through a prefiltration step to provide a first filtered mixed formulation, wherein the first filtered mixed formulation is run through a cation exchange chromatography (CEX) column to provide a second filtered mixed formulation; and
   ii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation.
35. An apparatus comprising a tandem series of CEX and SEC HPLC columns for use in analyzing a mixed therapeutic composition sample comprising a) a first antibody that is an anti-VEGF-A antibody conjugated to a polymer and b) a second antibody that is an anti-VEGF-A antibody that is not conjugated to the polymer, wherein the purification comprises;
   i. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; and
   ii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition.
36. An apparatus comprising a tandem series of CEX and SEC HPLC columns for use in analyzing a mixed therapeutically effective formulation sample comprising a) a first antibody that is an anti-VEGF-A antibody conjugated to a polymer and b) a second antibody that is an anti-VEGF-A antibody that is not conjugated to the polymer, wherein the purification comprises;
   i. a first run, wherein the mixed therapeutically effective formulation is run through a prefiltration step to provide a first filtered mixed therapeutically effective formulation, wherein the first filtered therapeutically effective formulation is run through a cation exchange chromatography (CEX) column to provide a second filtered mixed therapeutically effective formulation; and
   ii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation.
37. The apparatus of any one of the previous apparatus arrangements, wherein the polymer of the first antibody comprises a phosphorylcholine containing polymer, wherein the polymer is covalently bonded to the first antibody at a cysteine outside a variable region of the first antibody, and wherein said cysteine replaces a non-cysteine amino acid that occurs in a same position in a sequence, wherein the first antibody comprises a light chain and heavy chain, said heavy chain comprising an Fc region, wherein the cysteine is in the Fc region of the heavy chain, wherein the sequence of the heavy chain comprises SEQ ID NO: 1 (with or without the C-terminal lysine), and wherein the sequence of the light chain comprises SEQ ID NO: 2;
   wherein the antibody conjugate has the following structure:

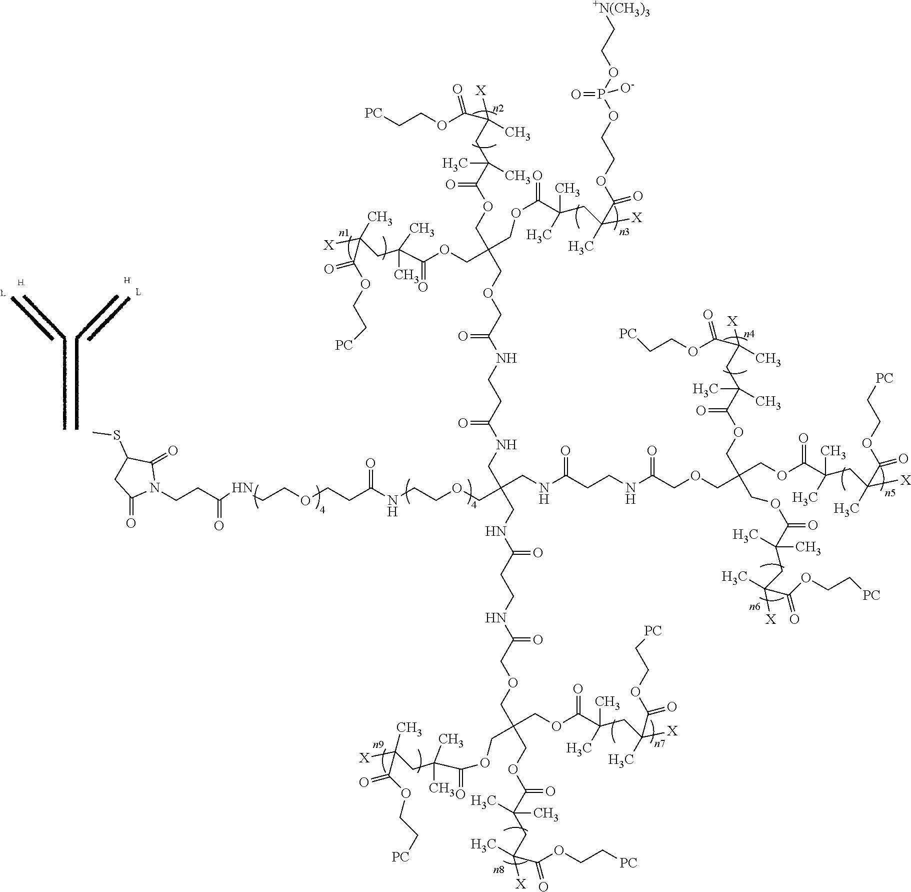

where X is a) —OR where R is —H, Methyl, ethyl, propyl, or isopropyl, b) —H, or c) a halide; (or optionally where X is a) —OR where R is —H, Methyl, ethyl, propyl, isopropyl, b) —H, or c) any halogen, including —Br, —Cl, or —I, d) —SCN, or e) —NCS;)
wherein:
each heavy chain of the first antibody is denoted by the letter H, and each light chain of the first antibody is denoted by the letter L;
the polymer is bonded to the first antibody through the sulfhydryl of a cysteine at position 449, as numbered in SEQ ID NO: 1, which bond is depicted on one of the heavy chains;
PC is

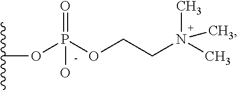

where the curvy line indicates the point of attachment to the rest of the polymer; and
n1, n2, n3, n4, n5, n6, n7, n8 and n9 are the same or different such that the sum of n1, n2, n3, n4, n5, n6, n7, n8 and n9 is 2500 plus or minus 15%.
38. The apparatus of any one of the previous apparatus arrangements, wherein the second antibody comprises a light chain and heavy chain, said heavy chain comprising an Fc region, wherein a cysteine is in the Fc region of the heavy chain, wherein the sequence of the heavy chain comprises SEQ ID NO: 1 (with or without the C-terminal lysine), and wherein the sequence of the light chain comprises SEQ ID NO: 2.
39. The apparatus of any one of the previous apparatus arrangements, wherein the heavy chain of the first and second antibodies comprises CDRH1: GYDFTHYGMN, CDRH2: WINTYTGEPTYAADFKR, and CDRH3: YPYYYGTSHWYFDV, and position 231 is T (via sequential counting as in SEQ ID NO: 1), and the light chain of the first and second antibodies comprises CDRL1: SASQDISNYLN (SEQ ID NO: 12), CDRL2: FTSSLHS (SEQ ID NO: 13), and CDRL3: QQYSTVPWT (SEQ ID NO: 14), and Kabat position 4 is L.
40. The apparatus of any one of the previous apparatus arrangements, wherein the injected mixed formulation sample travels sequentially through a prefilter, said CEX column, and said SEC HPLC column.
41. The apparatus of arrangement 40, wherein the CEX column is a Shodex SP825 column with internal diameter (i.d.)×length dimensions of 9.0×75 mm.
42. The apparatus of arrangement 40, wherein the SEC HPLC is a TSKgel G3000SWxl column with i.d.×length dimensions of 7.8×300 mm.
43. The apparatus of arrangement 40, wherein the isocratic running conditions of the columns comprise a flow rate of 0.1-5 ml/min.
44. The apparatus of arrangement 43, wherein the isoelectric running conditions of the columns comprise a flow rate of 0.5-1 ml/min.
45. The apparatus of any one of the previous apparatus arrangements, wherein the buffer comprises 20 mM sodium acetate at pH 3-6.
46. The apparatus of arrangement 45, wherein the buffer comprises 20 mM sodium acetate at pH 5.
47. The apparatus of any one of the previous apparatus arrangements, wherein the buffer comprises any amount between 50-500 mM NaCl.
48. The apparatus of arrangement 47, wherein the buffer comprises any amount between 90-120 mM NaCl.
49. The apparatus of any one of the previous apparatus arrangements, wherein the buffer comprises a conductivity of about 2-32 mS/cm.
50. The apparatus of arrangement 49, wherein the buffer comprises a conductivity of about 8.9 mS/cm.
51. The apparatus of any one of the previous apparatus arrangements, wherein the percent composition of the second antibody in the mixed formulation or composition sample relative to the total protein mass weight concentration of the first antibody and the second antibody in the formulation or composition sample is any percentage between 0-93%.
52. The apparatus of arrangement 51, wherein the percent composition of the second antibody in the mixed formulation or composition sample relative to the total protein mass weight concentration of the first antibody and the second antibody in the formulation or composition sample is any percentage between 0-50%.
53. The apparatus of arrangement 52, wherein the percent composition of the second antibody in the mixed formulation or composition sample relative to the total protein mass weight concentration of the first antibody and the second antibody in the formulation or composition sample is any percentage between 0-25%.
54. The apparatus of any one of the previous apparatus arrangements, wherein the mixed formulation or composition between 50 μg and 1,340 μg of protein.
55. The apparatus of any one of the previous apparatus arrangements, wherein the first run separates the first antibody from the second antibody and antibody aggregates.
56. The apparatus of arrangement 55, further comprising SEC analysis on the second filtered mixed formulation or composition of the first run performed by a second SEC HPLC column down-stream of the CEX column, wherein the second SEC HPLC column runs in parallel or subsequent to the first SEC HPLC column.
57. The apparatus of arrangement 56, wherein the second HPLC column is a Shodex SB-806 HQ or Shodex 806M-HQ column.
58. The apparatus of arrangement 56, wherein a switch valve allows for selection of the first or second SEC HPLC column.
59. The apparatus of any one of the previous arrangements, wherein the step for elution of CEX bound free protein during the second run comprises a pulse of high salt at 0.5-5M NaCl in water or in a buffer at pH 5.
60. The apparatus of arrangement 59, wherein the step for elution of CEX bound free protein during the second run comprises a pulse of high salt at 1M NaCl.
61. The apparatus of arrangement 59, wherein during the second run, the fourth filtered mixed formulation or composition is run through a prefiltration step to provide a fifth filtered mixed formulation or composition, wherein the fifth filtered mixed formulation or composition is run through a CEX column to provide a six filtered mixed formulation or composition, wherein the sixth mixed formulation or composition is run through a SEC HPLC column.
62. The apparatus of arrangement 61, wherein running the filtered mixed sample through a size exclusion exchange SEC HPLC column separates the second antibody from antibody aggregates.
63. The apparatus of arrangement 56, wherein the effluent of the first and/or second SEC HPLC columns is connected to a to a multi angle light scattering (MALS) detector.
64. The apparatus of arrangement 56, wherein an additional valve is installed downstream of the first and/or second SEC column and the eluted free protein peaks are collected using a fraction collector for icIEF analysis.
65. A method of analyzing a mixed formulation sample, the method comprising:
   i. providing a mixed formulation that comprises:
      a. a first antibody that is an anti-VEGF-A antibody conjugated to a polymer; and
      b. a second antibody that is an anti-VEGF-A antibody that is not conjugated to the polymer;
   ii. a first run, wherein the mixed formulation is run through a prefiltration step to provide a first filtered mixed formulation, wherein the first filtered mixed formulation is run through a cation exchange chromatography (CEX) column to provide a second filtered mixed formulation; and
   iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation; and
   wherein the method reduces the presence of antibody aggregates in the formulation; and
   wherein the polymer of the first antibody comprises a phosphorylcholine containing polymer, wherein the polymer is covalently bonded to the first antibody at a cysteine outside a variable region of the first antibody, and wherein said cysteine replaces a non-cysteine amino acid that occurs in a same position in a sequence, wherein the first antibody comprises a light chain and heavy chain, said heavy chain comprising an Fc region, wherein the cysteine is in the Fc region of the heavy chain, wherein the sequence of the heavy chain comprises SEQ ID NO: 1 (with or without the C-terminal lysine), and wherein the sequence of the light chain comprises SEQ ID NO: 2; wherein the antibody conjugate has the following structure:

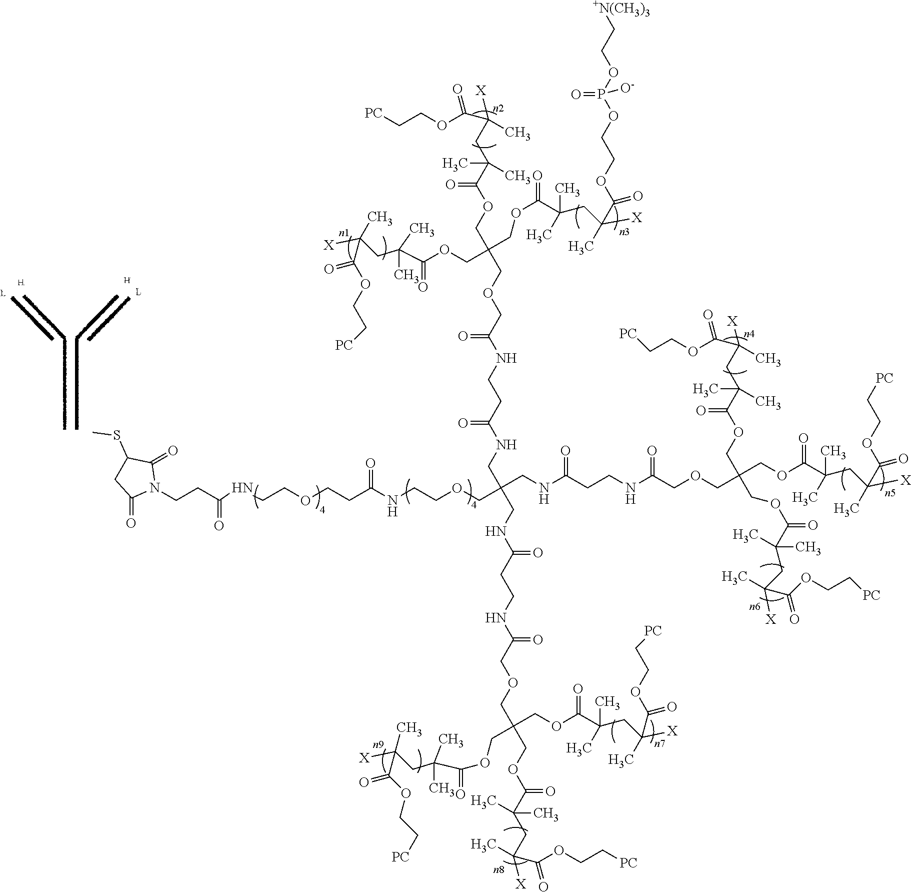

where X is a) —OR where R is —H, Methyl, ethyl, propyl, or isopropyl, b) —H, or c) a halide; (or optionally where X is a) —OR where R is —H, Methyl, ethyl, propyl, or isopropyl, b) —H, c) any halogen, including —Br, —Cl, or —I, d) —SCN, or e) —NCS;)
wherein:
each heavy chain of the first antibody is denoted by the letter H, and each light chain of the first antibody is denoted by the letter L;
the polymer is bonded to the first antibody through the sulfhydryl of a cysteine at position 449, as numbered in SEQ ID NO: 1, which bond is depicted on one of the heavy chains;
PC is

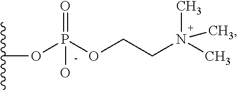

where the curvy line indicates the point of attachment to the rest of the polymer; and n1, n2, n3, n4, n5, n6, n7, n8 and n9 are the same or different such that the sum of n1, n2, n3, n4, n5, n6, n7, n8 and n9 is 2500 plus or minus 15%; and
wherein, the second antibody comprises a light chain and heavy chain, said heavy chain comprising an Fc region, wherein a cysteine is in the Fc region of the heavy chain, wherein the sequence of the heavy chain comprises SEQ ID NO: 1, and wherein the sequence of the light chain comprises SEQ ID NO: 2; and
wherein the mixed formulation sample travels sequentially through a prefilter, the CEX column, and the SEC HPLC column, wherein the tandem CEX and SEC HPLC columns are a Shodex SP825 column with internal diameter (i.d.)×length dimensions of 9.0×75 mm and a TSKgel G3000SWxl column with i.d.×length dimensions of 7.8×300 mm column arrangement;
wherein the isocratic running conditions comprise a flow rate of 0.5 ml/min, and the buffer comprises 20 mM sodium acetate, pH 5 any amount between 50 mM NaCl and 5M NaCl;
wherein the percentage of the second antibody is any percentage between 0% and 20%;
wherein the mixed formulation sample comprises any amount between 25 ug and 1340 ug of protein;
wherein the method further comprises assessment for high molecular weight aggregates within the purified mixed formulation by methods comprising SEC profile analysis and SDS PAGE gel; and
wherein the method further comprises assessment of the percentage of the second antibody in the purified mixed formulation by SEC profile analysis;
wherein the first run separates the first antibody from the second antibody and antibody aggregates,
wherein the step for elution of CEX bound free protein during the second run comprises a pulse of high salt at 1M NaCl,
wherein during the second run, the fourth filtered mixed formulation is run through a prefiltration step to provide a fifth filtered mixed formulation, wherein the fifth filtered mixed formulation is run through a CEX column to provide a six filtered mixed formulation, wherein the sixth mixed formulation is run through a SEC HPLC column; and
wherein running the filtered mixed sample through a size exclusion exchange SEC HPLC column separates the second antibody from antibody aggregates.
66. The method or apparatus of any one of the preceding arrangements, wherein the first, second, or first and second antibody comprises a light chain and a heavy chain, said heavy chain comprising an Fc region.
67. The method or apparatus of any one of the preceding arrangements, wherein a cysteine of the first, second, or first and second antibody is in the Fc region of the heavy chain.
68. The method or apparatus of any one of the preceding arrangements, wherein the first, second, or first and second antibody is an immunoglobulin G (IgG).
69. The method or apparatus of any one of the preceding arrangements, wherein the heavy chain of the first, second, or first and second antibody comprises: CDRH1: GYDFTHYGMN (SEQ ID NO: 9), CDRH2: WINTYTGEPTYAADFKR (SEQ ID NO: 10), and CDRH3: YPYYYGTSHWYFDV (SEQ ID NO: 11), and the light chain of the first, second, or first and second antibody comprises CDRL1: SASQDISNYLN (SEQ ID NO: 12), CDRL2: FTSSLHS (SEQ ID NO: 13), and CDRL3: QQYSTVPWT (SEQ ID NO: 14).
70. The method or apparatus of any one of the preceding arrangements, wherein the heavy chain isotype of the first, second, or first and second antibody is human IgG1.
71. The method or apparatus of any one of the preceding arrangements, wherein the heavy chain constant domain of the first, second, or first and second antibody has one or more mutations relative to the constant domain of human IgG1 to modulate effector function.
72. The method or apparatus of any one of the preceding arrangements, wherein the mutations of the first, second, or first and second antibody are to one or more of the following amino acid positions (EU numbering): E233X, L234X, L235X, G236X, G237X, A327X, A330X, and P331X wherein X is any natural or unnatural amino acid.
73. The method or apparatus of any one of the preceding arrangements, wherein the mutations of the first, second, or first and second antibody are selected from the group consisting of (EU numbering): E233P, L234V, L234A, L235A, G237A, A327G, A330S, and P331S.
74. The method or apparatus of any one of the preceding arrangements, comprising the following mutations of the first, second, or first and second antibody: L234A, L235A, and G237A (EU numbering).
75. The method or apparatus of any one of the preceding arrangements, wherein the cysteine of the first, second, or first and second antibody is in the antibody heavy chain and is Q347C (EU numbering) or L443C (EU numbering).
76. The method or apparatus of any one of the preceding arrangements, wherein the sequence of the antibody heavy chain of the first, second, or first and second antibody is SEQ ID NO: 1 (with or without the C-terminal lysine) and the sequence of the light chain of the first, second, or first and second antibody is SEQ ID NO: 2.
77. The method or apparatus of any one of the preceding arrangements, wherein the cysteine of the first, second, or first and second antibody is L443C (EU numbering).
78. The method or apparatus of any one of the preceding arrangements, wherein the polymer of the antibody conjugate has three or more arms or is synthesized with an initiator comprising 3 or more polymer initiation sites.
79. The method or apparatus of any one of the preceding arrangements, wherein the polymer of the antibody conjugate has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites.
80. The method or apparatus of any one of the preceding arrangements, wherein the polymer of the antibody conjugate has a polydispersity value (PDI) of less than about 1.2.
81. The method or apparatus of any one of the preceding arrangements, wherein the mixed formulation or composition comprises the second antibody at about 1% or more of a total molar amount of the conjugate and the second antibody, wherein the total molar amount comprises a sum of a first molar amount of the first antibody in the formulation or composition, and a second molar amount of the second antibody in the formulation or composition.
82. A method of analyzing a mixed formulation sample, the method comprising:
   i. providing a mixed formulation that comprises:
      a. a fusion protein conjugated to a polymer; and
      b. a second protein that is not conjugated to the polymer;
   ii. a first run, wherein the mixed formulation is run through a prefiltration step to provide a first filtered mixed formulation, wherein the first filtered mixed formulation is run through a cation exchange chromatography (CEX) column to provide a second filtered mixed formulation;
   iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation;
      a specific ionic strength of the solvent is used to allow partitioning of the mixed formulation in the CEX column; and
      wherein the method allows analysis of the mixed formulation based on the differential charge and size variant of the constituents.
83. A method of analyzing a mixed therapeutic composition sample, the method comprising:
   i. providing a mixed therapeutic composition that comprises:
      a. a fusion protein conjugated to a polymer; and
      b. a second protein that is not conjugated to the polymer;
   ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition;
   iii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition;
   iv. a specific ionic strength of the solvent is used to allow partitioning of the mixed therapeutic composition in the CEX column; and
      wherein the method allows analysis of the mixed therapeutic composition based on the differential charge and size variant of the constituents.
84. A method of analyzing a mixed therapeutically effective formulation sample, the method comprising:
   i. providing a mixed therapeutically effective formulation that comprises:
      a. a fusion protein conjugated to a polymer; and
      b. a second protein that is not conjugated to the polymer;
   ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation;
   iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation;
   iv. a specific ionic strength of the solvent is used to allow partitioning of the mixed therapeutically effective formulation in the CEX column; and
      wherein the method allows analysis of the mixed therapeutically effective formulation based on the differential charge and size variant of the constituents.
85. An apparatus comprising a tandem series of CEX and SEC HPLC columns for use in purifying a mixed formulation sample comprising a) a first fusion protein conjugated to a polymer and b) a second protein that is not conjugated to the polymer, wherein the purification comprises;
   i. a first run, wherein the mixed formulation is run through a prefiltration step to provide a first filtered mixed formulation, wherein the first filtered mixed formulation is run through a cation exchange chromatography (CEX) column to provide a second filtered mixed formulation; and
   ii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation.
86. An apparatus comprising a tandem series of CEX and SEC HPLC columns for use in purifying a mixed therapeutic composition sample comprising a) a first fusion protein conjugated to a polymer and b) a second protein that is not conjugated to the polymer, wherein the purification comprises;
   i. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; and
   ii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition.
87. An apparatus comprising a tandem series of CEX and SEC HPLC columns for use in purifying a mixed therapeutically effective formulation sample comprising a) a first fusion protein conjugated to a polymer and b) a second protein that is not conjugated to the polymer, wherein the purification comprises;
   i. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; and
   ii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation.
88. A method of analyzing a mixed formulation sample, the method comprising:
   i. providing a mixed formulation that comprises:
      a. a first antibody that is an anti-VEGF-A antibody conjugated to a polymer; and
      b. a second antibody that is an anti-VEGF-A antibody that is not conjugated to the polymer;
   ii. a first run, wherein the mixed formulation is run through a prefiltration step to provide a first filtered mixed formulation, wherein the first filtered mixed formulation is run through a cation exchange chromatography (CEX) column to provide a second filtered mixed formulation;
   iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation;
   iv. a specific ionic strength of the solvent is used to allow partitioning of the mixed formulation in the CEX column; and
      wherein the method allows analysis of the mixed formulation based on the differential charge and size variant of the constituents.
89. A method of analyzing a mixed therapeutic composition sample, the method comprising:
   i. providing a mixed therapeutic composition that comprises:
      a. a first antibody that is an anti-VEGF-A antibody conjugated to a polymer; and
      b. a second antibody that is an anti-VEGF-A antibody that is not conjugated to the polymer;
   ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition;
   iii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition;
   iv. a specific ionic strength of the solvent is used to allow partitioning of the mixed therapeutic composition in the CEX column; and
      wherein the method allows analysis of the mixed therapeutic composition based on the differential charge and size variant of the constituents.
90. A method of analyzing a mixed therapeutically effective formulation sample, the method comprising:
   i. providing a mixed therapeutically effective formulation that comprises:
      a. a first antibody that is an anti-VEGF-A antibody conjugated to a polymer; and
      b. a second antibody that is an anti-VEGF-A antibody that is not conjugated to the polymer;
   ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation;
   iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation;
   iv. a specific ionic strength of the solvent is used to allow partitioning of the mixed therapeutically effective formulation in the CEX column; and
      wherein the method allows analysis of the mixed therapeutically effective formulation based on the differential charge and size variant of the constituents.
91. A method of analyzing a mixed formulation sample, the method comprising:
   i. providing a mixed formulation that comprises:
      a. a first antibody that is an anti-VEGF-A antibody conjugated to a polymer; and
      b. a second antibody that is an anti-VEGF-A antibody that is not conjugated to the polymer;
   ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
   iii. a first run, wherein the mixed formulation is injected and run through the system of ii.
   iv. a second run, wherein a concentrated salt is injected to elute the bound fraction of the CEX column to be separated by the SEC column;
      wherein, a specific ionic strength of the solvent is used to allow partitioning of the mixed formulation in the CEX column; and
      wherein the method allows analysis of the mixed formulation based on the differential charge and size variant of the constituents.
92. A method of analyzing a mixed therapeutic composition sample, the method comprising:
   i. providing a mixed therapeutic composition that comprises:
      a. a first antibody that is an anti-VEGF-A antibody conjugated to a polymer; and
      b. a second antibody that is an anti-VEGF-A antibody that is not conjugated to the polymer;
   ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
   iii. a first run, wherein the mixed therapeutic composition is injected and run through the system of ii.
   iv. a second run, wherein a concentrated salt is injected to elute the bound fraction of the CEX column to be separated by the SEC column;
      wherein, a specific ionic strength of the solvent is used to allow partitioning of the mixed therapeutic composition in the CEX column; and
         wherein the method allows analysis of the mixed therapeutic composition based on the differential charge and size variant of the constituents.
93. A method of analyzing a mixed therapeutically effective formulation sample, the method comprising:
   i. providing a mixed therapeutically effective formulation that comprises:
      a. a first antibody that is an anti-VEGF-A antibody conjugated to a polymer; and
      b. a second antibody that is an anti-VEGF-A antibody that is not conjugated to the polymer;
   ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
   iii. a first run, wherein the mixed therapeutically effective formulation is injected and run through the system of ii.
   iv. a second run, wherein a concentrated salt is injected to elute the bound fraction of the CEX column to be separated by the SEC column;
      wherein, a specific ionic strength of the solvent is used to allow partitioning of the mixed therapeutically effective formulation in the CEX column; and
         wherein the method allows analysis of the mixed therapeutically effective formulation based on the differential charge and size variant of the constituents.
94. A method of analyzing a mixed formulation sample, the method comprising:
   i. providing a mixed formulation that comprises a combination of two protein moieties, wherein the first protein moiety (A) is conjugated to a polymer, and the second protein (B) moiety is not conjugated to a polymer;
   ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
   iii. a first run, wherein the mixed formulation is injected and run through the system of ii.
   iv. a second run, wherein a concentrated salt is injected to elute the bound fraction of the CEX column to be separated by the SEC column;
      wherein, a specific ionic strength of the solvent is used to allow partitioning of the mixed formulation in the CEX column; and
      wherein the method allows analysis of the mixed formulation based on the differential charge and size variant of the constituents.
95. A method of analyzing a mixed therapeutic composition sample, the method comprising:
   i. providing a mixed therapeutic composition that comprises a combination of two protein moieties, wherein the first protein moiety (A) is conjugated to a polymer, and the second protein (B) moiety is not conjugated to a polymer;
   ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
   iii. a first run, wherein the mixed therapeutic composition is injected and run through the system of ii.
   iv. a second run, wherein a concentrated salt is injected to elute the bound fraction of the CEX column to be separated by the SEC column;
      wherein, a specific ionic strength of the solvent is used to allow partitioning of the mixed therapeutic composition in the CEX column; and
      wherein the method allows analysis of the mixed therapeutic composition based on the differential charge and size variant of the constituents.
96. A method of analyzing a mixed therapeutically effective formulation sample, the method comprising:
   i. providing a mixed therapeutically effective formulation that comprises a combination of two protein moieties, wherein the first protein moiety (A) is conjugated to a polymer, and the second protein (B) moiety is not conjugated to a polymer;
   ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
   iii. a first run, wherein the mixed therapeutically effective formulation is injected and run through the system of ii.
   iv. a second run, wherein a concentrated salt is injected to elute the bound fraction of the CEX column to be separated by the SEC column;
   v, wherein, a specific ionic strength of the solvent is used to allow partitioning of the mixed therapeutically effective formulation in the CEX column; and
      wherein the method allows analysis of the mixed therapeutically effective formulation based on the differential charge and size variant of the constituents.
97. The method of arrangement 96, wherein A and B are the same protein moiety. 98. The method of arrangement 96, wherein A and B are different protein moieties.
99. The method of arrangement 96, wherein the protein moieties are antibodies.
100. The method of arrangement 96, wherein the polymer has 9 arms, and wherein the polymer has a molecular weight of between about 600,000 to about 1,000,000 Da.
101. The method of arrangement 96, wherein the MW of protein A is greater than or equal to the MW of protein B.
102. The method of arrangement 96, wherein the MW of protein A is less than or equal to the MW of protein B.
103. The method of arrangement 96, wherein the injected mixed formulation sample travels sequentially through a prefilter, said CEX column, and said SEC HPLC column.
104. The method of arrangement 103, wherein the CEX column is a Shodex SP825 CEX column with internal diameter (i.d.)×length dimensions of 9.0×75 mm.
105. The method of arrangement 103, wherein the SEC HPLC is a TSKgel G3000SWxl column with i.d.×length dimensions of 7.8×300 mm.
106. The method of arrangement 103, wherein the isocratic running conditions of the columns comprise a flow rate of 0.1-5 ml/min.
107. The method of arrangement 106, wherein the isocratic running conditions of the column comprise a flow rate of 0.5-1 ml/min.
108. The method of arrangement 103, wherein the buffer comprises 20 mM sodium acetate at pH 3-6.
109. The method of arrangement 108, wherein the buffer comprises 20 mM sodium acetate at pH 5.
110. The method of arrangement 103, wherein the buffer comprises any amount between 50-500 mM NaCl.
111. The method of arrangement 110, wherein the buffer comprises any amount between 90-120 mM NaCl.
112. The method of arrangement 103, wherein the buffer comprises a conductivity of about 2-32 mS/cm.
113. The method of arrangement 112, wherein the buffer comprises a conductivity about 8.9 mS/cm.
114. The method of arrangement 112, further comprising a second SEC HPLC column down-stream of the CEX column, wherein the second SEC HPLC column runs in parallel or subsequent to the first SEC HPLC column.
115. The method of arrangement 114, wherein the second HPLC column is a Shodex SB-806 HQ or Shodex 806M-HQ column.
116. The method of arrangement 115, wherein a switch valve allows for selection of the first or second SEC HPLC column.
117. The method of arrangement 112, wherein the effluent of the first and/or second SEC HPLC columns is connected to a to a multi angle light scattering (MALS) detector.
118. The method of arrangement 112, wherein an additional valve is installed downstream of the first and/or second SEC column and the eluted free protein peaks are collected using a fraction collector for other analytical techniques comprising icIEF, mass spectrometry, fluorescence detection, photodiode array (PDA) detector.
119. The method or apparatus of any one of the previous arrangements, wherein the molar ratio range of the second protein or antibody not conjugated to a polymer to the first protein or antibody conjugated to a polymer is any number between 0.01 and 55.
120. The method or apparatus of arrangement 119, wherein the molar ratio range of the second protein or antibody not conjugated to a polymer to the first protein or antibody conjugated to a polymer is any number between 0.01 and 16.
121. The method or apparatus of any one of the previous arrangements, wherein the second protein or antibody is added directly to the drug product of the first antibody or formulation to generate the mixed formulation.
122. The method or apparatus of any one of the previous arrangements, wherein the second protein or antibody is added to the CEX pool with the first protein or antibody and undergoes ultrafiltration and diafiltration to generate the mixed formulation.
123. The method or apparatus of arrangement 122, wherein the second protein or antibody undergoes decapping and refolding prior to being added to the CEX pool.
124. The method or apparatus of arrangement 123, wherein the decapped and refolded second protein or antibody undergoes further treatment with alkylation agents such as iodoacetamide (IAM) or N-ethylmaleimide (NEM) before being added to the CEX pool.
125. The method or apparatus of arrangement 122, wherein the mixed formulation further comprises an additional protein or antibody conjugated to a polymer.
126. The method of arrangement 99, wherein the antibody is a complement factor D (CFD) antibody, wherein the complement factor D antibody comprises a heavy chain and a light chain, wherein the heavy chain variable region (VH) comprises the amino acid sequence of any one of SEQ ID NOs: 15-47; and the light chain variable region (VL) comprises the amino acid sequence of any one of SEQ ID NOs: 48-80.
127. The method of arrangement 99, wherein the antibody comprises an antagonist IL-6 antibody fused to a VEGF trap, wherein the antibody comprises a heavy chain and a light chain, wherein the heavy chain variable region (VH) comprises the amino acid sequence of any one of SEQ ID NOs: 81-89; and the light chain variable region (VL) comprises the amino acid sequence of any one of SEQ ID NOs: 90-92.
128. The protein construct of arrangement 126, wherein the heavy and light chains are selected from the group comprising:
   (A) the heavy chain of SEQ ID NO:93 (with or without the C-terminal lysine), and the light chain of SEQ ID NO:94;
   (B) the heavy chain of SEQ ID NO:95 (with or without the C-terminal lysine), and the light chain of SEQ ID NO:96;
   (C) the heavy chain of SEQ ID NO:97 (with or without the C-terminal lysine), and the light chain of SEQ ID NO:98;
   (D) the heavy chain of SEQ ID NO:99 (with or without the C-terminal lysine), and the light chain of SEQ ID NO:100;
   (E) the heavy chain of SEQ ID NO:101 (with or without the C-terminal lysine), and the light chain of SEQ ID NO: 102;
   (F) the heavy chain of SEQ ID NO: 103 (with or without the C-terminal lysine), and the light chain of SEQ ID NO: 104;
   (G) the heavy chain of SEQ ID NO: 105 (with or without the C-terminal lysine), and the light chain of SEQ ID NO: 106;
   (H) the heavy chain of SEQ ID NO: 107 (with or without the C-terminal lysine), and the light chain of SEQ ID NO:108;
   (I) the heavy chain of SEQ ID NO:109 (with or without the C-terminal lysine), and the light chain of SEQ ID NO:110;
   (J) the heavy chain of SEQ ID NO:111 (with or without the C-terminal lysine), and the light chain of SEQ ID NO:112;
   (K) the heavy chain of SEQ ID NO:113 (with or without the C-terminal lysine), and the light chain of SEQ ID NO: 114;
   (L) the heavy chain of SEQ ID NO:115 (with or without the C-terminal lysine), and the light chain of SEQ ID NO:116;
   (M) the heavy chain of SEQ ID NO:117 (with or without the C-terminal lysine), and the light chain of SEQ ID NO:118;
   (N) the heavy chain of SEQ ID NO:119 (with or without the C-terminal lysine), and the light chain of SEQ ID NO:120;
   (O) the heavy chain of SEQ ID NO: 121 (with or without the C-terminal lysine), and the light chain of SEQ ID NO:122;
   (P) the heavy chain of SEQ ID NO:123 (with or without the C-terminal lysine), and the light chain of SEQ ID NO:124;
   (Q) the heavy chain of SEQ ID NO: 125 (with or without the C-terminal lysine), and the light chain of SEQ ID NO:126; and
   (R) the heavy chain of SEQ ID NO: 127 (with or without the C-terminal lysine), and the light chain of SEQ ID NO:128.
129. A method of analyzing a mixed formulation sample, the method comprising:
   i. providing a mixed formulation comprising a combination of two protein moieties, wherein the first protein moiety (A) is conjugated to a polymer, and the second protein moiety (B) is not conjugated to a polymer;
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
      wherein the method allows analysis of the mixed formulation based on the differential charge and size variant of the constituents.
130. A method of analyzing a mixed therapeutic composition sample, the method comprising:
   i. providing a mixed therapeutic composition comprising a combination of two protein moieties, wherein the first protein moiety (A) is conjugated to a polymer, and the second protein moiety (B) is not conjugated to a polymer;
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
      wherein the method allows analysis of the mixed therapeutic composition based on the differential charge and size variant of the constituents.
131. A method of analyzing a mixed therapeutically effective formulation sample, the method comprising:
   i. providing a mixed therapeutically effective formulation comprising a combination of two protein moieties, wherein the first protein moiety (A) is conjugated to a polymer, and the second protein moiety (B) is not conjugated to a polymer;
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
      wherein the method allows analysis of the mixed therapeutically effective formulation based on the differential charge and size variant of the constituents.
132. An apparatus comprising a tandem HPLC system for use in analyzing a mixed formulation sample comprising a combination of two protein moieties, wherein the first protein moiety (A) is conjugated to a polymer, and the second protein moiety (B) is not conjugated to a polymer, wherein the apparatus comprises:
   a) a HPLC pump connected to an auto-injector;
   b) a prefilter;
   c) cation-exchanger column (CEX);
   d) a switch valve enabling the effluent of the CEX to be directed to multiple possible targets,
   wherein the apparatus allows analysis of the mixed formulation based on the differential charge and size variant of the constituents.
133. An apparatus comprising a tandem HPLC system for use in analyzing a mixed therapeutic composition sample comprising a combination of two protein moieties, wherein the first protein moiety (A) is conjugated to a polymer, and the second protein moiety (B) is not conjugated to a polymer, wherein the apparatus comprises:
   a) a HPLC pump connected to an auto-injector;
   b) a prefilter;
   c) cation-exchanger column (CEX);
   d) a switch valve enabling the effluent of the CEX to be directed to multiple possible targets,
   wherein the apparatus allows analysis of the mixed therapeutic composition based on the differential charge and size variant of the constituents.
134. An apparatus comprising a tandem HPLC system for use in analyzing a mixed therapeutically effective formulation sample comprising a combination of two protein moieties, wherein the first protein moiety (A) is conjugated to a polymer, and the second protein moiety (B) is not conjugated to a polymer, wherein the apparatus comprises:
   a) a HPLC pump connected to an auto-injector;
   b) a prefilter;
   c) cation-exchanger column (CEX);
   d) a switch valve enabling the effluent of the CEX to be directed to multiple possible targets,
   wherein the apparatus allows analysis of the mixed therapeutically effective formulation based on the differential charge and size variant of the constituents.
135. A method of analyzing a mixed formulation sample, the method comprising:
   i. providing a mixed formulation comprising a combination of two protein moieties, wherein the first protein moiety (A) is conjugated to a polymer, and the second protein moiety (B) is not conjugated to a polymer; and
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
      wherein an additional pump is used to pulse elute the CEX-bound fraction upon
      wherein the method allows analysis of the mixed formulation based on the differential charge and size variant of the constituents; and
      wherein the sample is analyzed via a single continuous chromatography run.
136. A method of analyzing a mixed therapeutic composition sample, the method comprising:
   i. providing a mixed therapeutic composition comprising a combination of two protein moieties, wherein the first protein moiety (A) is conjugated to a polymer, and the second protein moiety (B) is not conjugated to a polymer; and
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
      wherein an additional pump is used to pulse elute the CEX-bound fraction upon
      wherein the method allows analysis of the mixed therapeutic composition based on the differential charge and size variant of the constituents; and
      wherein the sample is analyzed via a single continuous chromatography run.
137. A method of analyzing a mixed therapeutically effective formulation sample, the method comprising:
   i. providing a mixed therapeutically effective formulation comprising a combination of two protein moieties, wherein the first protein moiety (A) is conjugated to a polymer, and the second protein moiety (B) is not conjugated to a polymer; and
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
      wherein an additional pump is used to pulse elute the CEX-bound fraction upon
      wherein the method allows analysis of the mixed therapeutically effective formulation based on the differential charge and size variant of the constituents; and
      wherein the sample is analyzed via a single continuous chromatography run.
138. An apparatus comprising a tandem HPLC system for use in purifying a mixed formulation sample comprising a combination of two protein moieties, wherein the first protein moiety (A) is conjugated to a polymer, and the second protein moiety (B) is not conjugated to a polymer,
   wherein the apparatus comprises:
   a) a HPLC pump connected to an auto-injector;
   b) a prefilter;
   c) cation-exchanger column (CEX);
   d) a switch valve enabling the effluent of the CEX to be directed to multiple possible targets,
   wherein the apparatus allows analysis of the mixed formulation based on the differential charge and size variant of the constituents;
   wherein the sample is analyzed via a single continuous chromatography run.
139. An apparatus comprising a tandem HPLC system for use in purifying a mixed therapeutic composition sample comprising a combination of two protein moieties, wherein the first protein moiety (A) is conjugated to a polymer, and the second protein moiety (B) is not conjugated to a polymer,
   wherein the apparatus comprises:
   a) a HPLC pump connected to an auto-injector;
   b) a prefilter;
   c) cation-exchanger column (CEX);
   d) a switch valve enabling the effluent of the CEX to be directed to multiple possible targets,
   wherein the apparatus allows analysis of the mixed therapeutic composition based on the differential charge and size variant of the constituents;
   wherein the sample is analyzed via a single continuous chromatography run.
140. An apparatus comprising a tandem HPLC system for use in purifying a mixed therapeutically effective formulation sample comprising a combination of two protein moieties, wherein the first protein moiety (A) is conjugated to a polymer, and the second protein moiety (B) is not conjugated to a polymer,
   wherein the apparatus comprises:
   a) a HPLC pump connected to an auto-injector;
   b) a prefilter;
   c) cation-exchanger column (CEX);
   d) a switch valve enabling the effluent of the CEX to be directed to multiple possible targets,
   wherein the apparatus allows analysis of the mixed therapeutically effective formulation based on the differential charge and size variant of the constituents;
   wherein the sample is analyzed via a single continuous chromatography run.
141. The method or apparatus of any one of the previous of arrangements, wherein the switch valve is a multi-switch valve.
142. The method or apparatus of arrangement 131, wherein the multi-switch valve can direct the CEX effluent to:
   1) a bypass loop;
   2) a first SEC column; or
   3) a second SEC column.
143. The method or apparatus of any one of the previous of arrangements, wherein the tandem HPLC system further comprises an additional optional switch valve after the prefilter but before the CEX column.
144. The method or apparatus of any one of the previous of arrangements, wherein proteins A and B can be the same protein moiety.
145. The method or apparatus of any one of the previous of arrangements, wherein proteins A and B are different protein moieties.
146. The method or apparatus of any one of the previous of arrangements, wherein the protein moieties are antibodies.
147. The method or apparatus of any one of the previous of arrangements, wherein the polymer of protein A comprises a phosphorylcholine containing polymer, wherein the polymer is covalently bonded to protein A at a cysteine outside a variable region of protein A and wherein said cysteine replaces a non-cysteine amino acid that occurs in a same position in a sequence, wherein protein A comprises a light chain and heavy chain, said heavy chain comprising an Fc region, wherein the cysteine is in the Fc region of the heavy chain, wherein the sequence of the heavy chain comprises SEQ ID NO: 1 (with or without the C-terminal lysine), and wherein the sequence of the light chain comprises SEQ ID NO: 2;
   wherein A has the following structure:

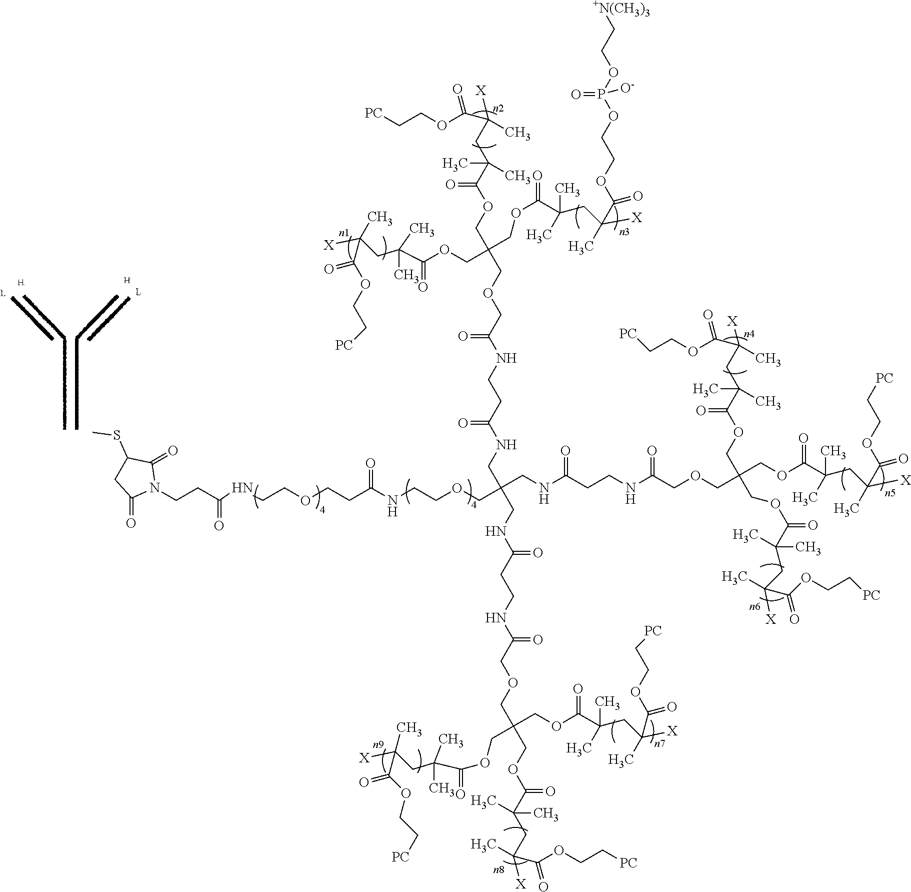

where X is a) —OR where R is —H, Methyl, ethyl, propyl, or isopropyl, b) —H, or c) a halide; (or optionally where X is a) —OR where R is —H, Methyl, ethyl, propyl, or isopropyl, b) —H, c) any halogen, including —Br, —Cl, or —I, d) —SCN, or e) —NCS;)
wherein:
each heavy chain of protein A is denoted by the letter H, and each light chain of A is denoted by the letter L;
the polymer is bonded to A through the sulfhydryl of a cysteine at position 449, as numbered in SEQ ID NO: 1, which bond is depicted on one of the heavy chains;
PC is

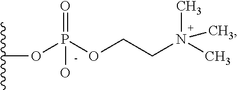

where the curvy line indicates the point of attachment to the rest of the polymer; and
n1, n2, n3, n4, n5, n6, n7, n8 and n9 are the same or different such that the sum of n1, n2, n3, n4, n5, n6, n7, n8 and n9 is 2500 plus or minus 15%; and
148. The method or apparatus of any one of the previous of arrangements, wherein protein B comprises a light chain and heavy chain, said heavy chain comprising an Fc region, wherein a cysteine is in the Fc region of the heavy chain, wherein the sequence of the heavy chain comprises SEQ ID NO: 1 (with or without the C-terminal lysine), and wherein the sequence of the light chain comprises SEQ ID NO: 2.
149. The method or apparatus of any one of the previous of arrangements, wherein the heavy chain of proteins A and B comprise CDRH1: GYDFTHYGMN, CDRH2: WINTYTGEPTYAADFKR, and CDRH3: YPYYYGTSHWYFDV, and position 231 is T (via sequential counting as in SEQ ID NO: 1), and the light chain of proteins A and B comprise CDRL1: SASQDISNYLN (SEQ ID NO: 12), CDRL2: FTSSLHS (SEQ ID NO: 13), and CDRL3: QQYSTVPWT (SEQ ID NO: 14), and Kabat position 4 is L.
150. The method or apparatus of any one of the previous of arrangements, wherein the polymer has 9 arms, and wherein the polymer has a molecular weight of between about 600,000 to about 1,000,000 Da.
151. The method or apparatus of any one of the previous of arrangements, wherein the MW of protein A is greater than or equal to the MW of protein B.
152. The method or apparatus of any one of the previous of arrangements, wherein the MW of protein A is less than or equal to the MW of protein B.
153. The method or apparatus of any one of arrangements 142-152, wherein the mixed formulation or composition sample travels sequentially through a prefilter, said CEX column, and either the first or the second SEC HPLC column.
154. The method or apparatus of arrangement 153, wherein the CEX column is a Shodex SP825 CEX column with internal diameter (i.d.)×length dimensions of 9.0×75 mm.
155. The method or apparatus of arrangement 153, wherein the first SEC HPLC is a TSKgel G3000SWxl column with i.d.×length dimensions of 7.8×300 mm.
156. The method or apparatus of arrangement 153, wherein the second SEC HPLC is a Shodex-806M_HQ column.
157. The method or apparatus of any one of the previous of arrangements, wherein the isocratic running conditions of the columns comprise a flow rate of 0.1-5 ml/min.
158. The method or apparatus of arrangement 157, wherein the isocratic running conditions of the columns comprise a flow rate of 0.5-1 ml/min.
159. The method or apparatus of any one of the previous of arrangements, wherein the buffer comprises 20 mM sodium acetate at pH 3-6.
160. The method or apparatus of arrangement 159, wherein the buffer comprises 20 mM sodium acetate at pH 5.
161. The method or apparatus of any one of the previous of arrangements, wherein the buffer comprises any amount between 50-500 mM NaCl.
162. The method or apparatus of arrangement 161, wherein the buffer comprises any amount between 90-120 mM NaCl.
163. The method or apparatus of any one of the previous of arrangements, further comprising assessment for high molecular weight aggregates within the purified mixed formulation or composition by methods comprising SEC profile analysis and SDS PAGE gel.
164. The method or apparatus of any one of the previous of arrangements, wherein the percentage of B in the mixed formulation or composition is any percentage between 0-93%.
165. The method or apparatus of arrangement 164, wherein the percentage of protein B in the mixed formulation or composition is any percentage between 0% and 50%.
166. The method or apparatus of arrangement 165, wherein the percentage of protein B in the mixed formulation composition is any percentage between 0% and 20%.
167. The method or apparatus of any one of the previous of arrangements, further comprising assessment of the percentage of protein B in the purified formulation or composition by SEC profile analysis.
168. The method or apparatus of arrangement 153, wherein the first run separates protein A from protein B and aggregates.
169. The method or apparatus of arrangement 153, wherein a flow splitter is installed before the first or the second SEC HPLC column.
170. The method or apparatus of arrangement 169, wherein the flow splitter allows an appropriate amount of loaded mixed formulation or composition sample to enter the SEC column for optimal resolution.
171. The method or apparatus of arrangement 153, wherein the effluent of the first and/or second SEC HPLC columns is connected to a multi angle light scattering (MALS) detector.
172. The method or apparatus of arrangement 153, wherein the step for elution of CEX bound protein comprises a pulse of high salt at 0.5-5M NaCl in water or in a buffer at pH 5.
173. The method or apparatus of arrangement 172, wherein the step for elution of CEX bound protein comprises a pulse of high salt at 1M NaCl.
174. The method or apparatus of arrangement 153, wherein high salt pulse elution buffer is applied through an additional pump that, wherein the pump functions in a step elution mode for a period of time, then returns to isocratic mode.
175. The method or apparatus of arrangement 153, wherein an additional valve is installed downstream of the first and/or second SEC column and the eluted free protein peaks are collected using a fraction collector for other analytical techniques comprising icIEF, mass spectrometry, fluorescence detection, photodiode array (PDA) detector.
176. The method or apparatus of any one of the previous of arrangements, wherein the molar ratio range of the second protein or antibody not conjugated to a polymer to the first protein or antibody conjugated to a polymer is any number between 0.01 and 55.
177. The method or apparatus of arrangement 176, wherein the molar ratio range of the second protein or antibody not conjugated to a polymer to the first protein or antibody conjugated to a polymer is any number between 0.01 and 16.
178. The method or apparatus of any one of the previous of arrangements, wherein the second protein or antibody is added directly to the drug product of the first protein or antibody to generate the mixed formulation or composition.
179. The method or apparatus of any one of the previous of arrangements, wherein the second protein or antibody is added to the CEX pool with the first protein or antibody and undergoes ultrafiltration and diafiltration to generate the mixed formulation or composition.
180. The method or apparatus of any one of the previous of arrangements, wherein the second protein or antibody undergoes decapping and refolding prior to being added to the CEX pool.
181. The method or apparatus of any one of the previous of arrangements, wherein the decapped and refolded second protein or antibody undergoes further treatment with alkylation agents such as iodoacetamide (IAM) or N-ethylmaleimide (NEM) before being added to the CEX pool.
182. The method or apparatus of any one of the previous of arrangements, wherein the mixed formulation or composition further comprises an additional protein or antibody conjugated to a polymer.
183. The method or apparatus of arrangement 146, wherein the antibody is a complement factor D (CFD) antibody,
   wherein the complement factor D antibody comprises a heavy chain and a light chain,
   wherein the heavy chain variable region (VH) comprises the amino acid sequence of any one of SEQ ID NOs: 15-47; and the light chain variable region (VL) comprises the amino acid sequence of any one of SEQ ID NOs: 48-80.
184. The method or apparatus of arrangement 146, wherein the antibody comprises an antagonist IL-6 antibody fused to a VEGF trap, wherein the antibody comprises a heavy chain and a light chain,
   wherein the heavy chain variable region (VH) comprises the amino acid sequence of any one of SEQ ID NOs: 81-89; and the light chain variable region (VL) comprises the amino acid sequence of any one of SEQ ID NOs: 90-92.
185. The method or apparatus of arrangement 146, wherein the heavy and light chains are selected from the group comprising:
   (A) the heavy chain of SEQ ID NO:93 (with or without the C-terminal lysine), and the light chain of SEQ ID NO:94;
   (B) the heavy chain of SEQ ID NO:95 (with or without the C-terminal lysine), and the light chain of SEQ ID NO:96;
   (C) the heavy chain of SEQ ID NO:97 (with or without the C-terminal lysine), and the light chain of SEQ ID NO:98;
   (D) the heavy chain of SEQ ID NO:99 (with or without the C-terminal lysine), and the light chain of SEQ ID NO:100;
   (E) the heavy chain of SEQ ID NO:101 (with or without the C-terminal lysine), and the light chain of SEQ ID NO:102;
   (F) the heavy chain of SEQ ID NO:103 (with or without the C-terminal lysine), and the light chain of SEQ ID NO: 104;
   (G) the heavy chain of SEQ ID NO: 105 (with or without the C-terminal lysine), and the light chain of SEQ ID NO:106;
   (H) the heavy chain of SEQ ID NO:107 (with or without the C-terminal lysine), and the light chain of SEQ ID NO:108;
   (I) the heavy chain of SEQ ID NO: 109 (with or without the C-terminal lysine), and the light chain of SEQ ID NO:110;
   (J) the heavy chain of SEQ ID NO:111 (with or without the C-terminal lysine), and the light chain of SEQ ID NO:112;
   (K) the heavy chain of SEQ ID NO:113 (with or without the C-terminal lysine), and the light chain of SEQ ID NO:114;
   (L) the heavy chain of SEQ ID NO:115 (with or without the C-terminal lysine), and the light chain of SEQ ID NO:116;
   (M) the heavy chain of SEQ ID NO:117 (with or without the C-terminal lysine), and the light chain of SEQ ID NO:118;
   (N) the heavy chain of SEQ ID NO:119 (with or without the C-terminal lysine), and the light chain of SEQ ID NO:120;
   (O) the heavy chain of SEQ ID NO: 121 (with or without the C-terminal lysine), and the light chain of SEQ ID NO: 122;
   (P) the heavy chain of SEQ ID NO: 123 (with or without the C-terminal lysine), and the light chain of SEQ ID NO:124;
   (Q) the heavy chain of SEQ ID NO:125 (with or without the C-terminal lysine), and the light chain of SEQ ID NO:126; and
   (R) the heavy chain of SEQ ID NO: 127 (with or without the C-terminal lysine), and the light chain of SEQ ID NO: 128.
186. The method or apparatus of arrangement 146, wherein protein A, B, or A and B comprise a light chain and a heavy chain, said heavy chain comprising an Fc region.
187. The method or apparatus of arrangement 146, wherein a cysteine of protein A, B, or A and B is in the Fc region of the heavy chain.
188. The method or apparatus of arrangement 146, wherein protein A, B, or A and B are an immunoglobulin G (IgG).
189. The method or apparatus of arrangement 146, wherein the heavy chain of protein A, B, or A and B comprises: CDRH1: GYDFTHYGMN (SEQ ID NO: 9), CDRH2: WINTYTGEPTYAADFKR (SEQ ID NO: 10), and CDRH3: YPYYYGTSHWYFDV (SEQ ID NO: 11), and the light chain of protein A, B, or A and B comprises CDRL1: SASQDISNYLN (SEQ ID NO: 12), CDRL2: FTSSLHS (SEQ ID NO: 13), and CDRL3: QQYSTVPWT (SEQ ID NO: 14).
190. The method or apparatus of arrangement 146 wherein the heavy chain isotype of protein A, B, or A and B is human IgG1.
191. The method or apparatus of arrangement 146, wherein the heavy chain constant domain of protein A, B, or A and B has one or more mutations relative to the constant domain of human IgG1 to modulate effector function.
192. The method or apparatus of arrangement 191, wherein the mutations of protein A, B, or A and B are to one or more of the following amino acid positions (EU numbering): E233X, L234X, L235X, G236X, G237X, A327X, A330X, and P331X wherein X is any natural or unnatural amino acid.
193. The method or apparatus of arrangement 191, wherein the mutations of protein A, B, or A and B are selected from the group consisting of (EU numbering): E233P, L234V, L234A, L235A, G237A, A327G, A330S, and P331S.
194. The method or apparatus of arrangement 191, comprising the following mutations of protein A, B, or A and B: L234A, L235A, and G237A (EU numbering).
195. The method or apparatus of arrangement 191, wherein the cysteine of protein A, B, or A and B is in the antibody heavy chain and is Q347C (EU numbering) or L443C (EU numbering).
196. The method or apparatus of arrangement 146, wherein the sequence of the antibody heavy chain of protein A, B, or A and B is SEQ ID NO: 1 (with or without the C-terminal lysine) and the sequence of the light chain of protein A, B, or A and B is SEQ ID NO: 2.
197. The method or apparatus of arrangement 146, wherein the cysteine of protein A, B, or A and B is L443C (EU numbering).
198. The method or apparatus of any one of the preceding arrangements, wherein the polymer of the conjugate has three or more arms or is synthesized with an initiator comprising 3 or more polymer initiation sites.
199. The method or apparatus of any one of the preceding arrangements, wherein the polymer of the conjugate has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites.
200. The method or apparatus of any one of the preceding arrangements, wherein the polymer of the conjugate has a polydispersity value (PDI) of less than about 1.2.
201. The method or apparatus of any one of the previous of arrangements, wherein the percent composition of (B) relative to the total protein mass weight concentration of (A) and (B) in the formulation or composition is any of the following: about 7% (B); about 10% (B); about 12.5% (B); about 15% (B); about 17.5% (B); about 20% (B); and about 25% (B).
202. The method or apparatus of any one of the previous of arrangements, wherein the mixed formulation or composition comprises the second protein at about 1% or more of a total molar amount of the conjugate and the second protein, wherein the total molar amount comprises a sum of the first molar amount and the second molar amount.
203. The method or apparatus of any one of the previous of arrangements, wherein the mixed formulation or composition comprises the second protein at about 1% or more of a total molar amount of the conjugate and the second protein, wherein the total molar amount comprises a sum of the first molar amount and the second molar amount.
204. The method or apparatus of any one of the previous of arrangements, wherein the mixed formulation or composition comprises the second protein at between about 5% to about 50% of the total molar amount of the conjugate and the second protein.
205. The method or apparatus of any one of the previous of arrangements, wherein a ratio of the molecular weight of the second protein to the polymer in the formulation or composition is at most 1:2.
206. The method or apparatus of any one of the previous of arrangements, wherein a ratio of the molecular weight of the second protein to the polymer in the formulation or composition is between about 1:2 and about 1:10.
207. The method or apparatus of any one of the previous of arrangements, wherein the mixed formulation or composition sample comprises any amount between 50 ug and 1340 ug of protein.
208. The method or apparatus of any one of the previous of arrangements, wherein the sample is loaded into the tandem HPLC system via a single injection.
209. The method or apparatus of any one of the previous of arrangements, wherein the sample is loaded into the tandem HPLC system via multiple injections.
210. A method of analyzing a mixed formulation sample, the method comprising:
   i. providing a mixed formulation comprising a combination of two protein moieties, wherein the first protein moiety (A) is conjugated to a polymer, and the second protein moiety (B) is not conjugated to a polymer; and
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
   wherein the switch valve can direct the CEX effluent to:
      1) a bypass loop;
      2) a first SEC column; or
      3) a second SEC column; and
      wherein the method allows analysis of the mixed formulation based on the differential charge and size variant of the constituents,
      wherein the sample is analyzed via a single continuous chromatography run, and
      wherein the conjugated sample is directed to the bypass loop and the unconjugated sample is directed to the first or second SEC column.
211. A method of analyzing a mixed therapeutic composition sample, the method comprising:
   i. providing a mixed therapeutic composition comprising a combination of two protein moieties, wherein the first protein moiety (A) is conjugated to a polymer, and the second protein moiety (B) is not conjugated to a polymer; and
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
   wherein the switch valve can direct the CEX effluent to:
      1) a bypass loop;
      2) a first SEC column; or
      3) a second SEC column; and
      wherein the method allows analysis of the mixed therapeutic composition based on the differential charge and size variant of the constituents,
      wherein the sample is analyzed via a single continuous chromatography run, and
      wherein the conjugated sample is directed to the bypass loop and the unconjugated sample is directed to the first or second SEC column.
212. A method of analyzing a mixed therapeutically effective formulation sample, the method comprising:
   i. providing a mixed therapeutically effective formulation comprising a combination of two protein moieties, wherein the first protein moiety (A) is conjugated to a polymer, and the second protein moiety (B) is not conjugated to a polymer; and
   ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
   wherein the switch valve can direct the CEX effluent to:
      1) a bypass loop;
      2) a first SEC column; or
      3) a second SEC column; and
      wherein the method allows analysis of the mixed therapeutically effective formulation based on the differential charge and size variant of the constituents,
      wherein the sample is analyzed via a single continuous chromatography run, and
      wherein the conjugated sample is directed to the bypass loop and the unconjugated sample is directed to the first or second SEC column.
213. An apparatus comprising a tandem series of HPLC system for use in analyzing a mixed formulation sample comprising (A) a first protein conjugated to a polymer and (B) a second protein that is not conjugated to the polymer, wherein, the HPLC system comprises:
   a) a HPLC pump connected to an auto-injector;
   b) a prefilter;
   c) cation-exchanger column (CEX);
   d) a switch valve enabling the effluent of the CEX to be directed to multiple possible targets, wherein the switch valve can direct the CEX effluent to:
   1) a bypass loop;
   2) a first SEC column; or
   3) a second SEC column; and
   wherein the apparatus allows analysis of the mixed formulation based on the differential charge and size variant of the constituents,
   wherein the sample is analyzed via a single continuous chromatography run; and
   wherein the conjugated sample is directed to the bypass loop and the unconjugated sample is directed to the first or second SEC column.
214. An apparatus comprising a tandem series of HPLC system for use in analyzing a mixed therapeutic composition sample comprising (A) a first protein conjugated to a polymer and (B) a second protein that is not conjugated to the polymer, wherein, the HPLC system comprises:
   a) a HPLC pump connected to an auto-injector;
   b) a prefilter;
   c) cation-exchanger column (CEX);
   d) a switch valve enabling the effluent of the CEX to be directed to multiple possible targets, wherein the switch valve can direct the CEX effluent to:
   1) a bypass loop;
   2) a first SEC column; or
   3) a second SEC column; and
   wherein the apparatus allows analysis of the mixed therapeutic composition based on the differential charge and size variant of the constituents,
   wherein the sample is analyzed via a single continuous chromatography run; and
   wherein the conjugated sample is directed to the bypass loop and the unconjugated sample is directed to the first or second SEC column.
215. An apparatus comprising a tandem series of HPLC system for use in analyzing a mixed therapeutically effective formulation sample comprising (A) a first protein conjugated to a polymer and (B) a second protein that is not conjugated to the polymer, wherein, the HPLC system comprises:
   a) a HPLC pump connected to an auto-injector;
   b) a prefilter;
   c) cation-exchanger column (CEX);
   d) a switch valve enabling the effluent of the CEX to be directed to multiple possible targets, wherein the switch valve can direct the CEX effluent to:
   1) a bypass loop;
   2) a first SEC column; or
   3) a second SEC column; and
   wherein the apparatus allows analysis of the mixed therapeutically effective formulation based on the differential charge and size variant of the constituents,
   wherein the sample is analyzed via a single continuous chromatography run; and
   wherein the conjugated sample is directed to the bypass loop and the unconjugated sample is directed to the first or second SEC column.
216. The method or apparatus of any one of the preceding arrangements, wherein the mixed formulation or composition comprises the second protein moiety at about 1% or more of a total molar amount of the conjugate and the second protein moiety, wherein the total molar amount comprises a sum of a first molar amount of the first protein moiety in the formulation or composition, and a second molar amount of the second protein moiety in the formulation or composition.
217. The method or apparatus of any one of the preceding arrangements, wherein the mixed formulation or composition comprises a pharmaceutically acceptable carrier.
218. The method or apparatus of arrangement 217, wherein the pharmaceutically acceptable carrier is acceptable for administering directly into an eye of a patient.
219. The method or apparatus of any one of the preceding arrangements, wherein the mixed formulation or composition comprises polysorbate 20.
220. The method or apparatus of any one of the preceding arrangements, wherein the mixed formulation or composition comprises about 0.01-0.05% (w/w) polysorbate 20.
221. The method or apparatus of any one of the preceding arrangements, wherein the first protein moiety or first antibody comprises any one of the amino sequences or a combination thereof set forth in Tables 1-3 and/or SEQ ID NOs: 1-161.
222. The method or apparatus of any one of the preceding arrangements, wherein the second protein moiety or second antibody comprises any one of the amino sequences or a combination thereof set forth in Tables 1-3 and/or SEQ ID NOs: 1-161.
223. The method or apparatus of any one of the previous arrangements, wherein the CEX HPLC is an IEC ProPac WCX-10 with i.d.×length dimensions of 4.0 mm×250 mm.
224. The method or apparatus of any one of the previous arrangements, wherein the SEC HPLC is a TSKgel G4000SWXL with i.d.×length dimensions of 7.8 mm×30 cm.
225. The method or apparatus of any one of the previous arrangements, wherein the first protein moiety (A), or first protein conjugated to the polymer (A) is OG1953.
226. The method or apparatus of any one of the previous arrangements, wherein the first protein moiety (A), or first protein conjugated to the polymer (A) is OG2074.
227. The method or apparatus of any one of the previous arrangements, wherein the second protein moiety (B), or second protein not conjugated to the polymer (B) is OG1950.
228. The method or apparatus of any one of the previous arrangements, wherein the second protein moiety (B), or second protein not conjugated to the polymer (B) is OG2072.

In any method or apparatus of the present disclosure, in some embodiments, the mixed formulation (or therapeutically acceptable composition) comprises a first molar amount of a conjugate having a first protein or protein moiety conjugated to a phosphorylcholine-containing polymer and a second molar amount of the second protein or protein moiety that is not conjugated to a phosphorylcholine-containing polymer, wherein the formulation or composition comprises the second protein at about 0.1% or more (e.g., at about 0.2%, at about 0.3%, at about 0.4%, at about 0.5% or more) of a total molar amount of the conjugate and the second protein, and a pharmaceutically acceptable carrier, wherein the total molar amount comprises a sum of the first molar amount and the second molar amount. In some embodiments, a mixed formulation (or therapeutically acceptable composition) comprises a conjugate having a first protein conjugated to a phosphorylcholine-containing polymer and a second protein the second protein that is not conjugated to a phosphorylcholine-containing polymer, and a pharmaceutically acceptable carrier, wherein the percent composition of the second protein relative to the total protein mass weight concentration of the first protein and the second protein in the composition is about 0.1% or more (e.g., about 0.2%, about 0.3%, about 0.4%, about 0.5% or more).

### EXAMPLES

### Example 1. Tandem HPLC Apparatus System and Operation for Mixed Sample Analysis

Analysis of a mixed population of fusion proteins and unconjugated proteins, specifically anti-VEGF-A antibody and anti-VEGF-A antibody conjugated to a polymer, was performed using a tandem HPLC apparatus as shown in FIG. 1. Various mixing scenarios for sample preparation are shown in TABLE 4.

TABLE 4

| | Free protein | Conjugate | Injection amount |
|---|---|---|---|
| Mixture | (mg/mL) -A | (mg/mL)* -B | (ug) (A + B) |
| 5% | 2.5 | 47.5 | 2000 |
| 7% | 3.75 | 46.25 | 1333 |
| 10% | 5 | 45 | 1000 |
| 15% | 7.5 | 42.5 | 666.7 |
| 20% | 10 | 40 | 500 |

*Concentration based on the protein moiety only
A. for analysis, 100 ug of free protein was injected per run
B. In order to fulfill 100 ug free protein injected per load, the amount of conjugate is vary based on the mixture percent. For example, 5% mixture will have 100 ug free protein and 1900 ug conjugate, it results in 2000 ug total injected amount.

The process involved two possible runs with run 1 comprising 1) preparing a mixed sample, 2) injecting the sample into a tandem series of CEX and SEC-HPLC columns, running the sample through a prefilter to remove insoluble particles, 3) passing the sample through a CEX HPLC column. Polymer conjugated protein would pass through the CEX column and could either be analyzed via SEC with the appropriate MW size column or otherwise removed from the sample solution. Run 2 involved 1) eluting protein bound to the CEX column using high salt (1M NaCl) and 2) separating eluted free protein and aggregates using SEC. The process was performed using a running buffer at pH 5 and 100 mM NaCl.

As shown in FIG. 2, multiple versions of the tandem HPLC apparatus can be employed. FIG. 2A shows a version including a solvent, a pump, prefilter, ion exchange column (IEX), SEC, and detector. In this iteration, the IEX can alternatively be a cation exchange column (CEX) or anion exchange column (AEX). Additionally, the first column before the SEC can also be a hydrophobic interaction column (HIC), affinity column, such as a dye column or talon column. Furthermore, as shown in FIG. 1 and FIG. 2B, a switch valve can be implemented downstream of the first column. The switch valve can be synchronized with the injector during autosampler programming and shunt flowthrough to different downstream columns, such as different sized SECs appropriate for analysis of the conjugated protein of run 1, or the eluted free protein and aggregates of run 2. As shown in FIG. 2B, SEC analysis can be followed by additional assessment, including ultraviolet (UV)/vis detection and multi angle light scattering (MALS) detection. Furthermore, an additional column switch valve can be implemented after these detectors and synchronized with the SEC running program to other analytical methods such as icIEF or Mass spec analysis (MS). The operation of the tandem spec apparatus comprising a CEX column to separate the conjugate from the free protein and aggregates is shown in FIG. 3.

### Example 2. Analysis of Mixed Sample Formulations with Tandem HPLC

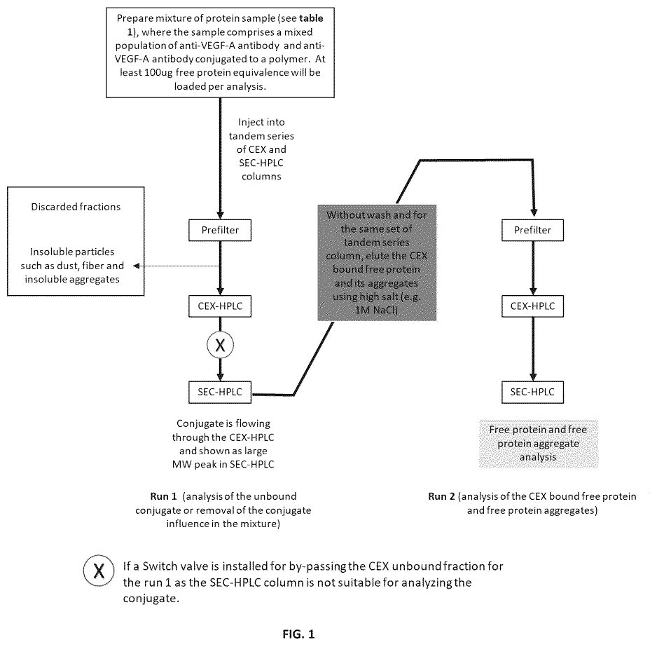

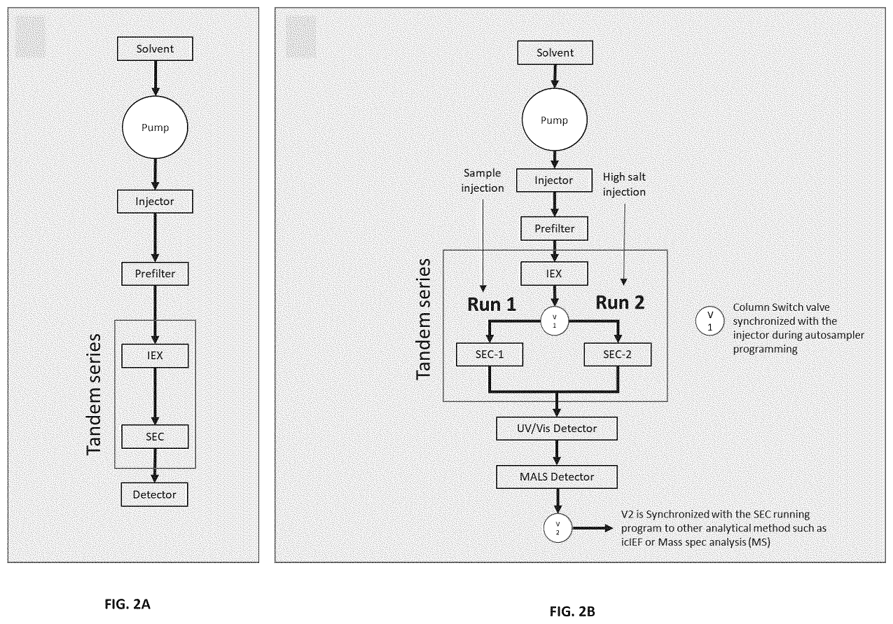

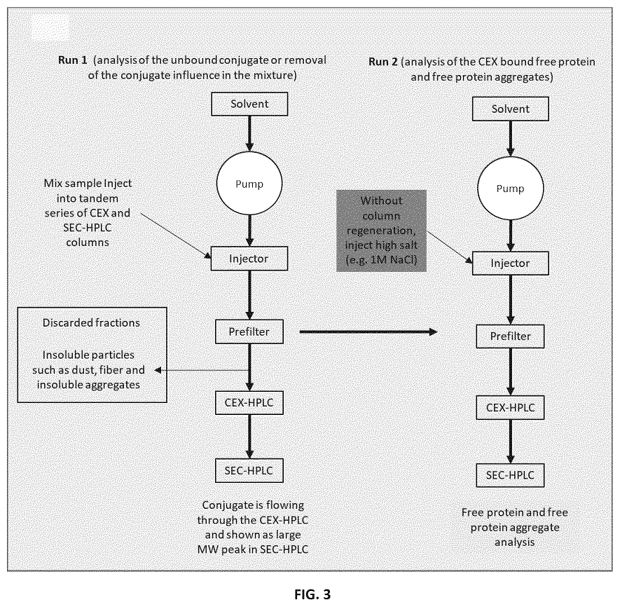

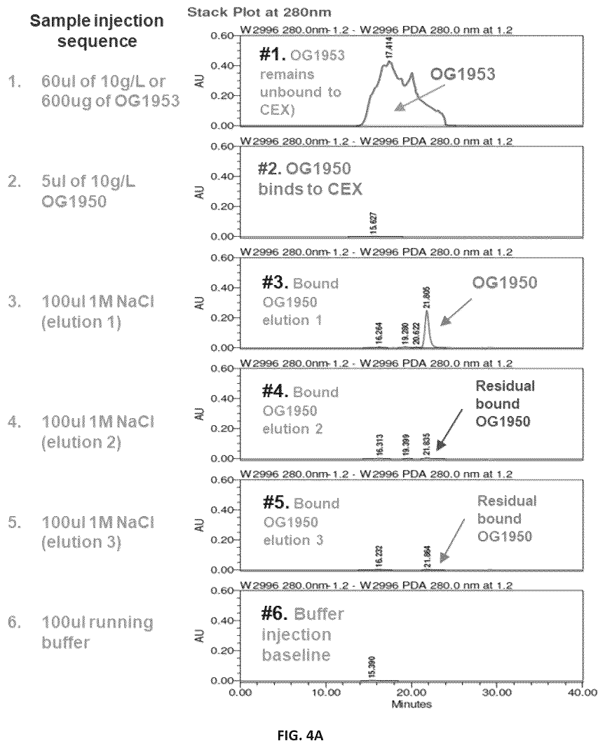

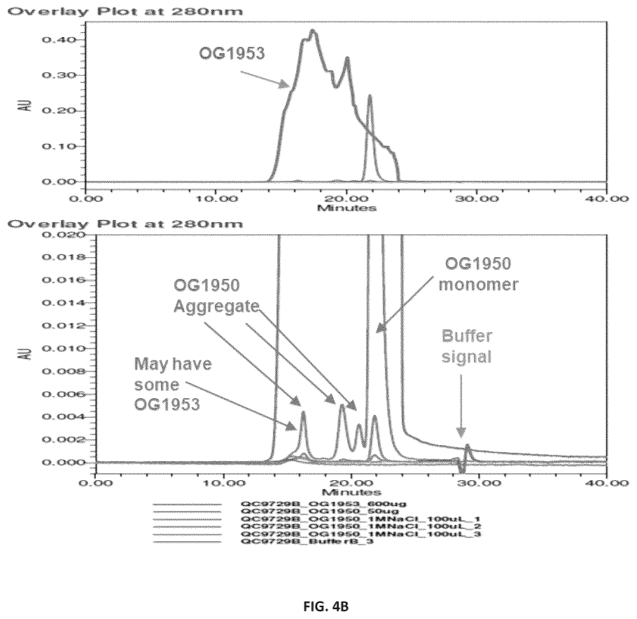

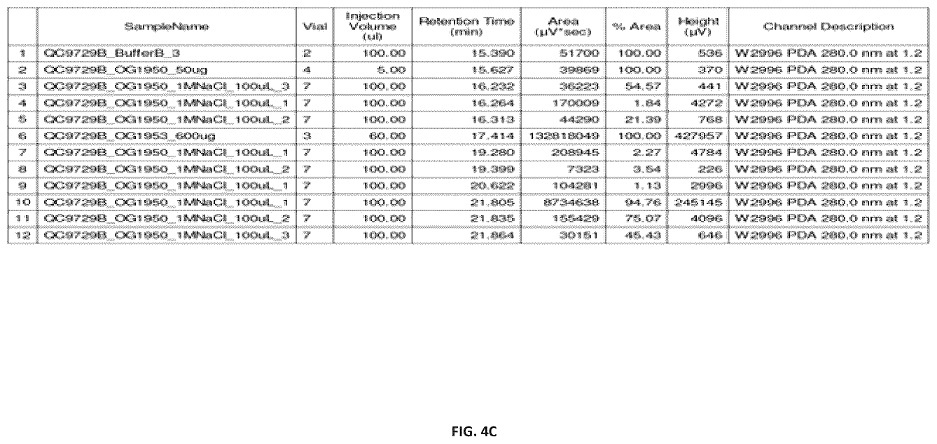

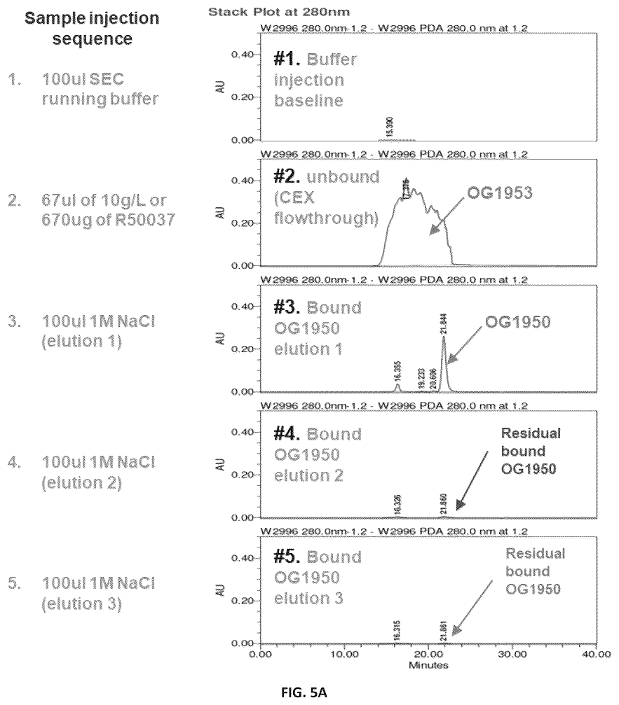

An investigation of the aggregation properties of the anti-VEGF-A antibody (OG1950) in a mixture with polymer conjugated anti-VEGF-A (OG1953) was performed, where the mixture, R50037 was comprised of OG1953+7.5% OG1950. The investigation used a tandem HPLC system as demonstrated in FIG. 4A where the first column was a CEX Shodex SP825 and the second column was a SEC-HPLC TSKG3000.

First, SEC profiles were obtained for samples containing known amounts of purified OG1953 and OG1950 loaded separately into a tandem series of CEX-SEC columns. As shown in FIG. 4A-4B, OG1953 remained unbound to the CEX column (FIG. 4A, panel 1), while the bulk of OG1950 and aggregates bound the column and were eluted in a single elution of 100 uL 1M NaCl (panel 3 of FIG. 4A). Additional sample information is included in FIG. 4C, including sample injection volume (uL), retention time (min), peak area (μV*sec), % area, and height (μV).

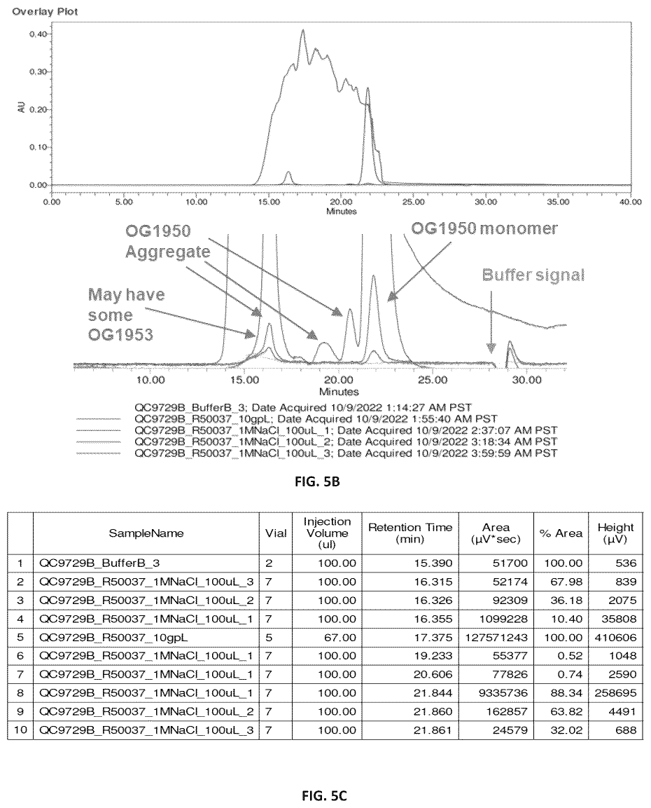

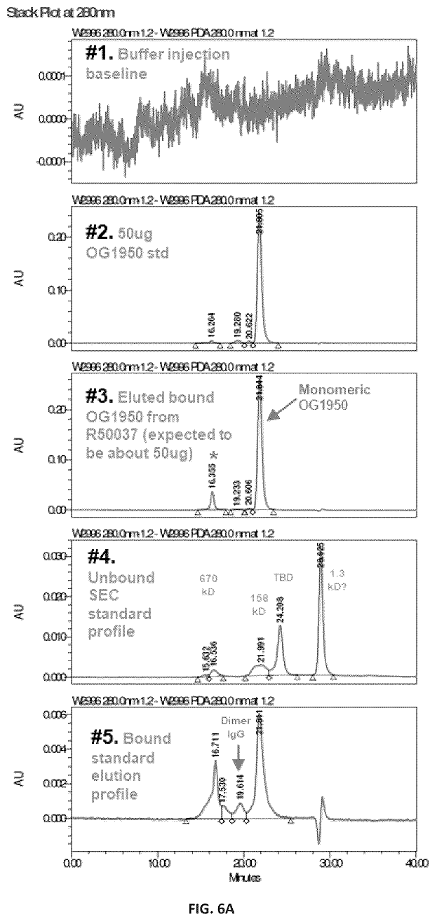

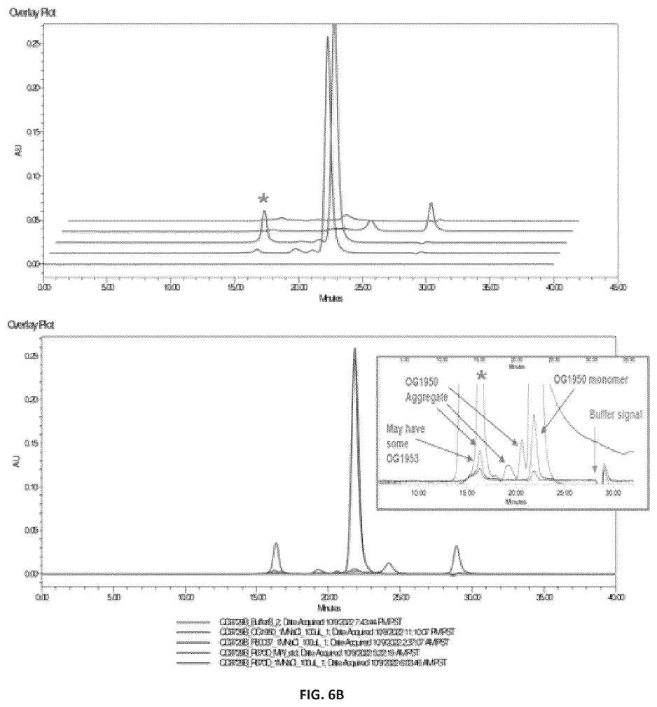

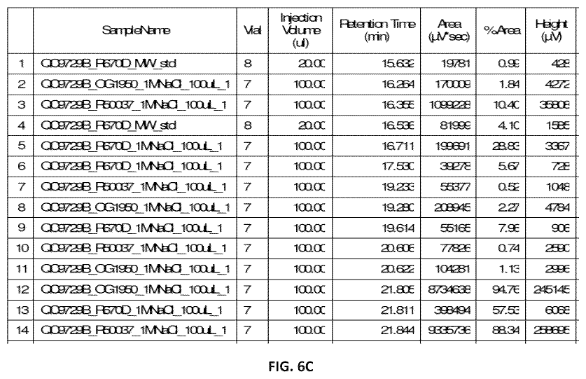

Following analysis of known amounts of unmixed OG1950 and OG1953 samples, CEX-SEC tandem series analysis of OG1953+7.5% OG1950 (OG1953_R50037) was performed. As shown in FIG. 5A-5B, as in the unmixed samples, the bulk of OG1953 is in the flowthrough (FIG. 5A, panel 2), while the bulk of OG1950 and aggregates bound the column and were eluted in a single elution of 100 μL 1M NaCl (panel 3 of FIG. 5A). Additional sample information is included in FIG. 5C, including sample injection volume (uL), retention time (min), peak area (μV*sec), % area, and height (μV). SEC profiles further characterizing the OG1950 peak recovered from R50037 (7.5%) are shown in FIG. 6. As shown in FIG. 6A, the 21.8 minute peak (FIG. 6A, panels 2 and 3) represents monomeric IgG (OG1950), as this peak aligns with the 158 kDa (IgG) peak in the BioRad calibration standard (FIG. 6A, panels 4 and 5). Injection of 670 ug of R50037 was expected to contain 50.25 ug OG1950 (670×0.075=50.25 ug). Injection of 50 ug of OG1950 resulted in single peak at 21.8 min with 94.78% monomeric form (FIG. 6A, panel 2). Comparison of the peak area at 21.8 min with the peak for monomeric OG1950 (FIG. 6A, panels 2 and 3) is highly similar at 8734638 and 9335736 uV*sec, respectively. The expected 50 ug peak area at 1 ml/min is 9275234, thus the result was consistent with the expectation. Aggregates of OG1950 also appeared to be recovered in the R50037 elution as shown in the small insert of FIG. 6B. Additional sample information is included in FIG. 6C, including sample injection volume (uL), retention time (min), peak area (μV*sec), % area, and height (μV).

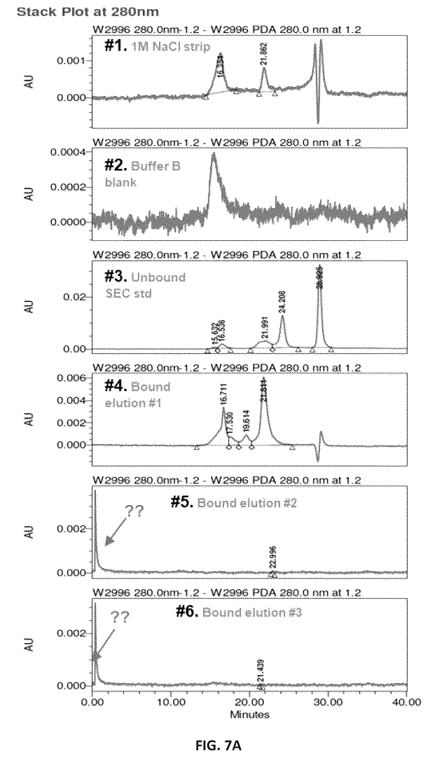

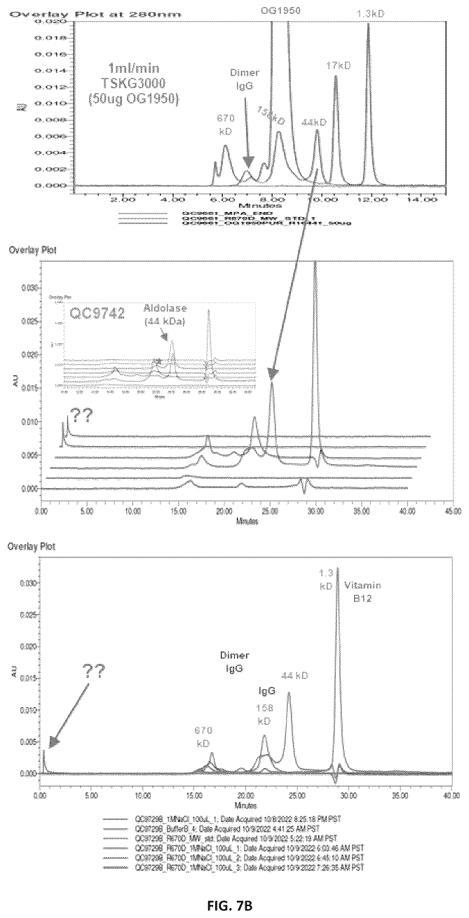

Subsequently, the SEC calibration standard was assessed using the 2D HPLC method (QC9661, QC9729B, & QC9742). As shown in FIG. 7A, panel 3, the 158 kD (IgG) and its dimeric peak at 19.614 min were missing as expected. The majority of 670 kDa and 17 kD peaks were also missing in the unbound fraction. FIG. 7B shows a typical OG1950PUR in a TSKG3000 column only with 1 ml/min flowrate. The SEC standards from 1.3 k-670 kDa standards are shown from right to left. The peak area for 50 ug is 4637617 uV*sec (x2=9275234 (corrected from 1 ml/min to 0.5 ml/min flowrate). The identity of the 25 min elution was identified as 44 kDa aldolase (injection of aldolase alone revealed its identity, see QC9742 insert in FIG. 7B). It should be noted that aldolase contains an unknown peak that comigrates with 158 kDa marker. This explains why there was a residual peak in the flowthrough SEC standard at the 158 kDa range which the IgG supposed to be bound to CEX column). The 640 kD, IgG and the dimer form were eluted (FIG. 7A, panel 4). Interestingly, there was an unknown peak (??) eluted at panel 5 and 6 with 1M NaCl elution (FIG. 7A). This was likely the missing MW marker that bound tightly to the CEX and barely eluted at 1M pulse injection. The delayed elution retarded the marker's elution in the SEC to a much later migration time than buffer peak at 30 min. Using higher salt at 2M with extended run time to 45 min may be able to address this. The surprising partition of SEC standard using current 2D method allowed for calibration of SEC performance for both CEX bound and unbound fraction.

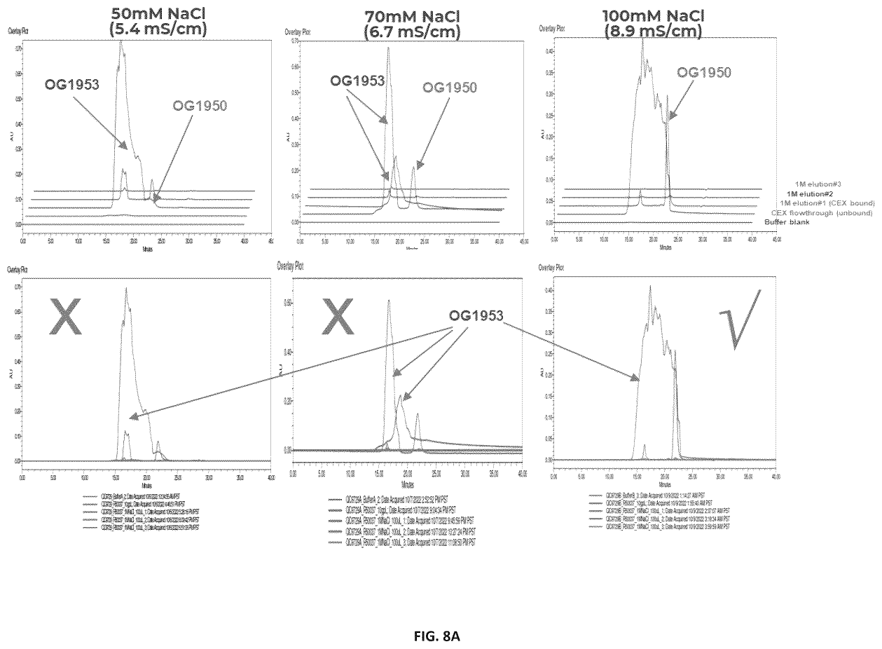

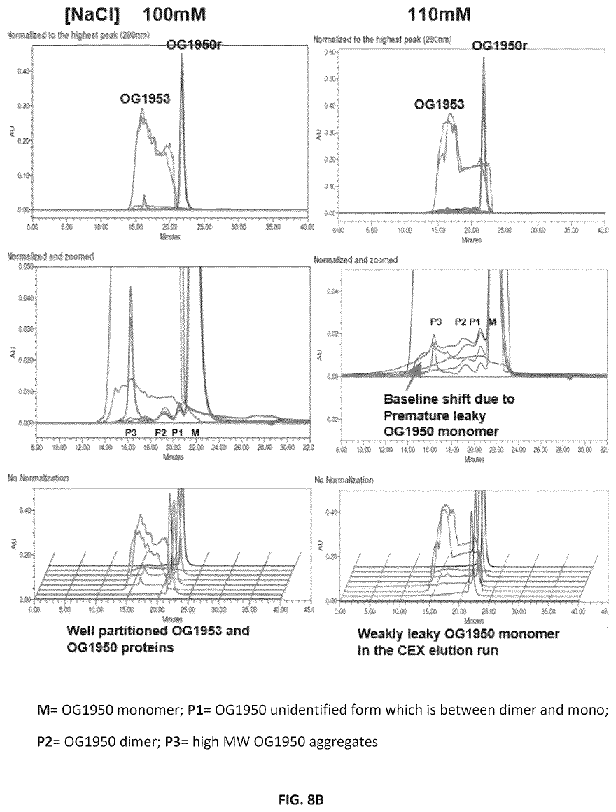

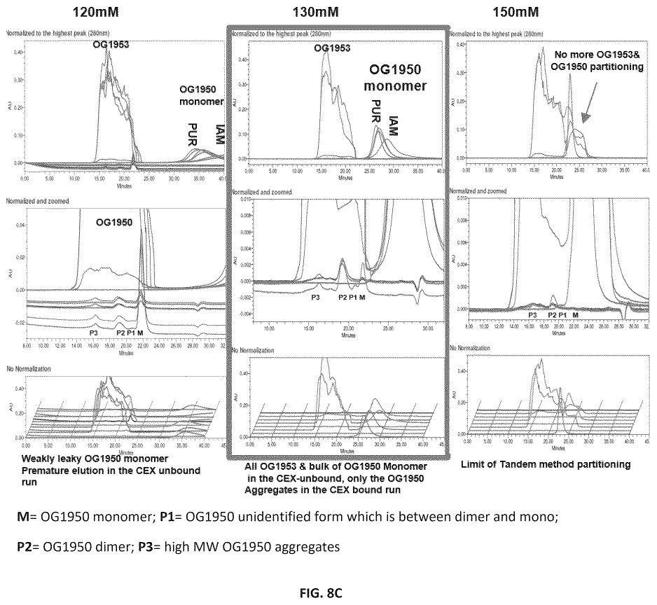

Next, further investigation was performed regarding how different NaCl concentrations and buffer conductivities affected partitioning of OG1950 and OG1953 in the CEX column. As shown in FIG. 8A, buffer with 100 mM NaCl and a conductivity of 8.9 mS/cm separated OG1950 and OG1953 well. However, buffer with 50 mM NaCl (5.4 mS/cm) and 70 mM NaCl (6.7 mS/cm) was not found to be suitable for bulk partitioning of OG1950 and OG1953 (FIG. 8A). Similarly, buffer with NaCl concentrations higher than 100 mM, such as 110 mM NaCl, showed a baseline shift issue and interfered with the resolution of the OG1950 elution profile. This was likely due to the subtle leaching of the bound OG1950 free protein fractions, which was evidenced when increasing salt concentrations such as at 120 mM, 130 mM and 150 mM NaCl resulted in an increase in the degree of premature leaching of the OG1950 free protein (FIG. 8B-C). Starting at 120 mM NaCl, the majority of OG1950 free protein eluted prematurely with only a trace amount eluted in the salt pulse elution phase. At much higher salt concentrations such as 130 mM and 150 mM NaCl, there was no partitioning of OG1953 conjugate and OG1950 free protein for using the Tandem method (FIG. 8C). It should also be noted that the pH can also play a critical role similar to the NaCl concentrations, for the examples described herein, the pH has been controlled at a narrow range of about 4.8-5.0.

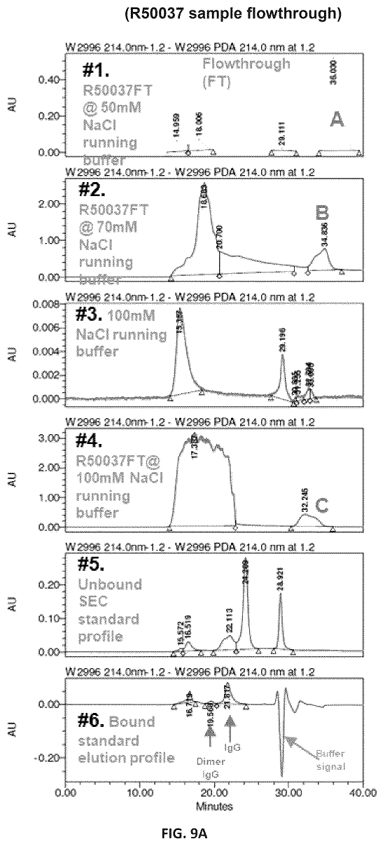

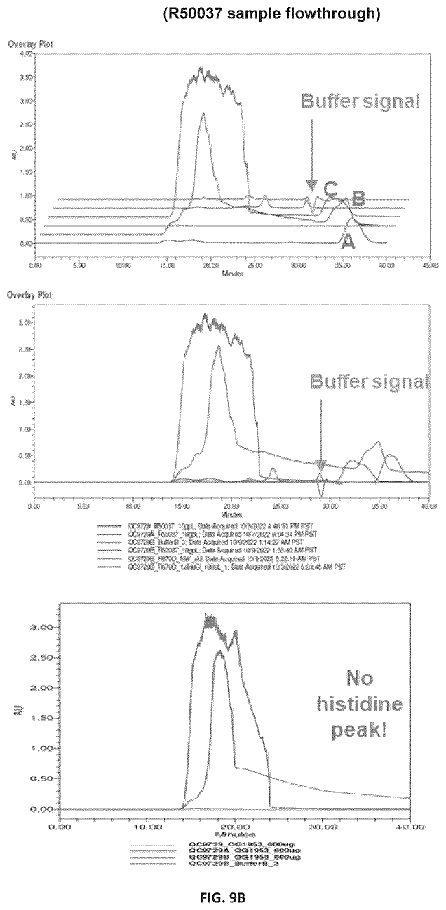

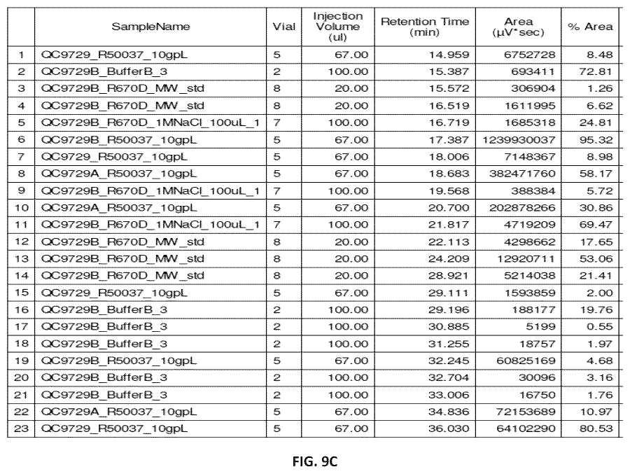

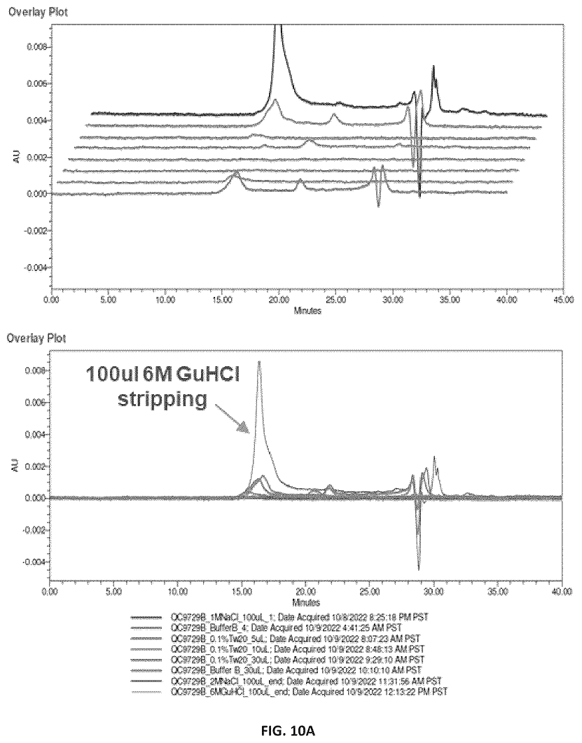

An additional peak was identified in the R50037 sample using the 2D method with 214 nm detection. A CEX interacting fraction was identified in the 214 nm profile, as shown in FIG. 9A, panels 1, 2, and 3 (peaks A, B, C), and FIG. 9B, top panel (peaks A, B, C). This fraction was in the flowthrough of all salt concentrations but with delayed elution in the SEC column. As this fraction was slightly retarded by the CEX column, it was most likely positively charged. Increasing salt concentration result in reduced interaction with CEX and therefore earlier elution time as shown by A>B>C. As shown in FIG. 9B panel 3, the peak was not detected in the OG1953 (current KSI301 reference standard). Histidine has a pKa of 6 and under the current pH 5 condition, it is protonated and positively charged. Therefore, the additional peak is likely the histidine peak, as R50037 is formulated in histidine buffer. Additional sample information is included in FIG. 9C, including sample injection volume (uL), retention time (min), peak area (μV*see), and % area.

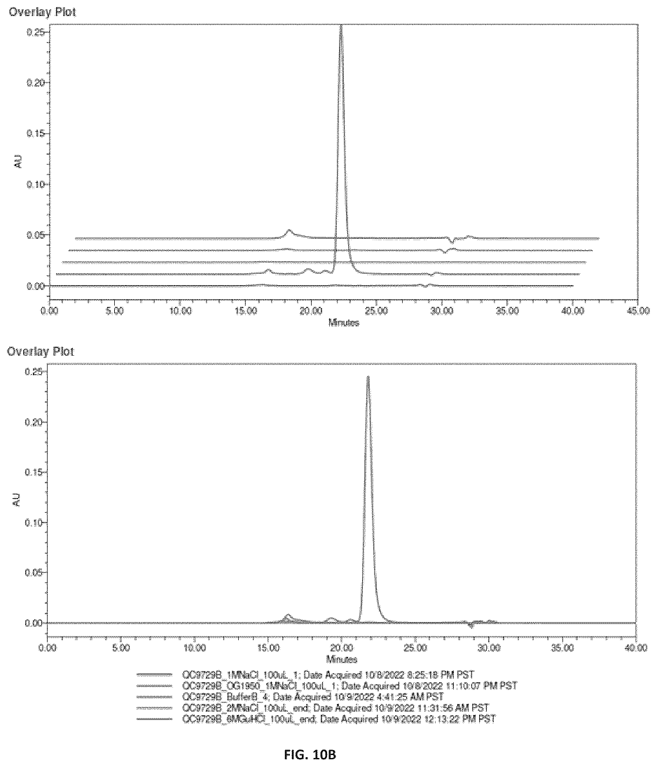

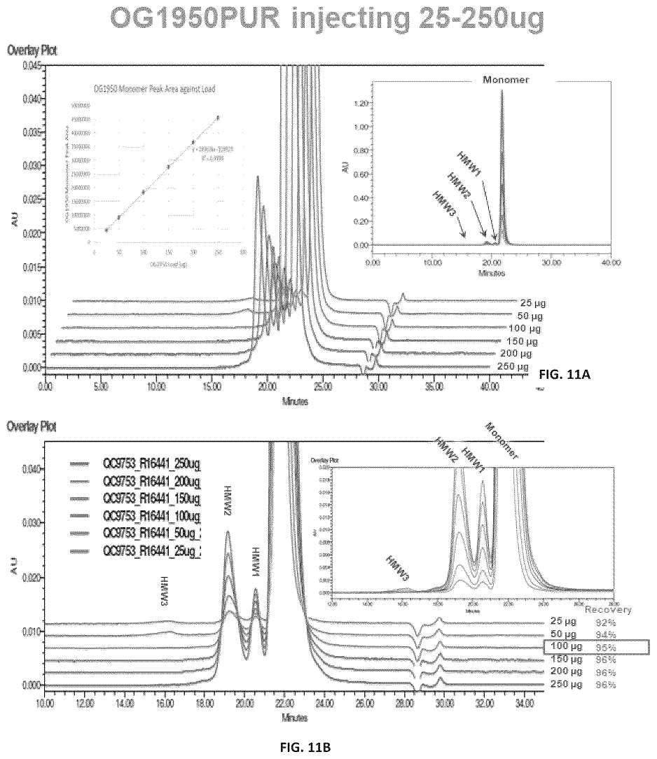

Next, sample carryover was evaluated for the tandem 2D HPLC system. The HPLC system was stripped with various buffers including 2M NaCl and 6M guanidine HCl after over 100 runs of various loading and elution. As shown in FIGS. 10A and 10B, the elution profile chromatograms show minimal residual bound material has been detected, especially with the 6M GuHCl stripping where some large MW aggregates were identified by the SEC profile. Therefore, the carry-over issue appears to be minimal for the current method.

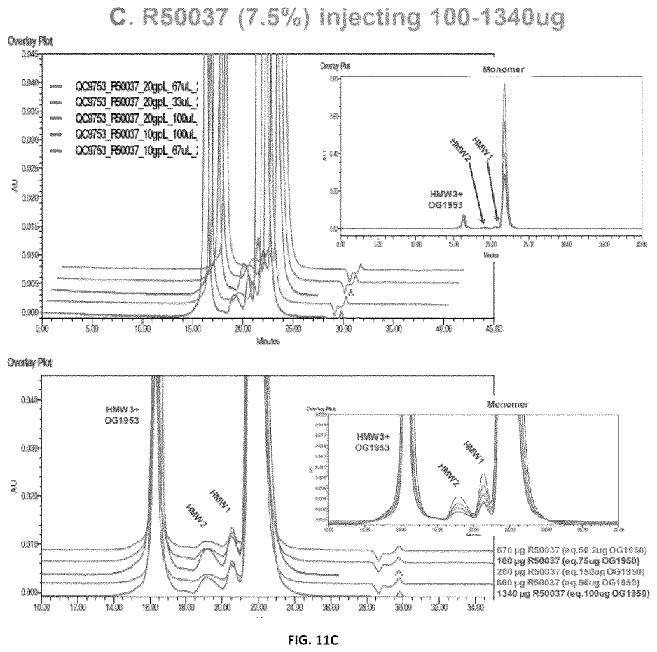

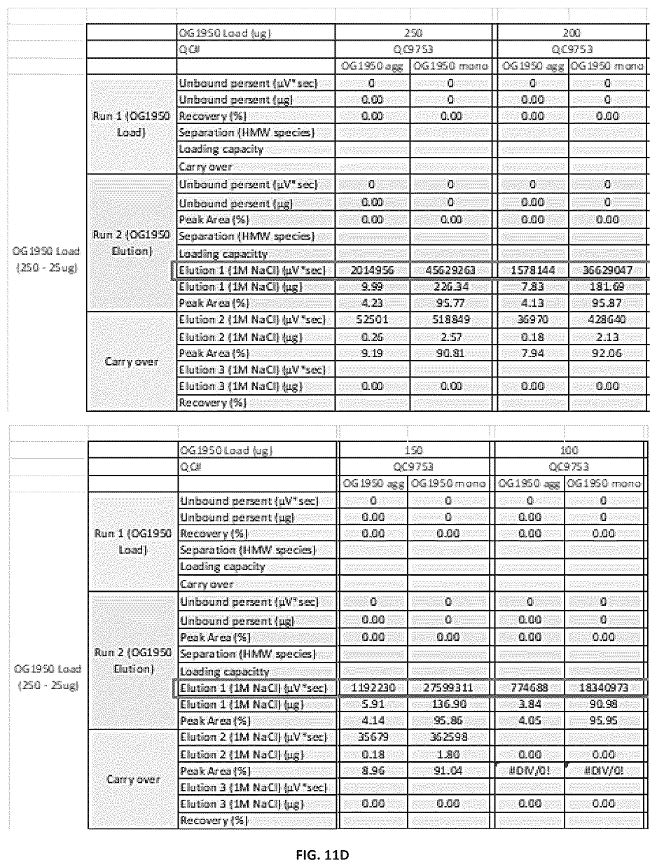

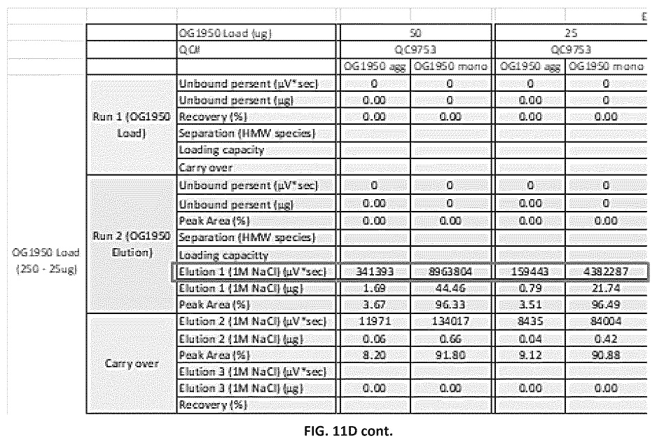

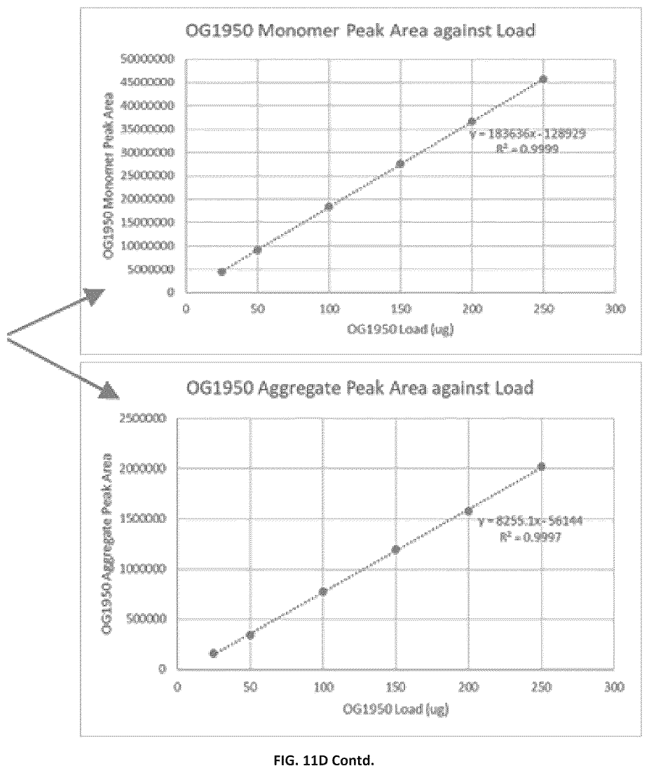

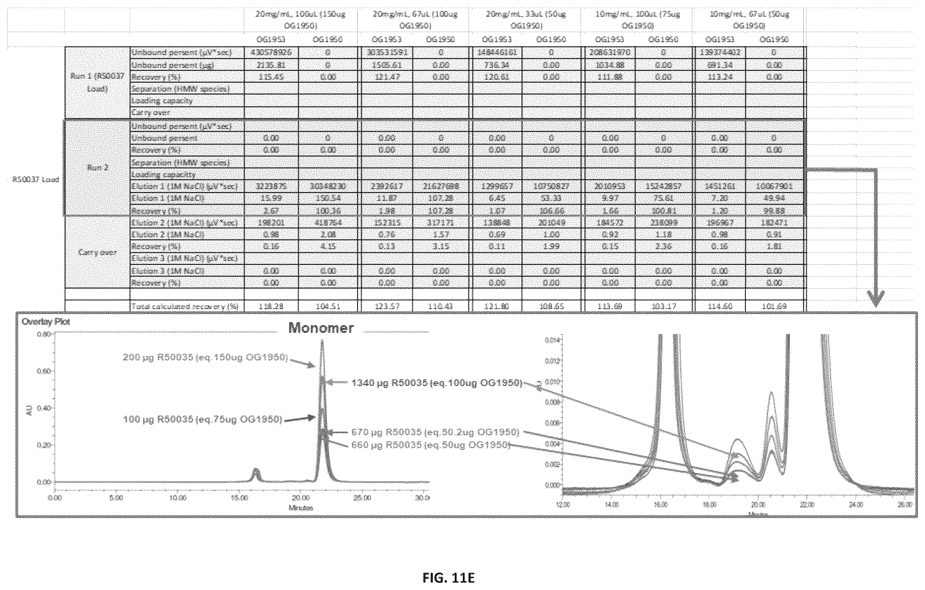

Next, sensitivity limit of detection (LOD), linearity, and recovery were evaluated for OG1950PUR and R50035. As shown in FIG. 11A, injection of 25-250 ug of OG1950PUR was tested in duplication and the result showed linearity with R2=0.999. As shown in FIG. 11B, at injection of ≥100 ug OG1950PUR, the recovery was ≥95%. Hence, 100 ug OG1950PUR or equivalence of OG1950 in the doped-in formulation (e.g. R50037) was selected for the current method. Injection of R50037 at various amounts showed dose dependent correlation of OG1950 peak recovery for monomer and various high MW peaks (HMW1 & 2) (FIG. 11C). Additional sample information including peak area and recovery data for run 1 run 2 and carry over samples for both the OG1950 load and R50037 load are summarized in FIGS. 11D and 11E respectively.

### Example 3—Experimental Composition Formulations for High Free OG1950 Up to 90% or with a OG468 Fab from 4.6-93%

The objectives of this experiment include:
(1) to test the potential range of the mix formulation beyond the currently tested 20% OG1950, such as 90%;
(2) to evaluate the mix formulation behavior of a 50 kDa Fab (OG468) and the range of mixing ratio from 4.6-93%;
(3) Applying the 2-dimensional (2D) chromatography method to demonstrate that the mix formulation forms a clear solution, it contains expected amount of free protein, and the free protein is a well-behaved protein with correct molecular weight and with migration behavior comparable to the pre-mixing form or standard.

TABLE 5 below represents the key parameters of the experiment. TABLE 6 is the reagent table and TABLE 7 is the experimental setup table.

TABLE 5

| | E2196_5688 | | | | |
|---|---|---|---|---|---|
| | #1 | #2 | #3 | #4 | #5 |
| Free protein | A | | B | | |
| [Conjugate, C] mg/mL* | 21.2 | 5.1 | 11.3 | 2.9 | 47.9 |
| [Free protein] mg/mL | 20.7 | 44.6 | 32.2 | 38.4 | 2.3 |
| [Total protein] mg/mL | 41.8** | 49.7 | 43.5 | 41.3 | 50.2 |
| [Free protein]/[Total | 49.4% | 89.9% | 74.1 | 93.0% | 4.6% |
| protein] (w/w) | | | | | |
| Molar ratio | 0.98 | 8.80 | 0.98 | 4.53 | 0.02 |
| Free protein/ | | | | | |
| conjugated form | | | | | |

*Only the protein moiety is included in the concentration. Polymer is not included in the concentration, which is an 850 kDa polymer.
**The final concentration is at 41.8 mg/ml after addition of 20 μl buffer to the original 100 μl total mixture.

TABLE 6
Reagent table:
Reagents

| | | | | | Total | Bioactive | |
|---|---|---|---|---|---|---|---|
| | | | Batch | Conc. | MW | MW | |
| ID | Name | Description | R# | (mg/mL) | (kDa) | (kDa) | Formulation |
| A | KSI- | KSI-301 Drug | R16346 | 50.8 | 950 | 146 | 12.5 mM Na- |
| | 301DP_{—} | Product | | | | | Phosphate pH 6.5, |
| | 105A | | | | | | 0.025% Tw20 |
| B | OG1950 | IgG | R16441 | 49.6 | 146 | 146 | 10 mM PBS pH 7.2 |
| | PUR | | | | | | |
| C | OG468 | Fab | R1136K | 40.8 | 50 | 50 | 10 mM sodium |
| | | | | | | | acetate pH 5.0 |
| D | OG1953_{—} | OG1953 + 15% | R50031 | 49.8 | NA | 146 | 50 mM sodium |
| | R50031 | OG1950 | | | | | acetate pH 5.0, |
| | | | | | | | 0.025% Tw20 |

TABLE 7
Reaction setup

| | | KSI- | | Free | | | Final | Final | Final | Total | | Molar |
|---|---|---|---|---|---|---|---|---|---|---|---|---|
| | | 301DP | Free | Protein | | Diluent | total | [KSI- | [Free | final | | ratio |
| Sample | KSI- | vol. | Protein | vol | Diluent | vol. | vol | 301] | protein] | conc. | % free | (free/ |
| ID | 301DP | (ul) | used | (ul) | used | (ul) | (ul) | mg/ml | mg/ml | (mg/ml) | protein | conj) |
| 1 | A | 50 | B | 50.0 | B143- | 20 | 120 | 21.2 | 20.7 | 41.8 | 49.4% | 0.98 |
| | | | | | 1* | | | | | | | |
| 2 | A | 10 | B | 90.1 | NA | 0.0 | 100 | 5.1 | 44.6 | 49.7 | 89.8% | 8.80 |
| 3 | A | 7 | C | 25.0 | NA | 0 | 32 | 11.1 | 31.8 | 43.0 | 74.1% | 0.02 |
| 4 | A | 1.5 | C | 25.0 | NA | 0 | 27 | 2.9 | 38.5 | 41.3 | 93.0% | 0.98 |
| 5 | A | 25 | C | 1.5 | NA | 0.0 | 27 | 47.9 | 2.3 | 50.2 | 4.6% | 4.53 |

*B143-1 contains 50 mM Sodium Acetate pH 5, 0.025% PS20

To set up the experiment, both the KSI-301DP and OG1950PUR protein were thawed, pH of the OG1950PUR protein was adjusted with 0.5M phosphoric acid to pH 5, then desired was added as shown in TABLE 7 to a clear PCR tube. To this vial, desired volume of KSI-301DP was added slowly and immediately mixed well. Mixture was observed for cloudiness, if present, B143-1 was added as dilution buffer as shown in sample #1. The mixture was incubated at ambient temperature for at least one day before sampling for 2D chromatography analysis.

Sampling is performed according to the TABLE 8 below:

TABLE 8
Sample Preparation for Analysis

| | Final | | Dilution | | |
|---|---|---|---|---|---|
| | working | Stock | buffer | Injected | ug free |
| vol for | conc. | needed | needed | vol | protein |
| inject (ul) | (mg/ml) | (ul) | (ul) | (ul) | inject |
| 100 | 10 | 48.4 | 51.6 | 10 | 100 |
| 100 | 10 | 22.4 | 77.6 | 10 | 100 |
| 30 | 10 | 9.4 | 20.6 | 15 | 150 |
| 30 | 10 | 7.8 | 22.2 | 15 | 150 |
| 30 | 2 | 26.0 | 4.0 | 65 | 130 |

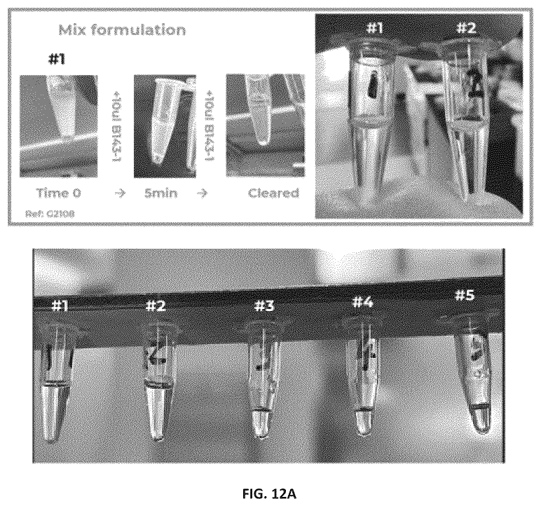

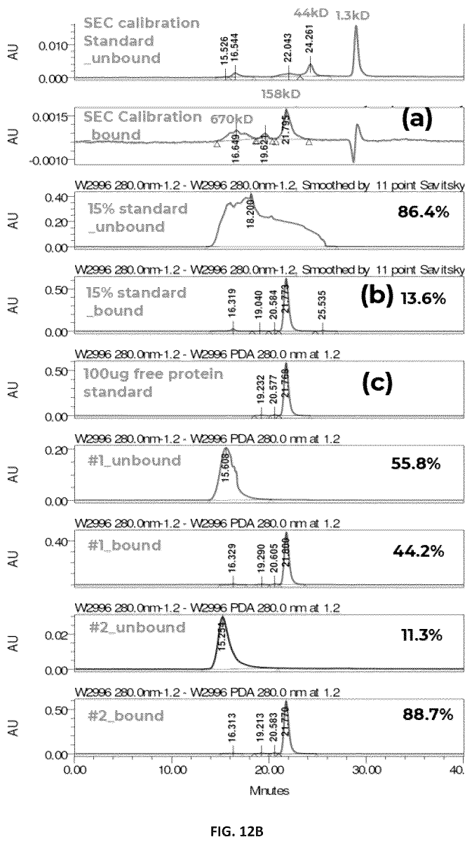

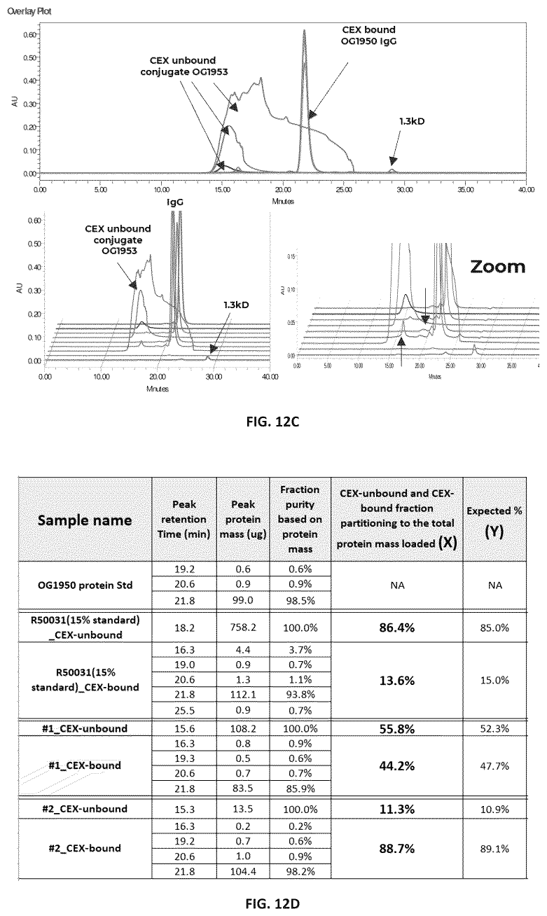

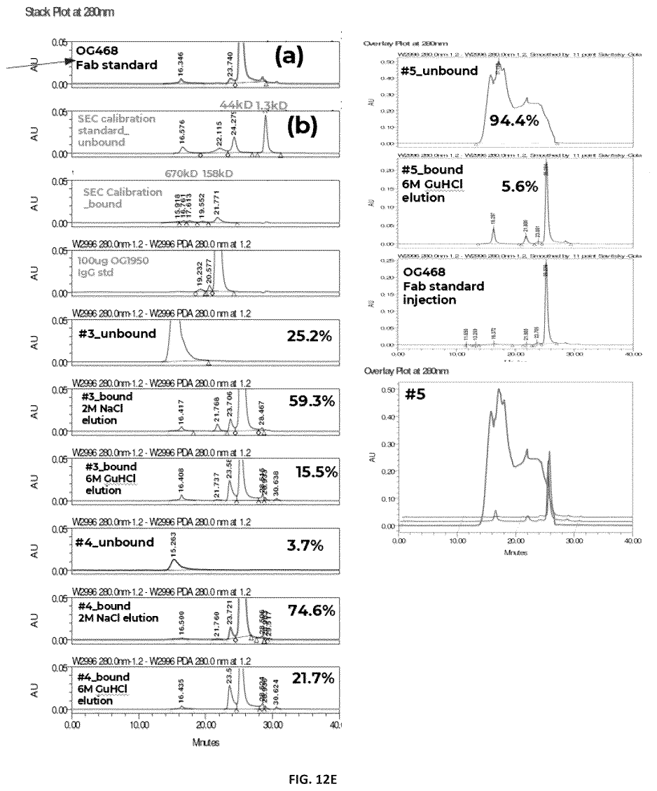

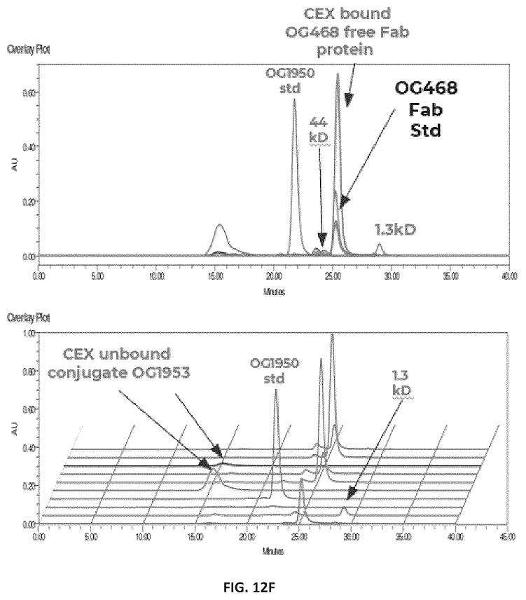

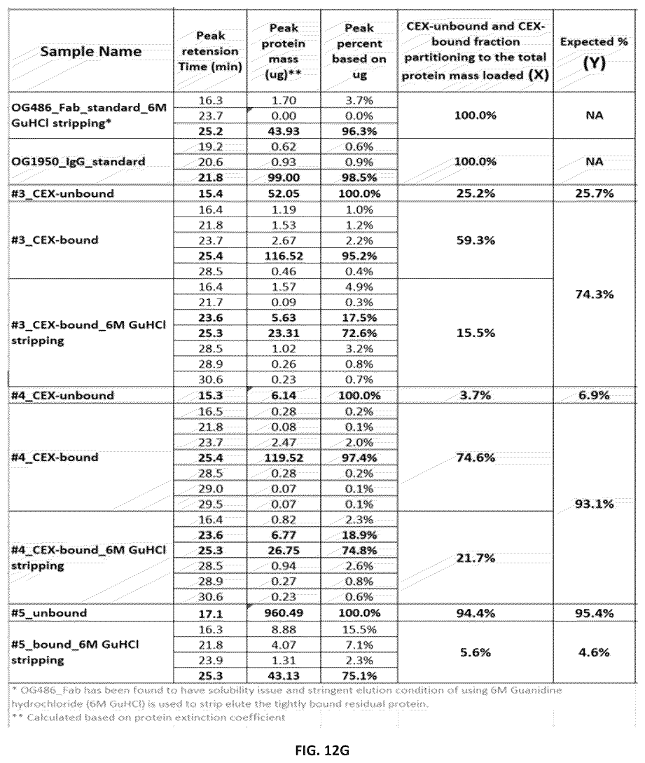

The specified volume for 2D chromatography analysis using a Waters HPLC system equipped with a 2695 Alliance Solvent management system and a 2785 Dual Wavelength detector was injected. The chromatogram was analyzed as shown in FIG. 12.B-G for detail result summary.

Findings: Mixture #1 (FIG. 12) was first cloudy but became cleared upon addition of 20 μL buffer. Mild precipitation was observed upon microfuge centrifugation at 5K rpm for 5 minutes. All other formulations (TABLE 6), #2-#5, showed no cloudiness at all.

FIG. 12B shows the sample analysis of the #1 & 2 samples using 2D method analysis. It shows elution peak(s) of free protein at the expected retention time of 21.8 min, whereas the conjugate was eluted as a broad peak at ~15.6 min. This was confirmed by (a) SEC calibration standard; (b) 15% mix formulation standard (R50031); and (c) injection OG1950 free protein standard injection. The percentage on the right of each chromatogram represents the partition of CEX-unbound (conjugate) to CEX-bound fraction (free IgG), they were in agreement with the expected percent free protein as shown in FIG. 12D. A close-up overlay of all curves in FIG. 12C show overlap of eluted IgG peaks which were aligned with both the standard IgG and IgG standard from the SEC calibration standard. (Bottom left) Offset overlay of various injections for clear illustration of separation between unbound CEX conjugate fraction and the CEX bound IgG fraction. (Bottom right) is a close-up view to show the aggregated form of IgG that is clearly detected as shown in the arrows. FIG. 12D is the peak analysis table showing agreement and recovery of the mixed in free protein in the expected ratio as compared to the conjugate unbound fraction. Similar result can also be concluded for samples #3-#4 for the OG468 Fab (see FIG. 12E-G).

### Example 4—Evaluation of Tandem Method Using Switch Valve for Multi-Column Combination Selection

A tandem series approach of orthogonally combining the existing CEX and SEC analytical method for a representative OG1953 drug product formulation is described in the preceding examples and designated SOP391_v1. SOP391_v1 was found to allow integrated analysis of the OG1953 conjugate and OG1950 free antibody population for the representative OG1953 drug product formulation, which was otherwise impossible by using CEX or SEC separately.

This experiment was performed to improve SOP391_v1 resolution, flexibility and performance. The experiment incorporates a 3-way switch valve enabling direction of the CEX effluent through 1) a bypass loop, 2) a Shodex 806M_HQ SEC column, or 3) a TSK G3000 SEC column (FIGS. 13A-13C). Separate acquisition methods were written for modes 1-3, enabling each method to be run during the sample injection queue which can direct the effluent to different downstream options. Flow path volumes for modes 2 and 3 were about 20-22 mL between the pump outlet and the UV detector flow cell.

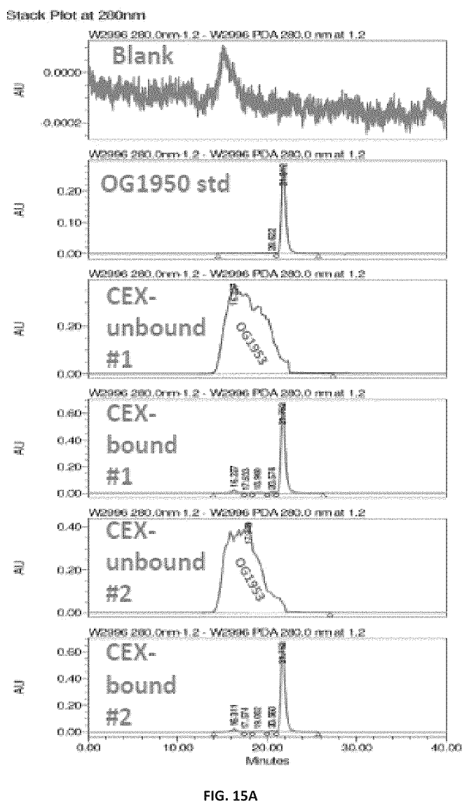

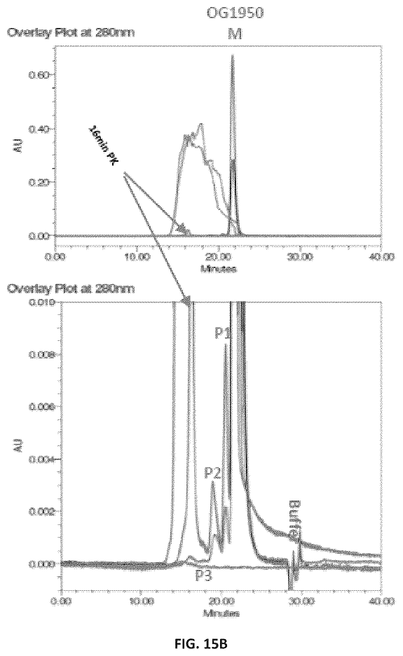

The test of multiple column combinations SOP391_v1-v6 was performed with a Waters 2695 Alliance system with 2487 Dual Wavelength detector and column heater with 3-way switch valve where all connection tubing was approximately 0.6 mm in diameter. The chromatographic set up included Buffer A (running buffer) comprised 20 mM sodium acetate pH5, 100 mM NaCl, an isocratic mode at 0.5 mL/minute, and a column prefilter. The column heater was set to 25° C. A dual wavelength UV detector was used for detection at 214 nm and 280 nm. A summary of the sample information including SEC standards, OG1950 antibody, and representative OG1953 drug product formulations with one including 20% OG1950+80% OG1953, and another with 7.5% OG1950+92.5% OG1953 as shown in FIG. 14A. The column flow path matrix for the samples is shown in FIG. 14B where each unique column combination is designated with a different SOP391 version (v1-v6). The data for SOP391_v1 was obtained using a different Waters HPLC system than the rest of the data.

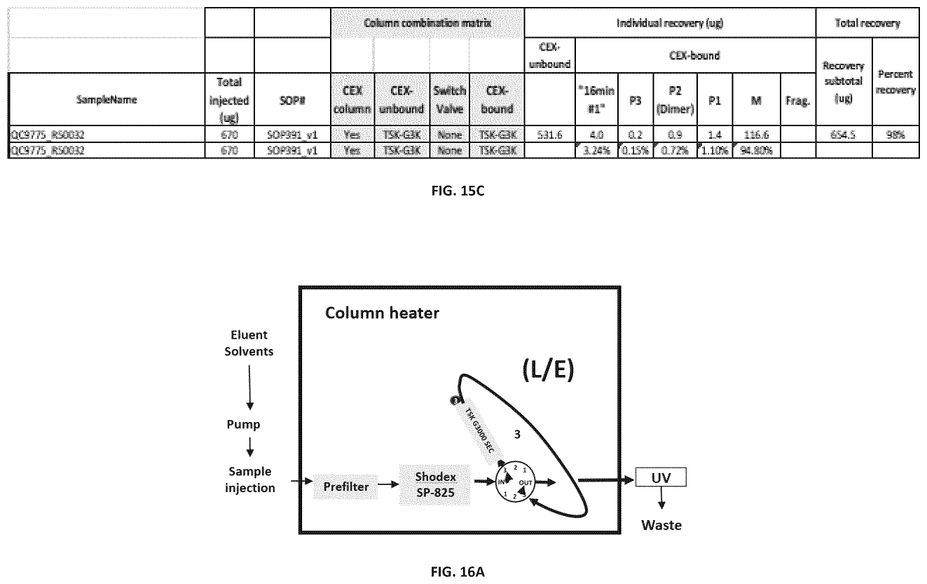

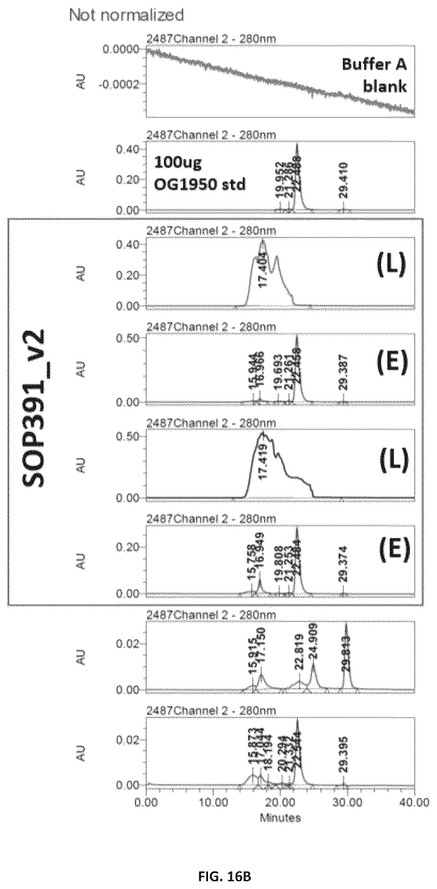

First the R50032 formulation (20% OG1950+80% OG1953) was analyzed via the SOP391_v1 featuring the tandem CEX+SEC HPLC method without the 3-way switch valve. Peaks for the CEX-bound, CEX-unbound, standard and blank are shown in FIG. 15A. FIG. 15B shows an overlay plot of the peaks shown in FIG. 15A. FIG. 15C shows mass recovery from the CEX-unbound fraction and mass recovery from the peaks of the CEX-bound fraction, as well as total recovery, a mass recovery of 98% was observed. The OG1953 conjugate was primarily in the CEX-unbound fraction, while the OG1950 free antibody and its aggregate and degraded forms were primarily in the CEX-bound fraction. The SEC profile of the CEX-bound fraction included a 16.3 minute peak accounting for 3.24% of the of the total CEX-bound pool. Additionally, the monomeric form of OG1950 is at 94.3% with 3 aggregate peaks observed as P1-P3.

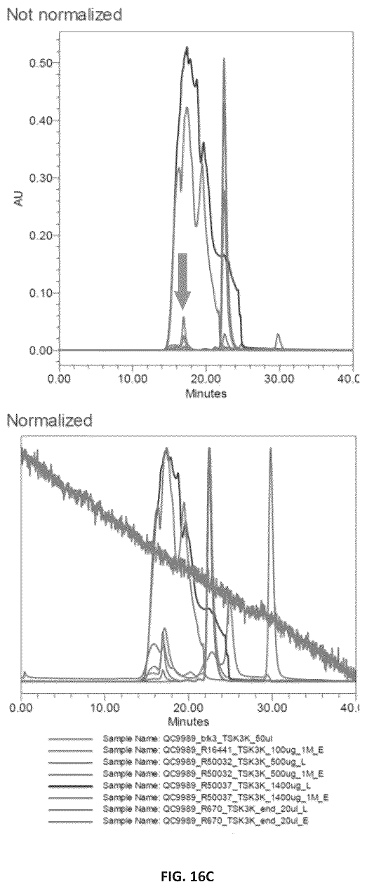

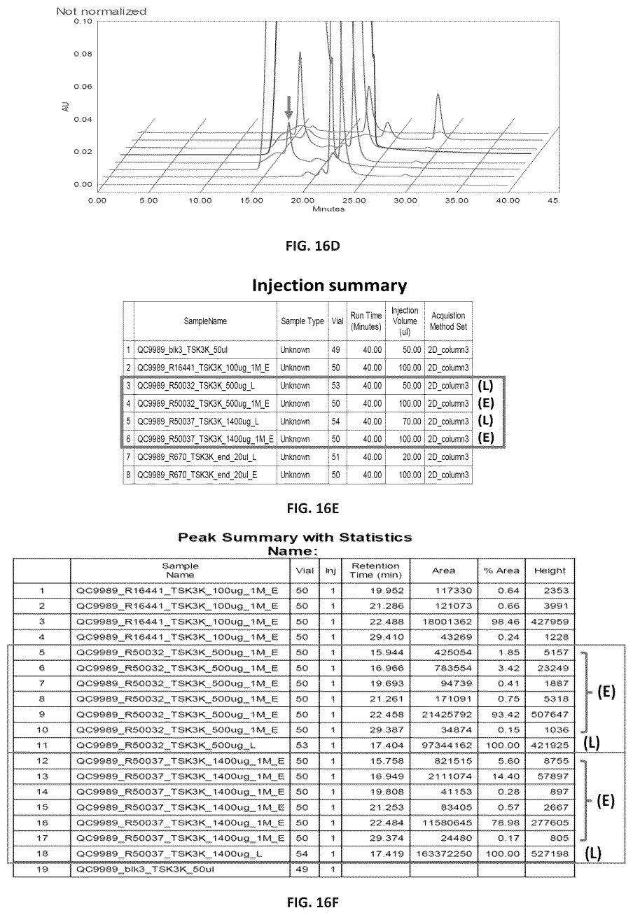

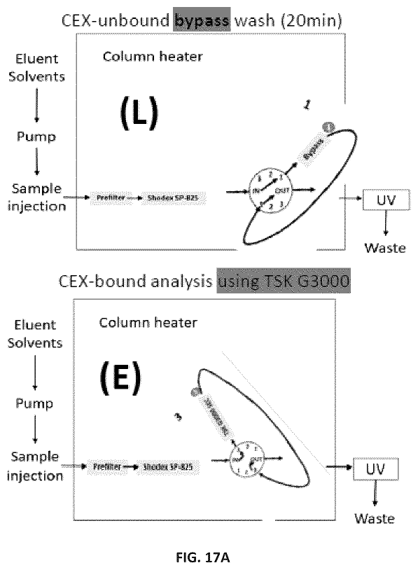

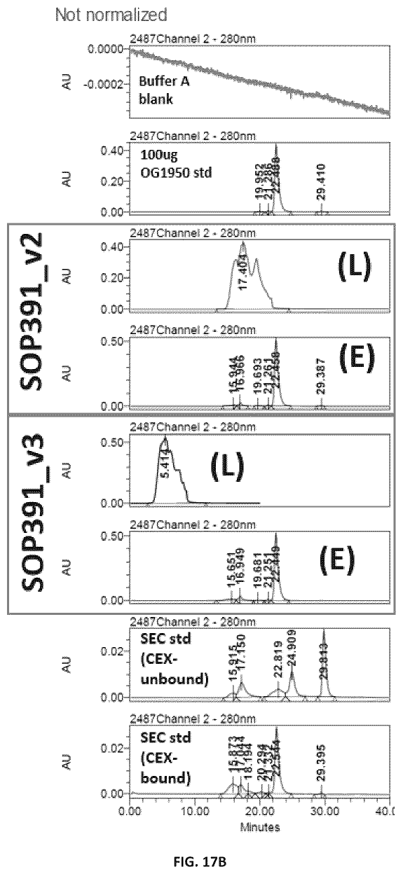

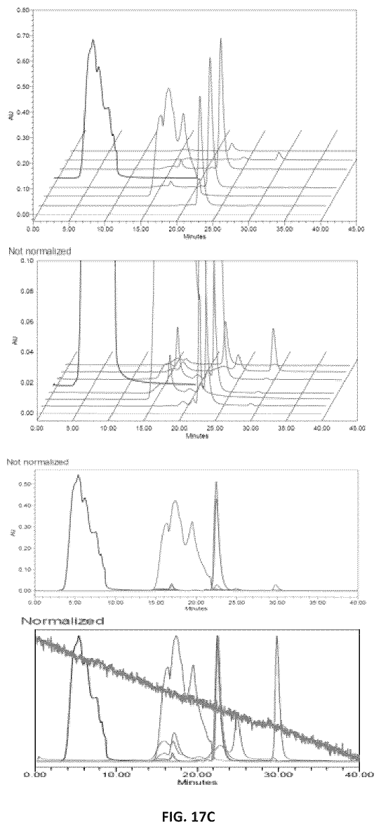

Next, it was of interest to determine if the results of the SOP391_v1 method could be reproduced in the valve switch setup designated SOP391_v2 where the R50032 and R50037 formulations were analyzed. The flow path through the 3-way switch valve in SOP391 v2 where the CEX-bound (E) and CEX-unbound (L) fractions pass through the TSK G3000 SEC column is shown in FIG. 16A. The peaks observed in the E and L fractions for both formulations and standard and blank are shown in FIG. 16B, and FIGS. 16C-16D depict overlay graphs of the individual peaks shown in FIG. 16B. The red arrows in FIG. 16C-16D indicate a peak at 16.966 minutes which accounts for 5.2% of the total CEX-bound fraction and corresponded to the 16 minute peak found in SOP391_v1. A summary of the injection queue including run time (minutes), injection volume (μl), and acquisition method for the E and L fractions of the R50032 and R50037 formulations is shown in FIG. 16E. A summary of the peak data including retention time (minutes) area, % area, height for the E and L fractions of the R50032 and R50037 formulations is shown in FIG. 16F. General performance was found to be maintained compared to SOP391_v1.

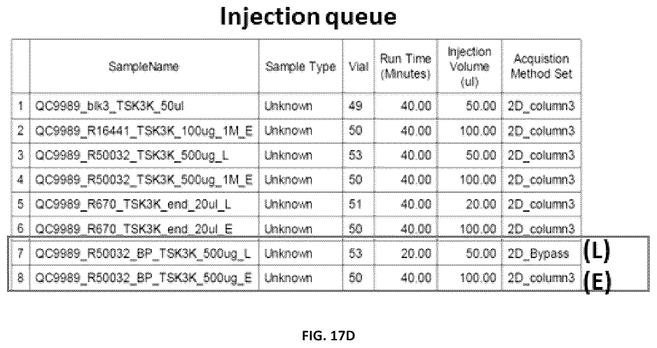

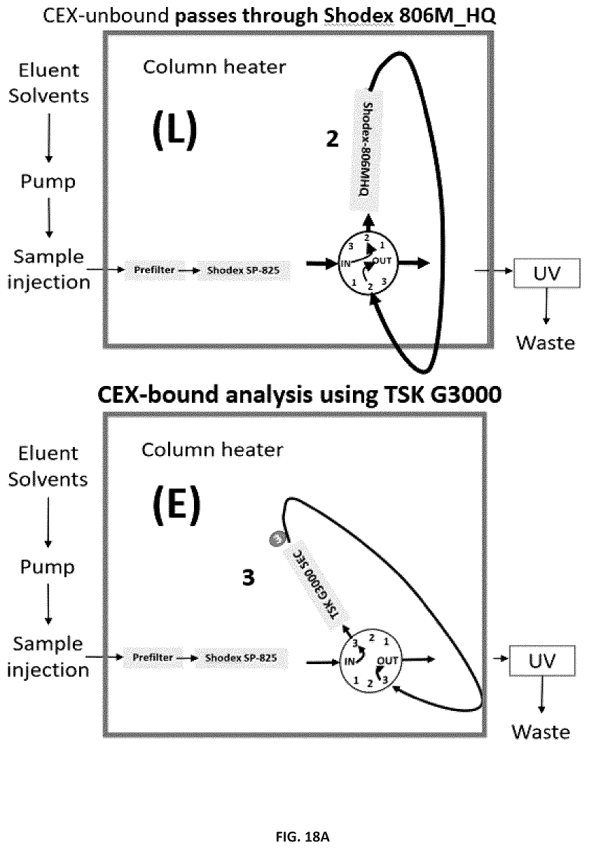

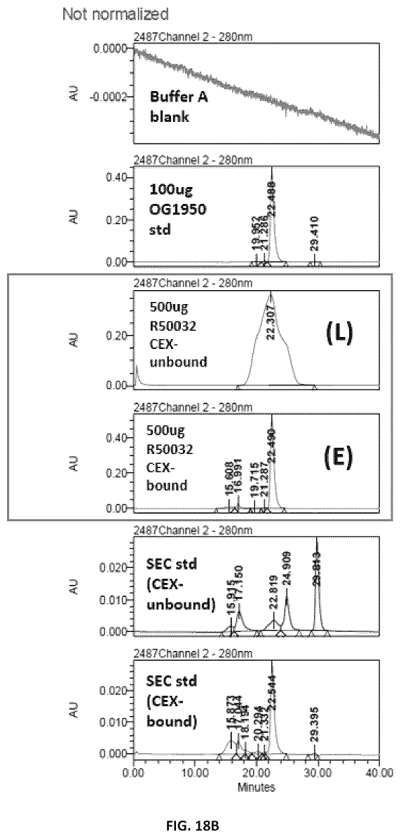

Next, the R50032 formulations were evaluated via method SOP391_v3 where the CEX-unbound fraction (L) was directed via bypass instead of flow through the TSK G3000 SEC column for analysis as performed for the CEX-bound fraction (E) and shown in FIG. 17A. The peaks observed in the E and L fractions are shown in FIG. 17B, which also compares the peaks of the E and L fractions between the SOP391_v2 and SOP391_v3 operation modes. Overlay graphs of the individual chromatogram shown in FIG. 17B are shown in FIG. 17C. A summary of the injection queue including run time (minutes), injection volume (μl), and acquisition method for the E and L fractions of the R50032 formulations is shown in FIG. 17D. Directing the CEX-unbound fraction through the bypass-loop resulted in a similar 16 minute profile compared to the SOP391_v2 mode, however, the peak area was doubled. The OG1950 free antibody peaks showed comparable results to SOP391 v2.

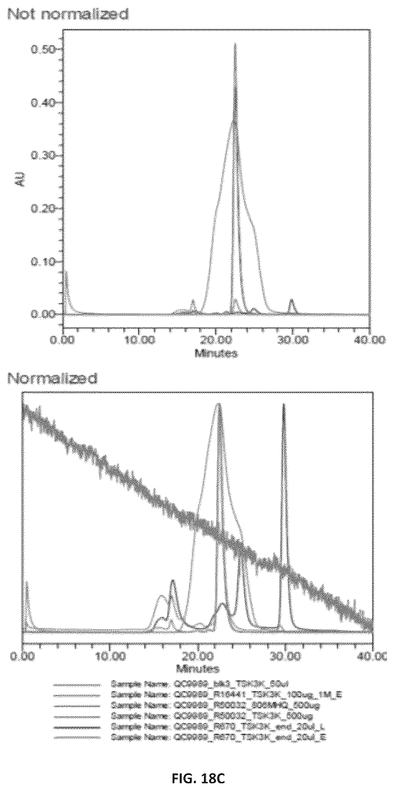

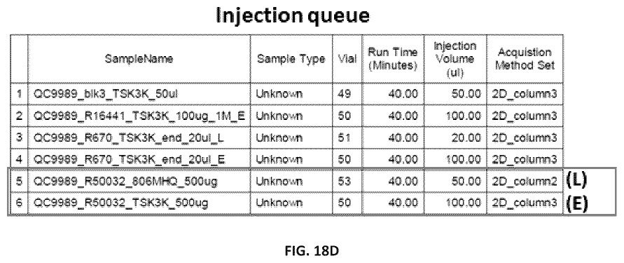

In the following experiment, the R50032 formulations were evaluated with the SOP391_v4 operation mode using the 3-way switch valve where the CEX-unbound fraction (L) passes through a Shodex 806M_HQ column and the CEX-bound (E) fraction was analyzed using a TSK G3000 column as shown in FIG. 18A. The peaks observed in the E and L fractions are shown in FIG. 18B. Overlay graphs of the individual chromatogram shown in FIG. 18B are shown in FIG. 18C. A summary of the injection queue including run time (minutes), injection volume (μl), and acquisition method for the E and L fractions of the R50032 formulations is shown in FIG. 18D. The chromatographic profile of the Shodex 806M_HQ column displayed a high degree of distortion, possibly from an overloading issue.

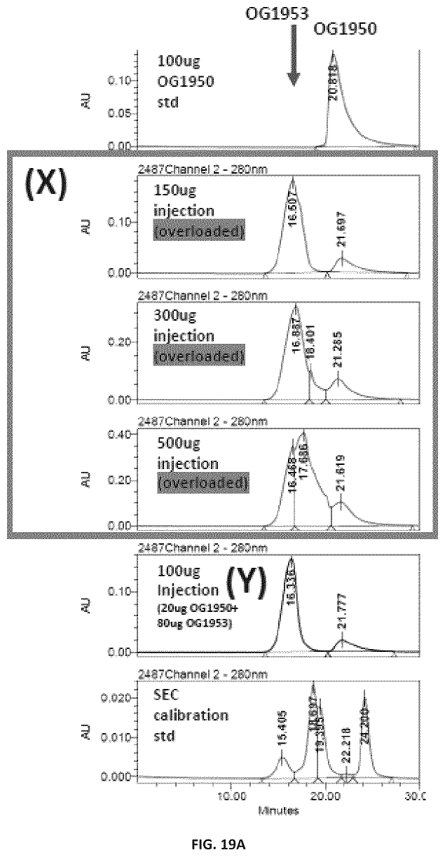

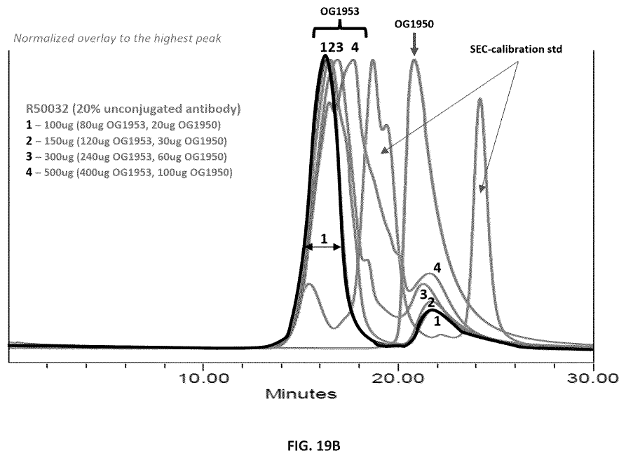

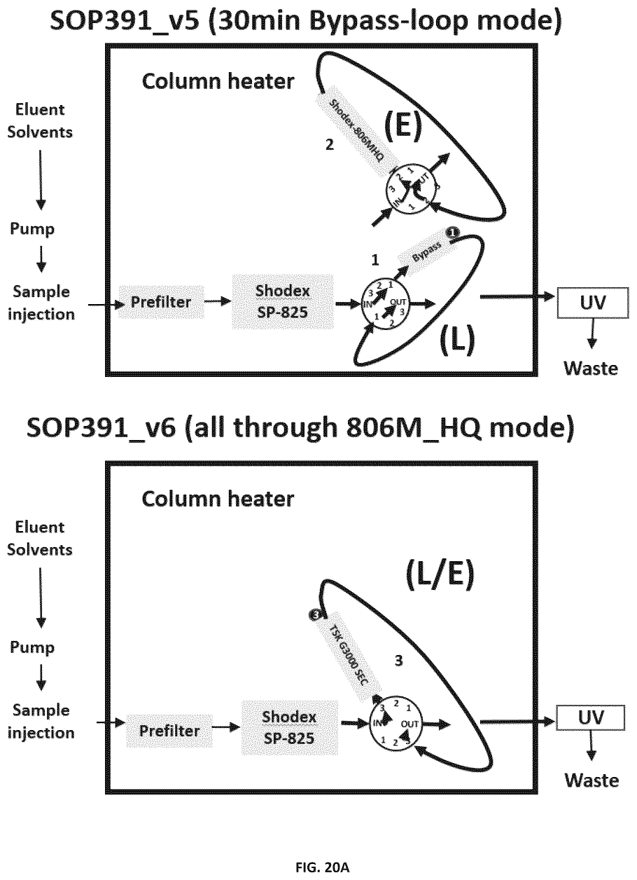

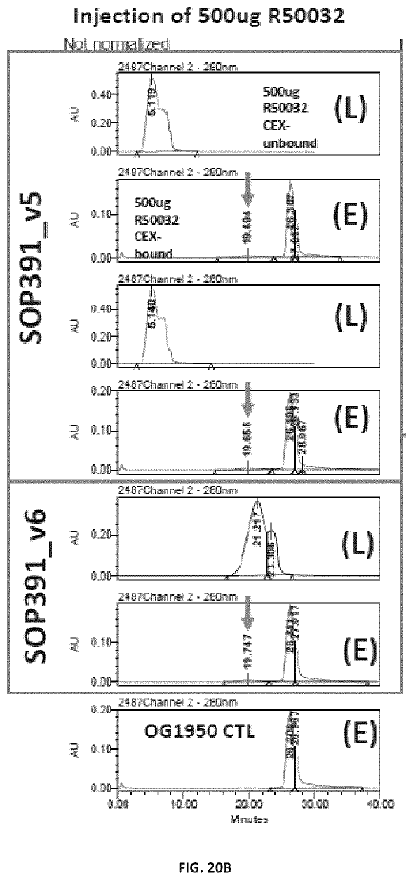

Further evaluation of the overloading issue seen in R50032 using the SOP391_v4 operation mode was performed in order to determine the appropriate amount of sample to inject without overloading the column. As shown in (X) of FIG. 19A, the Shodex 806M_HQ SEC column was overloaded when 100 μg of OG1950 and an additional 400 μg of OG1953 were injected, resulting in distortion and skewing of the elution peak. In (Y), a loading limit of 100 μg per injection allowed the OG1953 peak to elute as a single peak at 16.336 minutes and allows for baseline separation of OG1953 and OG1950. An overlay graph of the individual chromatogram shown in FIG. 19A is shown in FIG. 19B. It was determined that in order to analyze OG1953 in the mixture two separate injections were required: 1) Injection of 100 μg of the representative OG1953 drug product formulation for CEX-unbound analysis where the Shodex 806M HQ SEC column is used for analysis; and 2) injection of 500 μg for the CEX-bound fraction where the TSK G3000 column was used for OG1950 analysis.

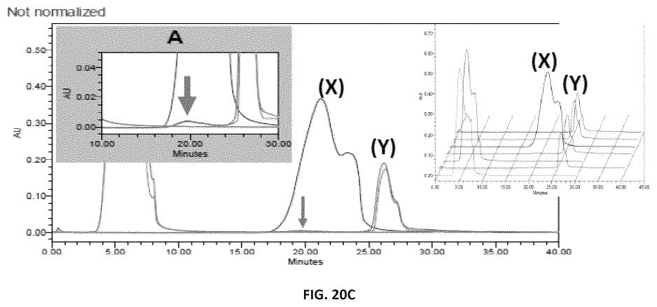

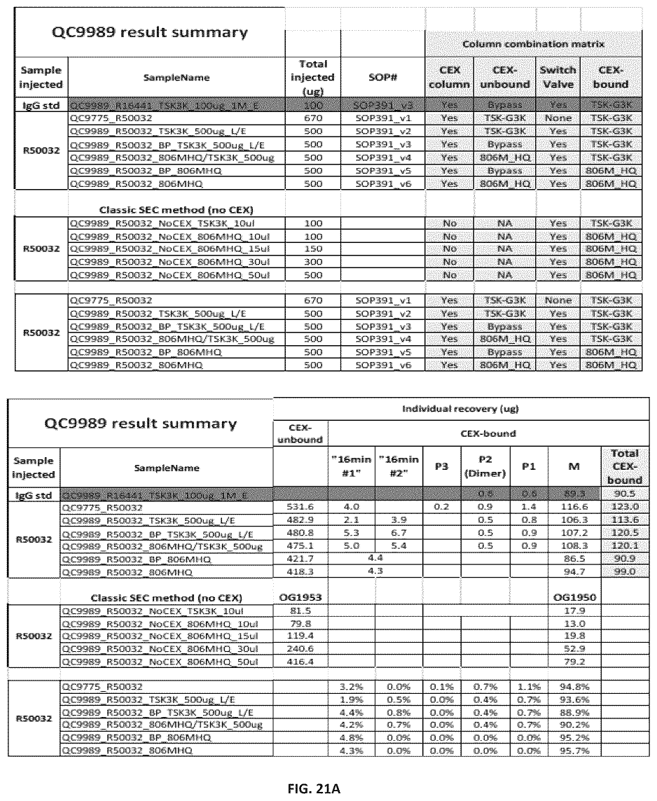

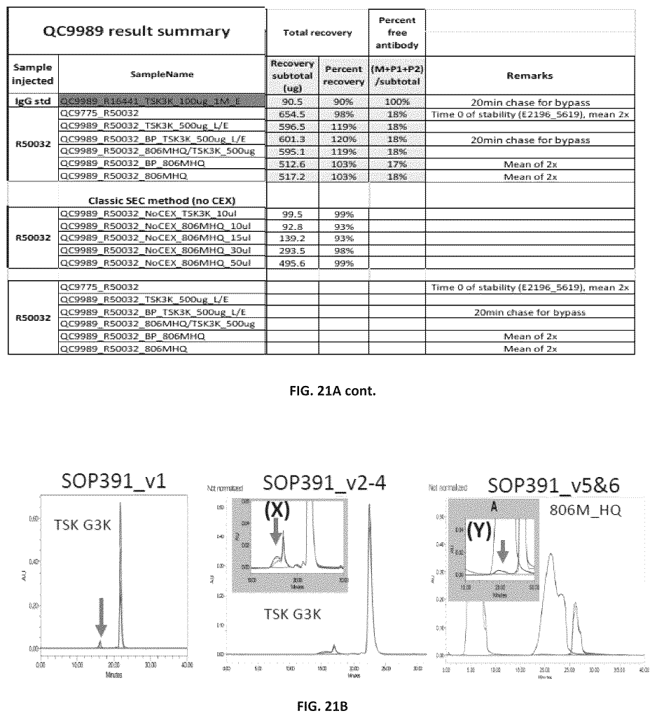

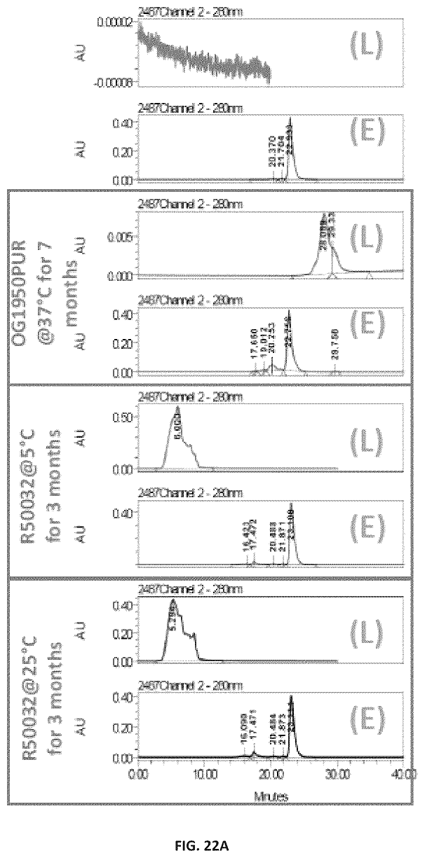

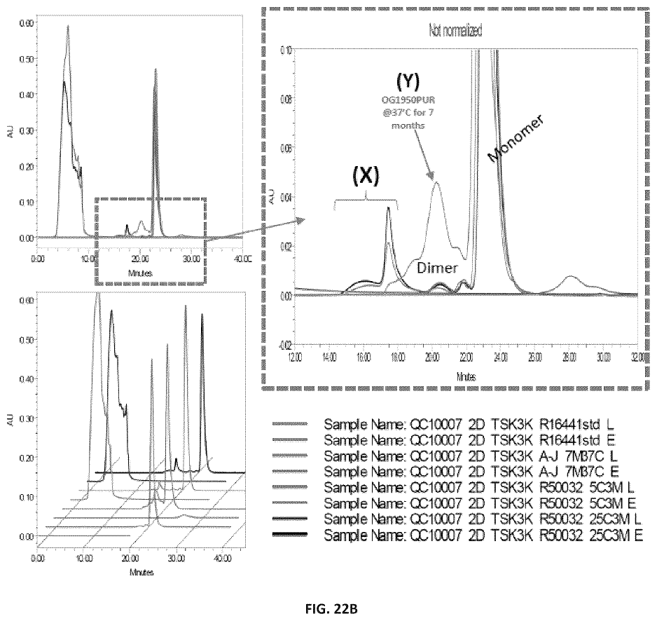

SOP391_v5 and v6 were used for indirect evaluation of the size and heterogeneity of the 16 minute peak. The flow path through the 3-way switch valve in SOP391_v5 where the CEX-unbound fraction (L) passes through the bypass loop and the CEX-bound (E) fraction is analyzed by a Shodex 806M_HQ column is shown in FIG. 20A. FIG. 20A also shows the flow path through the 3-way switch valve in SOP391_v6 where both the CEX-unbound fraction (L) and the CEX-bound (E) fraction are analyzed using a Shodex 806M_HQ column. Peaks observed in the E and L fractions for both the SOP391_v5 and SOP391_v6 operation modes are shown in FIG. 20B. Overlay graphs of the individual chromatogram shown in FIG. 20B are shown in FIG. 20C. The red arrows seen in FIG. 20B-20C indicate an equivalent to the fraction of SOP391_v1 seen at the 16 minute peak here eluted at approximately 20 minutes. (X) of FIG. 20C indicates the biphasic elution profile of the OG1953 conjugate. (Y) of FIG. 20C indicates a biphasic profile for OG1950 free protein control, a phenomenon that has also been observed in CEX bound fractions of the representative OG1953 drug product formulation.

The individual peak and total recovery data between the SOP391_v1-v6 operational modes for the R50032 formulation are summarized in FIG. 21A. A comparison between the 16 minute peak fraction observed in SOP391_v1 compared to; X: SOP391_v2-v4 where the 16 minute peak was further separated into two populations by the TSK G3000 SEC column; and Y: SOP391_v5-v6 where the 16 min peak coeluted with the bulk of OG1953 conjugate is shown in FIG. 21B. The 16 minute peak first seen in SOP391_v1 was found to elute at different retention times depending on the column, flow paths, and other chromatographic running conditions. SOP391_v1-v6 were found to have good overall recovery with comparable percent free antibody (OG1950) at 18%.

### Example 5—Evaluation of Sample Stability Using SOP391 v1

Finally, the tandem method of SOP391_v3 was used to evaluate stability of OG1950PUR and R50032 samples. Peaks observed in the CEX-unbound (L) and CEX-bound (E) fractions of an OG1950PUR sample stored at 37° C. for 7 months, an R50032 formulation stored at 5° C. for 3 months, and an R50032 formulation stored at 25° C. for 3 months are shown in FIG. 22A. Overlay graphs of the individual chromatogram of FIG. 22A, where X indicates the peaks for the R50032 formulations stored at 5° C. and 25° C. for 3 months, and Y indicates the OG1950PUR sample stored at 37° C. for 7 months are shown in FIG. 22B. Incubation of R50032 samples at 5° C. and 25° C. for 3 months resulted in 6% and 8% total 16-17 minute peaks respectively, as seen in (X) of FIG. 22B. Incubation of OG1950PUR at 37° C. for 7 months resulted in total aggregation of 14%, as seen in (Y) of FIG. 22B.

### Example 6—Overview of the Principle and Operation of the Tandem CEX-SEC Method

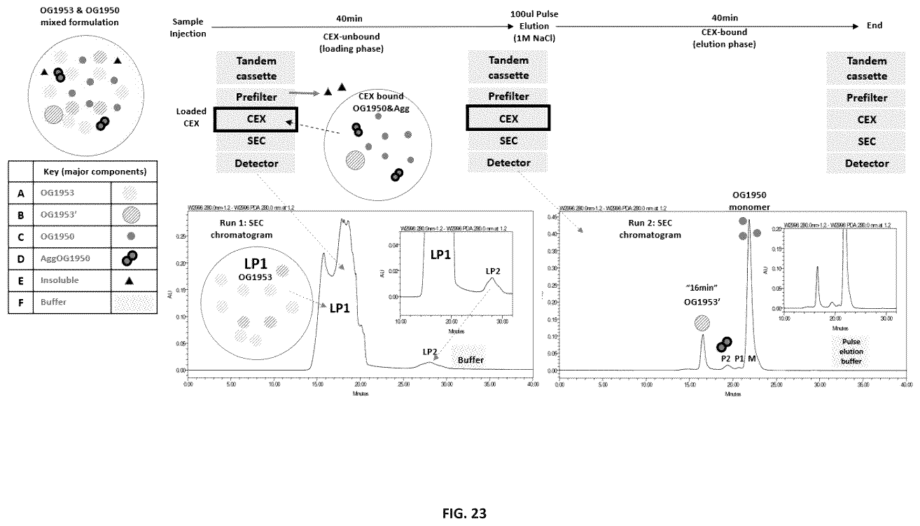

As seen in FIG. 23, the representative OG1953 drug product formulation has been found to comprise OG1953, OG1953′, OG1950, aggregated OG1950, and insoluble components within a buffer. The prefilter step of the tandem CEX-SEC method removes the insoluble components. In some embodiments, OG1953′ can include two degradation products and/or aggregates (LP2 and/or 16 min peak) that can be derived from the main OG1953 conjugate peak. In some embodiments, these two derivatives of OG1953 can also be found in a representative OG1953 drug product formulations and can demonstrate similar stability behavior to the OG1953 in the current mix formulation. The OG1950 and its aggregates bind the CEX column and the OG1953 is not bound to the column and can be analyzed by SEC in the first instance. A pulse of 1M NaCl can elute the OG1950 and aggregates from the CEX column and the OG1950, aggregated OG1950, and any OG1953 can then be distinguished by SEC in the second instance.

### Example 7—Stability and Formulation Evaluation of OG1953 Biconjugate Containing Various Amounts of Free OG1950 Protein (0-6 Months Sampling Result)

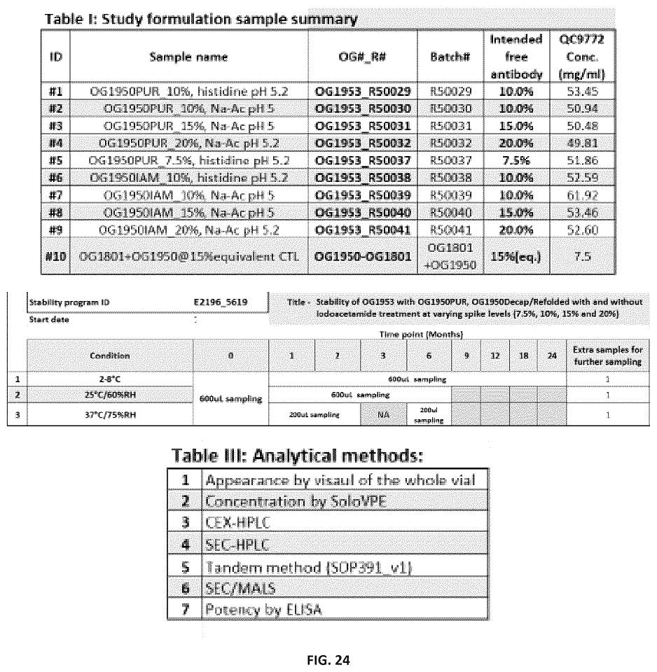

Next, a stability study was conducted to evaluate the stability of the OG1953 bioconjugate containing 7.5, 10, 15, and 20% free OG1950 protein at different temperatures over time as depicted in FIG. 24. Factors to be evaluated included: 1) visual turbidity; 2) aggregation or degradation (collectively referred to herein as impurities) at different temperatures, formulation and excipient using tandem (CEX and SEC) method, SEC-HPLC, and SEC/MALS; 3) change of percent free antibody (OG1950); and 4) potency. The study also compared and contrasted the impact of iodoacetamide (IAM) treatment (alkylation) on protein stability (samples #6-9) as compared to the original unmodified samples (samples #1-5). Additionally, the study evaluated the impact of OG1801 polymer on free antibody (OG1950) at 15% equivalent concentration to the 50 mg/ml total protein concentration (i.e. 7.5 mg/ml OG1950 with 227 mg/ml OG1801 polymer which is not accounted for in the total reported mass of the formulation) (sample #10). The study also evaluated the tandem analytical HPLC method performance in terms of recovery, robustness, specificity, reproducibility, and accuracy.

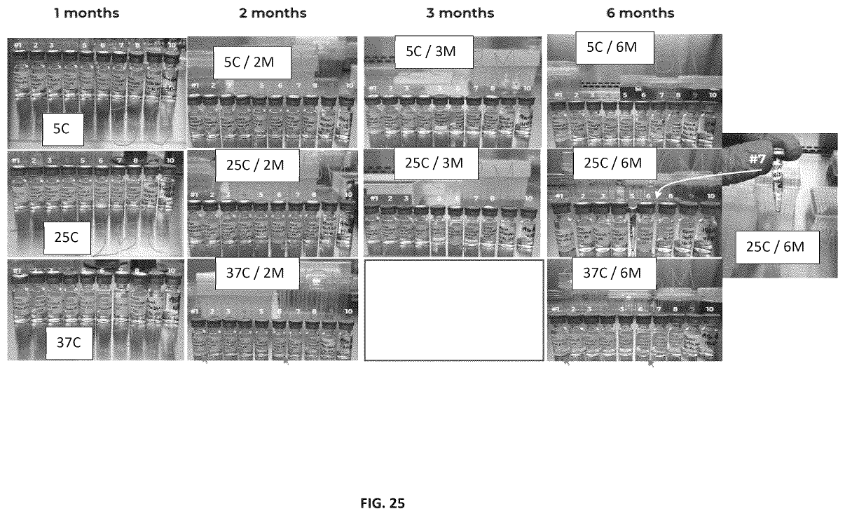

First, visual appearance of the samples was assessed after storage for 1, 2, 3, and 6 months at 5, 25, and 37° C. As shown in FIG. 25, most samples remained clear at 6 months for all temperatures except for the vials indicated by arrows. All cloudy samples were formulated in Histidine buffer with 10% free protein. However, the 7.5% sample #5 which was also formulated in Histidine buffer remained clear at all conditions.

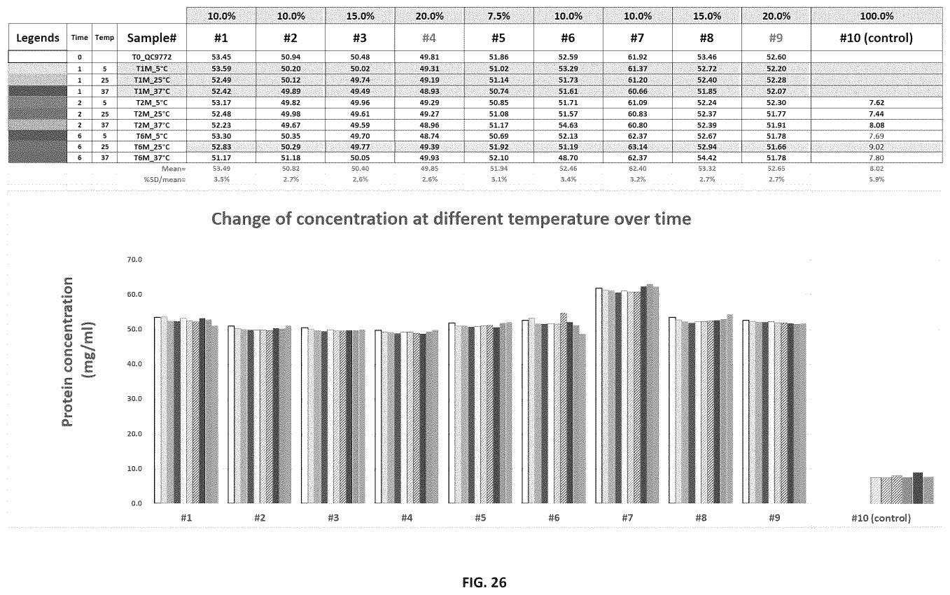

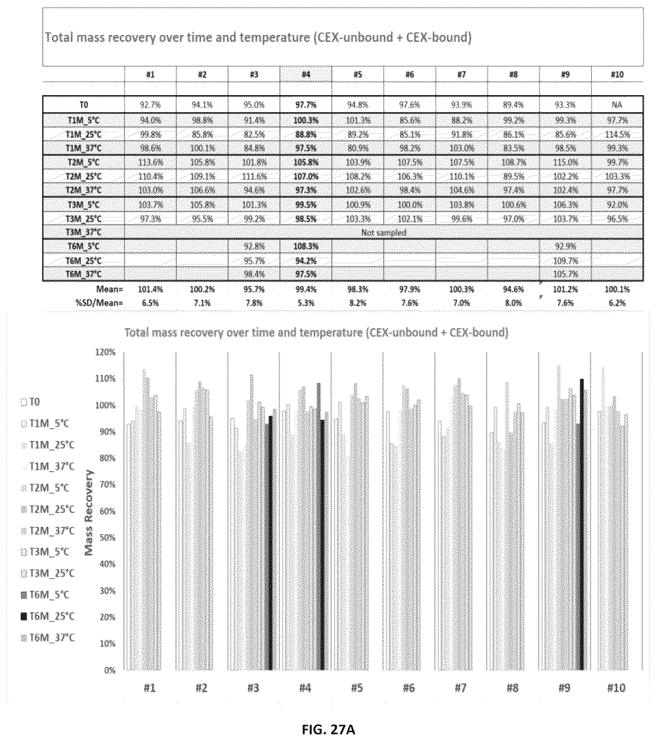

Protein concentration was determined for all samples stored at 5, 25, and 37° C. for 1, 2, and 6 months as seen in FIG. 26. An analysis of changes in protein concentration over time from 1 to 6 months was also performed, as seen in FIG. 26. All samples show no change in concentrations at all conditions.

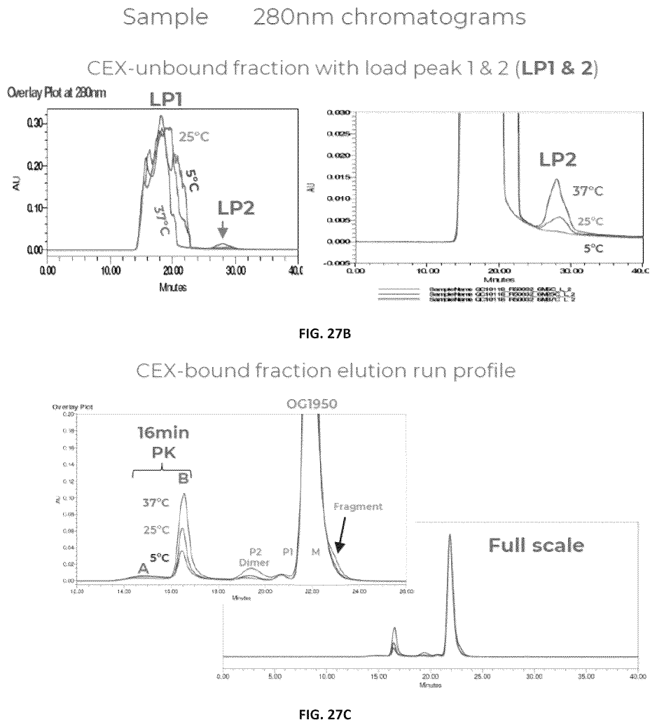

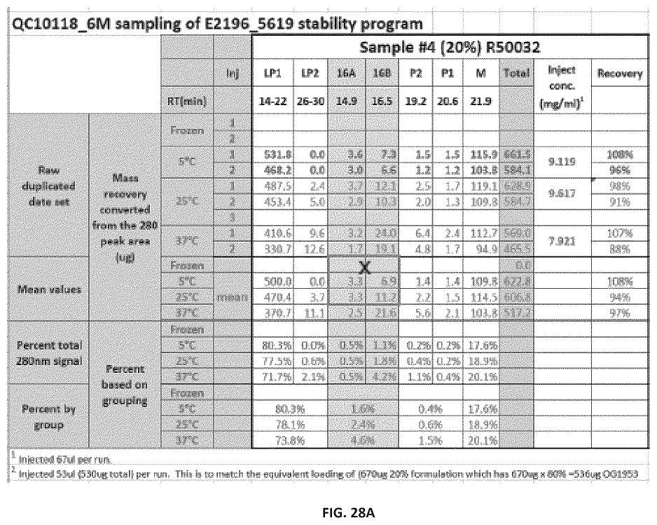

Sample recovery and elution profiles using Tandem method were determined for samples #1-10 stored at 5° C., 25° C., and 37° C. for 1, 2, 3, and 6 months, as seen in FIGS. 27A-C. Close to 100% mass recovery was seen in for all samples, especially for sample #4, as seen in FIG. 27A. As shown in FIG. 27B, the OG1953 conjugate peak (LP1) was found to decrease with the instability population as shown at about 26-30 minutes and designated LP2. As shown in FIG. 27C, the CEX-bound profile includes: 1) a 16 minute peak split into A and B; 2) P2, P1, and M are OG1950 free protein related peaks which represent dimer, P1 and monomer respectively; and 3) a fragment peak in the stress condition. All peaks described above displayed temperature dependent changes over time.

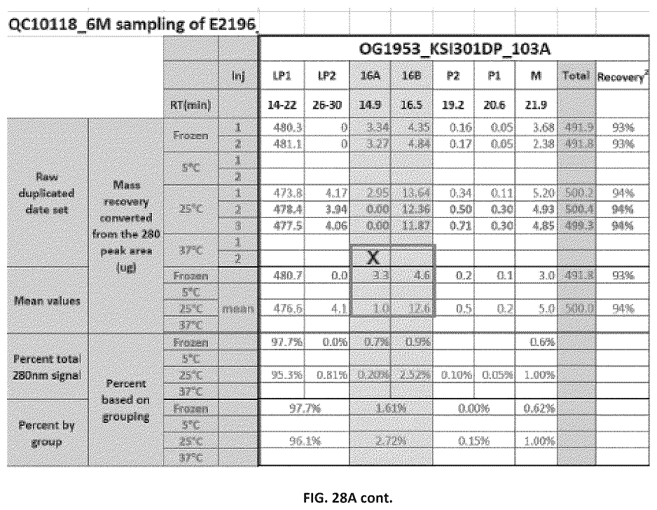

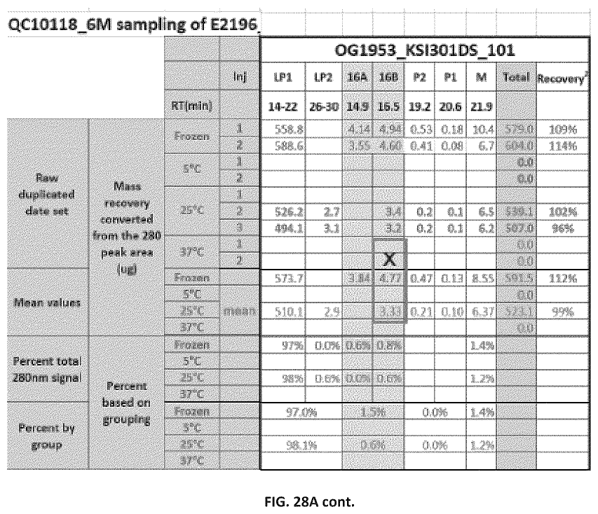

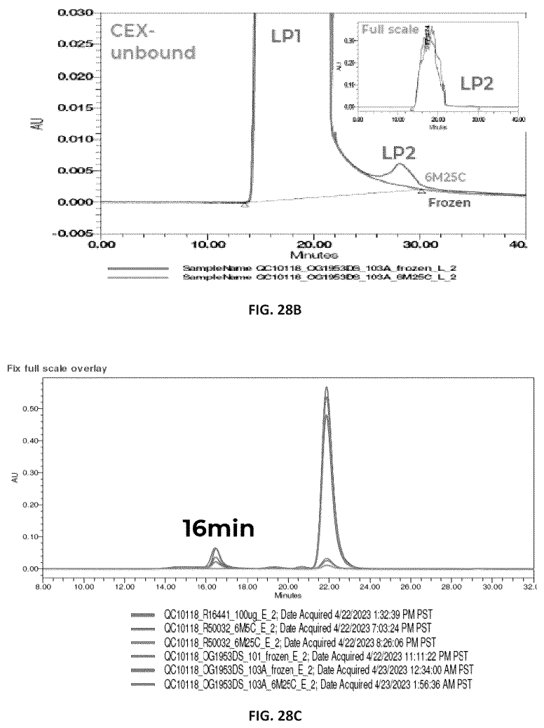

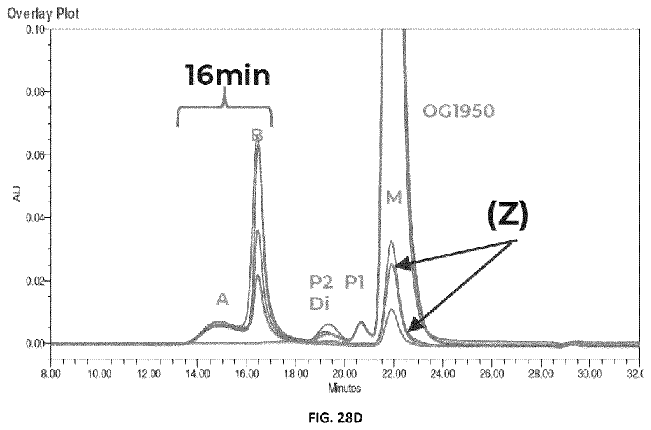

LP2, the 16 minute peak, and the monomeric free protein (M) peaks were compared next for sample #4 and representative OG1953 drug product clinical formulation. As seen in FIG. 28A, the LP2 and 16 minute peaks were both present in sample #4 and in the representative OG1953 drug product clinical formulation at 25° C. for 6 months as compared to the frozen or 5° C. samples. For both samples, the temperature dependent increase is approximately 3.9 μg and approximately 14 μg for LP2 and the 16 minute peak respectively. The results continue to suggest that the impurities (e.g., aggregates and/or degradation products) are derived from the OG1953 conjugate. (X) of FIG. 28A indicates the 16 minute fraction at 25° C. was not increased for batch 101 (drug substance (DS) at 20 mg/ml), but was increased by about 1.3× for 103A (drug product (DP) at 50 mg/ml) and sample #4. A chromatogram of the CEX-unbound fraction comparing the LP1 and LP2 peaks of the OG1953 KSI-301DP 103A frozen and 6M 25° C. samples is shown in FIG. 28B. Chromatograms of the CEX-bound fraction indicating the 16 minute peak and Z describing monomer form protein (M) peak are shown in FIGS. 28C-28D. As shown in FIG. 28D, (Z) indicates the monomeric form protein peak (M) at 22 minutes increased from 3 μg to 6 μg (67% increase) for batch 103A at 25° C. after 6 months as compared to the frozen sample. This has been observed previously as the malemide retro-reaction that resulted in protein looping out of the OG1953 conjugate.

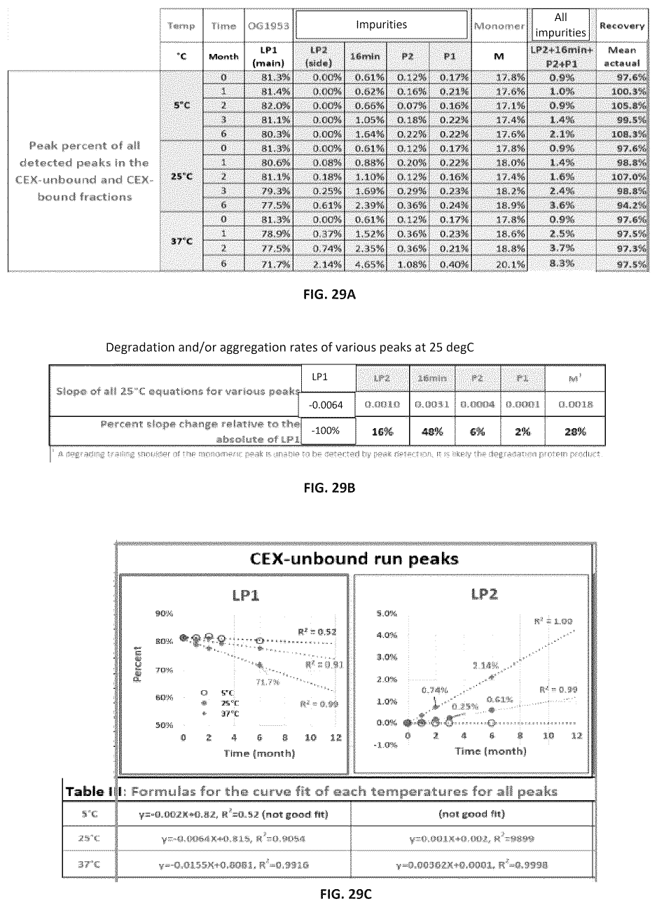

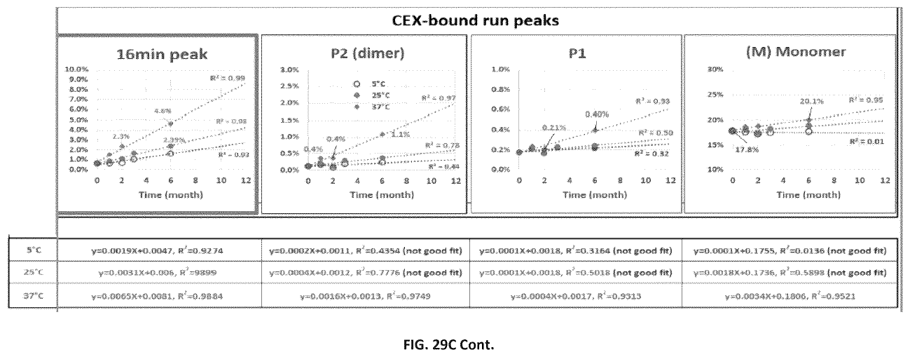

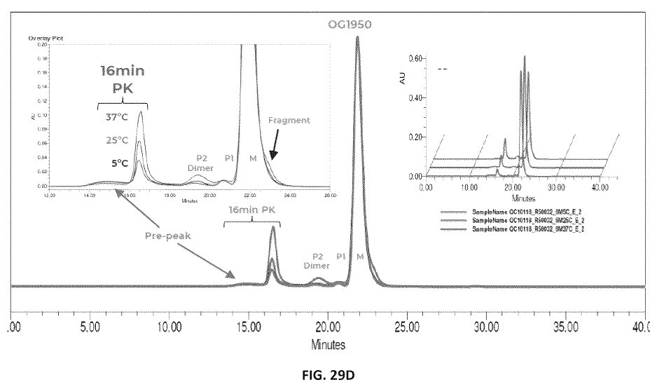

Next, kinetic analysis of various peak fractions of sample #4 at different time and temperatures was performed. As shown in FIG. 29A, the assay had close to 100% mass recovery which warrants the reliability of the assay for various peak percentages. As shown in FIG. 29B, the 16 minute peak fraction accounts for almost half of the instability, followed by the retro-reaction related monomeric form at 28%, the LP2 fraction at 16%, and the combined protein aggregates P1 and P2 at 8%. FIG. 29C includes the kinetic curve fit for the LP1 and LP2 peaks in the CEX-unbound fraction (OG1953 conjugates); and the 16 minute peak (OG1953 related fraction) OG1950 free protein fractions that includes monomer (M), dimer (P2), and a form larger than the monomer but smaller than the dimer (P1). The monomer peak contains a trailing shoulder (fragment) at stress temperatures (25° C. and 37° C.). The major instability factor was the OG1953 main peak, or LP1, it was the only fraction that had a downward trend. The rate of aggregation and/or degradation was at 0.64%/month and 1.55%/month for 25° C. and 37° C. respectively. Favorable stability was achieved at 5° C. As shown in FIGS. 29B, 29C and 29D, aggregation and/or degradation was accompanied with concomitant increases in various impurities (e.g., aggregates and/or degradation products) including LP2, 16 minute peak, P1, P2, and M. The OG1950 protein aggregates (P1 and P2) increased slowly.

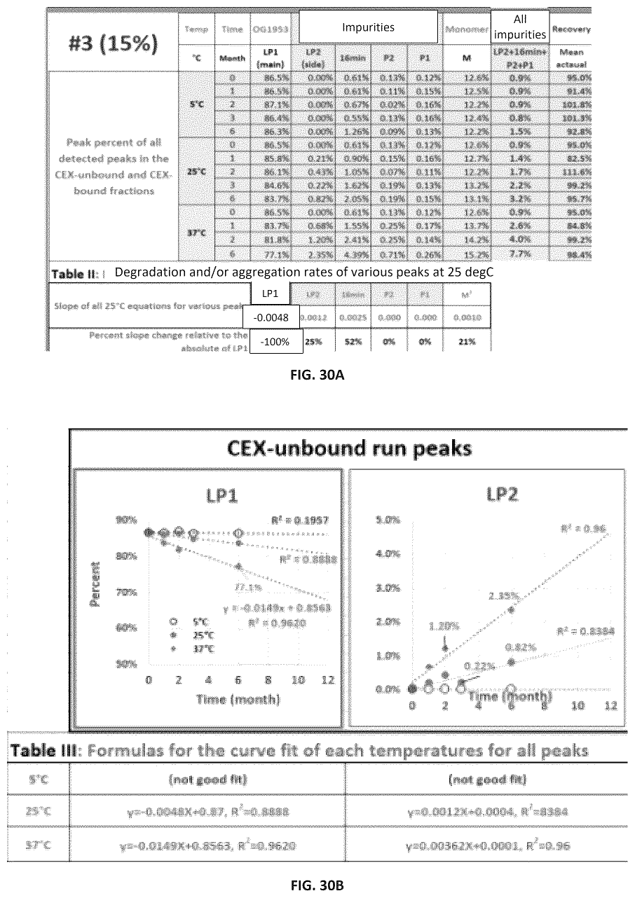

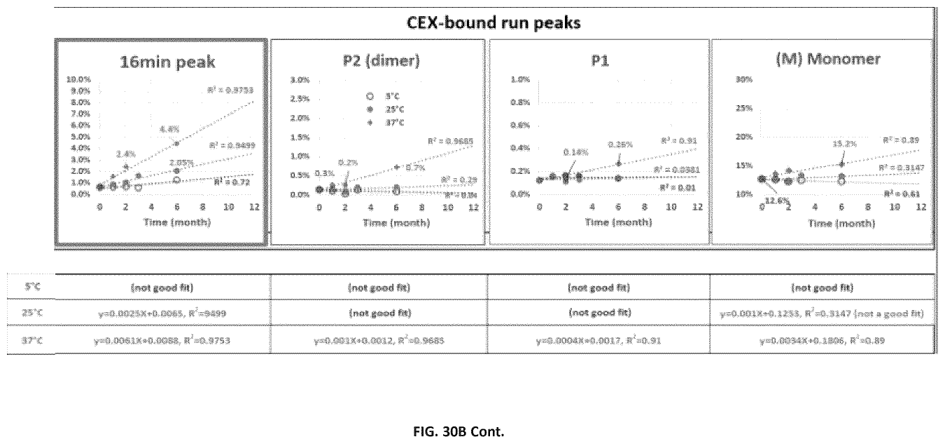

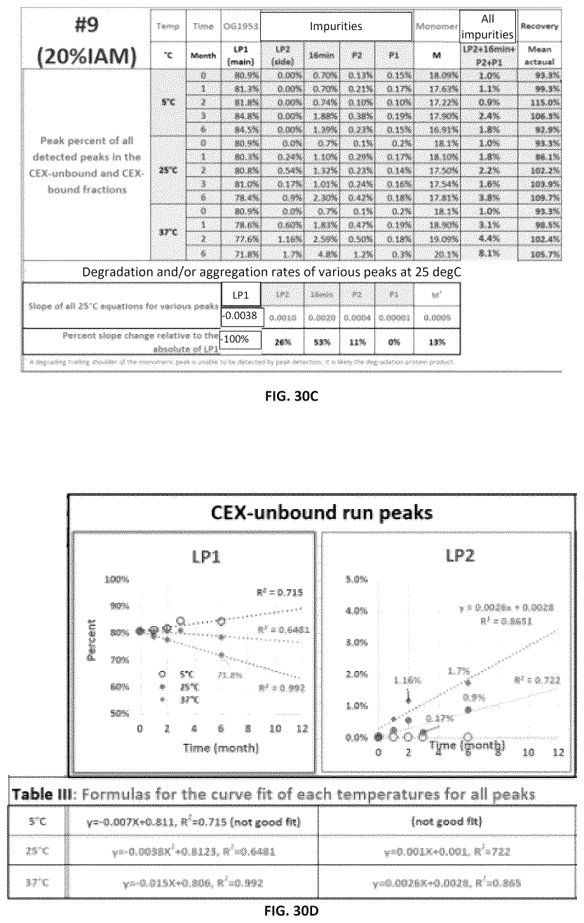

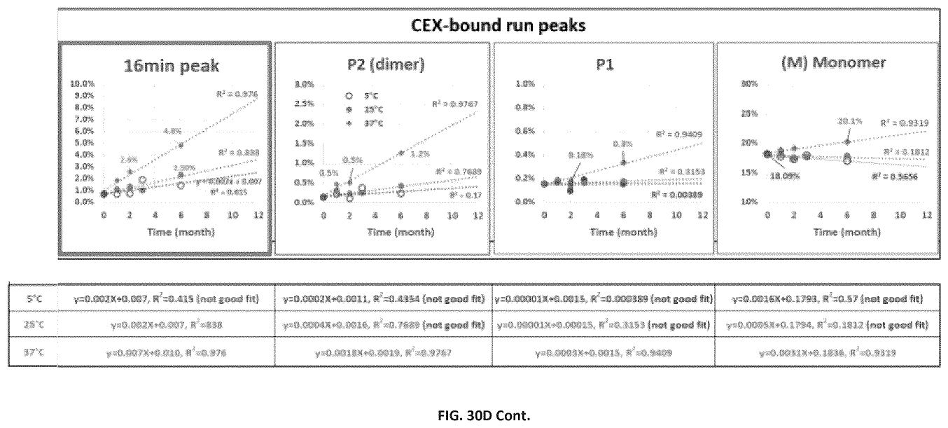

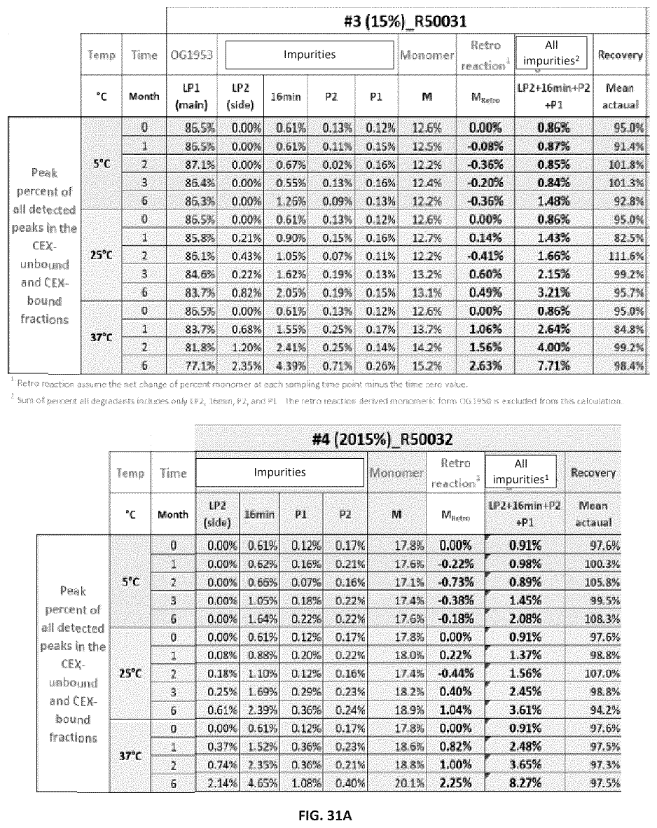

Next, kinetic analysis of various peak fractions of sample #3 (15%) and sample #9 (20% IAM) at different times and temperatures was performed, as shown in FIGS. 30A-30D. As shown in FIGS. 29A, 30A, and 30C, high recovery for all samples warrants the reliability of analysis by peak percent of all peaks across CEX-bound and CEX-unbound samples. The protein concentration of the working stock solution prior to the sample injection was determined by Nanodrop spectrophotometer, except the time 0 samples which use the theoretical value of 670 μg, the lower recovery at 93.3 of sample may be due to overestimation of working stock. As shown in FIGS. 29C, 30B and 30D, LP1 has a negative slope, whereas all other peaks including LP2, 16 minutes, P1, P2, and Monomer have a positive slope for samples #3, #4 and #9. This suggested that the LP1 peak is the primary source of impurities (e.g., aggregation and/or degradation) and results in the increase of all other impurities peaks. As shown in FIG. 29C, the slopes for 5° C., 25° C., and 37° C. were −0.002, −0.0064, and −0.0155 respectively, indicating that 25° C. and 37° C. increased degradation and/or aggregation rate by 32× and 77.5× respectively. Sample #9 showed a similar result to sample #4, as the IAM modification had a minimal effect on stability, as seen in FIG. 30D. Reducing the free protein to 15% in sample #3 resulted in 25% reduction in degradation and/or aggregation rate at 25° C., as shown in FIGS. 30A-30B. Increasing temperature of 5° C. to 37° C. over time resulted in increasing steepness of the slope for different sampling of all peaks, suggesting degradation and/or aggregation was time and temperature dependent.

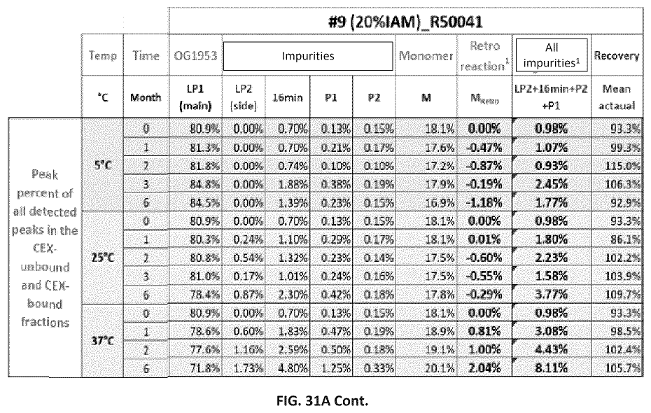

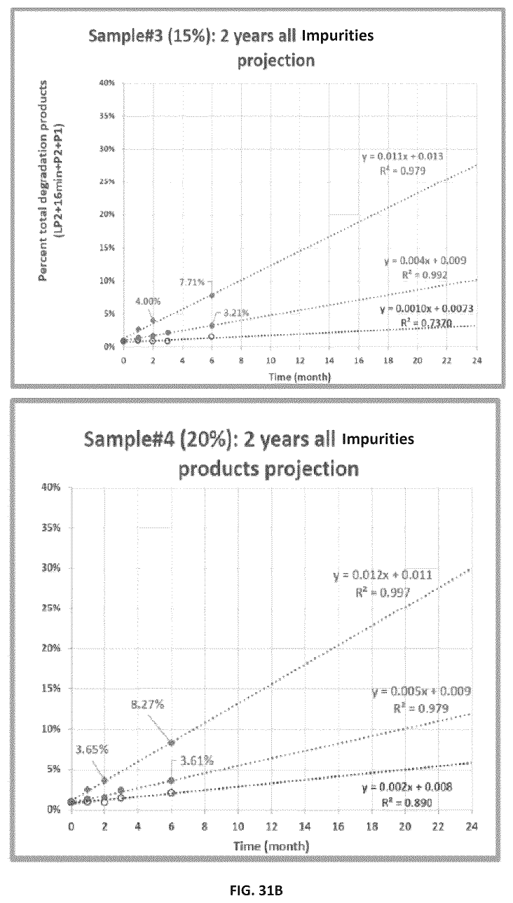

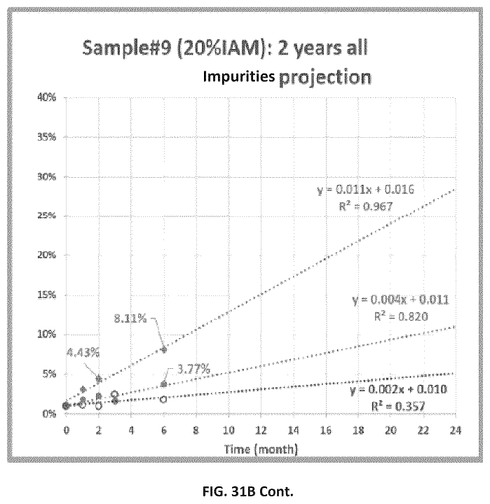

Based upon the stability data for samples #3, #4, and #9, at 0, 1, 2, 3, and 6 months, 2 year stability projections were generated at different temperatures stability data summarized in FIG. 31A. As shown in FIG. 31B, all three formulations showed excellent long-term stability, with less than 5% total impurities for at least 8 and 20 months at 25° C. and 5° C. respectively. The calculation excluded the retro-reaction derived monomeric form accumulation. The retro reaction was found to be negligible at 5° C. and increased dramatically at 25° C. and 37° C.

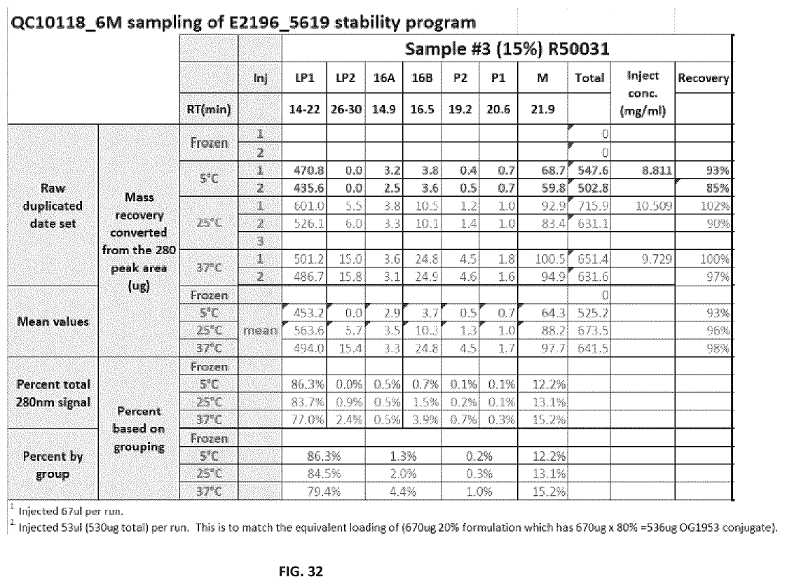

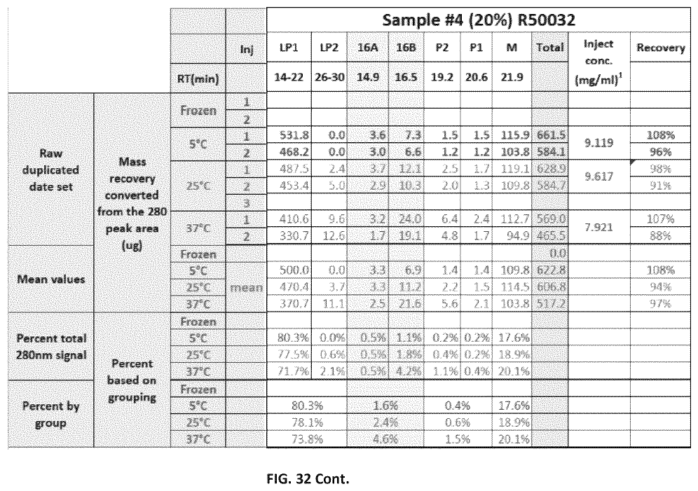

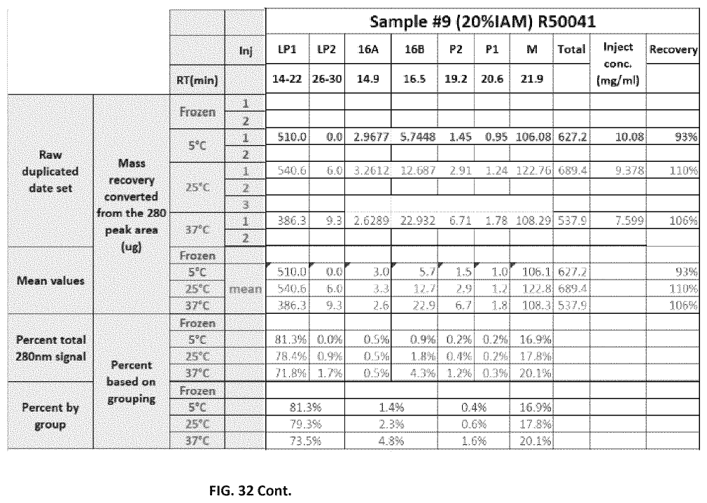

Additional mass recovery data for all injections of samples #3, 4, and 9 at different times and temperatures is summarized in FIG. 32.

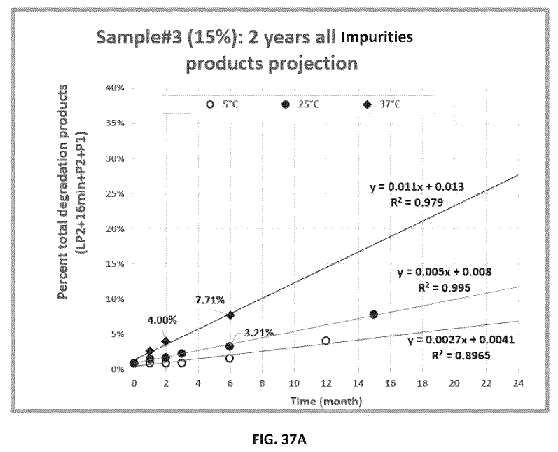

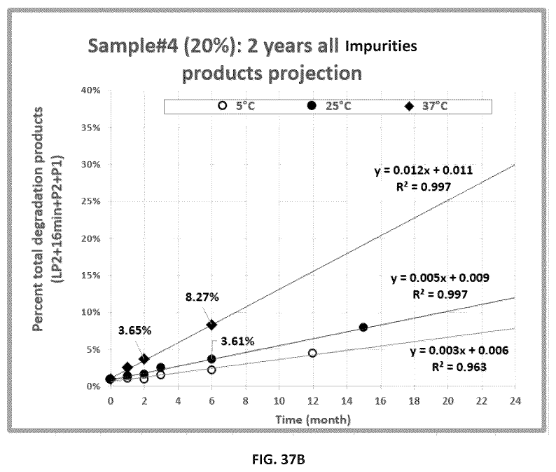

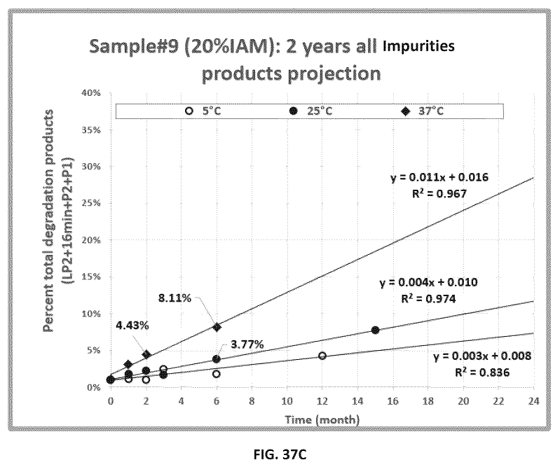

Additional samplings were performed at 12 and 15 months for 5° C. and 25° C., respectively, as shown in FIGS. 37A-37C. The data shows that the samples can be stored at 5° C. for up to a year with less than 5% degradation impurities detected.

### Example 8—Tandem Method with Single Injection and Single Continuous Run Mode

Previously described embodiments with or without a column switch between the CEX and SEC columns (SOP391_version 1-6) were operated in a discontinuous mode, wherein a single sample injection was separated into CEX-unbound and CEX-bound chromatography runs. The embodiments described in the current example combine the two runs into a single chromatographic run without interruption. These embodiments include the following modifications: The total run time was 80 min at a flowrate of 0.5 ml/min with isocratic run except at 40 min, there is a short pulse of 100% B buffer for 1 min. Both pump A and B were used in which pump A was to deliver the isocratic running buffer at 0.5 ml/min with 20 mM sodium acetate pH 5, 100 mM NaCl at all time; pump B buffer was to deliver a 1 min pulse at the same flowrate with 20 mM sodium acetate pH 5, 1M NaCl.

This version of the tandem HPLC method resulted in a continuous 80 min run which 0-40 min is the CEX-unbound phase run, which the bulk of unbound OG1953 conjugate peaks load peak 1&2 (LP1&2) were eluted first, then there was a 1 min 100% B pulse from 40.01-41 min to elute the CEX-bound fractions before the system was switched back to 100% A, the eluted CEX-bound fractions which included some OG1953 related conjugate impurity (which was previously called 16 min fraction), the aggregates of OG1950 (namely peak 1&2), monomeric form OG1950, and a fragment peak elutes as trailing shoulder of monomer peak. It has been shown that this version of SOP391_v1.1 (which is derived from the method #5 of the current embodiment) has the same recovery and resolution as compared to the original SOP391_v1. The salt pulse was found to highly influence method performance as seen by recovery and baseline.

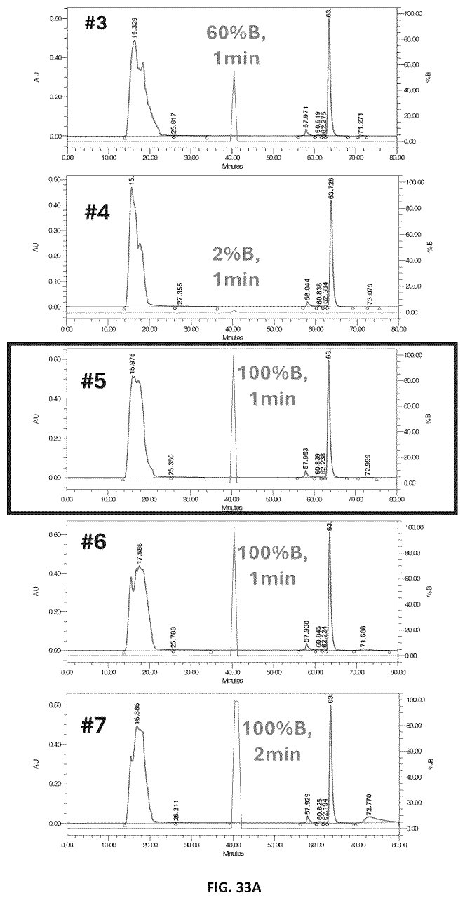

FIGS. 33A-33B graphs show 100% B buffer pulse elution profiles of the 20% antibody with 80% bioconjugate. As shown by FIGS. 33A-33B, 100% recovery was observed for all pulse elution methods except for method #4, which had low recovery due to insufficient buffer B pulse. Baseline pulse elution signal was observed for methods #6 & #7. Best performing method was observed for methods #3 and #5, where method #5 was preferred due to slightly better recovery.

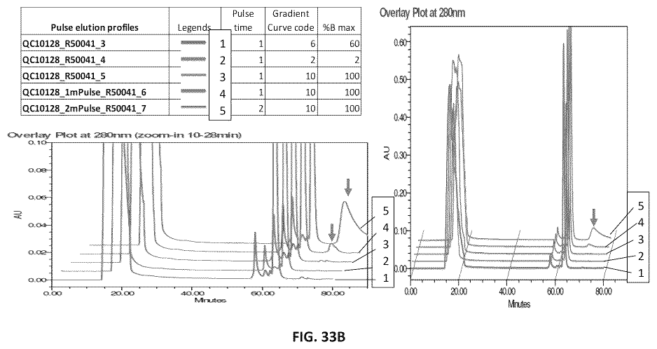

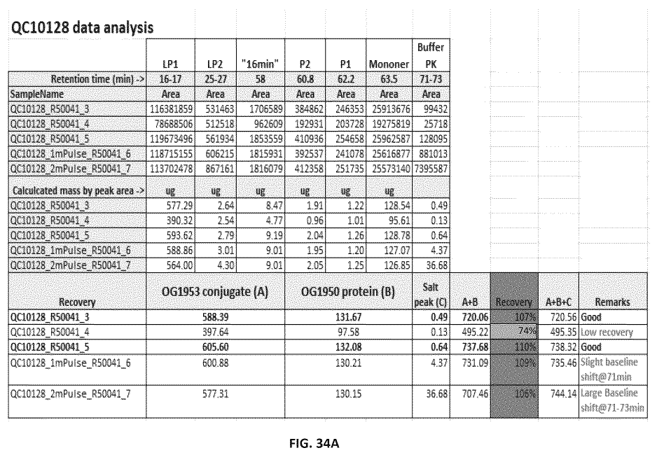

Individual chromatogram comparison of various salt pulse elution profiles are shown in FIGS. 34A-34B. 100% recovery has been observed for all pulse elution methods except for method #4, which has low recovery due to insufficient buffer B pulse. Baseline pulse elution signal has been observed for methods #6 & #7. The best performing method has been observed for #3 and #5, which the method #5 was preferred due to slightly better recovery.

### Example 9: Continuous 80 Minute Tandem Method Separation with PhotoDiol Array (PDA) Detection Set at 200-350 nm

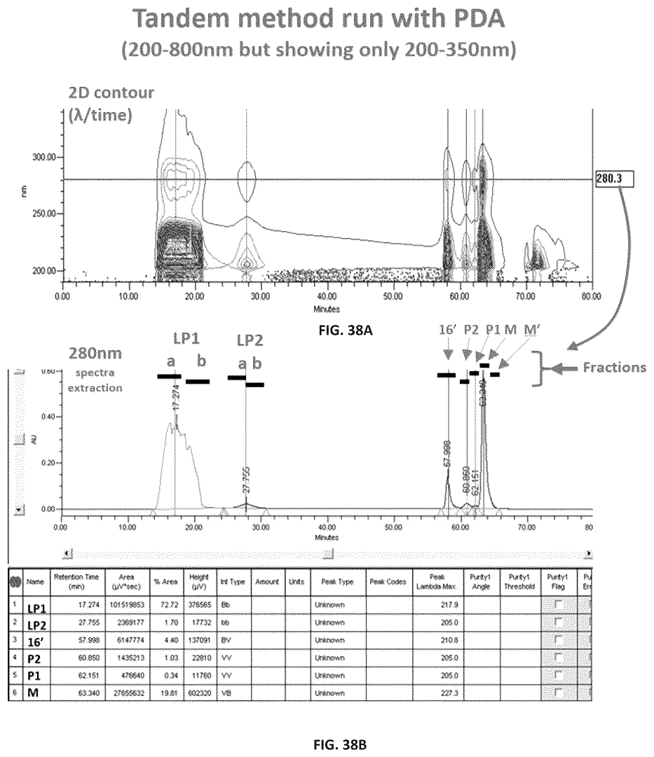

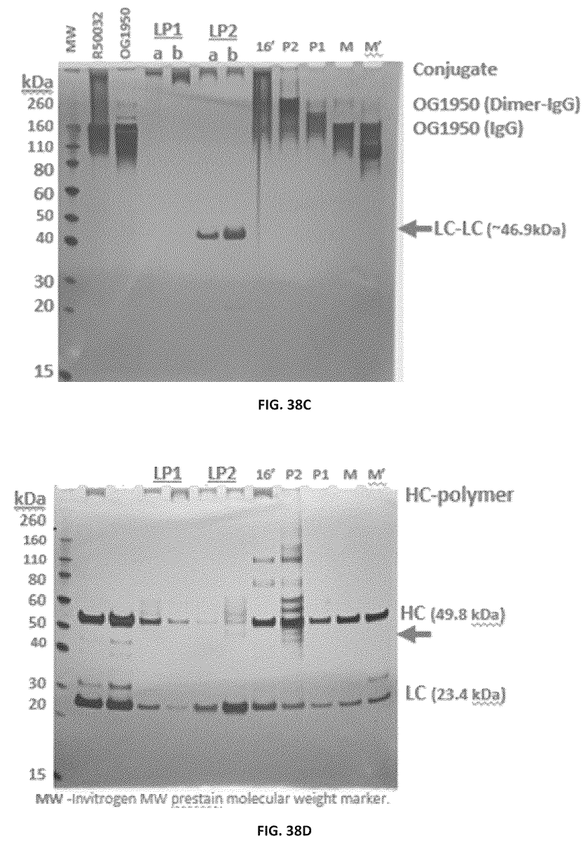

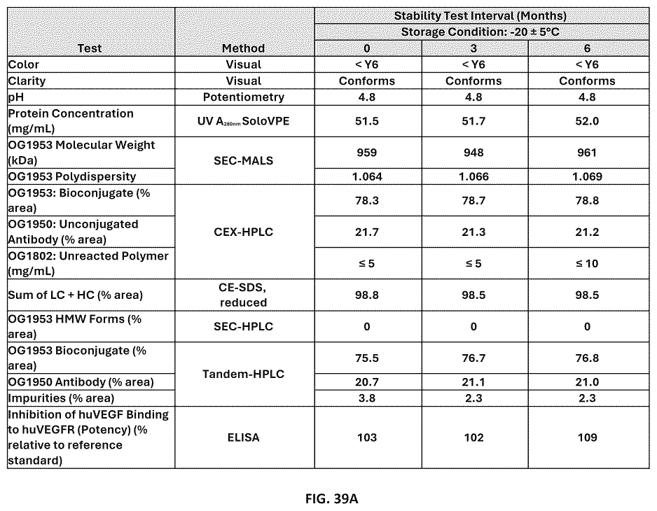

Tandem method with PDA detector set at 200-800 nm displayed discrete 280 nm protein peak absorbance for LP1, LP2, 16′, P2, P1 and monomeric IgG (M & M′). The 2D contour view of elution time versus wavelength is shown in FIG. 38A, and the extracted wavelength profile at 280 nm the peak identification of the various eluted fractions collected for further characterization are shown in FIG. 38B. Spectra extraction at 280 nm exhibited the typical 280 nm absorbance trace as in the continuous 80 minute Tandem method according to the SOP391 v7. In some embodiments, SOP391_v7 is a continuous 80 min run where the Loading and elution runs (L+E) are combined with a 1 min high salt pulse at about 40 min to elute the loaded antibody and aggregated forms which are separated immediately with the SEC from 41-80 min.

Eluted fractions were analyzed using SDS-PAGE under both non-reduced (FIG. 38C) and reduced (FIG. 38D) conditions, the gels were then stained with Silver Staining Kit to reveal the protein bands. The result included (1) LP1 peak for the OG1953 conjugate with a typically increased HC/LC ratio, as about 50% of the HC containing polymer (HC-polymer) were unable to enter the gel, whereas all the light chain (LC) remains unchanged. (2) LP2 contained a single protein migrating between 40-50 kD MW marker under non-reducing conditions, when reduced, the sample was confirmed to be the light chain (LC) which migrates like the standard. (3) the 16′ peak fraction was very heterogeneous, it may contain large MW aggregates of conjugate and protein aggregates, when reduced, there was HC, LC and HC-polymer barely migrating into the well. (4) P2 fraction contained predominantly dimers of OG1950 antibody with some spilled over aggregates from the 16′ peak fraction, this was evidenced by the presence of 110 kD and 80 kD bands under the reduced condition, it also contained some covalently aggregated species of IgG as evidenced by bands higher than the HC under reduced conditions. The presence of higher HC/LC ratio was evidence that majority of the aggregates in this fraction were IgG and not OG1953 conjugate. (5) P1 contained a diffused band with an apparent MW slightly higher than 160 kD MW marker, the identity of the form was unknown and it could be reduced to HC and LC as the predominant components. (6) The fraction eluting at 63.34 minutes represented the monomeric form OG1950 IgG (M) and the training fraction as represented by M′ contained some degrading form of OG1950 IgG.

Analytical Methods Description for both KSI-301 and KSI-501

COLOR AND CLARITY (APPEARANCE) BY VISUAL INSPECTION. Color of Drug Substance was examined visually against water and prepared color standards and conformed to Ph. Eur. 2.2.2 requirements. Sample results were reported versus the corresponding yellow color standards. Clarity (Appearance) of Drug Substance was examined against prepared opalescent reference suspensions and conformed to Ph. Eur. 2.2.1 requirements.

PH BY PHOTENTIOMETRY. The pH measurement analytical procedure was performed according to USP<791>/Ph. Eur. 2.2.3/JP 2.54.

PROTEIN CONCENTRATION BY UV A280NM. Absorbance at 280 nm was used to determine the protein concentration in the test sample using an extinction coefficient. The absorbance of the sample at different path lengths was measured the absorbance was ploted against the path length. A linear regression was performed where the slope (m, Abs mm-1) was proportional to the sample concentration, C=m/(extinction coefficient). The method conformed to USP<507>.

MOLECULAR WEIGHT AND POLYDISPERSITY BY SEC-MALS. Molecular weight and polydispersity of bioconjugate in Drug Substance samples was measured by coupling SEC-HPLC and Multi-Angle Static Light Scattering (MALS) detector, where the intensity of the scattered light was proportional to the concentration of the macromolecules in solution so that the absolute molar mass and size were determined for each eluting fraction from SEC-HPLC. Polydispersity measures the size distribution of molecules of bioconjugate in Drug Substance samples by comparing the ratio of weighted average molecular weight (Mw) to number average molecular weight (Mn).

BIOCONJGATE, UNCONJUGATED ANTIBODY, AND OG1802 UNREACTED POLYMER BY CEX-HPLC. CEX-HPLC was used to assess Drug Substance purity by separating the bioconjugate from unconjugated antibody, as well as from other product related species (if present). Each of the protein species of concern can be quantified and expressed as a percentage of the total integrated peak area. The area for the OG1802 unreacted polymer at 280 nm in DS was evaluated by comparing to the OG1802 Biopolymer Intermediate limit standards.

BIOCONJUGATE, BIOCONJUGATE HMW FORMS, AND BIOCONJUGATE LMW FORMS (INCLUDING ANTIBODY) BY SEC-HPLC. Size Exclusion Chromatography (SEC-HPLC) was used to assess Drug Substance purity by separating the bioconjugate from its high molecular weight (HMW) and low molecular weight (LMW) (including antibody) species. Each of the protein species of concern can be quantified and expressed as a percentage of the total integrated peak area.

BIOCONJUGATE, ANTIBODY, AND IMPURITIES BY TANDEM-HPLC. Tandem-HPLC is a tandem chromatography method that, in some embodiments, includes connecting a Cation Exchanger (CEX) HPLC column followed by a Size Exclusion Chromatography (SEC) column. The chromatography was accomplished in a single run which included i) binding of unconjugated antibody with CEX column and ii) elution of bound unconjugated antibody with a short pulse of buffer with high salt. Briefly, the ionic strength of the isocratic running buffer allowed the bioconjugate and the weakly bound antibody light chain and its aggregates to flow through the CEX column while the antibody/fusion protein monomer, antibody/fusion protein aggregates and a trace amount of tightly bound variants of bioconjugate remained bound to the CEX column (loading phase); a continuous chasing with mobile phase ensured the signal returns to the baseline; then a short pulse of high salt allowed the CEX-bound fraction to be eluted and be immediately separated by the SEC column.

POTENCY (INHIBITION OF HUMAN VEGF BINDING TO HUMAN VEGFR BY ELISA). The ability of KSI-301 Drug Substance samples to inhibit binding of huVEGF to huVEGFR immobilized on an ELISA plate was tested. In this method, increasing concentrations of KSI-301 Drug Substance were pre-incubated with biotin-labeled VEGF (bt_VEGF) and loaded onto a VEGFR coated plate, after which bound bt_VEGF was detected using streptavidin HRP. The IC50 of KSI-301 Drug Substance was calculated and compared to that of the reference standard.

POTENCY (BIOLOGICAL ACTIVITY USING VEGF REPORTER CELL LINE KDR/NFAT-RE HEK293). The potency of KSI-501 Drug Substance samples was measured in a reporter gene assay using the VEGF responsive cell line KDR/NFAT-RE HEK293. The analytical method was based on the capacity of KSI-501 to inhibit VEGF activity using the VEGF responsive cell line KDR/NFATRE HEK293. The KDR/NFAT-RE HEK293 cells have been engineered to express the NFAT response element upstream of Luc2P as well as exogenous KDR. When VEGF binds to the KDR/NFAT-RE HEK293 cells, the KDR transduces intracellular signals resulting in NFAT-RE-mediated luminescence. In this method, increasing concentrations of KSI-501 were preincubated with VEGF and loaded to KDR/NFAT-RE HEK293 cells. KSI-501 reduced activation of VEGF induced signaling and lead to reduction of NFAT-RE-mediated luminescence. The resulting luminescence data were used to fit 4-parameter sigmoidal dose-response curves. Parallel-line comparison of dose response curves generated with the reference material and curves generated with the test sample allowed for determination of the test sample's potency.

POTENCY (BIOLOGICAL ACTIVITY BY COMPETITIVE ELISA FOR MEASURING ANTI-IL6). The potency and identity of KSI-501 Drug Substance samples was measured by a competitive ELISA based on the ability of KSI-501 to block the binding of human IL6 ligand to their receptor IL6Ra. The potency was evaluated by quantifying remaining IL6/IL6Ra complexes using competitive ELISA. The inhibitory action was monitored in vitro by coincubation of recombinant IL6 and IL6Ra with increasing concentrations of KSI-501 and the determination of the remaining IL6/IL6Ra complex by an ELISA using an antibody pair specific for IL-6/IL-6Ra complex. The resulting data (Absorbance at 450 nm) were used to fit 4-parameter sigmoidal dose-response curves. Comparison of dose-response curves generated with the reference standard and curves generated with the test sample allowed for determination of the test sample's potency.

### Example 10: KSI-301 Stability Evaluation Out to 6 Months with Tandem-HPLC Method

Some embodiments of a formulation containing 80% OG1953 conjugate and 20% unconjugated OG1950 were scaled up and produced at large scale under GMP and were released for clinical trials. Briefly, KSI-301 Drug Substance was manufactured by conjugating OG1950 Antibody Intermediate with OG1802 Biopolymer Intermediate. The conjugation process began with reduction of the OG1950 Antibody Intermediate using tris(2-carboxyethyl) phosphine hydrochloride (TCEP-HCl)—an aqueous solution—followed by re-oxidation with dehydroascorbic acid. This was followed by the addition of OG1802 Biopolymer Intermediate to the decapped and re-oxidized OG1950 antibody and the conjugation of the OG1802 Biopolymer Intermediate to free cysteines occured. In the following steps, the crude conjugation product was purified with bind-and-elute polishing cation exchange (CEX) chromatography, followed by ultrafiltration, OG1950 Antibody Intermediate addition, diafiltration, and ultrafiltration steps to concentrate. OG1950 Antibody Intermediate was added and exchange with a final formulation buffer, and then concentrated to yield KSI-301 Drug Substance.

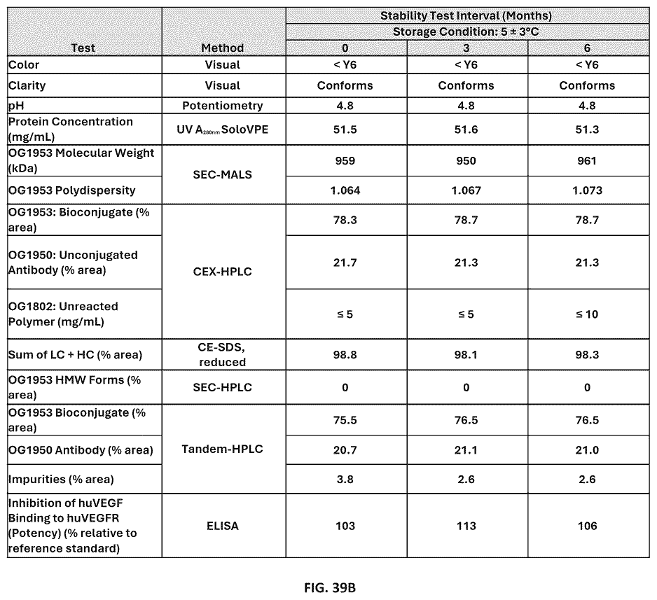

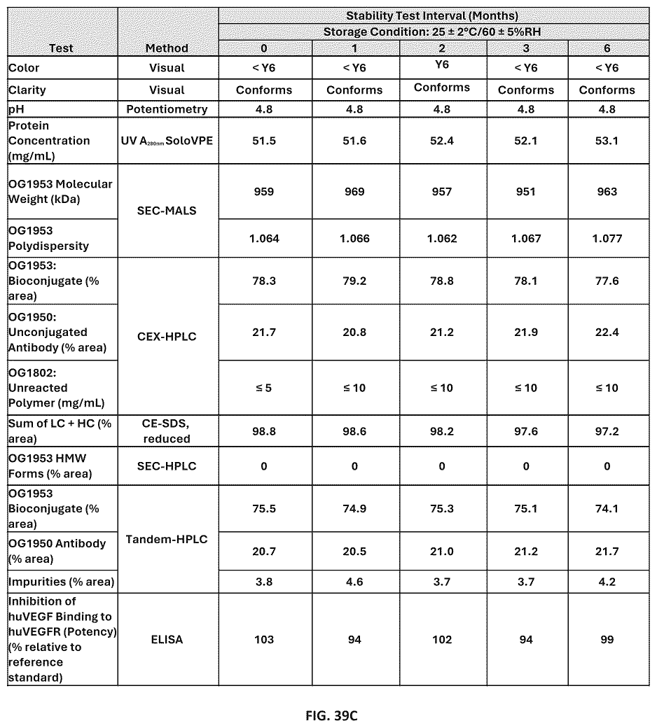

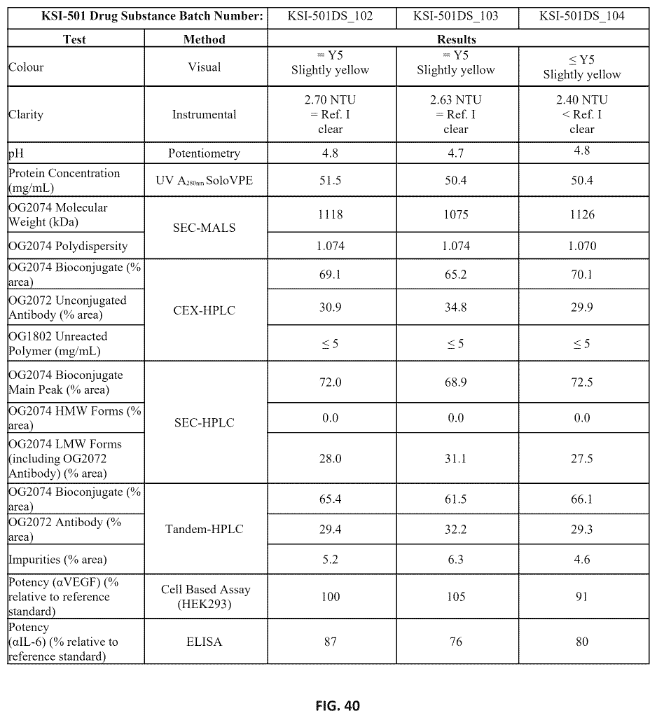

From the data shown in FIGS. 39A-39C, the quality of the drug substance continues to support a clear and stable formulation for intravitreal injectable medicine application. FIGS. 39A-39C show lot release (time 0) and long term stability up to 6 months. The product showed excellent stability at up to 6 months at ambient temperature or lower with impurity level below 5% when using Tandem-HPLC method. All other assays showed minimal to no product deterioration as compared to time 0 sampling. This data illustrates that the tandem method described herein is an excellent stability indicating assay able to provide excellent resolution for monitoring the various product related impurities over time at different temperatures.

### Example 11: KSI501_Batch 1-3 Lot Release Summary

The selected formulation containing 70% OG2074 conjugate and 30% unconjugated OG2072 was scaled up and three separate batches including KSI501_Batch 1-3 were produced at large scale under GMP and the were released for clinical trials. The quality of the drug substance continues to support a clear formulation for intravitreal injectable medicine application. FIG. 40 shows the time 0 (lot release) analysis summary of the KSI501_batch 1-3.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect disclosed herein can be used in combination with any other unless specifically indicated otherwise. Although some embodiments have been described in some detail by way of illustration and example for purposes of clarity and understanding, it will be apparent that certain changes and modifications may be practiced within the scope of the appended claims.

What is claimed is:

1. A method of analyzing a mixed formulation sample, the method comprising:
i. providing a mixed formulation that comprises:
a. a first antibody that is an anti-VEGF-A antibody conjugated to a polymer; and
b. a second antibody that is an anti-VEGF-A antibody that is not conjugated to the polymer;
ii. a first run, wherein the mixed formulation is run through a prefiltration step to provide a first filtered mixed formulation, wherein the first filtered mixed formulation is run through a cation exchange chromatography (CEX) column to provide a second filtered mixed formulation;
iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation;
iv. a specific ionic strength of the solvent is used to allow partitioning of the mixed formulation in the CEX column; and
wherein the method allows analysis of the mixed formulation based on the differential charge and size variant of the constituents.

2. A method of analyzing a mixed therapeutic composition sample, the method comprising:
i. providing a mixed therapeutic composition that comprises:
a. a first antibody that is an anti-VEGF-A antibody conjugated to a polymer; and
b. a second antibody that is an anti-VEGF-A antibody that is not conjugated to the polymer;
ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition;
iii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition;
iv. a specific ionic strength of the solvent is used to allow partitioning of the mixed therapeutic composition in the CEX column; and
wherein the method allows analysis of the mixed therapeutic composition based on the differential charge and size variant of the constituents.

3. A method of analyzing a mixed therapeutically effective formulation sample, the method comprising:
i. providing a mixed therapeutically effective formulation that comprises:
a. a first antibody that is an anti-VEGF-A antibody conjugated to a polymer; and
b. a second antibody that is an anti-VEGF-A antibody that is not conjugated to the polymer;
ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation;
iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation;
iv. a specific ionic strength of the solvent is used to allow partitioning of the mixed therapeutically effective formulation in the CEX column; and
wherein the method allows analysis of the mixed therapeutically effective formulation based on the differential charge and size variant of the constituents.

4. An apparatus comprising a tandem series of CEX and SEC HPLC columns for use in analyzing a mixed formulation sample comprising a) a first antibody that is an anti-VEGF-A antibody conjugated to a polymer and b) a second antibody that is an anti-VEGF-A antibody that is not conjugated to the polymer,
wherein the purification comprises;
i. a first run, wherein the mixed formulation is run through a prefiltration step to provide a first filtered mixed formulation, wherein the first filtered mixed formulation is run through a cation exchange chromatography (CEX) column to provide a second filtered mixed formulation; and
ii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation.

5. An apparatus comprising a tandem series of CEX and SEC HPLC columns for use in analyzing a mixed therapeutic composition sample comprising a) a first antibody that is an anti-VEGF-A antibody conjugated to a polymer and b) a second antibody that is an anti-VEGF-A antibody that is not conjugated to the polymer,
wherein the purification comprises;
i. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; and
ii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition.

6. An apparatus comprising a tandem series of CEX and SEC HPLC columns for use in analyzing a mixed therapeutically effective formulation sample comprising a) a first antibody that is an anti-VEGF-A antibody conjugated to a polymer and b) a second antibody that is an anti-VEGF-A antibody that is not conjugated to the polymer,
wherein the purification comprises;
i. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; and
ii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation.

7. A method of analyzing a mixed formulation sample, the method comprising:
i. providing a mixed formulation that comprises:
a. a first antibody that is an anti-VEGF-A antibody conjugated to a polymer; and
b. a second antibody that is an anti-VEGF-A antibody that is not conjugated to the polymer;
ii. a first run, wherein the mixed formulation is run through a prefiltration step to provide a first filtered mixed formulation, wherein the first filtered mixed formulation is run through a cation exchange chromatography (CEX) column to provide a second filtered mixed formulation; and
iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation; and
wherein the method reduces the presence of antibody aggregates in the formulation; and
wherein the polymer of the first antibody comprises a phosphorylcholine containing polymer, wherein the polymer is covalently bonded to the first antibody at a cysteine outside a variable region of the first antibody, and wherein said cysteine replaces a non-cysteine amino acid that occurs in a same position in a sequence, wherein the first antibody comprises a light chain and heavy chain, said heavy chain comprising an Fc region, wherein the cysteine is in the Fc region of the heavy chain, wherein the sequence of the heavy chain comprises SEQ ID NO: 1 (with or without the C-terminal lysine), and wherein the sequence of the light chain comprises SEQ ID NO: 2; wherein the antibody conjugate has the following structure: 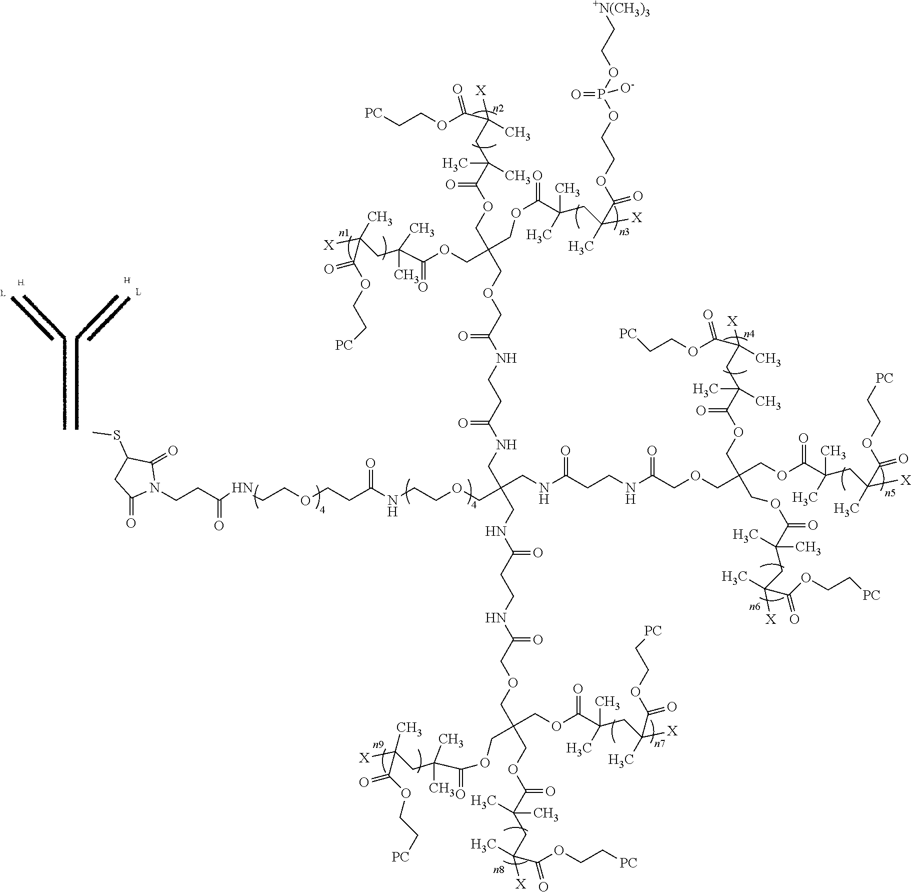
where X is a) —OR where R is-H, Methyl, ethyl, propyl, or isopropyl, b) —H, or c) a halide; (or optionally where X is a) —OR where R is —H, Methyl, ethyl, propyl, or isopropyl, b) —H, c) any halogen, including —Br, —Cl, or —I, d) —SCN, or e) —NCS;)
wherein:
each heavy chain of the first antibody is denoted by the letter H, and each light chain of the first antibody is denoted by the letter L;
the polymer is bonded to the first antibody through the sulfhydryl of a cysteine at position 449, as numbered in SEQ ID NO: 1, which bond is depicted on one of the heavy chains;
PC is 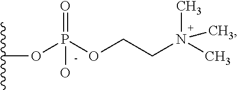
where the curvy line indicates the point of attachment to the rest of the polymer; and n1, n2, n3, n4, n5, n6, n7, n8 and n9 are the same or different such that the sum of n1, n2, n3, n4, n5, n6, n7, n8 and n9 is 2500 plus or minus 15%; and
wherein, the second antibody comprises a light chain and heavy chain, said heavy chain comprising an Fc region, wherein a cysteine is in the Fc region of the heavy chain, wherein the sequence of the heavy chain comprises SEQ ID NO: 1 (with or without the C-terminal lysine), and wherein the sequence of the light chain comprises SEQ ID NO: 2; and
wherein the mixed formulation sample travels sequentially through a prefilter, the CEX column, and the SEC HPLC column, wherein the tandem CEX and SEC HPLC columns are a Shodex SP825 column with internal diameter (i.d.)×length dimensions of 9.0×75 mm and a TSKgel G3000SWxl column with i.d.×length dimensions of 7.8×300 mm column arrangement;
wherein the isocratic running conditions comprise a flow rate of 0.5 ml/min, and the buffer comprises 20 mM sodium acetate, pH 5 any amount between 50 mM NaCl and 5M NaCl;
wherein the percentage of the second antibody is any percentage between 0% and 20%;
wherein the mixed formulation sample comprises any amount between 25 ug and 1340 ug of protein;
wherein the method further comprises assessment for high molecular weight aggregates within the purified mixed formulation by methods comprising SEC profile analysis and SDS PAGE gel; and
wherein the method further comprises assessment of the percentage of the second antibody in the purified mixed formulation by SEC profile analysis;
wherein the first run separates the first antibody from the second antibody and antibody aggregates,
wherein the step for elution of CEX bound free protein during the second run comprises a pulse of high salt at 1M NaCl,
wherein during the second run, the fourth filtered mixed formulation is run through a prefiltration step to provide a fifth filtered mixed formulation, wherein the fifth filtered mixed formulation is run through a CEX column to provide a six filtered mixed formulation, wherein the sixth mixed formulation is run through a SEC HPLC column; and
wherein running the filtered mixed sample through a size exclusion exchange SEC HPLC column separates the second antibody from antibody aggregates.

8. A method of analyzing a mixed formulation sample, the method comprising:
i. providing a mixed formulation that comprises:
a. a fusion protein conjugated to a polymer; and
b. a second protein that is not conjugated to the polymer;
ii. a first run, wherein the mixed formulation is run through a prefiltration step to provide a first filtered mixed formulation, wherein the first filtered mixed formulation is run through a cation exchange chromatography (CEX) column to provide a second filtered mixed formulation;
iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation;
a specific ionic strength of the solvent is used to allow partitioning of the mixed formulation in the CEX column; and
wherein the method allows analysis of the mixed formulation based on the differential charge and size variant of the constituents.

9. A method of analyzing a mixed therapeutic composition sample, the method comprising:
i. providing a mixed therapeutic composition that comprises:
a. a fusion protein conjugated to a polymer; and
b. a second protein that is not conjugated to the polymer;
ii. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition;
iii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition;
iv. a specific ionic strength of the solvent is used to allow partitioning of the mixed therapeutic composition in the CEX column; and
wherein the method allows analysis of the mixed therapeutic composition based on the differential charge and size variant of the constituents.

10. A method of analyzing a mixed therapeutically effective formulation sample, the method comprising:
i. providing a mixed therapeutically effective formulation that comprises:
a. a fusion protein conjugated to a polymer; and
b. a second protein that is not conjugated to the polymer;
ii. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation;
iii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation;
iv. a specific ionic strength of the solvent is used to allow partitioning of the mixed therapeutically effective formulation in the CEX column; and
wherein the method allows analysis of the mixed therapeutically effective formulation based on the differential charge and size variant of the constituents.

11. An apparatus comprising a tandem series of CEX and SEC HPLC columns for use in purifying a mixed formulation sample comprising a) a first fusion protein conjugated to a polymer and b) a second protein that is not conjugated to the polymer,
wherein the purification comprises;
i. a first run, wherein the mixed formulation is run through a prefiltration step to provide a first filtered mixed formulation, wherein the first filtered mixed formulation is run through a cation exchange chromatography (CEX) column to provide a second filtered mixed formulation; and
ii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation.

12. An apparatus comprising a tandem series of CEX and SEC HPLC columns for use in purifying a mixed therapeutic composition sample comprising a) a first fusion protein conjugated to a polymer and b) a second protein that is not conjugated to the polymer, wherein the purification comprises;
i. a first run, wherein the mixed therapeutic composition is run through a prefiltration step to provide a first filtered mixed therapeutic composition, wherein the first filtered mixed therapeutic composition is run through a cation exchange chromatography (CEX) column to provide a second filtered mixed therapeutic composition; and
ii. a second run, wherein the protein bound to the CEX column is eluted to provide a third filtered mixed therapeutic composition, wherein the third filtered mixed therapeutic composition is run through a size exclusion exchange (SEC) high performance liquid chromatography (HPLC) column to provide a fourth filtered mixed therapeutic composition.

13. An apparatus comprising a tandem series of CEX and SEC HPLC columns for use in purifying a mixed therapeutically effective formulation sample comprising a) a first fusion protein conjugated to a polymer and b) a second protein that is not conjugated to the polymer, wherein the purification comprises;
i. a first run, wherein the mixed therapeutically effective formulation is run through a prefiltration step to provide a first filtered mixed therapeutically effective formulation, wherein the first filtered mixed therapeutically effective formulation is run through a cation exchange chromatography (CEX) column to provide a second filtered mixed therapeutically effective formulation; and
ii. a second run, wherein the protein bound to the CEX column is eluted to provide a third filtered mixed therapeutically effective formulation, wherein the third filtered mixed therapeutically effective formulation is run through a size exclusion exchange (SEC) high performance liquid chromatography (HPLC) column to provide a fourth filtered mixed therapeutically effective formulation.

14. A method of analyzing a mixed formulation sample, the method comprising:
i. providing a mixed formulation that comprises:
a. a first antibody that is an anti-VEGF-A antibody conjugated to a polymer; and
b. a second antibody that is an anti-VEGF-A antibody that is not conjugated to the polymer;
ii. a first run, wherein the mixed formulation is run through a prefiltration step to provide a first filtered mixed formulation, wherein the first filtered mixed formulation is run through a cation exchange chromatography (CEX) column to provide a second filtered mixed formulation;
iii. a second run, wherein the protein bound to the CEX column is eluted to provide a third filtered mixed formulation, wherein the third filtered mixed formulation is run through a size exclusion exchange (SEC) high performance liquid chromatography (HPLC) column to provide a fourth filtered mixed formulation;
iv. a specific ionic strength of the solvent is used to allow partitioning of the mixed formulation in the CEX column; and
wherein the method allows analysis of the mixed formulation based on the differential charge and size variant of the constituents.

15. A method of analyzing a mixed formulation sample, the method comprising:
i. providing a mixed formulation that comprises:
a. a first antibody that is an anti-VEGF-A antibody conjugated to a polymer; and
b. a second antibody that is an anti-VEGF-A antibody that is not conjugated to the polymer;
ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
iii. a first run, wherein the mixed formulation is injected and run through the system of ii.
iv. a second run, wherein a concentrated salt is injected to elute the bound fraction of the CEX column to be separated by the SEC column;
wherein, a specific ionic strength of the solvent is used to allow partitioning of the mixed formulation in the CEX column; and
wherein the method allows analysis of the mixed formulation based on the differential charge and size variant of the constituents.

16. A method of analyzing a mixed formulation sample, the method comprising:
i. providing a mixed formulation that comprises a combination of two protein moieties, wherein the first protein moiety (A) is conjugated to a polymer, and the second protein (B) moiety is not conjugated to a polymer;
ii. loading the sample into an HPLC system where the HPLC pump is first connected to an auto-injector followed by a prefilter, which is then followed down-stream of two tandem serially connected columns, wherein the first column is a cation-exchanger column (CEX) and the second column down-stream of the CEX column is a size exclusion chromatography (SEC) column;
iii. a first run, wherein the mixed formulation is injected and run through the system of ii.
iv. a second run, wherein a concentrated salt is injected to elute the bound fraction of the CEX column to be separated by the SEC column;
wherein, a specific ionic strength of the solvent is used to allow partitioning of the mixed formulation in the CEX column; and
wherein the method allows analysis of the mixed formulation based on the differential charge and size variant of the constituents.

17. A method of analyzing a mixed formulation sample, the method comprising:
i. providing a mixed formulation comprising a combination of two protein moieties, wherein the first protein moiety (A) is conjugated to a polymer, and the second protein moiety (B) is not conjugated to a polymer;
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein the method allows analysis of the mixed formulation based on the differential charge and size variant of the constituents.

18. A method of analyzing a mixed therapeutic composition sample, the method comprising:
i. providing a mixed therapeutic composition comprising a combination of two protein moieties, wherein the first protein moiety (A) is conjugated to a polymer, and the second protein moiety (B) is not conjugated to a polymer;
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein the method allows analysis of the mixed therapeutic composition based on the differential charge and size variant of the constituents.

19. A method of analyzing a mixed therapeutically effective formulation sample, the method comprising:
i. providing a mixed therapeutically effective formulation comprising a combination of two protein moieties, wherein the first protein moiety (A) is conjugated to a polymer, and the second protein moiety (B) is not conjugated to a polymer;
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein the method allows analysis of the mixed therapeutically effective formulation based on the differential charge and size variant of the constituents.

20. An apparatus comprising a tandem HPLC system for use in analyzing a mixed formulation sample comprising a combination of two protein moieties, wherein the first protein moiety (A) is conjugated to a polymer, and the second protein moiety (B) is not conjugated to a polymer, wherein the apparatus comprises:
a) a HPLC pump connected to an auto-injector;
b) a prefilter;
c) cation-exchanger column (CEX);
d) a switch valve enabling the effluent of the CEX to be directed to multiple possible targets,
wherein the apparatus allows analysis of the mixed formulation based on the differential charge and size variant of the constituents.

21. An apparatus comprising a tandem HPLC system for use in analyzing a mixed therapeutic composition sample comprising a combination of two protein moieties, wherein the first protein moiety (A) is conjugated to a polymer, and the second protein moiety (B) is not conjugated to a polymer, wherein the apparatus comprises:
a) a HPLC pump connected to an auto-injector;
b) a prefilter;
c) cation-exchanger column (CEX);
d) a switch valve enabling the effluent of the CEX to be directed to multiple possible targets,
wherein the apparatus allows analysis of the mixed therapeutic composition based on the differential charge and size variant of the constituents.

22. An apparatus comprising a tandem HPLC system for use in analyzing a mixed therapeutically effective formulation sample comprising a combination of two protein moieties, wherein the first protein moiety (A) is conjugated to a polymer, and the second protein moiety (B) is not conjugated to a polymer, wherein the apparatus comprises:
a) a HPLC pump connected to an auto-injector;
b) a prefilter;
c) cation-exchanger column (CEX);
d) a switch valve enabling the effluent of the CEX to be directed to multiple possible targets,
wherein the apparatus allows analysis of the mixed therapeutically effective formulation based on the differential charge and size variant of the constituents.

23. A method of analyzing a mixed formulation sample, the method comprising:
i. providing a mixed formulation comprising a combination of two protein moieties, wherein the first protein moiety (A) is conjugated to a polymer, and the second protein moiety (B) is not conjugated to a polymer; and
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein an additional pump is used to pulse elute the CEX-bound fraction upon wherein the method allows analysis of the mixed formulation based on the differential charge and size variant of the constituents; and
wherein the sample is analyzed via a single continuous chromatography run.

24. A method of analyzing a mixed therapeutic composition sample, the method comprising:
i. providing a mixed therapeutic composition comprising a combination of two protein moieties, wherein the first protein moiety (A) is conjugated to a polymer, and the second protein moiety (B) is not conjugated to a polymer; and
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein an additional pump is used to pulse elute the CEX-bound fraction upon
wherein the method allows analysis of the mixed therapeutic composition based on the differential charge and size variant of the constituents; and
wherein the sample is analyzed via a single continuous chromatography run.

25. A method of analyzing a mixed therapeutically effective formulation sample, the method comprising:
i. providing a mixed therapeutically effective formulation comprising a combination of two protein moieties, wherein the first protein moiety (A) is conjugated to a polymer, and the second protein moiety (B) is not conjugated to a polymer; and
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein an additional pump is used to pulse elute the CEX-bound fraction upon
wherein the method allows analysis of the mixed therapeutically effective formulation based on the differential charge and size variant of the constituents; and
wherein the sample is analyzed via a single continuous chromatography run.

26. An apparatus comprising a tandem HPLC system for use in purifying a mixed formulation sample comprising a combination of two protein moieties, wherein the first protein moiety (A) is conjugated to a polymer, and the second protein moiety (B) is not conjugated to a polymer,
wherein the apparatus comprises:
a) a HPLC pump connected to an auto-injector;
b) a prefilter;
c) cation-exchanger column (CEX);
d) a switch valve enabling the effluent of the CEX to be directed to multiple possible targets,
wherein the apparatus allows analysis of the mixed formulation based on the differential charge and size variant of the constituents;
wherein the sample is analyzed via a single continuous chromatography run.

27. An apparatus comprising a tandem HPLC system for use in purifying a mixed therapeutic composition sample comprising a combination of two protein moieties, wherein the first protein moiety (A) is conjugated to a polymer, and the second protein moiety (B) is not conjugated to a polymer,
wherein the apparatus comprises:
a) a HPLC pump connected to an auto-injector;
b) a prefilter;
c) cation-exchanger column (CEX);
d) a switch valve enabling the effluent of the CEX to be directed to multiple possible targets,
wherein the apparatus allows analysis of the mixed therapeutic composition based on the differential charge and size variant of the constituents;
wherein the sample is analyzed via a single continuous chromatography run.

28. An apparatus comprising a tandem HPLC system for use in purifying a mixed therapeutically effective formulation sample comprising a combination of two protein moieties, wherein the first protein moiety (A) is conjugated to a polymer, and the second protein moiety (B) is not conjugated to a polymer,
wherein the apparatus comprises:
a) a HPLC pump connected to an auto-injector;
b) a prefilter;
c) cation-exchanger column (CEX);
d) a switch valve enabling the effluent of the CEX to be directed to multiple possible targets,
wherein the apparatus allows analysis of the mixed therapeutically effective formulation based on the differential charge and size variant of the constituents;
wherein the sample is analyzed via a single continuous chromatography run.

29. A method of analyzing a mixed formulation sample, the method comprising:
i. providing a mixed formulation comprising a combination of two protein moieties, wherein the first protein moiety (A) is conjugated to a polymer, and the second protein moiety (B) is not conjugated to a polymer; and
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein the switch valve can direct the CEX effluent to:
1) a bypass loop;
2) a first SEC column; or
3) a second SEC column; and
wherein the method allows analysis of the mixed formulation based on the differential charge and size variant of the constituents,
wherein the sample is analyzed via a single continuous chromatography run, and
wherein the conjugated sample is directed to the bypass loop and the unconjugated sample is directed to the first or second SEC column.

30. A method of analyzing a mixed therapeutic composition sample, the method comprising:
i. providing a mixed therapeutic composition comprising a combination of two protein moieties, wherein the first protein moiety (A) is conjugated to a polymer, and the second protein moiety (B) is not conjugated to a polymer; and
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein the switch valve can direct the CEX effluent to:
1) a bypass loop;
2) a first SEC column; or
3) a second SEC column; and
wherein the method allows analysis of the mixed therapeutic composition based on the differential charge and size variant of the constituents,
wherein the sample is analyzed via a single continuous chromatography run, and
wherein the conjugated sample is directed to the bypass loop and the unconjugated sample is directed to the first or second SEC column.

31. A method of analyzing a mixed therapeutically effective formulation sample, the method comprising:
i. providing a mixed therapeutically effective formulation comprising a combination of two protein moieties, wherein the first protein moiety (A) is conjugated to a polymer, and the second protein moiety (B) is not conjugated to a polymer; and
ii. loading the sample into a tandem HPLC system where the HPLC pump is first connected to an auto-injector followed by a prefilter, which is then followed by a cation-exchanger column (CEX) and a switch valve enabling the effluent of the CEX to be directed to multiple possible targets;
wherein the switch valve can direct the CEX effluent to:
1) a bypass loop;
2) a first SEC column; or
3) a second SEC column; and
wherein the method allows analysis of the mixed therapeutically effective formulation based on the differential charge and size variant of the constituents,
wherein the sample is analyzed via a single continuous chromatography run, and
wherein the conjugated sample is directed to the bypass loop and the unconjugated sample is directed to the first or second SEC column.

32. An apparatus comprising a tandem series of HPLC system for use in analyzing a mixed formulation sample comprising (A) a first protein conjugated to a polymer and (B) a second protein that is not conjugated to the polymer, wherein, the HPLC system comprises:
a) a HPLC pump connected to an auto-injector;
b) a prefilter;
c) cation-exchanger column (CEX);
d) a switch valve enabling the effluent of the CEX to be directed to multiple possible targets, wherein the switch valve can direct the CEX effluent to:
1) a bypass loop;
2) a first SEC column; or
3) a second SEC column; and
wherein the apparatus allows analysis of the mixed formulation based on the differential charge and size variant of the constituents,
wherein the sample is analyzed via a single continuous chromatography run; and
wherein the conjugated sample is directed to the bypass loop and the unconjugated sample is directed to the first or second SEC column.

33. An apparatus comprising a tandem series of HPLC system for use in analyzing a mixed therapeutic composition sample comprising (A) a first protein conjugated to a polymer and (B) a second protein that is not conjugated to the polymer, wherein, the HPLC system comprises:
a) a HPLC pump connected to an auto-injector;
b) a prefilter;
c) cation-exchanger column (CEX);
d) a switch valve enabling the effluent of the CEX to be directed to multiple possible targets, wherein the switch valve can direct the CEX effluent to:
1) a bypass loop;
2) a first SEC column; or
3) a second SEC column; and
wherein the apparatus allows analysis of the mixed therapeutic composition based on the differential charge and size variant of the constituents,
wherein the sample is analyzed via a single continuous chromatography run; and
wherein the conjugated sample is directed to the bypass loop and the unconjugated sample is directed to the first or second SEC column.

34. An apparatus comprising a tandem series of HPLC system for use in analyzing a mixed therapeutically effective formulation sample comprising (A) a first protein conjugated to a polymer and (B) a second protein that is not conjugated to the polymer, wherein, the HPLC system comprises:
a) a HPLC pump connected to an auto-injector;
b) a prefilter;
c) cation-exchanger column (CEX);
d) a switch valve enabling the effluent of the CEX to be directed to multiple possible targets, wherein the switch valve can direct the CEX effluent to:
1) a bypass loop;
2) a first SEC column; or
3) a second SEC column; and
wherein the apparatus allows analysis of the mixed therapeutically effective formulation based on the differential charge and size variant of the constituents,
wherein the sample is analyzed via a single continuous chromatography run; and
wherein the conjugated sample is directed to the bypass loop and the unconjugated sample is directed to the first or second SEC column.


SEQUENCE LISTING (161 sequences)

SEQ ID NO: 1 - protein, 453 aa, synthetic construct
  REGION 1..453: note = Anti-VEGF-A heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 2 - protein, 214 aa, synthetic construct
  REGION 1..214: note = Anti-VEGF-A light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 453 aa, synthetic construct
  REGION 1..453: note = Antigen binding protein components
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
  REGION 1..214: note = Antigen binding protein components
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 231 aa, synthetic construct
  REGION 1..231: note = Antigen binding protein components
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 6 - protein, 214 aa, synthetic construct; identical to SEQ ID NO: 2
  REGION 1..214: note = Antigen binding protein components

SEQ ID NO: 7 - DNA, 235 bp, synthetic construct
  misc_feature 1..235: note = Heavy chain
  source 1..235: mol_type = other DNA
atgaaagctgtggtgctggccgtggctctggtcttcctgacagggagccaggctgaggtgcagctggtggaatccggcggaggcctggtccagcctggcggatccctgagactgtcctgtgccgcctccggctacgacttcacccattacggcatgaactgggtccgacaggcccctggcaagggcctggaatgggtcggatggatcaacacctacaccggcgagcccacctacg

SEQ ID NO: 8 - DNA, 702 bp, synthetic construct
  misc_feature 1..702: note = Light chain
  source 1..702: mol_type = other DNA
atgggatggagctgtatcatcctcttcttggtggcaacagctacaggcgtgcactccgacatccagctgacccagtccccctccagcctgtccgcctctgtgggcgacagagtgaccatcacctgttccgccagccaggacatctccaactacctgaactggtatcagcagaagcccggcaaggcccccaaggtgctgatctacttcacctcctccctgcactccggcgtgccctccagattctccggctctggctccggcaccgactttaccctgaccatctccagcctgcagcccgaggacttcgccacctactactgccagcagtactccaccgtgccctggaccttcggccagggcaccaaggtggaaatcaagcggaccgtggccgctccctccgtgttcatcttcccaccctccgacgagcagctgaagtccggaaccgcctccgtcgtgtgcctgctgaacaacttctacccccgcgaggccaaggtgcagtggaaggtggacaacgccctgcagagcggcaactcccaggaatccgtcaccgagcaggactccaaggacagcacctactccctgtccagcaccctgaccctgtccaaggccgactacgagaagcacaaggtgtacgcctgcgaagtgacccaccagggcctcagctccccagtgaccaagtccttcaaccggggcgagtgctag

SEQ ID NO: 9 - protein, 10 aa, synthetic construct
  REGION 1..10: note = Antigen binding protein components
GYDFTHYGMN

SEQ ID NO: 10 - protein, 17 aa, synthetic construct
  REGION 1..17: note = Antigen binding protein components
WINTYTGEPTYAADFKR

SEQ ID NO: 11 - protein, 14 aa, synthetic construct
  REGION 1..14: note = Antigen binding protein components
YPYYYGTSHWYFDV

SEQ ID NO: 12 - protein, 11 aa, synthetic construct
  REGION 1..11: note = Antigen binding protein components
SASQDISNYLN

SEQ ID NO: 13 - protein, 7 aa, synthetic construct
  REGION 1..7: note = Antigen binding protein components
FTSSLHS

SEQ ID NO: 14 - protein, 9 aa, synthetic construct
  REGION 1..9: note = Antigen binding protein components
QQYSTVPWT

SEQ ID NO: 15 - protein, 120 aa, synthetic construct
  source 1..120: note = KCD002 variable heavy chain
EVKLVESEGGLVQPGSSMKLSCTASGFTFSDYYMAWVRQVPEKGLEWVGNINYDGSSTYYLDSLKSRFIISRDSAKNILYLQMSSLKSEDTATYFCARGEDFYLYAMDYWGQGTSVTVSS

SEQ ID NO: 16 - protein, 116 aa, synthetic construct
  source 1..116: note = KCD003 variable heavy chain
EVQLQQSRPELVKPGASVKIFCKASGYTFTDYYMNWMRQRHGETLEWIGDINPNNGDPSYNQKFKDKATLTVDKSSSTASMELRSLTSDDSAVYYCAREGPSFAYWGQGTLVTVSA

SEQ ID NO: 17 - protein, 116 aa, synthetic construct
  source 1..116: note = KCD005 variable heavy chain
EVQLQQSGPELVKPGASVKISCKASGYTFTDHYMNWVKQSHGKSLEWIGDINPNNGGTSCNQKFKGKATLTVDKSSSTAYMELRSLTSGDSAVYYCTREGASFAFWGQGTLVTVSA

SEQ ID NO: 18 - protein, 116 aa, synthetic construct
  source 1..116: note = KCD009 variable heavy chain
QIQLVQSGPELKKPGETVKISCKASGYIFRNYGMNWVKQGPGKGLKWMGWINTYTGEPTYADDFKGRFAFSLETSASTAYLQISNLKNEDTATYFCVRDGPGFAYWGQGTLVTVSA

SEQ ID NO: 19 - protein, 114 aa, synthetic construct
  source 1..114: note = KCD010 variable heavy chain
EVLLQQSGPELVKPGASVKIPCKASGYTFTDYDMDWVKQSHGKSLEWIGHINPNNGGTIYNQKFKGKATLTVDKSSSTAYMELRSLTSEDTAVYYCGTGDFAYWGHGTLVTVSA

SEQ ID NO: 20 - protein, 114 aa, synthetic construct
  source 1..114: note = KCD023 variable heavy chain
EVLLQQSGPELVKPGASVKIPCKASGYTFTDYNIDWVKQSHGKSLEWIGDINPNNGGINYNQKFKGKATLTVDKSSSTAYMELRSLTSEDTAVYYCGTGDYAYWGQGTLVTVSA

SEQ ID NO: 21 - protein, 118 aa, synthetic construct
  source 1..118: note = KCD036 variable heavy chain
EVQLQQSGPELVKPGASMKISCKASGYSFTGYTMTWVKQSHGKNLEWIGLINPYNGGTNYNQKFKGKATFTVDKSSSTAYMELLSLTSEDSAVYYCARRHYGSSWDYWGQGTTLTVSS

SEQ ID NO: 22 - protein, 113 aa, synthetic construct
  source 1..113: note = KCD040 variable heavy chain
EVQLQQSGAELVKPGASVKLSCTASDFNIKDTYMHWVMQRPEQGLEWIGKIDPANGNTEFDPKFQGKATITADTSSNTAYLQLTSLTSEDTAVYYCTRAMDYWGQGTSVTVSS

SEQ ID NO: 23 - protein, 113 aa, synthetic construct
  source 1..113: note = KCD042 variable heavy chain
EVQLQQSGAELVKPGASVRLSCTASGFNIKHTYIHWVSQRPEQGLEWIGKIDPANGNTKYDPKFQGKATITADTSSNTAYLQLSSLTSEDTAVYYCVNAMEYWGQGTSVTVSS

SEQ ID NO: 24 - protein, 113 aa, synthetic construct
  source 1..113: note = KCD044 variable heavy chain
EVQLQQSGAELVKPGASVRLSCTASGFNIKHTYMHWVSQRPERGLEWIGKIDPANGNTKYDPKFQGKATITADTSSNTVYLQLSSLTSEDTAVYYCLNAMEYWGQGTSVTVSS

SEQ ID NO: 25 - protein, 113 aa, synthetic construct
  source 1..113: note = KCD047 variable heavy chain
EVQLQQSGAEFVKPGASVRLSCTASGFNIKDTYMHWVKQRPEQGLEWIGRIDPANGYTKDDPKFQGKATITADTSSNTAYLQLSSLTSEDTAVYYCASAMDYWGQGTSVTVSS

SEQ ID NO: 26 - protein, 113 aa, synthetic construct
  source 1..113: note = KCD048 variable heavy chain
EVQLQQSGADLVKPGASVKLSCTASGFNIKATYMHWVRQRPEKGLEWIGRIDPANGHTIYDPQFQGKATITSDTSSNTAYLQLNSLTSEDTAVYYCAEAMDYWGQGTSVTVSS

SEQ ID NO: 27 - protein, 118 aa, synthetic construct
  source 1..118: note = KCD070 variable heavy chain
EIQLQQTGPELVKPGASVKISCKASGYSFTDYIILWVKQSHGKSLEWIGNINPYYDYTSYNLKFKGKATLTVDKSSSTAYMQLNSLTSEDSAVYYCARSDGYYGGDYWGQGTSVTVSS

SEQ ID NO: 28 - protein, 118 aa, synthetic construct
  source 1..118: note = KCD101 variable heavy chain
QVQLQQPGAELVRPGTSVKLSCKASGYTFTSYWMHWVKQRPGQGLEWIGVIDPSDSYTNYNQKFKGKATLTVDTSSSTAYMQLSSLTSEDSAVYYCGRNGYDGSMDYWGQGTSVTVSS

SEQ ID NO: 29 - protein, 118 aa, synthetic construct
  source 1..118: note = KCD102 variable heavy chain
QVQLQQPGAELVRPGTSVKLSCKASGYTFTSYWMHWVKQRPGQGLEWIGVIDPSDSYTNYNQKFKGKATLTVDTSSSTAYMQLSSLTSEDSAVYYCARNGYDGSMDYWGQGTSVTVSS

SEQ ID NO: 30 - protein, 118 aa, synthetic construct
  source 1..118: note = KCD103 variable heavy chain
QVQLQQPGAELVRPGTSVKLSCKASGYTFTSYWMHWVKQRPGQGLEWIGVIDPSDSYTKYNQKFKDKATLTVETSSSTAYMQLSSLTSEDSAVYYCAGNGYDGSMDYWGQGTSVTVSS

SEQ ID NO: 31 - protein, 118 aa, synthetic construct
  source 1..118: note = KCD104 variable heavy chain
QVQLQQPGAELVRPGTSVKLSCKASGYTFTSYWMHWVKQRPGQGLEWIGVIDPSDSYTYYNQKFKGKATLTVDTSSSTAYMQLSSLTSEDSAVYYCARNGYDGAMDYWGQGTSVTVSS

SEQ ID NO: 32 - protein, 116 aa, synthetic construct
  source 1..116: note = KCD118 variable heavy chain
EVQLQQSGPELVKPGASVKISCKAFGYTFTDYYKNWMRQRHGESLEWIGDINPNSGDANYNQKFKGKATLTVDKSSSTAYMELRSLTSEDSAVYYCAREGPSFAYWGHGTLVTVSA

SEQ ID NO: 33 - protein, 116 aa, synthetic construct
EVQLQQSGPELVKPGASVKISCKASGYTFTDYYTNWMRQRHGESLEWIGDINPNTGDTSYNQKFRVKATLTVDKSSGTAYMGLRSLTSEDSAVYYCTREGPSFAYWGQGTLVTVSA

SEQ ID NO: 34 - protein, 116 aa, synthetic construct
  source 1..116: note = KCD121 variable heavy chain
EVQLQQSGPELVKPGASVKISCKASGYTFTDYYKNWMRQRHGESLEWIGDINPNNGDTSYNQKFRGKATLTVDKSSSTAFMELRSLTSEDSAVYYCAREGPSFAYWGQGTLVTVSA

SEQ ID NO: 35 - protein, 116 aa, synthetic construct
  source 1..116: note = KCD122 variable heavy chain
EVQLQQSGPELVKPGASVKISCKASGYTFTDYYKNWMRQRHGESLEWIGDINPNNGDANYNQKFKGKATLTVDKSSSTAYMELRSLTSEDSAVYFCAREGPSFAYWGHGTLVTVSA

SEQ ID NO: 36 - protein, 116 aa, synthetic construct
  source 1..116: note = KCD123 variable heavy chain
EVHLQQSGPELVKPGASVKISCKASGYTFTDFYKNWMRQRHGESLEWIGDINPNNGGTNYNQKFKGKATLTVDKSSSTAYMELRSLTSEDSAVYYCAREGPSFAYWGQGTLVTVSA

SEQ ID NO: 37 - protein, 116 aa, synthetic construct
  source 1..116: note = KCD124 variable heavy chain
EVQLQQSGPELVKPGASVKISCKASGYTFTDHYMNWVKQSHGKSLEWIGDINPNNGGTSYNQKFKGKATLTVDKSSSTAYMELRSLTSGDSAVYYCTREGASFAFWGQGTLVTVSA

SEQ ID NO: 38 - protein, 122 aa, synthetic construct
  source 1..122: note = KCD131 variable heavy chain
QVQLQQSGPELVKPGASVKISCKASAYTFTDYYINWVKQRPGQGPEWIGWIFPGSNSTYSNEKFEVKATLTVDESSSTAYMLLSSLTSEDSAVYFCARLGYFGSSYHALDYWGQGTSVTVSS

SEQ ID NO: 39 - protein, 113 aa, synthetic construct
  source 1..113: note = KCD136 variable heavy chain
EVQLQQSVAELVRPGASVKLSCSASGFNIKNTYMHWVNQRPEQGLEWIGRIDPANGITKYAPNFQGKATITADTSSNTAYLQLSNLTSEDTAIYYCTRAMDYWGQGTSVTVSS

SEQ ID NO: 40 - protein, 116 aa, synthetic construct
  source 1..116: note = KCD200 variable heavy chain
EVQLQQSGPELVKPGASVKISCKASGYTFTSYYKNWMRQRHGESLEWIGDINPNSGDTAYNQKFKGKATLTVDRSSSTAYMELRSLTSEDSAVYYCAREGPSFAYWGQGTLVTVSA

SEQ ID NO: 41 - protein, 115 aa, synthetic construct
  source 1..115: note = KCD208 variable heavy chain
EVQLQQSVAELVRPGASVKLSCTVSGFNIKNTYMHWVKQRPEQGLEWIGRIDPANGDTTYAPKFQGKATITADTSSNSAYLHLSRLTSEDTAIYYCSLYDYDGYWGQGTTLTVSS

SEQ ID NO: 42 - protein, 115 aa, synthetic construct
  source 1..115: note = KCD214 variable heavy chain
EVQLQQSVAEFVRPGASVKLSCTASGFNIKNTYMHWVKQRPEQGLEWIGRIDPANGNTEYAPKFQGKATITADTSSNTAYLQLSSLTSEDTAIYYCALYDYDGYWGQGTTLTVSS

SEQ ID NO: 43 - protein, 119 aa, synthetic construct
  source 1..119: note = KCD220 variable heavy chain
QVQLQQSGAELMEPGASVKLSCKATGYTFTGYWIEWVKQRPGHGLEWIGETLPGSDSNNYNEKFKGKATFTADTSSNTAYMQLSSLTTEDSAIYYCARDYSNYWYFDVWGTGTTVTVSS

SEQ ID NO: 44 - protein, 119 aa, synthetic construct
  source 1..119: note = KCD224 variable heavy chain
QVQLKESGPGLVAPSQSLSITCTVSGFSLTSYGVDWIRQSPGKGLEWLGVIWGVGSTNYNSALKSRLSISKDNSKSQVFLKMNSLQTDDTAMYYCARSYDGSYWYFDVWGTGTTVTVSS

SEQ ID NO: 45 - protein, 116 aa, synthetic construct
  source 1..116: note = 119_TAF variable heavy chain
EVQLVESGGGLVQPGGSLRLSCAASGYTFTDYYMNWVRQAPGKGLEWIGDINPNTGDTSYNADFKRRFTFSLDTSKSTAYLQMNSLRAEDTAVYYCTREGPSFAYWGQGTLVTVSS

SEQ ID NO: 46 - protein, 117 aa, synthetic construct
  source 1..117: note = 119_TAF Germ variable heavy chain
EVQLVESGGGLVQPGGSLRLSCAASGYTFTDYYMSWVRQAPGKGLEWIGDINPNTGDTSYNADSVKGRFTISRDNSKNTLYLQMNSLRAEDTAVYYCTREGPSFAYWGQGTLVTVSS

SEQ ID NO: 47 - protein, 107 aa, synthetic construct
  source 1..107: note = 119_Human Germ variable heavy chain
EVKKPGASVKVSCKASGYTFTDYYMHWVRQAPGQGLEWIGDINPNTGDTSYNQKFQGRVTSTRDTSISTAYMELSRLRSDDTVVYYCTREGPSFAYWGQGTLVTVSS

SEQ ID NO: 48 - protein, 107 aa, synthetic construct
  source 1..107: note = KCD002 variable light chain
DIQMTQSPASLSASVGETVTITCRASENIHSYLAWYQQKQGKSPQLIVYNTKTLAEGVPSRFSGSGSGTQFSLKINSLQPEDFGSYYCQHHYGIPPTFGGGTKLEIK

SEQ ID NO: 49 - protein, 112 aa, synthetic construct
  source 1..112: note = KCD003 variable light chain
DVLMTQTPLSLPVSLGEQASISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 50 - protein, 112 aa, synthetic construct
  source 1..112: note = KCD005 variable light chain
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPVTFGAGTKLELK

SEQ ID NO: 51 - protein, 112 aa, synthetic construct
  source 1..112: note = KCD009 variable light chain
DVLMTQTPLSLPVSLGDQASISCRSSLIIEHSDGNTYLEWYLQKPGQSPKLLIYKVSNRFSGVPDRFSGSGSGTDFTLKISRVEADDLGVYYCFQGSHVPVTFGAGTNLELK

SEQ ID NO: 52 - protein, 108 aa, synthetic construct
  source 1..108: note = KCD010 variable light chain
QIVLTQSPAIMSVSPGEKVTLTCSASSSVSSSYLYWYQKKPGSSPKLWIYSTSNLASGVPARFSGSGSGTSYSLTISSMEAEDAASYFCHQWSSYPPTFGAGTKLELK

SEQ ID NO: 53 - protein, 108 aa, synthetic construct
  source 1..108: note = KCD023 variable light chain
QIVLTQSPAFMSASPGEKVTLTCSASSSVSSSYLYWYQQKPGSSPKLWIYSTSNLASGVPGRFSGSGSGTSYSLTISSMEAEDAASYFCHQWTSYPPTFGAGTKLELK

SEQ ID NO: 54 - protein, 107 aa, synthetic construct
  source 1..107: note = KCD036 variable light chain
DIKLTQSPSSMYASLGERVTITCKASQDINTYLSWFQQKPGKSPKTLIYRANRLVDGVPSRFSGSGSGQDYSLTISSLEYEEMGIYYCLQYDEFPYTFGGGTKLEIK

SEQ ID NO: 55 - protein, 106 aa, synthetic construct
  source 1..106: note = KCD040 variable light chain
QIVLTQSPAIMSASPGEKVTMTCSAGSSVSYMYWYQQKPGSSPRVLIYDTSNLASGVPVRFSGSGSGTSYSLTISRMEAEDAATYYCQQWSNYPYTFGGGTKLEIK

SEQ ID NO: 56 - protein, 106 aa, synthetic construct
  source 1..106: note = KCD042 variable light chain
QSVLTQSPAIMSASPGEKVTMTCSANSSVSDMYWFQQRPGSSPRLLIYDTSNLASGVPVRFSGSGSGTSYSLTISRMEAEDAATYYCQQWSTYPWTFGGGTKLEIK

SEQ ID NO: 57 - protein, 106 aa, synthetic construct
  source 1..106: note = KCD044 variable light chain
QSVLTQSPAIMSASPGEKVTMTCSANSSVSDMYWYQQRPGSSPRLLIYDTSNLASGVPVRFSGSGSGTSYSLTISRMEAEDAATYYCQQWSTYPWTFGGGTKLEIK

SEQ ID NO: 58 - protein, 106 aa, synthetic construct
  source 1..106: note = KCD047 variable light chain
QIVLTQSPAVMSASPGEKVAMTCSASSSVTYMYWYQQKPGSSPRLLIYDTSNLASGVPVRFSGSGSGTSYSLTISRMEAEDAATYYCQQWSTYPFPFGSGTKLEIK

SEQ ID NO: 59 - protein, 106 aa, synthetic construct
  source 1..106: note = KCD048 variable light chain
QIVLTQSPAIMSASPGEKVTLTCSATSSVSYMYWYQQKPGSSPRLLIYDTSNLASGVPVRFSGSGSGTSYSLTISRMEAEDDATYYCQQWSNYPFTFGGGTKLEIK

SEQ ID NO: 60 - protein, 107 aa, synthetic construct
  source 1..107: note = KCD070 variable light chain
DIVMTQSHKFMSTSVGDRVSITCKASQDVGTAVAWYQQKPGQSPKLLIYWASTRHTGVPDRFTGSGSGTDFTLTINNVQSEDLADYFCQQYSSYPWTFGGGTTLEIK

SEQ ID NO: 61 - protein, 107 aa, synthetic construct
  source 1..107: note = KCD101 variable light chain
DIQMTQTTSSLSASLGDRVTISCRASQDISNYLNWYQQKPDGTVKLLIYYPSRLHSGVPSRFSGSGSGTDYSLTISNLEQEDFATYFCQQGNTLPYTFGGGTKLEIK

SEQ ID NO: 62 - protein, 107 aa, synthetic construct
  source 1..107: note = KCD102 variable light chain
EIQMTQTTSSLSASLGDRVTISCRASQDISNYLNWYQQKPDGTVKLLIYYPSRLHSGVPSRFSGSGSGTDYSLTISNLEQEDFATYFCQQGNTLPYTFGGGTKLEIK

SEQ ID NO: 63 - protein, 107 aa, synthetic construct
  source 1..107: note = KCD103 variable light chain
DIQMTQTTSSLSASLGDRVTISCRASQDISNSLNWYQQKPDGTVKLLIYYTSRLHSRVPSRFSGSGSGTDYSLTISNLDQEDIATYFCQQANTLPYTFGGGTKLEIK

SEQ ID NO: 64 - protein, 107 aa, synthetic construct; identical to SEQ ID NO: 62
  source 1..107: note = KCD104 variable light chain

SEQ ID NO: 65 - protein, 112 aa, synthetic construct
  source 1..112: note = KCD118 variable light chain
DVLMTQTPLSLPVSLGDQASISCRSSQTIVHSNGDTYLEWYLQKPGQSPNLLIYKVSNRFSGVPDRFSGSGSGTDFTLKISRVEAEDLGIYYCFQGSHVPPTFGGGTKLEIK

SEQ ID NO: 66 - protein, 112 aa, synthetic construct
  source 1..112: note = KCD119 variable light chain
DVLMTQTPLSLPVSLGDQASISCRSSQTIVHSNGDTYLEWYLQKPGQSPNLLIYKVSNRFSGVPDRFSGSGSGTDFTLKISRVEAEDLGVYYCFQGSHVPPTFGGGTTLEIK

SEQ ID NO: 67 - protein, 112 aa, synthetic construct
  source 1..112: note = KCD121 variable light chain
DVLMTQTPLSLPVSLGDQASISCRSNQTIVHSNGDTYLEWYLQKPGQSPNLLIYKVSNRFSGVPDRFSGSGSGTDFTLRISRVEAEDLGVYYCFQGSHVPPTFGGGTKLEIK

SEQ ID NO: 68 - protein, 112 aa, synthetic construct
  source 1..112: note = KCD122 variable light chain
DVLMTQTPLSLPVSLGDQASISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 69 - protein, 112 aa, synthetic construct; identical to SEQ ID NO: 68
  source 1..112: note = KCD123 variable light chain

SEQ ID NO: 70 - protein, 112 aa, synthetic construct
  source 1..112: note = KCD124 variable light chain
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPLTFGAGTKLELK

SEQ ID NO: 71 - protein, 107 aa, synthetic construct
  source 1..107: note = KCD131 variable light chain
DIQMTQSPASLSVSVGETVTITCRASENIYSHLAWFQQKQGKSPRLLVYSATNLPDGVPSRFSGSGSGTQYSLKINILQSEDFGSYYCQHFWGTPWTFGGGTKLEIK

SEQ ID NO: 72 - protein, 106 aa, synthetic construct
  source 1..106: note = KCD136 variable light chain
QIVLTQSPAIMSASPGEKVTMTCSASSSVSYMYWYQQKPGSSPRLLIYDISNLASGVPVRFSGSGSGTSYSLTISRMEAEDAATYYCQQWDTYPWTFGGGTKLEIK

SEQ ID NO: 73 - protein, 112 aa, synthetic construct
  source 1..112: note = KCD200 variable light chain
DVLMTQTPLSLPVSLGDQVSISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 74 - protein, 108 aa, synthetic construct
  source 1..108: note = KCD208 variable light chain
EIVLTQSPALMAASPGEKVTITCSVSSSISSSSLHWYRQKSGTSPKPWIYGTSHLASGVPVRFSGSGSGTSYSLTISSMEAEDAATYYCQQWDTYPWTFGGGTKLEIK

SEQ ID NO: 75 - protein, 108 aa, synthetic construct
  source 1..108: note = KCD214 variable light chain
EIVLTQSPALMAASPGEKVTITCRVSSSISSSSLHWYQQKSGTSPKPWIYGTSNLASGVPVRFSGSRSGTSYSLTISSMEAEDAATYYCQQWSDYPWTFGGGTKLEIK

SEQ ID NO: 76 - protein, 106 aa, synthetic construct
  source 1..106: note = KCD220 variable light chain
DIQMTQTTSSLSASLGDRVTISCRASQDISNYLNWYQQKPDGTVKLLIYYTSNLHSGVPSRFSGSGSGTDYSLTISNLEQEDIATYFCQQDSKHRTFGGGTKLEIK

SEQ ID NO: 77 - protein, 107 aa, synthetic construct
  source 1..107: note = KCD224 variable light chain
DIQMTQTTSSLSASLGDRVTISCRASQVISNYLNWYQQKPDGTVKLLIYYTSRLHSGVPSRFSGSGSGTDYSLTISNLEPEDIATYYCQQYSKLPYTFGSGTKLEIK

SEQ ID NO: 78 - protein, 112 aa, synthetic construct
  source 1..112: note = 119_TAF variable light chain
DIQLTQSPSSLSASVGDRVTITCRSSQTIVHSNGDTYLEWYQQKPGKAPNLLIYKVSNRFSGVPSRFSGSGSGTDFTLTISSLQPEDFATYYCFQGSHVPPTFGQGTKVEIK

SEQ ID NO: 79 - protein, 112 aa, synthetic construct
  source 1..112: note = 119_TAF Germ variable light chain
DIQMTQSPSSLSASVGDRVTITCRSSQTIVHSNGDTYLEWYQQKPGKAPNLLIYKVSNRFSGVPSRFSGSGSGTDFTLTISSLQPEDFATYYCFQGSHVPPTFGQGTKVEIK

SEQ ID NO: 80 - protein, 112 aa, synthetic construct
  source 1..112: note = 119_Human Germ variable light chain
DVVMTQSPLSLPVTLGQPASISCRSSQTIVHSNGDTYLEWFQQRPGQSPNLLIYKVSNRFSGVPDRFSGSGSGTDFTLKISRVEAEDVGVYYCFQGSHVPPTFGGGTKVEIK

SEQ ID NO: 81 - protein, 118 aa, synthetic construct
  source 1..118: note = I IL-6 variable heavy chain
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVS

SEQ ID NO: 82 - protein, 119 aa, synthetic construct
  source 1..119: note = II IL-6 variable heavy chain
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSS

SEQ ID NO: 83 - protein, 119 aa, synthetic construct
  source 1..119: note = IIa IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSS

SEQ ID NO: 84 - protein, 119 aa, synthetic construct
  source 1..119: note = IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQSWGYYALDIWGQGTLVTVSS

SEQ ID NO: 85 - protein, 119 aa, synthetic construct
  source 1..119: note = IIc IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQGWGYYALDIWGQGTLVTVSS

SEQ ID NO: 86 - protein, 119 aa, synthetic construct
  source 1..119: note = IId IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQTWGYYALDIWGQGTLVTVSS

SEQ ID NO: 87 - protein, 119 aa, synthetic construct
  source 1..119: note = IIe Il-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQVWGYYALDIWGQGTLVTVSS

SEQ ID NO: 88 - protein, 119 aa, synthetic construct
  source 1..119: note = IIf IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQQWGYYALDIWGQGTLVTVSS

SEQ ID NO: 89 - protein, 119 aa, synthetic construct
  source 1..119: note = IIg IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQKWGYYALDIWGQGTLVTVSS

SEQ ID NO: 90 - protein, 106 aa, synthetic construct
  source 1..106: note = III IL-6 variable light chain
DIQLTQSPSSLSASVGDRVTITCSASISVSYLYWYQQKPGKAPKLLIYDDSSLASGVPSRFSGSGSGTDFTLTISSLQPEDFATYYCQQWSGYPYTFGQGTKVEIK

SEQ ID NO: 91 - protein, 106 aa, synthetic construct
  source 1..106: note = IV IL-6 variable light chain
DIQLTQSPSSLSASVGDRVTITCSASISVSYLYWYQQKPGKAPKLLIYDASSLASGVPSRFSGSGSGTDFTLTISSLQPEDFATYYCQQWSGYPYTFGQGTKVEIK

SEQ ID NO: 92 - protein, 106 aa, synthetic construct
  source 1..106: note = V IL-6 variable light chain
DIQLTQSPSSLSASVGDRVTITCSASISVSYLYWYQQKPGKAPKLLIYDDSNLASGVPSRFSGSGSGTDFTLTISSLQPEDFATYYCQQWSGYPYTFGQGTKVEIK

SEQ ID NO: 93 - protein, 664 aa, synthetic construct
  source 1..664: note = A heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 94 - protein, 213 aa, synthetic construct
  source 1..213: note = A light chain
DIQLTQSPSSLSASVGDRVTITCSASISVSYLYWYQQKPGKAPKLLIYDDSSLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 664 aa, synthetic construct; identical to SEQ ID NO: 93
  source 1..664: note = B heavy chain

SEQ ID NO: 96 - protein, 213 aa, synthetic construct
  source 1..213: note = B light chain
DIQLTQSPSSLSASVGDRVTITCSASISVSYLYWYQQKPGKAPKLLIYDASSLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 664 aa, synthetic construct; identical to SEQ ID NO: 93
  source 1..664: note = C heavy chain

SEQ ID NO: 98 - protein, 213 aa, synthetic construct
  source 1..213: note = C light chain
DIQLTQSPSSLSASVGDRVTITCSASISVSYLYWYQQKPGKAPKLLIYDDSNLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 664 aa, synthetic construct
  source 1..664: note = D heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 100 - protein, 213 aa, synthetic construct; identical to SEQ ID NO: 94
  source 1..213: note = D light chain

SEQ ID NO: 101 - protein, 664 aa, synthetic construct; identical to SEQ ID NO: 99
  source 1..664: note = E heavy chain

SEQ ID NO: 102 - protein, 213 aa, synthetic construct; identical to SEQ ID NO: 96
  source 1..213: note = E light chain

SEQ ID NO: 103 - protein, 664 aa, synthetic construct; identical to SEQ ID NO: 99
  source 1..664: note = F heavy chain

SEQ ID NO: 104 - protein, 213 aa, synthetic construct; identical to SEQ ID NO: 98
  source 1..213: note = F light chain

SEQ ID NO: 105 - protein, 664 aa, synthetic construct
  source 1..664: note = G heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 106 - protein, 213 aa, synthetic construct; identical to SEQ ID NO: 94
  source 1..213: note = G light chain

SEQ ID NO: 107 - protein, 664 aa, synthetic construct; identical to SEQ ID NO: 105
  source 1..664: note = H heavy chain

SEQ ID NO: 108 - protein, 213 aa, synthetic construct; identical to SEQ ID NO: 96
  source 1..213: note = H light chain

SEQ ID NO: 109 - protein, 664 aa, synthetic construct; identical to SEQ ID NO: 105
  source 1..664: note = I heavy chain

SEQ ID NO: 110 - protein, 213 aa, synthetic construct; identical to SEQ ID NO: 98
  source 1..213: note = I light chain

SEQ ID NO: 111 - protein, 664 aa, synthetic construct
  source 1..664: note = J heavy chain
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 112 - protein, 213 aa, synthetic construct; identical to SEQ ID NO: 94
  source 1..213: note = J light chain

SEQ ID NO: 113 - protein, 664 aa, synthetic construct; identical to SEQ ID NO: 111
  source 1..664: note = K heavy chain

SEQ ID NO: 114 - protein, 213 aa, synthetic construct; identical to SEQ ID NO: 96
  source 1..213: note = K light chain

SEQ ID NO: 115 - protein, 664 aa, synthetic construct; identical to SEQ ID NO: 111
  source 1..664: note = L heavy chain

SEQ ID NO: 116 - protein, 213 aa, synthetic construct; identical to SEQ ID NO: 98
  source 1..213: note = L light chain

SEQ ID NO: 117 - protein, 664 aa, synthetic construct
  source 1..664: note = M heavy chain
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 118 - protein, 213 aa, synthetic construct; identical to SEQ ID NO: 94
  source 1..213: note = M light chain

SEQ ID NO: 119 - protein, 664 aa, synthetic construct; identical to SEQ ID NO: 117
  source 1..664: note = N heavy chain

SEQ ID NO: 120 - protein, 213 aa, synthetic construct; identical to SEQ ID NO: 96
  source 1..213: note = N light chain

SEQ ID NO: 121 - protein, 664 aa, synthetic construct; identical to SEQ ID NO: 117
  source 1..664: note = O heavy chain

SEQ ID NO: 122 - protein, 213 aa, synthetic construct; identical to SEQ ID NO: 98
  source 1..213: note = O light chain

SEQ ID NO: 123 - protein, 664 aa, synthetic construct
  source 1..664: note = P heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 124 - protein, 213 aa, synthetic construct; identical to SEQ ID NO: 94
  source 1..213: note = P light chain

SEQ ID NO: 125 - protein, 664 aa, synthetic construct; identical to SEQ ID NO: 123
  source 1..664: note = Q heavy chain

SEQ ID NO: 126 - protein, 213 aa, synthetic construct; identical to SEQ ID NO: 96
  source 1..213: note = Q light chain

SEQ ID NO: 127 - protein, 664 aa, synthetic construct; identical to SEQ ID NO: 123
  source 1..664: note = R heavy chain

SEQ ID NO: 128 - protein, 213 aa, synthetic construct; identical to SEQ ID NO: 98
  source 1..213: note = R light chain

SEQ ID NO: 129 - protein, 446 aa, synthetic construct
  source 1..446: note = Exemplary human IgG1 heavy chain
EVQLVESGGGLVQPGGSLRLSCAASGYTFTDYYINWVRQAPGKGLEWIGDINPITGDTDYNADFKRRFTFSLDTSKSTAYLQMSSLRAEDTAVYYCTREGPSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 130 - protein, 219 aa, synthetic construct
  source 1..219: note = Exemplary human kappa light chain
DIQLTQSPSSLSASVGDRVTITCRSSQTIVHSNGDTYLEWYQQKPGKAPNLLIRKVSNRFSGVPSRFSGSGSGTDFTLTISSLQPEDFATYYCFQGSHVPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 745 aa, synthetic construct
  source 1..745: note = Synthetic polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 132 - protein, 214 aa, synthetic construct; identical to SEQ ID NO: 2
  source 1..214: note = Synthetic polypeptide

SEQ ID NO: 133 - protein, 205 aa, Synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 134 - protein, 5 aa, synthetic construct
PFAMH

SEQ ID NO: 135 - protein, 17 aa, synthetic construct
KISPGGSWTYYSDTVTD

SEQ ID NO: 136 - protein, 10 aa, synthetic construct
QAWGYYALDI

SEQ ID NO: 137 - protein, 10 aa, synthetic construct
SASISVSYLY

SEQ ID NO: 138 - protein, 7 aa, synthetic construct
DDSSLAS

SEQ ID NO: 139 - protein, 9 aa, synthetic construct
QQWSGYPYT

SEQ ID NO: 140 - protein, 5 aa, synthetic construct
FYHVH

SEQ ID NO: 141 - protein, 16 aa, synthetic construct
SIYTSGYTEYASALES

SEQ ID NO: 142 - protein, 11 aa, synthetic construct
EGLQRVGVLDA

SEQ ID NO: 143 - protein, 11 aa, synthetic construct
EGLQRVGVFDA

SEQ ID NO: 144 - protein, 11 aa, synthetic construct
EGLQRVGVMDA

SEQ ID NO: 145 - protein, 16 aa, synthetic construct
RSSQSLLDEAGETYLA

SEQ ID NO: 146 - protein, 7 aa, synthetic construct
EVSLLES

SEQ ID NO: 147 - protein, 9 aa, synthetic construct
QQATYFPYT

SEQ ID NO: 148 - protein, 8 aa, synthetic construct
GFSLTFYH

SEQ ID NO: 149 - protein, 7 aa, synthetic construct
IYTSGYT

SEQ ID NO: 150 - protein, 13 aa, synthetic construct
AREGLQRVGVFDA

SEQ ID NO: 151 - protein, 13 aa, synthetic construct
AREGLQRVGVMDA

SEQ ID NO: 152 - protein, 13 aa, synthetic construct
AREGLQRVGVLDA

SEQ ID NO: 153 - protein, 11 aa, synthetic construct
QSLLDEAGETY

SEQ ID NO: 154 - protein, 7 aa, synthetic construct
GFSLTFY

SEQ ID NO: 155 - protein, 5 aa, synthetic construct
YTSGY

SEQ ID NO: 156 - protein, 119 aa, synthetic construct
QVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVFDAWGQGTLVTVSS

SEQ ID NO: 157 - protein, 119 aa, synthetic construct
QVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVMDAWGQGTLVTVSS

SEQ ID NO: 158 - protein, 119 aa, synthetic construct
QVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVLDAWGQGTLVTVSS

SEQ ID NO: 159 - protein, 119 aa, synthetic construct
EVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVMDAWGQGTLVTVSS

SEQ ID NO: 160 - protein, 119 aa, synthetic construct
EVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVLDAWGQGTLVTVSS

SEQ ID NO: 161 - protein, 112 aa, synthetic construct
DIQLTQSPSSLSASVGDRVTITCRSSQSLLDEAGETYLAWFQQKPGKAPKLLIYEVSLLESGVPSRFSGSGSGTDFTLTISSLQPEDFATYYCQQATYFPYTFGQGTKVEIK